SEQUENCE LISTING (188 sequences)

SEQ ID NO: 1 - protein, 109 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLA

SEQ ID NO: 2 - protein, 76 aa, Homo sapiens
TEQCDCGSLCQHHACCDENCILKAKAECSDGPCCHKCKFHRKGYPCCPSSRSCDLPEFCNGTSALCPNNRHKQDGS

SEQ ID NO: 3 - protein, 81 aa, Homo sapiens
GEECDCGTEQDCALIGETCCDIATCRFKAGSNCAEGPCCENCLFMSKERMCRPSFEECDLPEYCNGSSASCPENHYVQTGH

SEQ ID NO: 4 - protein, 81 aa, Homo sapiens
TEQCDCGYKEACTHKKCCNPADCTLVRSAECGTGSCCNNKTCTIHERGHVCRKSVDMCDFPEYCNGTSEFCVPDVKAADLE

SEQ ID NO: 5 - protein, 78 aa, Homo sapiens
REECDCGSFKQCYASYCCQSDCHLTPGSICHIGECCTNCSFSPPGTLCRPIQNICDLPEYCHGTTVTCPANVYMQDGT

SEQ ID NO: 6 - protein, 78 aa, Homo sapiens
GEECDCGPAQECTNPCCDAHTCVLKPGFTCAEGECCESCQIKKAGSICRPAKDECDFPEMCTGHSPACPKDQFRVNGF

SEQ ID NO: 7 - protein, 78 aa, Homo sapiens
GEQCDCGPPEDCRNRCCNSTTCQLAEGAQCAHGTCCQECKVKPAGELCRPKKDMCDLEEFCDGRHPECPEDAFQENGT

SEQ ID NO: 8 - protein, 79 aa, Homo sapiens
GEECDCGTPKECELDPCCEGSTCKLKSFAECAYGDCCKDCRFLPGGTLCRGKTSECDVPEYCNGSSQFCQPDVFIQNGY

SEQ ID NO: 9 - protein, 86 aa, Homo sapiens
GEECDCGYSDQCKDECCFDANQPEGRKCKLKPGKQCSPSQGPCCTAQCAFKSKSEKCRDDSDCAREGICNGFTALCPASDPKPNFT

SEQ ID NO: 10 - protein, 79 aa, Homo sapiens
GEECDCGSVQECSRAGGNCCKKCTLTHDAMCSDGLCCRRCKYEPRGVSCREAVNECDIAETCTGDSSQCPPNLHKLDGY

SEQ ID NO: 11 - protein, 78 aa, Homo sapiens
GEECDCGEPEECMNRCCNATTCTLKPDAVCAHGLCCEDCQLKPAGTACRDSSNSCDLPEFCTGASPHCPANVYLHDGH

SEQ ID NO: 12 - protein, 79 aa, Homo sapiens
GEQCDCGFLDDCVDPCCDSLTCQLRPGAQCASDGPCCQNCQLRPSGWQCRPTRGDCDLPEFCPGDSSQCPPDVSLGDGE

SEQ ID NO: 13 - protein, 80 aa, Homo sapiens
GEECDPGIMYLNNDTCCNSDCTLKEGVQCSDRNSPCCKNCQFETAQKKCQEAINATCKGVSYCTGNSSECPPPGNAEDDT

SEQ ID NO: 14 - protein, 81 aa, Homo sapiens
NEECDCGNKNECQFKKCCDYNTCKLKGSVKCGSGPCCTSKCELSIAGTPCRKSIDPECDFTEYCNGTSSNCVPDTYALNGR

SEQ ID NO: 15 - protein, 78 aa, Homo sapiens
GEECDCGEEEECNNPCCNASNCTLRPGAECAHGSCCHQCKLLAPGTLCREQARQCDLPEFCTGKSPHCPTNFYQMDGT

SEQ ID NO: 16 - protein, 78 aa, Homo sapiens
GEECDCGTIRQCAKDPCCLLNCTLHPGAACAFGICCKDCKFLPSGTLCRQQVGECDLPEWCNGTSHQCPDDVYVQDGI

SEQ ID NO: 17 - protein, 78 aa, Homo sapiens
EEQCDCGSVQQCEQDACCLLNCTLRPGAACAFGLCCKDCKFMPSGELCRQEVNECDLPEWCNGTSHQCPEDRYVQDGI

SEQ ID NO: 18 - protein, 79 aa, Homo sapiens
GEECDCGTPAECVLEGAECCKKCTLTQDSQCSDGLCCKKCKFQPMGTVCREAVNDCDIRETCSGNSSQCAPNIHKMDGY

SEQ ID NO: 19 - protein, 78 aa, Homo sapiens
GEECDCGFHVECYGLCCKKCSLSNGAHCSDGPCCNNTSCLFQPRGYECRDAVNECDITEYCTGDSGQCPPNLHKQDGY

SEQ ID NO: 20 - protein, 78 aa, Homo sapiens
GEDCDCGTSEECTNICCDAKTCKIKATFQCALGECCEKCQFKKAGMVCRPAKDECDLPEMCNGKSGNCPDDRFQVNGF

SEQ ID NO: 21 - protein, 78 aa, Homo sapiens
GEECDCGPLKHCAKDPCCLSNCTLTDGSTCAFGLCCKDCKFLPSGKVCRKEVNECDLPEWCNGTSHKCPDDFYVEDGI

SEQ ID NO: 22 - protein, 78 aa, Homo sapiens
NEECDCGSTEECQKDRCCQSNCKLQPGANCSIGLCCHDCRFRPSGYVCRQEGNECDLAEYCDGNSSSCPNDVYKQDGT

SEQ ID NO: 23 - protein, 80 aa, Homo sapiens
NEICDCGTEAQCGPASCCDFRTCVLKDGAKCYKGLCCKDCQILQSGVECRPKAHPECDIAENCNGSSPECGPDITLINGL

SEQ ID NO: 24 - protein, 78 aa, Homo sapiens
GEECDCGPGQECRDLCCFAHNCSLRPGAQCAHGDCCVRCLLKPAGALCRQAMGDCDLPEFCTGTSSHCPPDVYLLDGS

SEQ ID NO: 25 - protein, 70 aa, Trimeresurus flavoviridis
GEECDCGSPSNPCCDAATCKLRPGAQCADGLCCDQCRFKKKRTICRIARGDFPDDRCTGQSADCPRWNDL

SEQ ID NO: 26 - protein, 72 aa, Artificial Sequence
  Description of Artificial Sequence Synthetic polypeptide
GEQCDCGSLHACCDENCILKAKAECSDGPCCHKCKFHRKGYPCRPSSRSDLPEFCNGTSALCPNNRHKQDGS

SEQ ID NO: 27 - protein, 77 aa, Artificial Sequence
  Description of Artificial Sequence Synthetic polypeptide
GEECDCGTEQDIGETCCDIATCRFKAGSNCAEGPCCENCLFMSKERMCRPSFEEDLPEYCNGSSASCPENHYVQTGH

SEQ ID NO: 28 - protein, 77 aa, Artificial Sequence
  Description of Artificial Sequence Synthetic polypeptide
GEQCDCGYKEAKKCCNPADCTLVRSAECGTGSCCNNKTCTIHERGHVCRKSVDMDFPEYCNGTSEFCVPDVKAADLE

SEQ ID NO: 29 - protein, 74 aa, Artificial Sequence
  Description of Artificial Sequence Synthetic polypeptide
GEECDCGSFKQSYCCQSDCHLTPGSICHIGECCTNCSFSPPGTLCRPIQNIDLPEYCHGTTVTCPANVYMQDGT

SEQ ID NO: 30 - protein, 74 aa, Artificial Sequence
  Description of Artificial Sequence Synthetic polypeptide
GEECDCGPAQEPCCDAHTCVLKPGFTCAEGECCESCQIKKAGSICRPAKDEDFPEMCTGHSPACPKDQFRVNGF

SEQ ID NO: 31 - protein, 74 aa, Artificial Sequence
  Description of Artificial Sequence Synthetic polypeptide
GEQCDCGPPEDRCCNSTTCQLAEGAQCAHGTCCQECKVKPAGELCRPKKDMDLEEFCDGRHPECPEDAFQENGT

SEQ ID NO: 32 - protein, 75 aa, Artificial Sequence
  Description of Artificial Sequence Synthetic polypeptide
GEECDCGTPKEDPCCEGSTCKLKSFAECAYGDCCKDCRFLPGGTLCRGKTSEDVPEYCNGSSQFCQPDVFIQNGY

SEQ ID NO: 33 - protein, 82 aa, Artificial Sequence
  Description of Artificial Sequence Synthetic polypeptide
GEECDCGYSDQECCFDANQPEGRKCKLKPGKQCSPSQGPCCTAQCAFKSKSEKCRDDSDAREGICNGFTALCPASDPKPNFT

SEQ ID NO: 34 - protein, 75 aa, Artificial Sequence
  Description of Artificial Sequence Synthetic polypeptide
GEECDCGSVQEAGGNCCKKCTLTHDAMCSDGLCCRRCKYEPRGVSCREAVNEDIAETCTGDSSQCPPNLHKLDGY

SEQ ID NO: 35 - protein, 74 aa, Artificial Sequence
  Description of Artificial Sequence Synthetic polypeptide
GEECDCGEPEERCCNATTCTLKPDAVCAHGLCCEDCQLKPAGTACRDSSNSDLPEFCTGASPHCPANVYLHDGH

SEQ ID NO: 36 - protein, 75 aa, Artificial Sequence
  Description of Artificial Sequence Synthetic polypeptide
GEQCDCGFLDDPCCDSLTCQLRPGAQCASDGPCCQNCQLRPSGWQCRPTRGDDLPEFCPGDSSQCPPDVSLGDGE

SEQ ID NO: 37 - protein, 77 aa, Artificial Sequence
  Description of Artificial Sequence Synthetic polypeptide
GEECDCGIMYLDTCCNSDCTLKEGVQCSDRNSPCCKNCQFETAQKKCQEAINATKGVSYCTGNSSECPPPGNAEDDT

SEQ ID NO: 38 - protein, 77 aa, Artificial Sequence
  Description of Artificial Sequence Synthetic polypeptide
GEECDCGNKNEKKCCDYNTCKLKGSVKCGSGPCCTSKCELSIAGTPCRKSIDPEDFTEYCNGTSSNCVPDTYALNGR

SEQ ID NO: 39 - protein, 74 aa, Artificial Sequence
  Description of Artificial Sequence Synthetic polypeptide
GEECDCGEEEEPCCNASNCTLRPGAECAHGSCCHQCKLLAPGTLCREQARQDLPEFCTGKSPHCPTNFYQMDGT

SEQ ID NO: 40 - protein, 74 aa, Artificial Sequence
  Description of Artificial Sequence Synthetic polypeptide
GEECDCGTIRQDPCCLLNCTLHPGAACAFGICCKDCKFLPSGTLCRQQVGEDLPEWCNGTSHQCPDDVYVQDGI

SEQ ID NO: 41 - protein, 74 aa, Artificial Sequence
  Description of Artificial Sequence Synthetic polypeptide
GEQCDCGSVQQDACCLLNCTLRPGAACAFGLCCKDCKFMPSGELCRQEVNEDLPEWCNGTSHQCPEDRYVQDGI

SEQ ID NO: 42 - protein, 75 aa, Artificial Sequence
  Description of Artificial Sequence Synthetic polypeptide
GEECDCGTPAEEGAECCKKCTLTQDSQCSDGLCCKKCKFQPMGTVCREAVNDDIRETCSGNSSQCAPNIHKMDGY

SEQ ID NO: 43 - protein, 74 aa, Artificial Sequence
  Description of Artificial Sequence Synthetic polypeptide
GEECDCGFHVELCCKKCSLSNGAHCSDGPCCNNTSCLFQPRGYECRDAVNEDITEYCTGDSGQCPPNLHKQDGY

SEQ ID NO: 44 - protein, 74 aa, Artificial Sequence
  Description of Artificial Sequence Synthetic polypeptide
GEDCDCGTSEEICCDAKTCKIKATFQCALGECCEKCQFKKAGMVCRPAKDEDLPEMCNGKSGNCPDDRFQVNGF

SEQ ID NO: 45 - protein, 74 aa, Artificial Sequence
  Description of Artificial Sequence Synthetic polypeptide
GEECDCGPLKHDPCCLSNCTLTDGSTCAFGLCCKDCKFLPSGKVCRKEVNEDLPEWCNGTSHKCPDDFYVEDGI

SEQ ID NO: 46 - protein, 74 aa, Artificial Sequence
  Description of Artificial Sequence Synthetic polypeptide
GEECDCGSTEEDRCCQSNCKLQPGANCSIGLCCHDCRFRPSGYVCRQEGNEDLAEYCDGNSSSCPNDVYKQDGT

SEQ ID NO: 47 - protein, 76 aa, Artificial Sequence
  Description of Artificial Sequence Synthetic polypeptide
GEICDCGTEAQASCCDFRTCVLKDGAKCYKGLCCKDCQILQSGVECRPKAHPEDIAENCNGSSPECGPDITLINGL

SEQ ID NO: 48 - protein, 74 aa, Artificial Sequence
  Description of Artificial Sequence Synthetic polypeptide
GEECDCGPGQELCCFAHNCSLRPGAQCAHGDCCVRCLLKPAGALCRQAMGDDLPEFCTGTSSHCPPDVYLLDGS

SEQ ID NO: 49 - protein, 90 aa, Unknown
  Description of Unknown Snake venom VAP1 polypeptide
SPAVCGNYFVEVGEECDCGSPRTCRDPCCDATTCKLRQGAQCAEGLCCDQCRFKGAGTECRAAKDECDMADVCTGRSAECTDRFQRNGQP

SEQ ID NO: 50 - protein, 90 aa, Unknown
  Description of Unknown Snake venom Catrocollastatin polypeptide
SPPVCGNELLEVGEECDCGTPENCQNECCDAATCKLKSGSQCGHGDCCEQCKFSKSGTECRASMSECDPAEHCTGQSSECPADVFHKNGQ

SEQ ID NO: 51 - protein, 83 aa, Unknown
  Description of Unknown Snake venom Salmosin3 polypeptide
SPPVCGNYYPEVGEDCDCGPPANCQNPCCDAATCGLTTGSQCAEGLCCDQCRLKKAGTICRKARGDNPDDRCTGQSGVCPRNT

SEQ ID NO: 52 - protein, 83 aa, Unknown
  Description of Unknown Snake venom Bitistatin polypeptide
SPPVCGNKILEEGEECDCGSPANCQDRCCNAATCKLTPGSQCNHGECCDQCKFKKAGTVCRIARGDWNDDYCTGKSSDCPWNH

SEQ ID NO: 53 - protein, 70 aa, Trimeresurus flavoviridis; identical to SEQ ID NO: 25

SEQ ID NO: 54 - DNA, 213 bp, Artificial Sequence
  Description of Artificial Sequence Synthetic polynucleotide
gagcagtgtgactgtggttctctacatgcatgctgtgatgaaaactgtatactgaaggcgaaagcagagtgcagtgatggtccatgttgtcataagtgtaaatttcaccgtaagggatatccttgcaggccttctagtcgttccgatctcccagaattttgcaatggtacatctgcattatgccccaacaacaggcataagcaagatggctca

SEQ ID NO: 55 - DNA, 231 bp, Artificial Sequence
  Description of Artificial Sequence Synthetic polynucleotide
ggagaggagtgtgactgtgggactgaacaggatattggagaaacatgctgtgatattgccacatgtagatttaaagccggttcaaactgtgctgaaggaccatgctgcgaaaactgtctatttatgtcaaaagaaagaatgtgtaggccttcctttgaagaagacctccctgaatattgcaatggatcatctgcatcatgcccagaaaaccactatgttcagactgggcat

SEQ ID NO: 56 - DNA, 228 bp, Artificial Sequence
  Description of Artificial Sequence Synthetic polynucleotide
gagcagtgtgattgtggctataaagaggcaaaaaaatgctgtaatcctgcagattgtactctagttagatctgcagaatgtggcactggatcatgctgtaacaataaaacttgtacgatccacgaaagaggccatgtctgcagaaaaagtgtagatatggattttccagaatattgcaatggaacatctgagttttgtgtacctgatgtgaaagctgctgatttagaa

SEQ ID NO: 57 - DNA, 219 bp, Artificial Sequence
  Description of Artificial Sequence Synthetic polynucleotide
gaggaatgtgactgtggctccttcaagcagagttattgctgccaaagtgactgtcacttaacaccggggagcatctgccatataggagagtgctgtacaaactgcagcttctccccaccagggactctctgcagacctatccaaaatatagaccttccagagtactgtcacgggaccaccgtgacatgtcccgcaaacgtttatatgcaagatggaacc

SEQ ID NO: 58 - DNA, 222 bp, Artificial Sequence
  Description of Artificial Sequence Synthetic polynucleotide
ggtgaagagtgtgactgtggccctgctcaggagccttgctgtgatgcacacacatgtgtactgaagccaggatttacttgtgcagaaggagaatgctgtgaatcttgtcagataaaaaaagcagggtccatatgcagaccggcgaaagatgaagattttcctgagatgtgcactggccactcgcctgcctgtcctaaggaccagttcagggtcaatggattt

SEQ ID NO: 59 - DNA, 222 bp, Artificial Sequence
  Description of Artificial Sequence Synthetic polynucleotide
ggggagcagtgcgactgcggcccccccgaggaccgctgctgcaactctaccacctgccagctggctgagggggcccagtgtgcgcacggtacctgctgccaggagtgcaaggtgaagccggctggtgagctgtgccgtcccaagaaggacatggacctcgaggagttctgtgacggccggcaccctgagtgcccggaagacgccttccaggagaacggcacg

SEQ ID NO: 60 - DNA, 225 bp, Artificial Sequence
  Description of Artificial Sequence Synthetic polynucleotide
ggggaagagtgtgactgtggtactccaaaggaagacccttgctgcgaaggaagtacctgtaagcttaaatcatttgctgagtgtgcatatggtgactgttgtaaagactgtcggttccttccaggaggtactttatgccgaggaaaaaccagtgaggatgttccagagtactgcaatggttcttctcagttctgtcagccagatgtttttattcagaatggatat

SEQ ID NO: 61 - DNA, 246 bp, Artificial Sequence
  Description of Artificial Sequence Synthetic polynucleotide
ggtgaagaatgtgattgtggctatagtgaccaggaatgctgcttcgatgcaaatcaaccagagggaagaaaatgcaaactgaaacctgggaaacagtgcagtccaagtcaaggtccttgttgtacagcacagtgtgcattcaagtcaaagtctgagaagtgtcgggatgattcagacgcaagggaaggaatatgtaatggcttcacagctctctgcccagcatctgaccctaaaccaaacttcaca

SEQ ID NO: 62 - DNA, 225 bp, Artificial Sequence
  Description of Artificial Sequence Synthetic polynucleotide
ggggaggagtgcgactgcggctcggtgcaggaggcaggtggcaactgctgcaagaaatgcaccctgactcacgacgccatgtgcagcgacgggctctgctgtcgccgctgcaagtacgaaccacggggtgtgtcctgccgagaggccgtgaacgaggacatcgcggagacctgcaccggggactctagccagtgcccgcctaacctgcacaagctggacggttac

SEQ ID NO: 63 - DNA, 222 bp, Artificial Sequence
  Description of Artificial Sequence Synthetic polynucleotide
ggagaggagtgtgactgtggggagccagaggaacgctgctgcaatgccaccacctgtaccctgaagccggacgctgtgtgcgcacatgggctgtgctgtgaagactgccagctgaagcctgcaggaacagcgtgcagggactccagcaactccgacctcccagagttctgcacaggggccagccctcactgcccagccaacgtgtacctgcacgatgggcac

SEQ ID NO: 64 - DNA, 225 bp, Artificial Sequence
  Description of Artificial Sequence Synthetic polynucleotide
ggcgagcagtgtgactgtggcttcctggatgacccctgctgtgattctttgacctgccagctgaggccaggtgcacagtgtgcatctgacggaccctgttgtcaaaattgccagctgcgcccgtctggctggcagtgtcgtcctaccagaggggatgacttgcctgaattctgcccaggagacagctcccagtgtccccctgatgtcagcctaggggatggcgag

SEQ ID NO: 65 - DNA, 231 bp, Artificial Sequence
  Description of Artificial Sequence Synthetic polynucleotide
ggagaagagtgtgattgtggcatcatgtatctggacacctgctgcaacagcgactgcacgttgaaggaaggtgtccagtgcagtgacaggaacagtccttgctgtaaaaactgtcagtttgagactgcccagaagaagtgccaggaggcgattaatgctactaaaggcgtgtcctactgcacaggtaatagcagtgagtgcccgcctccaggaaatgctgaagatgacact

SEQ ID NO: 66 - DNA, 228 bp, Artificial Sequence
  Description of Artificial Sequence Synthetic polynucleotide
gaagaatgtgactgtggtaataaaaatgaaaagaagtgctgtgattataacacatgtaaactgaagggctcagtaaaatgtggttctggaccatgttgtacatcaaagtgtgagttgtcaatagcaggcactccatgtagaaagagtattgatccagaggattttacagagtactgcaatggaacctctagtaattgtgttcctgacacttatgcattgaatggccgt

SEQ ID NO: 67 - DNA, 222 bp, Artificial Sequence
  Description of Artificial Sequence Synthetic polynucleotide
ggggaagagtgtgactgtggagaagaagaggaaccctgctgcaatgcctctaattgtaccctgaggccgggggcggagtgtgctcacggctcctgctgccaccagtgtaagctgttggctcctgggaccctgtgccgcgagcaggccaggcaggacctcccggagttctgtacgggcaagtctccccactgccctaccaacttctaccagatggatggtacc

SEQ ID NO: 68 - DNA, 222 bp, Artificial Sequence
  Description of Artificial Sequence Synthetic polynucleotide
ggggaggaatgtgactgtggaaccatacggcaggatccctgttgtctgttaaactgtactctacatcctggggctgcttgtgcttttggaatatgttgcaaagactgcaaatttctgccatcaggaactttatgtagacaacaagttggtgaagaccttccagagtggtgcaatgggacatcccatcaatgcccagatgatgtgtatgtgcaggacgggatc

SEQ ID NO: 69 - DNA, 219 bp, Artificial Sequence
  Description of Artificial Sequence Synthetic polynucleotide
gagcagtgtgactgtggatccgtacagcaggacgcctgttgtctgttgaactgcactctaaggcctggggctgcctgtgcttttgggctttgttgcaaagactgcaagttcatgccatcaggggaactctgtagacaagaggtcaatgaagaccttccagaatggtgcaatggaacatctcatcagtgtccagaagatagatatgtgcaggacgggatc

SEQ ID NO: 70 - DNA, 225 bp, Artificial Sequence
  Description of Artificial Sequence Synthetic polynucleotide
ggagaggagtgtgattgtggaaccccggccgaagaaggagcagagtgttgtaagaaatgcaccttgactcaagactctcaatgcagtgacggtctttgctgtaaaaagtgcaagtttcagcctatgggcactgtgtgccgagaagcagtaaatgatgatattcgtgaaacgtgctcaggaaattcaagccagtgtgcccctaatattcataaaatggatggatat

SEQ ID NO: 71 - DNA, 222 bp, Artificial Sequence
  Description of Artificial Sequence Synthetic polynucleotide
ggggaggagtgtgattgtggttttcatgtggaattatgctgtaagaaatgttccctctccaacggggctcactgcagcgacgggccctgctgtaacaatacctcatgtctttttcagccacgagggtatgaatgccgggatgctgtgaacgaggatattactgaatattgtactggagactctggtcagtgcccaccaaatcttcataagcaagacggatat

SEQ ID NO: 72 - DNA, 222 bp, Artificial Sequence
  Description of Artificial Sequence Synthetic polynucleotide
ggagaggactgtgattgtgggacatctgaggaaatttgctgtgatgctaagacatgtaaaatcaaagcaacttttcaatgtgcattaggagaatgttgtgaaaaatgccaatttaaaaaggctgggatggtgtgcagaccagcaaaagatgaggacctgcctgaaatgtgtaatggtaaatctggtaattgtcctgatgatagattccaagtcaatggcttc

SEQ ID NO: 73 - DNA, 222 bp, Artificial Sequence
  Description of Artificial Sequence Synthetic polynucleotide
ggagaagagtgtgactgtggacctttaaagcatgatccctgctgtctgtcaaattgcactctgactgatggttctacttgtgcttttgggctttgttgcaaagactgcaagttcctaccatcagggaaagtgtgtagaaaggaggtcaatgaagatcttccagagtggtgcaatggtacttcccataagtgcccagatgacttttatgtggaagatggaatt

SEQ ID NO: 74 - DNA, 219 bp, Artificial Sequence
  Description of Artificial Sequence Synthetic polynucleotide
gaggaatgtgactgtggttccacagaggaggatcggtgttgccaatcaaattgtaagttgcaaccaggtgccaactgtagcattggactttgctgtcatgattgtcggtttcgtccatctggatacgtgtgtaggcaggaaggaaatgaagaccttgcagagtactgcgacgggaattcaagttcctgcccaaatgacgtttataagcaggatggaacc

SEQ ID NO: 75 - DNA, 225 bp, Artificial Sequence
  Description of Artificial Sequence Synthetic polynucleotide
gaaatctgtgattgtggtactgaggctcaagcaagctgttgtgattttcgaacttgtgtactgaaagacggagcaaaatgttataaaggactgtgctgcaaagactgtcaaattttacaatcaggcgttgaatgtaggccgaaagcacatcctgaagacatcgctgaaaattgtaatggaagctcaccagaatgtggtcctgacataactttaatcaatggactt

SEQ ID NO: 76 - DNA, 222 bp, Artificial Sequence
  Description of Artificial Sequence Synthetic polynucleotide
ggcgaggagtgtgactgcggccctggccaggagctctgctgctttgctcacaactgctcgctgcgcccgggggcccagtgcgcccacggggactgctgcgtgcgctgcctgctgaagccggctggagcgctgtgccgccaggccatgggtgacgacctccctgagttttgcacgggcacctcctcccactgtcccccagacgtttacctactggacggctca

SEQ ID NO: 77 - DNA, 54 bp, Artificial Sequence
  Description of Artificial Sequence Synthetic primer
gttccagatctcgaaaacctgtattttcagggcgagcagtgtgactgtggttct

SEQ ID NO: 78 - DNA, 45 bp, Artificial Sequence
  Description of Artificial Sequence Synthetic primer
gttattcgccatggcttatgagccatcttgcttatgcctgttgtt

SEQ ID NO: 79 - DNA, 57 bp, Artificial Sequence
  Description of Artificial Sequence Synthetic primer
gttccagatctcgaaaacctgtattttcagggagaggagtgtgactgtgggactgaa

SEQ ID NO: 80 - DNA, 45 bp, Artificial Sequence
  Description of Artificial Sequence Synthetic primer
gttattcgccatggcttaatgcccagtctgaacatagtggttttc

SEQ ID NO: 81 - DNA, 56 bp, Artificial Sequence
  Description of Artificial Sequence Synthetic primer
gttccgccatggctgaaaacctgtattttcagggtgagcagtgtgattgtggctat

SEQ ID NO: 82 - DNA, 44 bp, Artificial Sequence
  Description of Artificial Sequence Synthetic primer
gttattcggaattcttattctaaatcagcagctttcacatcagg

SEQ ID NO: 83 - DNA, 54 bp, Artificial Sequence
  Description of Artificial Sequence Synthetic primer
gttccagatctcgaaaacctgtattttcagggcgaggaatgtgactgtggctcc

SEQ ID NO: 84 - DNA, 45 bp, Artificial Sequence
  Description of Artificial Sequence Synthetic primer
gttattcgccatggcttaggttccatcttgcatataaacgtttgc

SEQ ID NO: 85 - DNA, 57 bp, Artificial Sequence
  Description of Artificial Sequence Synthetic primer
gttccagatctcgaaaacctgtattttcagggtgaagagtgtgactgtggccctgct

SEQ ID NO: 86 - DNA, 45 bp, Artificial Sequence
  Description of Artificial Sequence Synthetic primer
gttattcgccatggcttaaaatccattgaccctgaactggtcctt

SEQ ID NO: 87 - DNA, 57 bp, Artificial Sequence
  Description of Artificial Sequence Synthetic primer
gttccagatctcgaaaacctgtattttcagggggagcagtgcgactgcggccccccc

SEQ ID NO: 88 - DNA, 45 bp, Artificial Sequence
  Description of Artificial Sequence Synthetic primer
gttattcgccatggcttacgtgccgttctcctggaaggcgtcttc

SEQ ID NO: 89 - DNA, 57 bp, Artificial Sequence
  Description of Artificial Sequence Synthetic primer
gttccagatctcgaaaacctgtattttcagggggaagagtgtgactgtggtactcca

SEQ ID NO: 90 - DNA, 45 bp, Artificial Sequence
  Description of Artificial Sequence Synthetic primer
gttattcgccatggcttaatatccattctgaataaaaacatctgg

SEQ ID NO: 91 - DNA, 57 bp, Artificial Sequence
  Description of Artificial Sequence Synthetic primer
gttccagatctcgaaaacctgtattttcagggtgaagaatgtgattgtggctatagt

SEQ ID NO: 92 - DNA, 45 bp, Artificial Sequence
  Description of Artificial Sequence Synthetic primer
gttattcgccatggcttatgtgaagtttggtttagggtcagatgc

SEQ ID NO: 93 - DNA, 57 bp, Artificial Sequence
  Description of Artificial Sequence Synthetic primer
gttccagatctcgaaaacctgtattttcagggggaggagtgcgactgcggctcggtg

SEQ ID NO: 94 - DNA, 45 bp, Artificial Sequence
  Description of Artificial Sequence Synthetic primer
gttattcgccatggcttagtaaccgtccagcttgtgcaggttagg

SEQ ID NO: 95 - DNA, 57 bp, Artificial Sequence
  Description of Artificial Sequence Synthetic primer
gttccagatctcgaaaacctgtattttcagggagaggagtgtgactgtggggagcca

SEQ ID NO: 96 - DNA, 45 bp, Artificial Sequence
  Description of Artificial Sequence Synthetic primer
gttattcgccatggcttagtgcccatcgtgcaggtacacgttggc

SEQ ID NO: 97 - DNA, 57 bp, Artificial Sequence
  Description of Artificial Sequence Synthetic primer
gttccagatctcgaaaacctgtattttcagggcgagcagtgtgactgtggcttcctg

SEQ ID NO: 98 - DNA, 45 bp, Artificial Sequence
  Description of Artificial Sequence Synthetic primer
gttattcgccatggcttactcgccatcccctaggctgacatcagg

SEQ ID NO: 99 - DNA, 57 bp, Artificial Sequence
  Description of Artificial Sequence Synthetic primer
gttccagatctcgaaaacctgtattttcagggagaagagtgtgattgtggcatcatg

SEQ ID NO: 100 - DNA, 45 bp, Artificial Sequence
  Description of Artificial Sequence Synthetic primer
gttattcgccatggcttaagtgtcatcttcagcatttcctggagg

SEQ ID NO: 101 - DNA, 54 bp, Artificial Sequence
  Description of Artificial Sequence Synthetic primer
gttccagatctcgaaaacctgtattttcagggcgaagaatgtgactgtggtaat

SEQ ID NO: 102 - DNA, 45 bp, Artificial Sequence
  Description of Artificial Sequence Synthetic primer
gttattcgccatggcttaacggccattcaatgcataagtgtcagg

SEQ ID NO: 103 - DNA, 57 bp, Artificial Sequence
  Description of Artificial Sequence Synthetic primer
gttccagatctcgaaaacctgtattttcagggggaagagtgtgactgtggagaagaa

SEQ ID NO: 104 - DNA, 45 bp, Artificial Sequence
  Description of Artificial Sequence Synthetic primer
gttattcgccatggcttaggtaccatccatctggtagaagttggt

SEQ ID NO: 105 - DNA, 57 bp, Artificial Sequence
  Description of Artificial Sequence Synthetic primer
gttccagatctcgaaaacctgtattttcagggggaggaatgtgactgtggaaccata

SEQ ID NO: 106 - DNA, 45 bp, Artificial Sequence
  Description of Artificial Sequence Synthetic primer
gttattcgccatggcttagatcccgtcctgcacatacacatcatc

SEQ ID NO: 107 - DNA, 54 bp, Artificial Sequence
  Description of Artificial Sequence Synthetic primer
gttccagatctcgaaaacctgtattttcagggcgagcagtgtgactgtggatcc

SEQ ID NO: 108 - DNA, 45 bp, Artificial Sequence
  Description of Artificial Sequence Synthetic primer
gttattcgccatggcttagatcccgtcctgcacatatctatcttc

SEQ ID NO: 109 - DNA, 57 bp, Artificial Sequence
  Description of Artificial Sequence Synthetic primer
gttccagatctcgaaaacctgtattttcagggagaggagtgtgattgtggaaccccg

SEQ ID NO: 110 - DNA, 45 bp, Artificial Sequence
  Description of Artificial Sequence Synthetic primer
gttattcgccatggcttaatatccatccattttatgaatattagg

SEQ ID NO: 111 - DNA, 57 bp, Artificial Sequence
  Description of Artificial Sequence Synthetic primer
gttccagatctcgaaaacctgtattttcagggggaggagtgtgattgtggttttcat

SEQ ID NO: 112 - DNA, 45 bp, Artificial Sequence
  Description of Artificial Sequence Synthetic primer
gttattcgccatggcttaatatccgtcttgcttatgaagatttgg

SEQ ID NO: 113 - DNA, 57 bp, Artificial Sequence
  Description of Artificial Sequence Synthetic primer
gttccagatctcgaaaacctgtattttcagggagaggactgtgattgtgggacatct

SEQ ID NO: 114 - DNA, 45 bp, Artificial Sequence
  Description of Artificial Sequence Synthetic primer
gttattcgccatggcttagaagccattgacttggaatctatcatc

SEQ ID NO: 115 - DNA, 59 bp, Artificial Sequence
  Description of Artificial Sequence Synthetic primer
gttccgccatggctgaaaacctgtattttcagggagaagagtgtgactgtggaccttta

SEQ ID NO: 116 - DNA, 44 bp, Artificial Sequence
  Description of Artificial Sequence Synthetic primer
gttattcggaattcttaaattccatcttccacataaaagtcatc

SEQ ID NO: 117 - DNA, 54 bp, Artificial Sequence
  Description of Artificial Sequence Synthetic primer
gttccagatctcgaaaacctgtattttcagggcgaggaatgtgactgtggttcc

SEQ ID NO: 118 - DNA, 45 bp, Artificial Sequence
  Description of Artificial Sequence Synthetic primer
gttattcgccatggcttaggttccatcctgcttataaacgtcatt

SEQ ID NO: 119 - DNA, 54 bp, Artificial Sequence
  Description of Artificial Sequence Synthetic primer
gttccagatctcgaaaacctgtattttcagggcgaaatctgtgattgtggtact

SEQ ID NO: 120 - DNA, 45 bp, Artificial Sequence
  Description of Artificial Sequence Synthetic primer
gttattcgccatggcttaaagtccattgattaaagttatgtcagg

SEQ ID NO: 121 - DNA, 57 bp, Artificial Sequence
  Description of Artificial Sequence Synthetic primer
gttccagatctcgaaaacctgtattttcagggcgaggagtgtgactgcggccctggc

SEQ ID NO: 122 - DNA, 45 bp, Artificial Sequence
  Description of Artificial Sequence Synthetic primer
gttattcggaattccttatgagccgtccagtaggtaaacgtctgg

SEQ ID NO: 123 - protein, 229 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEQCDCGSLHACCDENCILKAKAECSDGPCCHKCKFHRKGYPCRPSSRSDLPEFCNGTSALCPNNRHKQDGS

SEQ ID NO: 124 - protein, 234 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEECDCGTEQDIGETCCDIATCRFKAGSNCAEGPCCENCLFMSKERMCRPSFEEDLPEYCNGSSASCPENHYVQTGH

SEQ ID NO: 125 - protein, 244 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGTDDDDKAMAENLYFQGEQCDCGYKEAKKCCNPADCTLVRSAECGTGSCCNNKTCTIHERGHVCRKSVDMDFPEYCNGTSEFCVPDVKAADLE

SEQ ID NO: 126 - protein, 231 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEECDCGSFKQSYCCQSDCHLTPGSICHIGECCTNCSFSPPGTLCRPIQNIDLPEYCHGTTVTCPANVYMQDGT

SEQ ID NO: 127 - protein, 231 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEECDCGPAQEPCCDAHTCVLKPGFTCAEGECCESCQIKKAGSICRPAKDEDFPEMCTGHSPACPKDQFRVNGF

SEQ ID NO: 128 - protein, 231 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEQCDCGPPEDRCCNSTTCQLAEGAQCAHGTCCQECKVKPAGELCRPKKDMDLEEFCDGRHPECPEDAFQENGT

SEQ ID NO: 129 - protein, 232 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEECDCGTPKEDPCCEGSTCKLKSFAECAYGDCCKDCRFLPGGTLCRGKTSEDVPEYCNGSSQFCQPDVFIQNGY

SEQ ID NO: 130 - protein, 239 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEECDCGYSDQECCFDANQPEGRKCKLKPGKQCSPSQGPCCTAQCAFKSKSEKCRDDSDAREGICNGFTALCPASDPKPNFT

SEQ ID NO: 131 - protein, 232 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEECDCGSVQEAGGNCCKKCTLTHDAMCSDGLCCRRCKYEPRGVSCREAVNEDIAETCTGDSSQCPPNLHKLDGY

SEQ ID NO: 132 - protein, 231 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEECDCGEPEERCCNATTCTLKPDAVCAHGLCCEDCQLKPAGTACRDSSNSDLPEFCTGASPHCPANVYLHDGH

SEQ ID NO: 133 - protein, 232 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEQCDCGFLDDPCCDSLTCQLRPGAQCASDGPCCQNCQLRPSGWQCRPTRGDDLPEFCPGDSSQCPPDVSLGDGE

SEQ ID NO: 134 - protein, 234 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEECDCGIMYLDTCCNSDCTLKEGVQCSDRNSPCCKNCQFETAQKKCQEAINATKGVSYCTGNSSECPPPGNAEDDT

SEQ ID NO: 135 - protein, 234 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEECDCGNKNEKKCCDYNTCKLKGSVKCGSGPCCTSKCELSIAGTPCRKSIDPEDFTEYCNGTSSNCVPDTYALNGR

SEQ ID NO: 136 - protein, 231 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEECDCGEEEEPCCNASNCTLRPGAECAHGSCCHQCKLLAPGTLCREQARQDLPEFCTGKSPHCPTNFYQMDGT

SEQ ID NO: 137 - protein, 231 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEECDCGTIRQDPCCLLNCTLHPGAACAFGICCKDCKFLPSGTLCRQQVGEDLPEWCNGTSHQCPDDVYVQDGI

SEQ ID NO: 138 - protein, 231 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEQCDCGSVQQDACCLLNCTLRPGAACAFGLCCKDCKFMPSGELCRQEVNEDLPEWCNGTSHQCPEDRYVQDGI

SEQ ID NO: 139 - protein, 232 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEECDCGTPAEEGAECCKKCTLTQDSQCSDGLCCKKCKFQPMGTVCREAVNDDIRETCSGNSSQCAPNIHKMDGY

SEQ ID NO: 140 - protein, 231 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEECDCGFHVELCCKKCSLSNGAHCSDGPCCNNTSCLFQPRGYECRDAVNEDITEYCTGDSGQCPPNLHKQDGY

SEQ ID NO: 141 - protein, 231 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEDCDCGTSEEICCDAKTCKIKATFQCALGECCEKCQFKKAGMVCRPAKDEDLPEMCNGKSGNCPDDRFQVNGF

SEQ ID NO: 142 - protein, 241 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGTDDDDKAMAENLYFQGEECDCGPLKHDPCCLSNCTLTDGSTCAFGLCCKDCKFLPSGKVCRKEVNEDLPEWCNGTSHKCPDDFYVEDGI

SEQ ID NO: 143 - protein, 231 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEECDCGSTEEDRCCQSNCKLQPGANCSIGLCCHDCRFRPSGYVCRQEGNEDLAEYCDGNSSSCPNDVYKQDGT

SEQ ID NO: 144 - protein, 233 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEICDCGTEAQASCCDFRTCVLKDGAKCYKGLCCKDCQILQSGVECRPKAHPEDIAENCNGSSPECGPDITLINGL

SEQ ID NO: 145 - protein, 231 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEECDCGPGQELCCFAHNCSLRPGAQCAHGDCCVRCLLKPAGALCRQAMGDDLPEFCTGTSSHCPPDVYLLDGS

SEQ ID NO: 146 - DNA, 2840 bp, Homo sapiens
aaactgccgaaggccaggagagaagcaggaagagaaacacggcttgaagttctgtaagaatcagagttgggcaagaactgccaataagggagccagcgtagatctgagtactaaacacctgagcctccgagggtttgtgtgcgagggagagctctgctgatggacataggcccagaatcattaactgatttatttgggccatgtcactggtggcattattaaaagactctgccaacatcctgctttatggaaaaaccaagtggccttggaagaggctaagataaagtttcaaacttgggctccacagaagtggaacttaaggctggggctagtaccaggaccttcatgtatcaggttagagattttgatgcttttggtgatttttgtgccaagcatgtactgtcacctgggatcaatctattactctttctatgaaataattattccaaagaggctgacagtccagggaggagatagcccagtggaaggactgtcctacttgttacttatgcaaggccagaagcacctggttcatctgaaggtgaagagaaaccattttgtgaataactttccagtctacagttaccacaatggcctcctggggcaagaatcgcctttcatctcacatgactgccactatgaaggctacatagaaggagtgtcaggttcttttgtttctgtcaacatctgtgcaggtctcaggggcacatcctcctgattaaggaggaaaaatcttacagcattgagcccatggactcttcaagacggtttgaacatgtgttatacaccatggcacatcaagcgtgagtctcctgtggtgtctactagctggcaacaagggagcaggaagcctcatgatctacaggcactgtcctacttgtggtcacacaaaaagtacgtggagatgtttgtcgtggtcaacaaccagcggttccagatgtggggcagtaacgtcaatgagacggtccagacagtagtggatgtcattgctctggccaacagcttcactaggggaataaacacagaggtggtgctggctggaatggagatttggaccgagggggacctaatagatgtcacagtggacttgcaaatcacactcaggaatttcaatcactggagacaagagatgctcttccatcgtgcaaaacacgatgttgcccacatgatcgttgggcatcaccctggacagaatatgggccaggcctttctcagtggtgcctgctcaagcggttttgcggcagctgttgaatccttccatcatgaagatgtgctgttgtttgcagccctgatggcccatgagctcgggcacaacctgggtattcagcacgaccactcggcctgcttttgtaaagataagcacttttgcctcatgcatgaaaatatcacaaaagaaagtggcttcagcagctgcagctctgactacttctaccagttccttcgagaacacaaaggggcctgcctatttaacaagccacggcccaggggccgcaagcgtagggattctgcctgtggaaatggtgtggtggaggacacggagcagtgtgactgtggttctctatgtcagcatcatgcatgctgtgatgaaaactgtatactgaaggcgaaagcagagtgcagtgatggtccatgttgtcataagtgtaaatttcaccgtaagggatatccttgctgtccttctagtcgttcctgtgatctcccagaattttgcaatggtacatctgcattatgccccaacaacaggcataagcaagatggctcaaaatgtcatacaatttacgagtgccttaaagttcattgtatggaccctaataatcagtgcttacaattatatggatatggtgcaaaatcagcctcacaagagtgttacaattcaatgaacagcaaaggggaccaatttggaaactgtggcatttctaccagtcctgggtcacaatatgttcggtgttcagatggtaatatattttgtgggaaacttatatgttcaggtattacaggcttaccaaaaatcaatctccaacatacaatgattcaggtccctcagggagatggctcatgttggagcatggatgcctatatgagtactgacattcctgatgaaggagatgtgcacaatggcacttactgtgcaccaaacaaagtctgcctgaattccgcctgcacagataaaaccccagtgatttctgcctgcaacccagaaaaaacgtgtaatgggaagggagtttgtaatgatttagggcactgccactgtaatgaagggcatgccccccctgactgtgttactgcaggaagtggaggtagtgtggacagtggccctcctggtaagctaggtgggacaccttcaggagaaggtgaaaatcacaatatgactcattccagacgtgaagaacatgctgtagacatgatgatattatcattcattatactttttataatattattattaagtacaattatttgatctgcttgcttaaaaaatcaccagaggctgccccggcagaagctcctccagcagtggctccaccaccggccccagaaataaagccagaagcagcagaagtggccacagaagaaaaagaagagaaggaggaagaaaaagaagaggaggaggaggaagaagaggaggaagaatcagattcctaaggttagaaatagggagatgaagccaagtatatcaaactcctcaagtactgagtgggaatgagatgtttggtgaagtagaaatagatagctttagtggctctgactcagatatactgatgtagaaggaaggattctttcacttttattatttattttggtaattaaatttacattaatttaatatgttaaatatttta

SEQ ID NO: 147 - DNA, 2657 bp, Homo sapiens
gcgtcatctcgcgcttccaactgccctgtaaccaccaactgccattattccggctgggacccaggacttcaagccatgtggcgcgtcttgtttctgctcagcgggctcggcgggctgcggatggacagtaattttgatagtttacctgtgcaaattacagttccggagaaaatacggtcaataataaaggaaggaattgaatcgcaggcatcctacaaaattgtaattgaagggaaaccatatactgtgaatttaatgcaaaaaaactttttaccccataattttagagtttacagttatagtggcacaggaattatgaaaccacttgaccaagattttcagaatttctgccactaccaagggtatattgaaggttatccaaaatctgtggtgatggttagcacatgtactggactcaggggcgtactacagtttgaaaatgttagttatggaatagaacccctggagtcttcagttggctttgaacatgtaatttaccaagtaaaacataagaaagcagatgtttccttatataatgagaaggatattgaatcaagagatctgtcctttaaattacaaagcgtagagccacagcaagattttgcaaagtatatagaaatgcatgttatagttgaaaaacaattgtataatcatatggggtctgatacaactgttgtcgctcaaaaagttttccagttgattggattgacgaatgctatttttgtttcatttaatattacaattattctgtcttcattggagctttggatagatgaaaataaaattgcaaccactggagaagctaatgagttattacacacatttttaagatggaaaacatcttatcttgttttacgtcctcatgatgtggcatttttacttgtttacagagaaaagtcaaattatgttggtgcaacctttcaagggaagatgtgtgatgcaaactatgcaggaggtgttgttctgcaccccagaaccataagtctggaatcacttgcagttattttagctcaattattgagccttagtatggggatcacttatgatgacattaacaaatgccagtgctcaggagctgtctgcattatgaatccagaagcaattcatttcagtggtgtgaagatctttagtaactgcagcttcgaagactttgcacattttatttcaaagcagaagtcccagtgtcttcacaatcagcctcgcttagatccttttttcaaacagcaagcagtgtgtggtaatgcaaagctggaagcaggagaggagtgtgactgtgggactgaacaggattgtgcccttattggagaaacatgctgtgatattgccacatgtagatttaaagccggttcaaactgtgctgaaggaccatgctgcgaaaactgtctatttatgtcaaaagaaagaatgtgtaggccttcctttgaagaatgcgacctccctgaatattgcaatggatcatctgcatcatgcccagaaaaccactatgttcagactgggcatccgtgtggactgaatcaatggatctgtatagatggagtttgtatgagtggggataaacaatgtacagacacatttggcaaagaagtagagtttggcccttcagaatgttattctcaccttaattcaaagactgatgtatctggaaactgtggtataagtgattcaggatacacacagtgtgaagctgacaatctgcagtgcggaaaattaatatgtaaatatgtaggtaaatttttattacaaattccaagagccactattatttatgccaacataagtggacatctctgcattgctgtggaatttgccagtgatcatgcagacagccaaaagatgtggataaaagatggaacttcttgtggttcaaataaggtttgcaggaatcaaagatgtgtgagttcttcatacttgggttatgattgtactactgacaaatgcaatgatagaggtgtatgcaataacaaaaagcactgtcactgtagtgcttcatatttacctccagattgctcagttcaatcagatctatggcctggtgggagtattgacagtggcaattttccacctgtagctataccagccagactccctgaaaggcgctacattgagaacatttaccattccaaaccaatgagatggccatttttcttattcattcctttctttattattttctgtgtactgattgctataatggtgaaagttaatttccaaaggaaaaaatggagaactgaggactattcaagcgatgagcaacctgaaagtgagagtgaacctaaagggtagtctggacaacagagatgccatgatatcacttcttctagagtaattatctgtgatggatggacacaaaaaaatggaaagaaaagaatgtacattacctggtttcctgggattcaaacctgcatattgtgattttaatttgaccagaaaatatgatatatatgtataatttcacagataatttacttatttaaaaatgcatgataatgagttttacattacaaatttctgtttttttaaagttatcttacgctatttctgttggttagtagacactaattctgtcagtaggggcatggtataaggaaatatcataatgtaatgaggtggtactatgattaaaagccactgttacatttcaaaaaaaaaaaaaaaaa

SEQ ID NO: 148 - DNA, 2642 bp, Homo sapiens
gggatccacggagctgggtccccgccgcgccccgcggaccccacggcccttccgctccgctcggcctccaccaccctggcctcgagctgcagccgttccaaggacaacggccatccctgtggctgggccgccggctacgcaatgctgtctctcttgctgatcctctcaggcctgggccggctgacctccgcgggcccacgccacttattccatcaaaattgaggggaaaccatacacttcctgcttgaaaaacagtcatttttacacccacatttcctggtgtatttatacaacaaatcaggaacattgtatgcagattcttcattttcaaagggtcattgcttttaccaaggatatgctgcagacattccaaaatcagttgtggcactacgcacctgttctggactcagaggattactgcagttagataatatcagttatggtattgaaccattggaatcttcacctacatataagcatgtagtttatcgaattaaaaatgatgctattggtcattcctcctttcaagaaaattatcctgtggctcaatatatagatcagtcctacaggattcttgtcaaatcagacataaattcaggtgccatgctatcgaaaagaactctgaaaatacaaatcattatggataaagccatgtatgcttatatgggctccacggtggcagttgcagttgagaaagttttccaaatctttggtcttatcaacactatgttttcccagcttaatatgacagttatgctgtcttccttggagatctggtcagatcaaaataagatttcaacaagtggtcatgctgatgaaatactacagcgatttttgccttggaaacaaaaatttttgtttcaaaggtctcatgatatgacatacttattaatttataggaaccattctacttatgtgggagcaacatatcacggaatggcatgcgatccaaagtttgcaacaggaattgctctgtatccaaagaaaatcactgcagaggccttttcagttgttatggcacagttgcttggaattaatctgggattgacatatgatgatatctacaattgttactgtccaggaactacatgcataatgaatcctgatgcgatatgatcccatggtatgaagttttttagcagttgcagcatggatggatttaaacggatagttctgcagcctgaacttaaatgttttcaggataaaacagtttcaaaaatgacttaccgaaaatcttcaacttgtggcaatggaattttggaacctacagagcagtgtgattgtggctataaagagagacaggatttcaccatgttggccaggctagtctcaaacttcggacctcaggttatccacctgcctcagcctctcaaggtgctgggattacaaaccatacacaaccccattttgaaaaggagaaatagacaagaagaaatgggtaacaggcctcaagcaagtctgaaacccaagagagccaatgatggacattcagactcctgaataatttcctttgatgacatgtcctgcatcttctataaacaggcatgcactcataaaaaatgctgtaatcctgcagattgtactctagttagatctgcagaatgtggcactggatcatgctgtaacaataaaacttgtacgatccacgaaagaggccatgtctgcagaaaaagtgtagatatgtgtgattttccagaatattgcaatggaacatctgagttttgtgtacctgatgtgaaagctgctgatttagaatactgcagtaataagactagctattgctttaaaggagtatgcagagaaagggatagacagtgttcacagttatttggaaaatttgctaagtctgctaatcttctgtgtacagaagaagtgaattttcaaaatgacaaatttggaaactgtggttcccgttgtgatttttttgatatcctttgtggaaagattgtttgtcactggatacattcagaactagtaccaatgacagacttagacatacaatatacttaccttagaggtcacgtatgtttgtccgcacatgcaagaaatggttcaaaacaatcggagacctatacagaagatataactgcatgtggccaacacaaggtatgtcactcccgagaatgcagaaattttagtgaactaaatataacaaaatatactacaaattgtggacagaatgggatttgcaatgaacatttccattgtcaatgtgatcctggttatgctcctccagattgtgagccagcaatgtcatcaccaggaggaagtatcaatgatggattttggcttacagtagaaaaaagtgtacccttgcttccaaaacgacgtgctgctcctaaaaacaatggtcttttgatcagtttctacattttttcacctttgctcattttaattgctattgttagtcttaaatggaataaaatgaagagatttcggagcaaagtgggaacagtaagcagcagatctatctcagaagacagcggtagtaacagcagccagtcacagagttaaagtaaccaagtgataaaatcaaagccatcggatgaagaaaggatattaccaagaaactcacgattaacttaaaactttcaacttcacagaaaaaaataaatcatatgactaaaagcataaaaaaaaaa

SEQ ID NO: 149 - DNA, 1842 bp, Homo sapiens
gttctctgtcagggtctccctgggagggacgcagccaccgcagctggttggggcctggcttcgcccaggacagtcctttcctttcccattgtctttggatgactatcgctgggctgggacatgaggcgggcagaggcgcgggtcacccttaggacccccctcttgctgctggggctctgggcgctcctggctccggtccggtgttctcaaggccgtcccttgtggcactatgcctcctccgaggtggtgattcccaggaaggagacacaccatagcaaaggccttcagtttcccggctggctgtcctacagcctgtgttttggggtcaaagacacgtcattcacatgcggaggaaacaccttctttggcctagacatctgctggtgacaactcaggatgaccaaggagtcttgcagatgggtgacccctacatccctccagactgctagtacctcggctacctggaggaggtgcctctgtccatggtcaccgtcgacacgtgctatggggacctcagaggcatcatgaggctggacgaccttgcgtacgaaatcaaacccctccaggattcccgcaggccggtgtgtttttgaaacacttgctcctgtgtataacgaaaccatgacaacggttcgctgtggaaacctcatagtggaggggagggaggaatgtgactgtggctccttcaagcagtgttatgccagttattgctgccaaagtgactgtcacttaacaccggggagcatctgccatataggagagtgctgtacaaactgcagcttctccccaccagggactctctgcagacctatccaaaatatatgtgaccttccagagtactgtcacgggaccaccgtgacatgtcccgcaaacgtttatatgcaagatggaaccccgtgcactgaagaaggctactgctatcgtgggaactgcactgatcgcaatgtgctctgcaaggcgatctttggtgtcagtgctgaggatgctcccgaggtctgctatgacataaatcttgaaagctaccgatttggacattgtattagacaacaaacatatctcagctaccaggcttgtgcaggaatagataagttttgtggaagactgcagtgtaccaatgtgacccatcttccccggctgcaggaacgtgtttcattccatcactcagtgagaggagggtttcagtgttttggactggatgaacaccatgcaacagacacgactgatgttgggcgtgtgatagatggcactccttgtgttcatggaaacttctgtaataacgcccagtgcaatgtgactatcacttcactgggctacaactgccaccctcagaagtgcggtcatagaggagtctgcaacaacagaaggaactgccattgccatataggctgggatcctccactgtgcctaagaagaggtgctggtgggagtgtcaacagcgggccacctccaaaaagaacacgttccgtcaaacaaagccagcaatcagtgatgtatctgagagtggtctttggtcgtatttacgccttcataattgcactgctctttgggacagccaaaaatgtgcgaactatcaggaccaccaccgttaaggaagggacagttactaaccccgaataacactaattcagcctcccgatccctgtaaagatacagagaatataacagcaaaatctatgaaacaggatcaggggaagggatggcaaagctcaagtccacatttcttgaagtccacaggaagcacagggtcctgtttcacatcacagggaaacgggaggcattggcttctgtcccaggttcttgtaggtcgctgatgctcactctgaaataaatcttcaaaaacac

SEQ ID NO: 150 - DNA, 2612 bp, Homo sapiens
aaaactgcccacctatctgtgaggtgcttcatccctgcagtggaagtgaggaggaagaaaggtgaactccttttctcaagcacttctgctctcctctaccagaatcactcagaatgcttcccgggtgtatattcttgatgattttactcattcctcaggttaaagaaaagttcatccttggagtagagggtcaacaactggttcgtcctaaaaagcttcctctgatacagaagcgagatactggacacacccatgatgatgacatactgaaaacgtatgaagaagaattgttgtatgaaataaaactaaatagaaaaaccttagtccttcatcttctaagatccagggagttcctaggctcaaattacagtgaaacattctactccatgaaaggagaagcgttcaccaggcatcctcagatcatggatcattgtttttaccaaggatccatagtacacgaatatgattcagctgccagtatcagtacgtgtaatggtctaaggggattcttcagaataaacgaccaaagatacctcattgaaccagtgaaatactcagatgagggagaacatttggtgttcaaatataacctgagggtgccgtatggtgccaattattcctgtacagagcttaattttaccagaaaaactgttccaggggataatgaatctgaagaagactccaaaataaaaggcatccatgatgaaaagtatgttgaattgttcattgttgctgatgatactgtgtatcgcagaaatggtcatcctcacaataaactaaggaaccgaatttggggaatggtcaattttgtcaacatgatttataaaaccttaaacatccatgtgacgttggttggcattgaaatatggacacatgaagataaaatagaactatattcaaatatagaaactaccttattgcgtttttcattttggcaagaaaagatccttaaaacacggaaggattttgatcatgttgtattactcagtgggaagtggctctactcacatgtgcaaggaatttcttatccagggggtatgtgcctgccctattattccaccagtatcattaaggatcttttacctgacacaaacataattgcaaacagaatggcacatcaactggggcataaccttgggatgcagcatgacgagttcccatgcacctgtccttcaggaaaatgcgtgatggacagtgatggaagcattcctgcactgaaattcagtaaatgcagccaaaaccaataccaccagtacttgaaggattataagccaacatgcatgctcaacattccatttccttacaattttcatgatttccaattttgtggaaacaagaagttggatgagggtgaagagtgtgactgtggccctgctcaggagtgtactaatccttgctgtgatgcacacacatgtgtactgaagccaggatttacttgtgcagaaggagaatgctgtgaatcttgtcagataaaaaaagcagggtccatatgcagaccggcgaaagatgaatgtgattttcctgagatgtgcactggccactcgcctgcctgtcctaaggaccagttcagggtcaatggatttccttgcaagaactcagaaggctactgtttcatggggaaatgtccaactcgtgaggatcagtgctctgaactatttgatgatgaggcaatagagagtcatgatatctgctacaagatgaatacaaaaggaaataaatttggatactgcaaaaacaaggaaaacagatttcttccctgtgaggagaaagatgtcagatgtggaaagatctactgcactggaggggagctttcctctctccttggagaagacaagacttatcaccttaaggatccccagaagaatgctactgtcaaatgcaaaactatttttttataccatgattctacagacattggcctggtggcgtcaggaacaaaatgtggagagggaatggtgtgcaacaatggtgaatgtctaaacatggaaaaggtctatatctcaaccaattgcccctctcagtgcaatgaaaatcctgtggatggccacggactccagtgccactgtgaggaaggacaggcacctgtagcctgtgaagaaaccttacatgttaccaatatcaccatcttggttgttgtgcttgtcctggttattgtcggtatcggagttcttatactattagttcgttaccgaaaatgtatcaagttgaagcaagttcagagcccacctacagaaaccctgggagtggagaacaaaggatactttggtgatgagcagcagataaggactgagccaatcctgccagaaattcatttcctaaataaacctgcaagtaaagattcaagaggaatcgcagatcccaatcaaagtgccaagtgagcttgaagttggatatccaaaatggccgtgcaagcttaggctggggattctggatgcaacgtctttacaaccttacctagatatctgctactcacatttttggtagtgtttcaaacgttctttatccagacagacaatgtttaagagaaacaacttatttctgttaatatttaccggtagaattcacaccctctatcataaacatatgctgcagaaaaaaaaaaaaaaaaaaaa

SEQ ID NO: 151 - DNA, 3316 bp, Homo sapiens
gcggaaaagagcctcgggccaggagcgcaggaaccagaccgtgtcccgcggggctgtcacctccgcctctgctccccgacccggccatgcgcggcctcgggctctggctgctgggcgcgatgatgctgcctgcgattgcccccagccggccctgggccctcatggagcagtatgaggtcgtgttgccgtggcgtctgccaggcccccgagtccgccgagctctgccctcccacttgggcctgcacccagagagggtgagctacgtccttggggccacagggcacaacttcaccctccacctgcggaagaacagggacctgctgggctccggctacacagagacctatacggctgccaatggctccgaggtgacggagcagcctcgcgggcaggaccactgcttctaccagggccacgtagaggggtacccggactcagccgccagcctcagcacctgtgccggcctcaggggtttcttccaggtggggtcagacctgcacctgatcgagcccctggatgaaggtggcgagggcggacggcacgccgtgtaccaggctgagcacctgctgcagacggccgggacctgcggggtcagcgacgacagcctgggcagcctcctgggaccccggacggcagccgtcttcaggcctcggcccggggactctctgccatcccgagagacccgctacgtggagctgtatgtggtcgtggacaatgcagagttccagatgctggggagcgaagcagccgtgcgtcatcgggtgctggaggtggtgaatcacgtggacaagctatatcagaaactcaacttccgtgtggtcctggtgggcctggagatttggaatagtcaggacaggttccacgtcagccccgaccccagtgtcacactggagaacctcctgacctggcaggcacggcaacggacacggcggcacctgcatgacaacgtacagctcatcacgggtgtcgacttcaccgggactaccgtggggtttgccagggtgtccgccatgtgctcccacagctcaggggctgtgaaccaggaccacagcaagaaccccgtgggcgtggcctgtaccatggcccatgagatgggccacaacctgggcatggaccatgatgagaacgtccagggctgccgctgccaggaacgcttcgaggccggccgctgcatcatggcgggcagcattggctccagtttccccaggatgttcagtgactgcagccaggcctacctggagagctttttggagcggccgcagtcggtgtgcctcgccaacgcccctgacctcagccacctggtgggcggccccgtgtgtgggaacctgtttgtggagcgtggggagcagtgcgactgcggcccccccgaggactgccggaaccgctgctgcaactctaccacctgccagctggctgagggggcccagtgtgcgcacggtacctgctgccaggagtgcaaggtgaagccggctggtgagctgtgccgtcccaagaaggacatgtgtgacctcgaggagttctgtgacggccggcaccctgagtgcccggaagacgccttccaggagaacggcacgccctgctccgggggctactgctacaacggggcctgtcccacactggcccagcagtgccaggccttctgggggccaggtgggcaggctgccgaggagtcctgcttctcctatgacatcctaccaggctgcaaggccagccggtacagggctgacatgtgtggcgttctgcagtgcaagggtgggcagcagcccctggggcgtgccatctgcatcgtggatgtgtgccacgcgctcaccacagaggatggcactgcgtatgaaccagtgcccgagggcacccggtgtggaccagagaaggtttgctggaaaggacgttgccaggacttacacgtttacagatccagcaactgctctgcccagtgccacaaccatggggtgtgcaaccacaagcaggagtgccactgccacgcgggctgggccccgccccactgcgcgaagctgctgactgaggtgcacgcagcgtccgggagcctccccgtcttcgtggtggtggttctggtgctcctggcagttgtgctggtcaccctggcaggcatcatcgtctaccgcaaagcccggagccgcatcctgagcaggaacgtggctcccaagaccacaatggggcgctccaaccccctgttccaccaggctgccagccgcgtgccggccaagggcggggctccagccccatccaggggcccccaagagctggtccccaccacccacccgggccagcccgcccgacacccggcctcctcggtggctctgaagaggccgccccctgctcctccggtcactgtgtccagcccacccttcccagttcctgtctacacccggcaggcaccaaagcaggtcatcaagccaacgttcgcacccccagtgcccccagtcaaacccggggctggtgcggccaaccctggtccagctgagggtgctgttggcccaaaggttgccctgaagccccccatccagaggaagcaaggagccggagctcccacagcaccctaggggggcacctgcgcctgtgtggaaatttggagaagttgcggcagagaagccatgcgttccagcattccacggtccagctagtgccgctcagccctagaccctgactttgcaggctcagctgctgttctaacctcaggaatgcatctacctgagaggctcctgctgtccacgccctcagccaattccttctccccgccttggccacgtgtagccccagctgtctgcaggcaccaggctgggatgagctgtgtgcttgcgggtgcgtgtgtgtgtacgtgtctccaggtggccgctggtctcccgctgtgttcaggaggccacatatacagcccctcccagccacacctgcccctgctctggggcctgctgagccggctgccctgggcacccggttccaggcagcacagacgtggggcatccccagaaagactccatcccaggaccaggttcccctgcgtgctcttcgagagggtgtcagtgagcagactgcaccccaagctcccgactccaggtcccctgatcttggggcctgtttcccatgggattcaagagggacagccccagctttgtgtgtgtttaagcttaggaatcgcctttatggaaagggctatgtgggagagtcagctatcttgtctggttttcttgagacctcagatgtgtgttcagcagggctgaaagcttttattctttaataatgagaaatgtatattttactaataaattattgaccgagttctgtaaaaaaaaaaaaaaaa

SEQ ID NO: 152 - DNA, 4111 bp, Homo sapiens
cggcagggttggaaaatgatggaagaggcggaggtggaggcgaccgagtgctgagaggaacctgcggaatcggccgagatggggtctggcgcgcgctttccctcggggacccttcgtgtccggtggttgctgttgcttggcctggtgggcccagtcctcggtgcggcgcggccaggctttcaacagacctcacatctttcttcttatgaaattataactccttggagattaactagagaaagaagagaagcccctaggccctattcaaaacaagtatcttatgttattcaggctgaaggaaaagagcatattattcacttggaaaggaacaaagaccttttgcctgaagattttgtggtttatacttacaacaaggaagggactttaatcactgaccatcccaatatacagaatcattgtcattatcggggctatgtggagggagttcataattcatccattgctcttagcgactgttttggactcagaggattgctgcatttagagaatgcgagttatgggattgaacccctgcagaacagctctcattttgagcacatcatttatcgaatggatgatgtctacaaagagcctctgaaatgtggagtttccaacaaggatatagagaaagaaactgcaaaggatgaagaggaagagcctcccagcatgactcagctacttcgaagaagaagagctgtcttgccacagacccggtatgtggagctgttcattgtcgtagacaaggaaaggtatgacatgatgggaagaaatcagactgctgtgagagaagagatgattctcctggcaaactacttggatagtatgtatattatgttaaatattcgaattgtgctagttggactggagatttggaccaatggaaacctgatcaacatagttgggggtgctggtgatgtgctggggaacttcgtgcagtggcgggaaaagtttcttatcacacgtcggagacatgacagtgcacagctagttctaaagaaaggttttggtggaactgcaggaatggcatttgtgggaacagtgtgttcaaggagccacgcaggcgggattaatgtgtttggacaaatcactgtggagacatttgcttccattgttgctcatgaattgggtcataatcttggaatgaatcacgatgatgggagagattgttcctgtggagcaaagagctgcatcatgaattcaggagcatcgggttccagaaactttagcagttgcagtgcagaggactttgagaagttaactttaaataaaggaggaaactgccttcttaatattccaaagcctgatgaagcctatagtgctccctcctgtggtaataagttggtggacgctggggaagagtgtgactgtggtactccaaaggaatgtgaattggacccttgctgcgaaggaagtacctgtaagcttaaatcatttgctgagtgtgcatatggtgactgttgtaaagactgtcggttccttccaggaggtactttatgccgaggaaaaaccagtgagtgtgatgttccagagtactgcaatggttcttctcagttctgtcagccagatgtttttattcagaatggatatccttgccagaataacaaagcctattgctacaacggcatgtgccagtattatgatgctcaatgtcaagtcatctttggctcaaaagccaaggctgcccccaaagattgtttcattgaagtgaattctaaaggtgacagatttggcaattgtggtttctctggcaatgaatacaagaagtgtgccactgggaatgctttgtgtggaaagcttcagtgtgagaatgtacaagagatacctgtatttggaattgtgcctgctattattcaaacgcctagtcgaggcaccaaatgttggggtgtggatttccagctaggatcagatgttccagatcctgggatggttaacgaaggcacaaaatgtggtgctggaaagatctgtagaaacttccagtgtgtagatgcttctgttctgaattatgactgtgatgttcagaaaaagtgtcatggacatggggtatgtaatagcaataagaattgtcactgtgaaaatggctgggctcccccaaattgtgagactaaaggatacggaggaagtgtggacagtggacctacatacaatgaaatgaatactgcattgagggacggacttctggtcttcttcttcctaattgttccccttattgtctgtgctatttttatcttcatcaagagggatcaactgtggagaagctacttcagaaagaagagatcacaaacatatgagtcagatggcaaaaatcaagcaaacccttctagacagccggggagtgttcctcgacatgtttctccagtgacacctcccagagaagttcctatatatgcaaacagatttgcagtaccaacctatgcagccaagcaacctcagcagttcccatcaaggccacctccaccacaaccgaaagtatcatctcagggaaacttaattcctgcccgtcctgctcctgcacctcctttatatagttccctcacttgatttttttaaccttctttttgcaaatgtcttcagggaactgagctaatactttttttttttcttgatgttttcttgaaaagcctttctgttgcaactatgaatgaaaacaaaacaccacaaaacagacttcactaacacagaaaaacagaaactgagtgtgagagttgtgaaatacaaggaaatgcagtaaagccagggaatttacaataacatttccgtttccatcattgaataagtcttattcagtcatcggtgaggttaatgcactaatcatggattttttgaacatgttattgcagtgattctcaaattaactgtattggtgtaagatttttgtcattaagtgtttaagtgttattctgaattttctaccttagttatcattaatgtagttcctcattgaacatgtgataatctaatacctgtgaaaactgactaatcagctgccaataatatctaatatttttcatcatgcacgaattaataatcatcatactctagaatcttgtctgtcactcactacatgaataagcaaatattgtcttcaaaagaatgcacaagaaccacaattaagatgtcatattattttgaaagtacaaaatatactaaaagagtgtgtgtgtattcacgcagttactcgcttccatttttatgacctttcaactataggtaataactcttagagaaattaatttaatattagaatttctattatgaatcatgtgaaagcatgacattcgttcacaatagcactattttaaataaattataagctttaaggtacgaagtatttaatagatctaatcaaatatgttgattcatggctataataaagcaggagcaattataaaatcttcaatcaattgaacttttacaaaaccacttgagaatttcatgagcactttaaaatctgaactttcaaagcttgctattaaatcatttagaatgtttacatttactaaggtgtgctgggtcatgtaaaatattagacactaatattttcatagaaattaggctggagaaagaaggaagaaatggttttcttaaatacctacaaaaaagttactgtggtatctatgagttatcatcttagctgtgttaaaaatgaatttttactatggcagatatggtatggatcgtaaaattttaagcactaaaaattttttcataacctttcataataaagtttaataataggtttattaactgaatttcattagttttttaaaagtgtttttggtttgtgtatatatacatatacaaatacaacatttacaataaataaaatacttgaaattctcttttgtgtctcctagtagcttcctactcaactatttataatctcattaattaaaaagttataattttagataaaaattctagtcaaatttttacagatattatctcactaattttcagacttttgccaaagtgtgcacaatggctttttgttaataaagaacagattagttttgaagaaggcaaaaatttcagttttctgaagacagcatgttattttaacaatcaagtatacatattaaaaattgtgagcaatctcaaaaaaaaaaaaaaa

SEQ ID NO: 153 - DNA, 3927 bp, Homo sapiens
gcggcggcaggcctagcagcacgggaaccgtcccccgcgcgcatgcgcgcgcccctgaagcgcctgggggacgggtatgggcgggaggtaggggcgcggctccgcgtgccagttgggtgcccgcgcgtcacgtggtgaggaaggaggcggaggtctgagtttcgaaggagggggggagagaagagggaacgagcaagggaaggaaagcggggaaaggaggaaggaaacgaacgagggggagggaggtccctgttttggaggagctaggagcgttgccggcccctgaagtggagcgagagggaggtgcttcgccgtttctcctgccaggggaggtcccggcttcccgtggaggctccggaccaagccccttcagcttctccctccggatcgatgtgctgctgttaacccgtgaggaggcggcggcggcggcagcggcagcggaagatggtgttgctgagagtgttaattctgctcctctcctgggcggcggggatgggaggtcagtatgggaatcctttaaataaatatatcagacattatgaaggattatcttacaatgtggattcattacaccaaaaacaccagcgtgccaaaagagcagtctcacatgaagaccaatttttacgtctagatttccatgcccatggaagacatttcaacctacgaatgaagagggacacttcccttttcagtgatgaatttaaagtagaaacatcaaataaagtacttgattatgatacctctcatatttacactggacatatttatggtgaagaaggaagttttagccatgggtctgttattgatggaagatttgaaggattcatccagactcgtggtggcacattttatgttgagccagcagagagatatattaaagaccgaactctgccatttcactctgtcatttatcatgaagatgatattaactatccccataaatacggtcctcaggggggctgtgcagatcattcagtatttgaaagaatgaggaaataccagatgactggtgtagaggaagtaacacagatacctcaagaagaacatgctgctaatggtccagaacttctgaggaaaaaacgtacaacttcagctgaaaaaaatacttgtcagctttatattcagactgatcatttgttctttaaatattacggaacacgagaagctgtgattgcccagatatccagtcatgttaaagcgattgatacaatttaccagaccacagacttctccggaatccgtaacatcagtttcatggtgaaacgcataagaatcaatacaactgctgatgagaaggaccctacaaatcctttccgtttcccaaatattggtgtggagaagtttctggaattgaattctgagcagaatcatgatgactactgtttggcctatgtcttcacagaccgagattttgatgatggcgtacttggtctggcttgggttggagcaccttcaggaagctctggaggaatatgtgaaaaaagtaaactctattcagatggtaagaagaagtccttaaacactggaattattactgttcagaactatgggtctcatgtacctcccaaagtctctcacattacttttgctcacgaagttggacataactttggatccccacatgattctggaacagagtgcacaccaggagaatctaagaatttgggtcaaaaagaaaatggcaattacatcatgtatgcaagagcaacatctggggacaaacttaacaacaataaattctcactctgtagtattagaaatataagccaagttcttgagaagaagagaaacaactgttttgttgaatctggccaacctatttgtggaaatggaatggtagaacaaggtgaagaatgtgattgtggctatagtgaccagtgtaaagatgaatgctgcttcgatgcaaatcaaccagagggaagaaaatgcaaactgaaacctgggaaacagtgcagtccaagtcaaggtccttgttgtacagcacagtgtgcattcaagtcaaagtctgagaagtgtcgggatgattcagactgtgcaagggaaggaatatgtaatggcttcacagctctctgcccagcatctgaccctaaaccaaacttcacagactgtaataggcatacacaagtgtgcattaatgggcaatgtgcaggttctatctgtgagaaatatggcttagaggagtgtacgtgtgccagttctgatggcaaagatgataaagaattatgccatgtatgctgtatgaagaaaatggacccatcaacttgtgccagtacagggtctgtgcagtggagtaggcacttcagtggtcgaaccatcaccctgcaacctggatccccttgcaacgattttagaggttactgtgatgttttcatgcggtgcagattagtagatgctgatggtcctctagctaggcttaaaaaagcaatttttagtccagagctctatgaaaacattgctgaatggattgtggctcattggtgggcagtattacttatgggaattgctctgatcatgctaatggctggatttattaagatatgcagtgttcatactccaagtagtaatccaaagttgcctcctcctaaaccacttccaggcactttaaagaggaggagacctccacagcccattcagcaaccccagcgtcagcggccccgagagagttatcaaatgggacacatgagacgctaactgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgcattttcttcacttgcaggcaaacttggctctcaataaacttttaccacaaattgaaataaatatatttttttcaactgccaatcaaggctaggaggctcgaccacctcaacattggagacatcacttgccaatgtacataccttgttatatgcagacatgtatttcttacgtacactgtacttctgtgtgcaattgtaaacagaaattgcaatatggatgtttctttgtattataaaatttttccgctcttaattaaaaattactgtttaattgacatactcaggataacagagaatggtggtattcagtggtccaggattctgtaatgctttacacaggcagttttgaaatgaaaatcaatttacctttctgttacgatggagttggttttgatactcattttttctttatcacatggctgctacgggcacaagtgactatactgaagaacacagttaagtgttgtgcaaactggacatagcagcacatactacttcagagttcatgatgtagatgtctggtttctgcttacgtcttttaaactttctaattcaattccatttttcaattaataggtgaaattttattcatgctttgatagaaattatgtcaatgaaatgattctttttatttgtagcctacttatttgtgtttttcatatatctgaaatatgctaattatgttttctgtctgatatggaaaagaaaagctgtgtctttatcaaaatatttaaacggttttttcagcatatcatcactgatcattggtaaccactaaagatgagtaatttgcttaagtagtagttaaaattgtagataggccttctgacattttttttcctaaaatttttaacagcattgaaggtgaaacagcacaatgtcccattccaaatttatttttgaaacagatgtaaataattggcattttaaagag

SEQ ID NO: 154 - DNA, 4402 bp, Homo sapiens
atgaggctgctgcggcgctgggcgttcgcggctctgctgctgtcgctgctccccacgcccggtcttgggacccaaggtcctgctggagctctgcgatgggggggcttaccccagctgggaggcccaggagcccctgaggtcacggaacccagccgtctggttagggagagctccgggggagaggtccgaaagcagcagctggacacaagggtccgccaggagccaccagggggcccgcctgtccatctggcccaggtgagtttcgtcatcccagccttcaactcaaacttcaccctggacctggagctgaaccaccacctcctctcctcgcaatacgtggagcgccacttcagccgggaggggacaacccagcacagcaccggggctggagaccactgctactaccaggggaagctccgggggaacccgcactccttcgccgccctctccacctgccaggggctgcatggggtcttctctgatgggaacttgacttacatcgtggagccccaagaggtggctggaccttggggagcccctcagggaccccttccccacctcatttaccggacccctctcctcccagatcccctcggatgcagggaaccaggctgcctgtttgctgtgcctgcccagtcggctcctccaaaccggccgaggctgagaaggaaaaggcaggtccgccggggccaccctacagtgcacagtgaaaccaagtatgtggagctaattgtgatcaacgaccaccagctgttcgagcagatgcgacagtcggtggtcctcaccagcaactttgccaagtccgtggtgaacctggccgatgtgatatacaaggagcagctcaacactcgcatcgtcctggttgccatggaaacatgggcagatggggacaagatccaggtgcaggatgacctcctggagaccctggcccggctcatggtctaccgacgggagggtctgcctgagcccagtgatgccacccacctcttctcgggcaggaccttccagagcacgagcagcggggcagcctacgtggggggcatatgctccctgtcccacggcgggggtgtgaacgagtacggcaacatgggggcgatggccgtgacccttgcccagacgctgggacagaacctgggcatgatgtggaacaaacaccggagctcggcaggggactgcaagtgtccagacatctggctgggctgcatcatggaggacactgggttctacctgccccgcaagttctcgcgctgtagcatcgacgagtacaaccagtttctgcaggagggtggtggcagctgcctcttcaacaagcccctcaagctcctggaccccccagagtgcgggaacggcttcgtggaggcaggggaggagtgcgactgcggctcggtgcaggagtgcagccgcgcaggtggcaactgctgcaagaaatgcaccctgactcacgacgccatgtgcagcgacgggctctgctgtcgccgctgcaagtacgaaccacggggtgtgtcctgccgagaggccgtgaacgagtgcgacatcgcggagacctgcaccggggactctagccagtgcccgcctaacctgcacaagctggacggttactactgtgaccatgagcagggccgctgctacggaggtcgctgcaaaacccgggaccggcagtgccaggttctttggggccatgcggctgctgatcgcttctgctacgagaagctgaatgtggaggggacggagcgtgggagctgtgggcgcaagggatctggctgggtccagtgcagtaagcaggacgtgctgtgtggcttcctcctctgtgtcaacatctctggagctcctcggctaggggacctggtgggagacatcagtagtgtcaccttctaccaccagggcaaggagctggactgcaggggaggccacgtgcagctggcggacggctctgacctgagctatgtggaggatggcacagcctgcgggcctaacatgttgtgcctggaccatcgctgcctgccagcttctgccttcaacttcagcacctgccccggcagtggggagcgccggatttgctcccaccacggggtctgcagcaatgaagggaagtgcatctgtcagccagactggacaggcaaagactgcagtatccataaccccctgcccacgtccccacccacgggggagacggagagatataaaggtcccagcggcaccaacatcatcattggctccattgctggggctgtcctggttgcagccatcgtcctgggcggcacgggctggggatttaaaaacattcgccgaggaaggtccggaggggcctaagtgccaccctcctccctccaagcctggcacccaccgtctcggccctgaaccacgaggctgcccccatccagccacggagggaggcaccatgcaaatgtcttccaggtccaaacccttcaactcctggctccgcaggggtttgggtgggggctgtggccctgcccttggcaccaccagggtggaccaggcctggagggcacttcctccacagtcccccacccacctcctgcggctcagccttgcacacccactgccccgtgtgaatgtagcttccacctcatggattgccacagctcaactcgggggcgcctggagggatgcccccaggcagccaccagtggacctagcctggatggcccctccttgcaaccaggcagctgagaccagggtcttacctctctgggacctagggggacggggctgacatctacattttttaaaactgaatcttaatcgatgaatgtaaactcgggggtgctggggccagggcagatgtggggatgttttgacatttacaggagggcccggagaaactgaggtatggccatgccctagaccctccccaaggatgaccacacccgaagtcctgtcactgagcacagtcaggggctgggcatcccagcttgcccccgcttagccccgctgagcttggaggaagtatgagtgctgattcaaaccaaagctgcctgtgccatgcccaaggcctaggttatgggtacggcaaccacatgtcccagatcgtctccaattcgaaaacaaccgtcctgctgtccctgtcaggacacatggattttggcagggcggggggggttctagaaaatatagttcctataataaaatggcaccttccccctttcaagaagggtgattctggggccgactcagggtttaggtgccccctggtgtggcctagatgtccccacctgggccatctttctggggaggactctcccaggtagggaaggccagaggtggcccagtgcctggagggttagggtctctgcctgggatatgcaagaggaagtaggaaagggaggtctcatggatgatcctaggctgctagaagtccttaaggccccatctagtccattccactccctacccccattccagagccgagtagtaagtttacagatgtttcccccattacgtacccccacccatccctgctgcagcgagcctgagagccaggtagagccaggcacagctcctcagtcttctcacacagtcctgccggtggccttccctcatgacccttgcttgggagggtggagcactggctccttgaccctaaaaggtagctggcaggggcaagatgggggccagctacctaatggatgaaagccacaagtgaatacagttcttgtcaccagggttgccctgccctcactcggcagggagttctgacaccccagggcccgtgagctacctgcttgagcccctgtttctggggcaccttcgaggaggcgttgtggagggcatcgccccctgtttattcacaacaccctcaggggcaaacaggcctgggacccgctgacaccattttgggtagctggatgcacccgacagcagtggggtccacacactgagctccagctggcactgcccactcaagggctgagtggaggggcccctccggccagctctgctccaccagccctgcaagctgatgcagggcgggggaagggctgggtgttgcactattgctgcgctgccttaaggcatctgtcctctggtggtgcacccgtgcacacaggtacagtgcatctgggcacagcttttggatccacacctctgcacaagtgtgaatacctctgcacatatgggcgtatctgtgtgtgctcgtgtatatggggtggggaacatgagacttcctgtgaccagtccaccctggctcccagctgtctgtatcctcctgccccgccctggcgagtgcctaccctggcagaacccagggaggagtggaggctgcctctgcctgggcctccacacagcatcctgtacatacgccacctgggctgggggtggggaggcagggccaggagcatcgattaaagatcacatcctggggcttccagggagctcacaccaa

SEQ ID NO: 155 - DNA, 6093 bp, Homo sapiens
acgccgccaactggcgggggtgcgggggagacaataatttgttccgcggtaataagaacggtgactgctggccgtggatccatttcacaggcctgccttctctcactaacgctcttcctagtccccgggccaactcggacagtttgctcatttattgcaacggtcaaggctggcttgtgccagaacggcgcgcgcgcgcgcacgcacgcacacacacggggggaaacttttttaaaaatgaaaggctagaagagctcagcggcggcgcgggcgctgcgcgagggctccggagctgactcgccgaggcaggaaatccctccggtcgcgacgcccggccccggctcggcgcccgcgtgggatggtgcagcgctcgccgccgggcccgagagctgctgcactgaaggccggcgacgatggcagcgcgcccgctgcccgtgtcccccgcccgcgccctcctgctcgccctggccggtgctctgctcgcgccctgcgaggcccgaggggtgagcttatggaaccaaggaagagctgatgaagttgtcagtgcctctgttgggagtggggacctctggatcccagtgaagagcttcgactccaagaatcatccagaagtgctgaatattcgactacaacgggaaagcaaagaactgatcataaatctggaaagaaatgaaggtctcattgccagcagtttcacggaaacccactatctgcaagacggtactgatgtctccctcgctcgaaattacacggtaattctgggtcactgttactaccatggacatgtacggggatattctgattcagcagtcagtctcagcacgtgttctggtctcaggggacttattgtgtttgaaaatgaaagctatgtcttagaaccaatgaaaagtgcaaccaacagatacaaactcttcccagcgaagaagctgaaaagcgtccggggatcatgtggatcacatcacaacacaccaaacctcgctgcaaagaatgtgtttccaccaccctctcagacatgggcaagaaggcataaaagagagaccctcaaggcaactaagtatgtggagctggtgatcgtggcagacaaccgagagtttcagaggcaaggaaaagatctggaaaaagttaagcagcgattaatagagattgctaatcacgttgacaagttttacagaccactgaacattcggatcgtgttggtaggcgtggaagtgtggaatgacatggacaaatgctctgtaagtcaggacccattcaccagcctccatgaatttctggactggaggaagatgaagcttctacctcgcaaatcccatgacaatgcgcagcttgtcagtggggtttatttccaagggaccaccatcggcatggccccaatcatgagcatgtgcacggcagaccagtctgggggaattgtcatggaccattcagacaatccccttggtgcagccgtgaccctggcacatgagctgggccacaatttcgggatgaatcatgacacactggacaggggctgtagctgtcaaatggcggttgagaaaggaggctgcatcatgaacgcttccaccgggtacccatttcccatggtgttcagcagttgcagcaggaaggacttggagaccagcctggagaaaggaatgggggtgtgcctgtttaacctgccggaagtcagggagtctttcgggggccagaagtgtgggaacagatttgtggaagaaggagaggagtgtgactgtggggagccagaggaatgtatgaatcgctgctgcaatgccaccacctgtaccctgaagccggacgctgtgtgcgcacatgggctgtgctgtgaagactgccagctgaagcctgcaggaacagcgtgcagggactccagcaactcctgtgacctcccagagttctgcacaggggccagccctcactgcccagccaacgtgtacctgcacgatgggcactcatgtcaggatgtggacggctactgctacaatggcatctgccagactcacgagcagcagtgtgtcacgctctggggaccaggtgctaaacctgcccctgggatctgctttgagagagtcaattctgcaggtgatccttatggcaactgtggcaaagtctcgaagagttcctttgccaaatgcgagatgagagatgctaaatgtggaaaaatccagtgtcaaggaggtgccagccggccagtcattggtaccaatgccgtttccatagaaacaaacatccccctgcagcaaggaggccggattctgtgccgggggacccacgtgtacttgggcgatgacatgccggacccagggcttgtgcttgcaggcacaaagtgtgcagatggaaaaatctgcctgaatcgtcaatgtcaaaatattagtgtctttggggttcacgagtgtgcaatgcagtgccacggcagaggggtgtgcaacaacaggaagaactgccactgcgaggcccactgggcacctcccttctgtgacaagtttggctttggaggaagcacagacagcggccccatccggcaagcagataaccaaggtttaaccataggaattctggtgaccatcctgtgtcttcttgctgccggatttgtggtttatctcaaaaggaagaccttgatacgactgctgtttacaaataagaagaccaccattgaaaaactaaggtgtgtgcgcccttcccggccaccccgtggcttccaaccctgtcaggctcacctcggccaccttggaaaaggcctgatgaggaagccgccagattcctacccaccgaaggacaatcccaggagattgctgcagtgtcagaatgttgacatcagcagacccctcaacggcctgaatgtccctcagccccagtcaactcagcgagtgcttcctcccctccaccgggctccacgtgcacctagcgtccctgccagacccctgccagccaagcctgcacttaggcaggcccaggggacctgtaagccaaacccccctcagaagcctctgcctgcagatcctctggccagaacaactcggctcactcatgccttggccaggaccccaggacaatgggagactgggctccgcctggcacccctcagacctgctccacaatatccacaccaagtgcccagatccacccacaccgcctatattaagtgagaagccgacaccttttttcaacagtgaagacagaagtttgcactatctttcagctccagttggagttttttgtaccaacttttaggattttttttaatgtttaaaacatcattactataagaactttgagctactgccgtcagtgctgtgctgtgctatggtgctctgtctacttgctcaggtacttgtaaattattaatttatgcagaatgttgattacagtgcagtgcgctgtagtaggcatttttaccatcactgagttttccatggcaggaaggcttgttgtgcttttagtattttagtgaacttgaaatatcctgcttgatgggattctggacaggatgtgtttgctttctgatcaaggccttattggaaagcagtcccccaactacccccagctgtgcttatggtaccagatgcagctcaagagatcccaagtagaatctcagttgattttctggattccccatctcaggccagagccaaggggcttcaggtccaggctgtgtttggctttcagggaggccctgtgccccttgacaactggcaggcaggctcccagggacacctgggagaaatctggcttctggccaggaagctttggtgagaacctgggttgcagacaggaatcttaaggtgtagccacaccaggatagagactggaacactagacaagccagaacttgaccctgagctgaccagccgtgagcatgtttggaaggggtctgtagtgtcactcaaggcggtgcttgatagaaatgccaagcacttctttttctcgctgtcctttctagagcactgccaccagtaggttatttagcttgggaaaggtggtgtttctgtaagaaacctactgcccaggcactgcaaaccgccacctccctatactgcttggagctgagcaaatcaccacaaactgtaatacaatgatcctgtattcagacagatgaggctttccatgggaccacaactattttcagatgtgaaccattaaccagatctagtcaatcaagtctgtttactgcaaggttcaacttattaacaattaggcagactctttatgcttgcaaaaactacaaccaatggaatgtgatgttcatgggtatagttcatgtctgctatcattattcgtagatattggacaaagaaccttctctatggggcatcctctttttccaacttggctgcaggaatctttaaaagatgcttttaacagagtctgaacctatttcttaaacacttgcaacctacctgttgagcatcacagaatgtgataaggaaatcaacttgcttatcaacttcctaaatattatgagatgctggcttgggcagcatccccttgaactcttcactcttcaaatgcctgactagggagccatgtttcacaaggtctttaaagtgactaatggcatgagaaatacaaaaatactcagataaggtaaaatgccatgatgcctctgtcttctggactggttttcacattagaagacaattgacaacagttacataattcactctgagtgttttatgagaaagccttcttttgggggtcaacagttttcctatgctttgaaacagaaaaatatgtaccaagaatcttggtttgccttccagaaaacaaaactgcatttcactttcccggtgttccccactgtatctaggcaacatagtattcatgactatggataaactaaacacgtgacacaaacacacacaaaagggaacccagctctaatacattccaactcgtatagcatgcatctgtttattctatagttattaagttctttaaaatgtaaagccatgctggaaaataatactgctgagatacatacagaattactgtaactgattacacttggtaattgtactaaagccaaacatatatatactattaaaaaggtttacagaattttatggtgcattacgtgggcattgtctttttagatgcccaaatccttagatctggcatgttagcccttcctccaattataagaggatatgaactgagtttttcttttgttgtttgttcttagctgtaattcctatgcttctatttcagagagccaggagagtttgatattaaaggaggttaaaactgtgatcttatgccatgtcatcaatggccacttaggggccatggctgatgacacattcttatctctacagtactaatgtgttattatagagccatgcattttatttctgaataagaacatatttaaactaatattcccttacaatatggacagtattaatccttccaagatgcagtatttatcaagtgaagcatatttagcagcaaattccattttaacataacttaggaaccaataaccagggtgttttgtggttgggggaggcacggggtggagtattcttttttatatcctcaaaacaaaaaaaatcaatacttatatttcaatggcaatctagtatttttttaaaagactgtataggcatgaataatagaggtggtttgagttttgtagggccatcacctggaaagtcaatgtgactagacacaaagtagcccagaggctacttttcttcctacagcttattatagttgtaggttctatgacctcacttcatgggttccaggcaattccgctgaaaggtttgtctcctgaaattttttaagtttgttttcctgacacatgtaatcagatgtgtagcaaccgagggaaacgaagcctaacattctccattgtggaaatacacacaggaggttacatttcacagcgtggatttttccagcttacacatgtgggatgacatcacagaaaccacaaaagcagcaaattaaactgtaggagagtcaatactcctgacgagtctcggggggggggcatttttatgccttcttaactttatgagaattctcaggctgaactataggccattgttcccaggcaaatcaatacatcaatgcatcctcaaaaaaaaa

SEQ ID NO: 156 - DNA, 2970 bp, Homo sapiens
gcgcggcgctgctgggttctccgaggcgacctggccgccggccgctcctccgcgcgctgttccgcacttgctgccctcgcccggcccggagcgccgctgccatgcggctggcgctgctctgggccctggggctcctgggcgcgggcagccctctgccttcctggccgctcccaaatataggtggcactgaggagcagcaggcagagtcagagaaggccccgagggagcccttggagccccaggtccttcaggacgatctcccaattagcctcaaaaaggtgcttcagaccagtctgcctgagcccctgaggatcaagttggagctggacggtgacagtcatatcctggagctgctacagaatagggagttggtcccaggccgcccaaccctggtgtggtaccagcccgatggcactcgggtggtcagtgagggacacactttggagaactgctgctaccagggaagagtgcggggatatgcaggctcctgggtgtccatctgcacctgctctgggctcagaggcttggtggtcctgaccccagagagaagctataccctggagcaggggcctggggaccttcagggtcctcccattatttcgcgaatccaagatctccacctgccaggccacacctgtgccctgagctggcgggaatctgtacacactcagaagccaccagagcaccccctgggacagcgccacattcgccggaggcgggatgtggtaacagagaccaagactgtggagttggtgattgtggctgatcactcggaggcccagaaataccgggacttccagcacctgctaaaccgcacactggaagtggccctcttgctggacacattcttccggcccctgaatgtacgagtggcactagtgggcctggaggcctggacccagcgtgacctggtggagatcagcccaaacccagctgtcaccctcgaaaacttcctccactggcgcagggcacatttgctgcctcgattgccccatgacagtgcccagctggtgactggtacttcattctctgggcctacggtgggcatggccattcagaactccatctgttctcctgacttctcaggaggtgtgaacatggaccactccaccagcatcctgggagtcgcctcctccatagcccatgagttgggccacagcctgggcctggaccatgatttgcctgggaatagctgcccctgtccaggtccagccccagccaagacctgcatcatggaggcctccacagacttcctaccaggcctgaacttcagcaactgcagccgacgggccctggagaaagccctcctggatggaatgggcagctgcctcttcgaacggctgcctagcctaccccctatggctgctttctgcggaaatatgtttgtggagccgggcgagcagtgtgactgtggcttcctggatgactgcgtcgatccctgctgtgattctttgacctgccagctgaggccaggtgcacagtgtgcatctgacggaccctgttgtcaaaattgccagctgcgcccgtctggctggcagtgtcgtcctaccagaggggattgtgacttgcctgaattctgcccaggagacagctcccagtgtccccctgatgtcagcctaggggatggcgagccctgcgctggcgggcaagctgtgtgcatgcacgggcgttgtgcctcctatgcccagcagtgccagtcactttggggacctggagcccagcccgctgcgccactttgcctccagacagctaatactcggggaaatgcttttgggagctgtgggcgcaaccccagtggcagttatgtgtcctgcacccctagagatgccatttgtgggcagctccagtgccagacaggtaggacccagcctctgctgggctccatccgggatctactctgggagacaatagatgtgaatgggactgagctgaactgcagctgggtgcacctggacctgggcagtgatgtggcccagcccctcctgactctgcctggcacagcctgtggccctggcctggtgtgtatagaccatcgatgccagcgtgtggatctcctgggggcacaggaatgtcgaagcaaatgccatggacatggggtctgtgacagcaacaggcactgctactgtgaggagggctgggcaccccctgactgcaccactcagctcaaagcaaccagctccctgaccacagggctgctcctcagcctcctggtcttattggtcctggtgatgcttggtgccagctactggtaccgtgcccgcctgcaccagcgactctgccagctcaagggacccacctgccagtacagggcagcccaatctggtccctctgaacggccaggacctccgcagagggccctgctggcacgaggcactaagcaggctagtgctctcagcttcccggcccccccttccaggccgctgccgcctgaccctgtgtccaagagactccaggctgagctggctgaccgacccaatccccctacccgccctctgcccgctgacccggtggtgagaagcccgaagtctcaggggccagccaagcccccacccccaaggaagccactgcctgccgacccccagggccggtgcccatcgggtgacctgcccggcccaggggctggaatcccgcccctagtggtaccctccagaccagcgccaccgcctccgacagtgtcctcgctctacctctgacctctccggaggttccgctgcctccaagccggacttagggcttcaagaggcgggcgtgccctctggagtcccctaccatgactgaaggcgccagagactggcggtgtcttaagactccgggcaccgccacgcgctgtcaagcaacactctgcggacctgccggcgtagttgcagcgggggcttggggaggggctgggggttggacgggattgaggaaggtccgcacagcctgtctctgctcagttgcaataaacgtgacatcttgggagcgttcaaa

SEQ ID NO: 157 - DNA, 3572 bp, Homo sapiens
acctgcacttctgggggcgtcgagcctggcggtagaatcttcccagtaggcggcgcgggagggaaaagaggattgaggggctaggccgggcggatcccgtcctcccccgatgtgagcagttttccgaaaccccgtcaggcgaaggctgcccagagaggtggagtcggtagcggggccgggaacatgaggcagtctctcctattcctgaccagcgtggttcctttcgtgctggcgccgcgacctccggatgacccgggcttcggcccccaccagagactcgagaagcttgattctttgctctcagactacgatattctctctttatctaatatccagcagcattcggtaagaaaaagagatctacagacttcaacacatgtagaaacactactaactttttcagctttgaaaaggcattttaaattatacctgacatcaagtactgaacgtttttcacaaaatttcaaggtcgtggtggtggatggtaaaaacgaaagcgagtacactgtaaaatggcaggacttcttcactggacacgtggttggtgagcctgactctagggttctagcccacataagagatgatgatgttataatcagaatcaacacagatggggccgaatataacatagagccactttggagatttgttaatgataccaaagacaaaagaatgttagtttataaatctgaagatatcaagaatgtttcacgtttgcagtctccaaaagtgtgtggttatttaaaagtggataatgaagagttgctcccaaaagggttagtagacagagaaccacctgaagagcttgttcatcgagtgaaaagaagagctgacccagatcccatgaagaacacgtgtaaattattggtggtagcagatcatcgcttctacagatacatgggcagaggggaagagagtacaactacaaattacttaatagagctaattgacagagttgatgacatctatcggaacacttcatgggataatgcaggttttaaaggctatggaatacagatagagcagattcgcattctcaagtctccacaagaggtaaaacctggtgaaaagcactacaacatggcaaaaagttacccaaatgaagaaaaggatgcttgggatgtgaagatgttgctagagcaatttagctttgatatagctgaggaagcatctaaagtttgcttggcacaccttttcacataccaagattttgatatgggaactcttggattagcttatgttggctctcccagagcaaacagccatggaggtgtttgtccaaaggcttattatagcccagttgggaagaaaaatatctatttgaatagtggtttgacgagcacaaagaattatggtaaaaccatccttacaaaggaagctgacctggttacaactcatgaattgggacataattttggagcagaacatgatccggatggtctagcagaatgtgccccgaatgaggaccagggagggaaatatgtcatgtatcccatagctgtgagtggcgatcacgagaacaataagatgttttcaaactgcagtaaacaatcaatctataagaccattgaaagtaaggcccaggagtgttttcaagaacgcagcaataaagtttgtgggaactcgagggtggatgaaggagaagagtgtgatcctggcatcatgtatctgaacaacgacacctgctgcaacagcgactgcacgttgaaggaaggtgtccagtgcagtgacaggaacagtccttgctgtaaaaactgtcagtttgagactgcccagaagaagtgccaggaggcgattaatgctacttgcaaaggcgtgtcctactgcacaggtaatagcagtgagtgcccgcctccaggaaatgctgaagatgacactgtttgcttggatcttggcaagtgtaaggatgggaaatgcatccctttctgcgagagggaacagcagctggagtcctgtgcatgtaatgaaactgacaactcctgcaaggtgtgctgcagggacctttctggccgctgtgtgccctatgtcgatgctgaacaaaagaacttatttttgaggaaaggaaagccctgtacagtaggattttgtgacatgaatggcaaatgtgagaaacgagtacaggatgtaattgaacgattttgggatttcattgaccagctgagcatcaatacttttggaaagtttttagcagacaacatcgttgggtctgtcctggttttctccttgatattttggattcctttcagcattcttgtccattgtgtggataagaaattggataaacagtatgaatctctgtctctgtttcaccccagtaacgtcgaaatgctgagcagcatggattctgcatcggttcgcattatcaaaccctttcctgcgccccagactccaggccgcctgcagcctgcccctgtgatcccttcggcgccagcagctccaaaactggaccaccagagaatggacaccatccaggaagaccccagcacagactcacatatggacgaggatgggtttgagaaggaccccttcccaaatagcagcacagctgccaagtcatttgaggatctcacggaccatccggtcaccagaagtgaaaaggctgcctcctttaaactgcagcgtcagaatcgtgttgacagcaaagaaacagagtgctaatttagttctcagctcttctgacttaagtgtgcaaaatatttttatagatttgacctacaaatcaatcacagcttgtattttgtgaagactgggaagtgacttagcagatgctggtcatgtgtttgaacttcctgcaggtaaacagttcttgtgtggtttggcccttctccttttgaaaaggtaaggtgaaggtgaatctagcttattttgaggctttcaggttttagtttttaaaatatcttttgacctgtggtgcaaaagcagaaaatacagctggattgggttatgaatatttacgtttttgtaaattaatcttttatattgataacagcactgactagggaaatgatcagtttttttttatacactgtaatgaaccgctgaatatgaggcatttggcatttatttgtgatgacaactggaatagttttttttttttttttttttttttgccttcaactaaaaacaaaggagataaatctagtatacattgtctctaaattgtgggtctatttctagttattacccagagtttttatgtagcagggaaaatatatatctaaatttagaaatcatttgggttaatatggctcttcataattctaagactaatgctctctagaaacctaaccacctaccttacagtgagggctatacatggtagccagttgaatttatggaatctaccaactgtttagggccctgatttgctgggcagtttttctgtattttataagtatcttcatgtatccctgttactgatagggatacatgctcttagaaaattcactattggctgggagtggtggctcatgcctgtaatcccagcacttggagaggctgaggttgcgccactacactccagcctgggtgacagagtgagactctgcctcaaaaaaaaaaaaaaaaaaaaa

SEQ ID NO: 158 - DNA, 2399 bp, Homo sapiens
aacgctgctcaacggtctctgtccttggctgtggctcctgcgctctggctgagccatgttccttctcctcgccctcctcactgagcttggaagactgcaagcccacgaaggttctgaaggaatatttctgcatgtcacagttccacggaagattaagtcaaatgacagtgaagtttcagagaggaagatgatttacatcattacaattgatggacaaccttacactctacatctcggaaaacaatcattcttaccccagaactttttggtttatacatataatgaaactggatctttgcattctgtgtctccatattttatgatgcattgccattaccaaggatatgctgccgaatttccaaattcatttgtgacactcagtatatgttctggtctcaggggatttctccagtttgaaaatatcagttatggaattgaaccagtagaatcttcagcaagatttgagcatataatttatcaaatgaaaaataatgatccaaatgtatccattttagcagtaaattacagtcatatttggcagaaagaccagccctacaaagttcctttaaactcacagataaaaaatctttcaaaactattaccccaatatctggaaatatacattatagtggaaaaagctttgtatgattatatgggatctgaaatgatggctgtaacacaaaaaattgtccaggttattgggcttgtcaacactatgtttacccagttcaaattgactgttatactgtcttccttggaattgtggtcaaatgaaaaccagatttccaccagtggggatgctgatgatatattacaaagatttttggcatggaaacgggactatctcatcctacggccccatgacatagcatacttacttgtttacaggaaacatcctaaatatgtgggagcaacatttcctggcactgtatgcaataaaagctatgatgcaggtattgctatgtatccagatgcaataggtttggagggattttcggttattatagctcaactgcttggccttaatgtaggattaacatatgatgacatcactcagtgtttctgtctgagagctacatgcatcatgaatcatgaagcagtgagtgccagtggtagaaagatttttagcaactgcagcatgcacgactatagatattttgtttcaaaatttgagactaaatgccttcagaagctttcaaatttgcaaccattacatcaaaatcaaccagtgtgtggtaatgggattttggaatccaatgaagaatgtgactgtggtaataaaaatgaatgtcaatttaagaagtgctgtgattataacacatgtaaactgaagggctcagtaaaatgtggttctggaccatgttgtacatcaaagtgtgagttgtcaatagcaggcactccatgtagaaagagtattgatccagagtgtgattttacagagtactgcaatggaacctctagtaattgtgttcctgacacttatgcattgaatggccgtttgtgcaagttgggaactgcctattgctataacggacaatgtcaaactactgataaccagtgtgccaagatatttggaaaaggtgctcaaggtgctccatttgcctgttttaaagaagttaattctctgcatgaaagatctgaaaactgtggttttaaaaattcacaaccattaccttgtgaacggaaggatgttctctgtggaaaattagcttgtgttcagccacataaaaatgctaataaaagtgacgctcaatctacagtttattcatatattcaagaccatgtatgtgtatctatagccactggttcctccatgagatcagatggaacagacaatgcctatgtggctgatggcaccatgtgtggtccagaaatgtactgtgtaaataaaacctgcagaaaagttcatttaatgggatataactgtaatgccaccacaaaatgcaaagggaaagggatatgtaataattttggtaattgtcaatgcttccctggacatagacctccagattgtaaattccagtttggttccccagggggtagtattgatgatggaaattttcagaaatctggtgacttttatactgaaaaaggctacaatacacactggaacaactggtttattctgagtttctgcatttttctgccgtttttcatagttttcaccactgtgatctttaaaagaaatgaaataagtaaatcatgtaacagagagaatgcagagtataatcgtaattcatccgttgtatcagaaagcgatgacgtgggacattaatattgcacagaacttccatagcaaataacctaaaggaacgaatgtgctttatttataaccttacgttatccccaatgcattgtaaatgtcaaacttttggaaaataaagcctgcgtgccctccc

SEQ ID NO: 159 - DNA, 6506 bp, Homo sapiens
agacgcgcctccaccgccgggcagtgggcaggtatggctgagggcgtgtgagcgccgagcgctaagggccgccgccaccatgccagggggcgcaggcgccgcccggctctgcttgctggcgtttgccctgcagcccctccggccgcgggcggcgcgggagcctggatggacaagaggaagtgaggaaggcagccccaagctgcagcatgaacttatcatacctcagtggaagacttcagaaagccccgtgagagaaaagcatccactcaaagctgagctcagggtaatggctgaggggcgagaactgatcctggacctggagaagaatgagcaactttttgctccttcctacacagaaacccattatacttcaagtggtaaccctcaaaccaccacacggaaattggaggatcactgcttttaccacggcacggtgagggagacagaactgtccagcgtcacgctcagcacttgccgaggaattagaggactgattacggtgagcagcaacctcagctacgtcatcgagcccctccctgacagcaagggccaacaccttatttacagatctgaacatctcaagccgcccccgggaaactgtgggttcgagcactccaagcccaccaccagggactgggctcttcagtttacacaacagaccaagaagcgacctcgcaggatgaaaagggaagatttaaactccatgaagtatgtggagctttacctcgtggctgattatttagagtttcagaagaatcgacgagaccaggacgccaccaaacacaagctcatagagatcgccaactatgttgataagttttaccgatccttgaacatccggattgctctcgtgggcttggaagtgtggacccacgggaacatgtgtgaagtttcagagaatccatattctaccctctggtcctttctcagttggaggcgcaagctgcttgcccagaagtaccatgacaacgcccaattaatcacgggcatgtccttccacggcaccaccatcggcctggcccccctcatggccatgtgctctgtgtaccagtctggaggagtcaacatggaccactccgagaatgccattggcgtggctgccaccatggcccacgagatgggccacaactttggcatgacccatgattctgcagattgctgctcggccagtgcggctgatggtgggtgcatcatggcagctgccactgggcacccctttcccaaagtgttcaatggatgcaacaggagggagctggacaggtatctgcagtcaggtggtggaatgtgtctctccaacatgccagacaccaggatgttgtatggaggccggaggtgtgggaacgggtatctggaagatggggaagagtgtgactgtggagaagaagaggaatgtaacaacccctgctgcaatgcctctaattgtaccctgaggccgggggcggagtgtgctcacggctcctgctgccaccagtgtaagctgttggctcctgggaccctgtgccgcgagcaggccaggcagtgtgacctcccggagttctgtacgggcaagtctccccactgccctaccaacttctaccagatggatggtaccccctgtgagggcggccaggcctactgctacaacggcatgtgcctcacctaccaggagcagtgccagcagctgtggggacccggagcccgacctgcccctgacctctgcttcgagaaggtgaatgtggcaggagacacctttggaaactgtggaaaggacatgaatggtgaacacaggaagtgcaacatgagagatgcgaagtgtgggaagatccagtgtcagagctctgaggcccggcccctggagtccaacgcggtgcccattgacaccactatcatcatgaatgggaggcagatccagtgccggggcacccacgtctaccgaggtcctgaggaggagggtgacatgctggacccagggctggtgatgactggaaccaagtgtggctacaaccatatttgctttgaggggcagtgcaggaacacctccttctttgaaactgaaggctgtgggaagaagtgcaatggccatggggtctgtaacaacaaccagaactgccactgcctgccgggctgggccccgcccttctgcaacacaccgggccacgggggcagtatcgacagtgggcctatgccccctgagagtgtgggtcctgtggtagctggagtgttggtggccatcttggtgctggcggtcctcatgctgatgtactactgctgcagacagaacaacaaactaggccaactcaagccctcagctctcccttccaagctgaggcaacagttcagttgtcccttcagggtttctcagaacagcgggactggtcatgccaacccaactttcaagctgcagacgccccagggcaagcgaaaggtgatcaacactccggaaatcctgcggaagccctcccagcctcctccccggccccctccagattatctgcgtggtgggtccccacctgcaccactgccagctcacctgagcagggctgctaggaactccccagggcccgggtctcaaatagagaggacggagtcgtccaggaggcctcctccaagccggccaattccccccgcaccaaattgcatcgtttcccaggacttctccaggcctcggccgccccagaaggcactcccggcaaacccagtgccaggccgcaggagcctccccaggccaggaggtgcatccccactgcggccccctggtgctggccctcagcagtcccggcctctggcagcacttgccccaaagtttccagaatacagatcacagagggctggagggatgattagctcgaaaatctagacctgtccaaggggcttctccctttccttgagctctctggacactgcagaggacccatggccatggaaccctgaagaagcatgtctggccgcctctgagctcctcccaccctcctccaggaacctccacatctccaaaaatctccctgttgactcagtgcctcctcggcttccttggaagcccagagggactatgatctgatggcctctaggtgttgttttgtgcaatatacagccccaggtagggaggggagagtatgaggagggtgactggcagcttctcctccagactcctagccccgaggtgctgatggagatgctcaaggccagcaagcccctcaggccagcacttcgcttgcagaagccatccattcactcctggggtgcagggcacgcaagagagcttcccattgcttctgctctcctcagaggtcccgggctggatggaggctggtacttacccaccccttttagcttttagggattaaggaagggtcaagccagccactgctgtggccctgcccagggcttggttgagggaacggcttctggctgtatggctgcatgtgacaagccacgtcccctcccacctctccccaaacccctgcatccctgtattcacacgggtcactctgactcagacaggtactattcgtaggcagtgtagacagcaggaggagcaccgggcttgggcttcctctgagccgtgatgccaaaggttgcgactcctgactctggataatttttagttgctctttgttttctctgccgcactttcctggtgccccacgcttttctctcttccttcccctctcattctccctctaatgtgtggtgctttggtgagcaaaccctcagcagtcctgaccttcgggtgaccaggtgcttgtgacctacaagtcagagtcctctctcacagtcggccactggatttccctcactggctctcaggagtgtgaccagagtagacttggggcatggccattggggtcatatgtttatttttcattgtgttttgtgacctcagcagggtgggggtcttcctccttactctaagctaaatctaggtgaggtttccccttagggagcccagctatttacaaagtacacacgagggagcaggctggtcattgacttcgggctggaccgttgccctctgagcagagaacagacccatttctgggagctgcccgagatcactggagaaggcagccagcagcagctgcactggaacagtcagagcagggagcctcttcctcaacccagctttttgtcattcacttccttttgttctctctctggtcactgcccttacctgaccctcacagaaagagagctctgagcaggtgagggggtctgcggtggctcctgtcttccctgcagcagggaaggagggccgtgtggtgctttgctagataggacggtttttgcaaagcacctggagatgtttgctgggagatagactcccactccacaaaggtgctgggtggctctccggacaggagctggcctgactctcactcctctgaggctttcctggggcctcctcccatcctgccatgagcaattgtttgctcttgaaaacctcactgcaaggctgaggctgagcttctgattcaccaccccagggcctccttatagttctctgcacacaataggtgcttcttggatgttcttgggtttggaaataagtggaaaatacgggatgtacccctgggggaaaagcctgggttgggtttagaaagatctcaggaaaatgagtttctcttccctcagggtggctgtgatacaggttccccatgtccttgccgtgggtcatccttgctgtgggtcatccttgctgtggagatccattccccacctttcctgtggcccaaccttttatttaaatgtgctaccctctgcctcaaggcttggttcctggaaagtaaaggtgaaaacatcccctttcacccctctgcaaaacaaacaagcaacatcctcaaaacccaaccccatgcctcacagagcttcctgtggcttctccagcctttctccctcacatcaggaggtagatagctctgaaatgacagcgccacagccatagtgactgcatgagccatctgaacctgcagtccaccctccctggaaccacaccagaaagagacctgggttgtcgttttcttgctttttgttttgttttgttttattattttcatatcacctccatcccataaagttgtactgtgaactggaagatggtggaatgttttggaatttgatagactttcggcaaccagttctactaatgcttcactcctggctctgttcagggaggctgcccaggaggaagactggccattatgcatccccttttctttccagtgcccagtatgctgttttgaggtgtcaaatacaaataaatctgggcttagggaaggagagaccttattccaaagcacgattgcagaaggggaaagggaatattgcaaaagggagaggaaggggccttatgggaatagtgaaaaggctcagaccgaccgatggcaagatctgcaagcgtctcaaagcccaggcagaaaaggacttttcttttattggaagaagtaaacatggctagaaagaaccacgttcagggaatgacgttgtgcccagcctttttttttttttttttttttttgtctccaggggaggggctgtttgctggctcaggctgaggatggcccaaagtccagggtctggtggggaggagggaagcttaactcaagtttgggttagtgagttagcaagctctttgtgcagatggggatgtaggtaaatctttttaaaagtgaaattaacctcctgccaattttacaacccaagaatttttttttaagggccttggagccatctctaaaacaaacctcaagggatttagtgccctgtctccctgtctctagaagccttagcctgggcacctggctcaatcttgtaactgcctgctagccatagattcctttcagccttgctgacttctccctataaaagtaaagcctttttctgccccagctctgagacacttgcagatcttaaggtctgagacttgctgattttctggttggagtgtttttttgtattgccatagtcccttccccctgaagcaatagcccctccccacctcctgcaatacgcctttccaatctttattggaagtctctccctgcctacttcctaatttattcttatttgacagagggtatggaagacttgcaatttgaaaactggggaccagttccaaagtcagtaattgtgttaaccacgtgtataacagctctgctggacacccaagaaagccatgggaacgccaactggaaaggtccccttccccaggggagcctgcgaaggagaggttctgtagaatccaagcccacatttccaaagtcacccccaacgcgtcctctcacaccgtccactgtgcgtttgtatgtgtctgggatccagggcaatgtgaattttctttttatttgggagattgttcacggaaaacagatcttcttctctcttgtccacctattaattgtttacaatatttgtacatctatgcaaaatacttgaatgggccatggtgccttttttccttgttagtatttaattaaaaatgaattgtttgtcatttgcaatgttaaaaaaaaaaaaaaaaaaaaaaaaaa

SEQ ID NO: 160 - DNA, 2807 bp, Homo sapiens
gggagttcagcagtgtgtttaagactagggtcatgcccacatcaatctcagattcccattttatctttcttttttggtatcactcctagtaccaagtcttgtgtctgtcaatatcctgtccaaaaaagaaaaacacaccaaggaaaattaatataggaaaaatttaaaaagttattagaggactgaaaatataaaaatggaacactgaaagacacagagtttttattttcagcactgcagctctgatggtccagctccaccaggacacagatccccagatccctaaaggtcagccatgcaccctgaacagctcagagggaggagccagaccagcagtgcctcacaccttgttctcttctgctctagacagatggctccataatgacagcttcataatggcagtgggtgagcccctggtgcacatcagggtcactcttctgctgctctggtttgggatgtttttgtctatttctggccactctcaggccaggccctcccagtatttcacttctccagaagtggtgatccctttgaaggtgatcagcaggggcagaggtgcaaaggctcctggatggctctcctatagcctgcggtttgggggacagagatacattgtccacatgagggtaaataagctgttgtttgctgcacaccttcctgtgttcacctacacagagcagcatgccctgctccaggatcagcccttcatccaggatgactgctactaccatggttatgtggagggggtccctgagtccttggttgcccttagtacctgttctgggggctttcttggaatgctacagataaatgaccttgtttatgaaatcaagccaattagtgtttctgccacatttgaacacctagtatataagatagacagtgatgatacacagtttccacctatgagatgtgggttaacagaagagaaaatagcacaccagatggagttgcaattgtcatataatttcactctgaagcaaagttcttttgtgggctggtggacccatcagcggtttgttgagctggtagtggtcgtggataatattagatatcttttctctcaaagtaatgcaacaacagtgcagcatgaagtatttaacgttgtcaatatagtggattccttctatcatcctttggaggttgatgtaattttgactggaattgatatatggactgcatcaaatccacttcctaccagtggagacctagataatgttttagaggacttttctatttggaagaattataaccttaataatcgactacaacatgatgttgcacatcttttcataaaagacacacaaggcatgaagcttggtgttgcctatgttaaaggaatatgccagaatccttttaatactggagttgatgtttttgaagacaacaggttggtcgtttttgcaattactttgggccacgagcttggtcataatttgggtatgcaacatgacacccagtggtgtgtgtgcgagctacagtggtgcataatgcatgcctatagaaaggtgacaactaaatttagcaactgcagttatgcccaatattgggacagtactatcagtagtggattatgtattcaaccgcctccatatccagggaatatatttagactgaagtactgtgggaatctagtggttgaagaaggggaggaatgtgactgtggaaccatacggcagtgtgcaaaagatccctgttgtctgttaaactgtactctacatcctggggctgcttgtgcttttggaatatgttgcaaagactgcaaatttctgccatcaggaactttatgtagacaacaagttggtgaatgtgaccttccagagtggtgcaatgggacatcccatcaatgcccagatgatgtgtatgtgcaggacgggatctcctgtaatgtgaatgccttctgctatgaaaagacgtgtaataaccatgatatacaatgtaaagagatttttggccaagatgcaaggagtgcatctcagagttgctaccaagaaatcaacacccaaggaaaccgtttcggtcactgtggtattgtaggcacaacatatgtaaaatgttggacccctgatatcatgtgtgggagggttcagtgtgaaaatgtgggagtaattcccaatctgatagagcattctacagtgcagcagtttcacctcaatgacaccacttgctggggcactgattatcatttagggatggctatacctgatattggtgaggtgaaagatggcacagtatgtggtccagaaaagatctgcatccgtaagaagtgtgccagtatggttcatctgtcacaagcctgtcagcctaagacctgcaacatgaggggaatctgcaacaacaaacaacactgtcactgcaaccatgaatgggcacccccatactgcaaggacaaaggctatggaggtagtgctgatagtggcccacctcctaagaacaacatggaaggattaaatgtgatgggaaagttgcgttacctgtcactattgtgccttcttcctttggttgcttttttattattttgcttacatgtgctttttaagaaacgcacaaaaagtaaagaagatgaagaaggataagagaaatgggaaaaagaaggagactaaactttatacttcatttttaatatccaattttttaatagaaaaatatgaagccatgtctcactgtttaaataaaacttcatggacatttcatgtcaggattgcaagcattagctatcacagcaaaggattcctagcctattcttacttactctacagtgtcttaagcaatattaaaggttccttttcccaaaaaaaaaaaaaaaa

SEQ ID NO: 161 - DNA, 2406 bp, Homo sapiens
atggcagtggatgggaccctcgtgtacatcagggtcactcttctgctgctctggcttggggtatttttgtctatttccggctactgtcaggctgggccctcccagcatttcacttccccggaagtggtgatccccttgaaggtgatcagcaggggcagaagtgcaaaggctcctggatggctctcctatagtctgcggtttgggggccagaaacacgttgttcatatgagggtcaagaagctcttagtttctagacacctcccagtgttcacctacacagatgagcgtgcactcctggaggatcagctcttcatcccagatgactgttactatcatggttacgtggagggtgcccctgagtctctggttgtgttcagtgcttgttttgggggctttcgaggagtattaaaaataagtggcctcacttatgaaattgaacccatcaggcactctgccacatttgaacacctggtttacaaagtaaacagtaatgagacacaattcccagctatgagatgtggcttaacagagaaggaagtagcacgccaacagttggaatttgaagaggctgagaactcagctctggaaccaaaatctgctggtgactggtggactcatgcatggtttctggagctagttgttgtggtgaaccatgatttcttcatttactctcaaagcaacatctcaaaggtgcaagaggatgtatttcttgttgtcaacatagtggattccatgtatcagcagttaggtacttacataattttgattggaattgaaatttggaatcaaggaaatgttttcccaatgacaagcatagaacaggtcctgaacgatttctctcaatggaaacaaatcagtctttcccagctacagcatgatgctgcacatatgttcataaaaaattcacttataagtatacttggcctagcctatgttgcaggaatatgtcgtccacctattgattgtggagttgataattttcaaggagatacctggtctctttttgccaacactgtggcccatgagttaggtcatacgttgggtatgcagcatgatgaagaattctgtttttgtggggaaagaggttgcatcatgaatacttttagagtgccagcagagaaattcaccaattgcagttacgctgattttatgaagaccaccttaaaccagggatcatgtctgcataatcctccaagattgggggaaatctttatgctaaagcgctgtgggaatggtgtggttgaaagagaagagcagtgtgactgtggatccgtacagcagtgtgaacaagacgcctgttgtctgttgaactgcactctaaggcctggggctgcctgtgcttttgggctttgttgcaaagactgcaagttcatgccatcaggggaactctgtagacaagaggtcaatgaatgtgaccttccagaatggtgcaatggaacatctcatcagtgtccagaagatagatatgtgcaggacgggatcccctgtagtgacagtgcctactgctatcaaaagaggtgtaataaccatgaccagcattgcagggagatttttggtaaagatgcaaaaagtgcatctcagaattgctataaagaaatcaactctcagggaaaccgttttggtcactgtggtataaatggcacaacatacctaaaatgtcatatctctgatgtcttttgtgggagagttcaatgtgagaatgtgagagacattcctcttctccaagatcattttactttgcagcacactcatatcaatggtgtcacctgctggggtattgactatcatttaaggatgaacatatctgacattggtgaagtgaaagatggtactgtgtgtggcccaggaaagatctgcatccataagaagtgtgtcagtctgtctgtcttgtcacatgtctgccttcctgagacctgcaatatgaaggggatctgcaataacaaacatcactgccactgtggctatgggtggtccccaccctactgccagcacagaggctatgggggcagtattgacagtggcccagcatctgcaaagagaggagtttttttgccgctgattgtgattccttctttgtctgttttgactttcctgtttactgtcgggcttcttatgtatctacgacaatgttctggtcccaaagaaactaaggctcattcatcaggttaagaaaatgtctctaacttaatattccatgcattagtacactttagtctcttggcagtagaaacattagtacatccctgaaactgagcacatttctgaccatttccagaaagctgcaaagatcttcccttacattagtaccacaaacattgtcattaagttcaagttattcttaacatgtttctatctattgtttattgttttaagcagcaaataaagctacatccttccctcccttta

SEQ ID NO: 162 - DNA, 9334 bp, Homo sapiens
actcgctcgctccccccgccagcggaagcgtccgcgaagcacaatgcagcactgagccgcggtggaggttgcagcgccacggccgccgcagcaccggccggggctgggtggaggtggccgcggggaccccgggggcgcggagcgagggaaacggactcggcggcgccggcatgaggagctgagcgtctcgggcgaggcgggctgacggcagcaccatgcaggcggcagtggctgtgtccgtgcccttcttgctgctctgtgtcctggggacctgccctccggcgcgctgcggccaggcaggagacgcctcattgatggagctagagaagaggaaggaaaaccgcttcgtggagcgccagagcatcgtgccactgcgcctcatctaccgctcgggcggcgaagacgaaagtcggcacgacgcgctcgacacgcgggtgcggggcgacctcggtggcccgcagttgactcatgttgaccaagcaagcttccaggttgatgcctttggaacgtcattcattctcgatgtcgtgctaaatcatgatttgctgtcctctgaatacatagagagacacattgaacatggaggcaagactgtggaagttaaaggaggagagcactgttactaccagggccatatccgaggaaaccctgactcatttgttgcattgtcaacatgccacggacttcatgggatgttctatgacgggaaccacacatatctcattgagccagaagaaaatgacactactcaagaggatttccattttcattcagtttacaaatccagactgtttgaattttccttggatgatcttccatctgaatttcagcaagtaaacattactccatcaaaatttattttgaagccaagaccaaaaaggagtaaacggcagcttcgtcgatatcctcgtaatgtagaagaagaaaccaaatacattgaactgatgattgtgaatgatcaccttatgtttaaaaaacatcggctttccgttgtacataccaatacctatgcgaaatctgtggtgaacatggcagatttaatatataaagaccaacttaagaccaggatagtattggttgctatggaaacctgggcgactgacaacaagtttgccatatctgaaaatccattgatcaccctacgtgagtttatgaaatacaggagggattttatcaaagagaaaagtgatgcagttcaccttttttcgggaagtcaatttgagagtagccggagcggggcagcttatattggtgggatttgctcgttgctgaaaggaggaggcgtgaatgaatttgggaaaactgatttaatggctgttacacttgcccagtcattagcccataatattggtattatctcagacaaaagaaagttagcaagtggtgaatgtaaatgcgaggacacgtggtccgggtgcataatgggagacactggctattatcttcctaaaaagttcacccagtgtaatattgaagagtatcatgacttcctgaatagtggaggtggtgcctgccttttcaacaaaccttctaagcttcttgatcctcctgagtgtggcaatggcttcattgaaactggagaggagtgtgattgtggaaccccggccgaatgtgtccttgaaggagcagagtgttgtaagaaatgcaccttgactcaagactctcaatgcagtgacggtctttgctgtaaaaagtgcaagtttcagcctatgggcactgtgtgccgagaagcagtaaatgattgtgatattcgtgaaacgtgctcaggaaattcaagccagtgtgcccctaatattcataaaatggatggatattcatgtgatggtgttcagggaatttgctttggaggaagatgcaaaaccagagatagacaatgcaaatacatttgggggcaaaaggtgacagcatcagacaaatattgctatgagaaactgaatattgaagggacggagaagggtaactgtgggaaagacaaagacacatggatacagtgcaacaaacgggatgtgctttgtggttaccttttgtgtaccaatattggcaatatcccaaggcttggagaactcgatggtgaaatcacatctactttagttgtgcagcaaggaagaacattaaactgcagtggtgggcatgttaagcttgaagaagatgtagatcttggctatgtggaagatgggacaccttgtggtccccaaatgatgtgcttagaacacaggtgtcttcctgtggcttctttcaactttagtacttgcttgagcagtaaagaaggcactatttgctcaggaaatggagtttgcagtaatgagctgaagtgtgtgtgtaacagacactggataggttctgattgcaacacttacttccctcacaatgatgatgcaaagactggtatcactctgtctggcaatggtgttgctggcaccaatatcataataggcataattgctggcaccattttagtgctggccctcatattaggaataactgcgtggggttataaaaactatcgagaacagagacagttaccccagggagattatgtaaaaaagcctggagatggtgactctttttatagcgacattcctcccggagtcagcacaaactcagcatctagttctaagaagaggtcaaatgggctctctcattcttggagtgaaaggattccagacacaaaacatatttcagacatctgtgaaaatgggcgacctcgaagtaactcttggcaaggtaacctgggaggcaacaaaaagaaaatcagaggcaaaagatttagacctcggtctaattcaactgagactttatctcctgccaagtctccttcttcatcaactgggtctattgcctccagcagaaaatacccttacccaatgcctccacttcctgatgaggacaagaaagtgaaccgacaaagtgccaggctatgggagacatccatttaagatcaactgtttacatgtgatacatcgaaaactgtttacttcaacttttatagaaacccaggctcatggaatcactgcaaatctatctgctcttcagacaatacgaagaccctctgagatgctacagaggagaggaagcggagtttcacatctggttaccattttctttttgtcattggcttaggatttaactaaccatgaaaagaactactgaaatattacactataacatggaacaataaaggtactggtatgttaatggataatccgcatgacagataatatgtagaaatattcataaagttaactcacatgacccaaatgtagcaagtttcctaaggtacaatagtggattcagaacttgacgttctgaggcacatcctcactgtaaacagtaatgctatatgcatgaagcttctgtttattgttttccatatttaaggaaacaacatcccataatagaaatgagcatgcagggctaaggcatataggatttttctgcaggactttaaagctttgaaaggccaatatcccataggctaactttaaacatgtatttttatttttgttttgttttttacttttcatatttatattagcatacaaggacaattgtatatatgtaacatttttaaaattttaaaaaaatgcagctgttacacacaagtgtattttgccaaatgcctaaaaattccgtcacaatcacatcatcgtcatccatccagtgatcttcaaagactataagcaggtaatgtaaatatagtggtcaatgctgtaaatgtgtctctccataattcgtatttgtccaaaacatgtgatatccctttaacctgtgcacagtctggaggcagttttattgagtgagtagttgagtgagagaacaaaatgcccagcaaaacttctggcctccacagtttgctgatgcaggagcccactctgctgccgatgggctgctccctggaaatcatctatcccatccgtccatcccatctcatcccagtgagccagcccctcagcagtgtgtgctgctgagattaagcaagtgcaccaaagacacatgagacaacgtactgccagtgagctggtagcttctggtttgccaatactctaaccctttgtggcctgaagttttgttccttggcaaaactttgtagtcacccttatctgtgaaatgaatgacttgttttaacaaaaacaactttgacactgttgattaagaatatttgtcatcactgggatgtgaatcccaacaagtcattctgcactgactctgaagcaagatgacggaagattctactgtttgagttgagcagcttcaaccattcataaagggttaagttagatctaaaccacagagatgcagcatctgcgaagttacactaacagctggtgggggaaagaatctgatgctttgaaacatatatttttaatccataggaaaataagattctattttaaaagacccttctttaaaaccaaagtttgatagctgttataatggcagatctgtcattccaaaagaaagctagctaccctagtgaggctggagattgatgggctcaatcacatggtctgctctgaagtatacttctgatttcctcatttgctgtgtgttcttaagaggaatggaatctgcagacaggttttcagatatactctcttaaatttgatcctctcccatagtatccatggacaaaatccatatcatcttcatatgtaatcaatttgattacatatcatgcccaaactgaagggccgagttagaaggcagcattttatactgtgtgtttatgtcttctgtaatggacctctcttttgaaagtctttgaaatgtttggaaagcaatttaatgatccaaaattacataatgtaaaacagcatttttcatgacaggaagggatgtgattgttgatatgaccataaagcgatccatcaggccaaggtttgggtgagtgttgggcagtgttgccacatagctcaagccaagaacagaagggcagacagattgaaagatgtagcaaataaagtgactttttaccaagatcactgtaacgtgcactgaatgtttcctggtactgagtgtacaaaaggctaatgctataaggtttattttgactataaaagagtttatagatttgaatataaggtgcctcatgtaaatatgcttcctagttacaggtacacaaactctaatttgaaagaatccacaaatggctagctgagaagttaacagatctctatcattcgtctgtgtgtctttcttccctcacctatttttacatgaaattattatttcaaatgaaaatcatgatcatgtgccttttgggagacatggtttctctagaggtatatagtttgtaattacccgccttggtaataaccttcctgtggaactgactaaaaatttttgttttatttgtagagattatgatttgaggtatacagtaactttaatgttctattcttggaatctactctatctcccatgtttaaacattcttagagccatttgtgggattcattgacaaatttatagtctcaagggagagtttcaagaagtatctatatgtatactcatggttggggttctagaggcattacatctaaatatttatgacttttcatccaccgaggtaggtagcattaaatatacaagcacagcattatttctctttaaatatctattttggagagaaactttgaggccgtatcacagtttatatcatgcaactaatatttatatttcccaatccactacaaaatcattgctaagcagaagagagcagttatttggcctttatgttccattgtatgcctttttcccctcttccagttttatgcattgataagactacagattggcaccctgctgcctgagaagttccatttatccatagtcattgcagtgcagtaaaccctgatttaaaggaacatttaagtctctccagtaagtgtgaacaggaaggcatgggatagaagagagtccttatggagtggactggccacatgaactgtcctccagccactctttatttagatgggaagtggcatggtcctctgaacgctgcaggctggaaatgatccccagggcacatgcaatctgtggaaaggaatacatcggacagtgcaacatggtgagggctttcaatttgctgtggttgctgtatttttaacaagacacccggagcttcgattaggaataacctatcattagagttgttcctttaaaaaatgttgatgaataaagcaaatgtgggtttaaaatgccaacttatttttcactttgatattggtgctttattaagtgataccatagtttctctctcttttccctgttactttaaaaagaattttaaaattgtgtttgtatataaggtcagtctggcatagaatttctgaaagtaacattaataggatttctaaagaagttgtcagaaagacaaaattggatattctctgcactttcagagaaggactatttatgtttgaatttcataacaggcttacatttcttttctattgattcccttgcatatctacataatttaatcctgtctattgtcccagatctcttgtggctactttacctgttaccttttttgggattttgttctttatgttttacagttaccttcggaccagaagatatatggtatttttattcacttttttcagtgttttaaaataaggtttgttcatttattcattttatccattcatttcctattgtcgtatagttctaaccatatatatagtactttaaatattttcttatgttattatttaacacaataataaattcttatctaaagaaaagattattttactttattttacttaaaggaaagagaatttattggcaggatattgagtagcttgtagagtatatggaaggggagagccaagttaggaactggcaggagcgaggagggacaagacagctgcctgcacagtctgataggacacatctggctgctgccactggatgcccacatgggcagcgtgttgtaaatatcaccctaccagttggtaagttggcatgaattttccatgaaacacttgactgccatttgcagcatcatatctactttttatgcacctaattatatttttcctttcacttttatgtttttttctaaaactgccacctaaatacctttccactccgttaaatgtatggagtcaagataaatttctcattcttgctctacagtttaagatgtataagaagtaaacacttccgtggaatagggcttaatttggggctcaaatgttttgttttcctcctgagatgtttttgaatggataagactactctggttttggatgcagcaacttttctcctggcatggaagaactaaggactttgataaacagtttcccattgatagtataatataatgaggggcccagtctgctcttgtgaaaattggctgagcggcagcagcccatcccagctgtgtgtgagtagccccgctgttaaaagtacacctgctgggaattgttggcacatgctcatagcatatgctctgtgggggaggtgcttttaattccaagaggagcttttctcccacatctgcggatgcagaacaaagatgcagcaggactttagaaaacaggagtgtttctactgacattttattcattgctttgttagtcccgcaaatggagcaacttctggttgtttgtaagcagaaaaacaaaacattcaaaaccaaaaacctaattagtacttttgtcctttatatccctaggatgctctagggagtagaggtggcaggcatctttatcttcacccactggcacattagcagcttatgcaacttaggaagtcactgggagatgagtcctaaaagcctgaatttcaggatggggaggagataagaagcagagaactttgggagaccatggggcatttagtcctggcatagacacagaatggatattcacaggaagattcctgaggccagatatgcaggagagcacacattgtcacaaggggtgtggtgggaagagaggtgctggatgcctaagacagacttctgtgcttggcagattgactgagtgttgactctgaatactttgtggtccagtccataataggccagtcactcctgcctgatgcatccctgttgcactgttcataaaacctgtatcatatggggaagcttcaaggctggcaggcttttcatcaagtaatcatcattccatgttcctacgtatccttgaaatcaaaaccaggcaatatgtgtgtttttggttttgttttacaaaaaggcatacaatgagacgaattcttaagataatgcacatacagaatcatcaataaagtttcaaagagtttatgaagaaaaggtattttcctttcttgtaaaaaatgttatatatatataatattctgatctgcattcaccttagggcttagcctcttattttatgaattttcaggctgtgatttgtgttggatggctaagctctaaaataatgcaaatagccccaatctttaaatatagcggtgctaagttgaacaagtaacacaatacagaaaatgctgatatgaatacttagtaataatacaaaagttcctgctaaattatatatgtgtatcactgcctgactagaaaccccacttttcttttctaatccagcacaaaatcaaactctgattctacaaccagtctttttaaagggcaaaaatgactcaacacctttgttttgtatggaaaacttttttttttacactaactgcaaaactgctttaaaaaaaggcttattcaggatagataagcatgtactccttttttaacttgctggaagacttgcccctccaaactagcacccccaaaagacaacttctttcagaaacggggtgttttacctaaacatagtagcttacatgttagccagcagtaggtcggcactagtgttttccacggttatcacctttgacaggtgatgtgcatctatagatagtggaagccaccccatgaggaggtgttaatagcagcatggtttcacttttggtaatcaggtaatcatgtgtatatacttagattcgcattattttaacatttctctgctactctgcacttcaggttcgttaagctattttaataattactggggttatggcaaacaccaatggaaatgtatatggcaactgctttcctgagcaagtgtgatttgttttatggctgttcaagttataaaattgttcttacattgtaggtaaacaaaatcttgatgtttttaaaggtcactgtaacttaaggttcaaatttctggcacagttttattagtattcacttcggaagctaataagataccatggttttctatgttactcccattgtaacattagtaaagtgactttcaataaaagatttatgttattttga

SEQ ID NO: 163 - DNA, 3059 bp, Homo sapiens
tcctctgcgtcccgccccgggagtggctgcgaggctaggcgagccgggaaagggggcgccgcccagccccgagccccgcgccccgtgccccgagcccggagccccctgcccgccgcggcaccatgcgcgccgagccggcgtgaccggctccgcccgcggccgccccgcagctagcccggcgctctcgccggccacacggagcggcgcccgggagctatgagccatgaagccgcccggcagcagctcgcggcagccgcccctggcgggctgcagccttgccggcgcttcctgcggcccccaacgcggccccgccggctcggtgcctgccagcgccccggcccgcacgccgccctgccgcctgcttctcgtccttctcctgctgcctccgctcgccgcctcgtcccggccccgcgcctggggggctgctgcgcccagcgctccgcattggaatgaaactgcagaaaaaaatttgggagtcctggcagatgaagacaatacattgcaacagaatagcagcagtaatatcagttacagcaatgcaatgcagaaagaaatcacactgccttcaagactcatatattacatcaaccaagactcggaaagcccttatcacgttcttgacacaaaggcaagacaccagcaaaaacataataaggctgtccatctggcccaggcaagcttccagattgaagccttcggctccaaattcattcttgacctcatactgaacaatggtttgttgtcttctgattatgtggagattcactacgaaaatgggaaaccacagtactctaagggtggagagcactgttactaccatggaagcatcagaggcgtcaaagactccaaggtggctctgtcaacctgcaatggacttcatggcatgtttgaagatgataccttcgtgtatatgatagagccactagagctggttcatgatgagaaaagcacaggtcgaccacatataatccagaaaaccttggcaggacagtattctaagcaaatgaagaatctcactatggaaagaggtgaccagtggccctttctctctgaattacagtggttgaaaagaaggaagagagcagtgaatccatcacgtggtatatttgaagaaatgaaatatttggaacttatgattgttaatgatcacaaaacgtataagaagcatcgctcttctcatgcacataccaacaactttgcaaagtccgtggtcaaccttgtggattctatttacaaggagcagctcaacaccagggttgtcctggtggctgtagagacctggactgagaaggatcagattgacatcaccaccaaccctgtgcagatgctccatgagttctcaaaataccggcagcgcattaagcagcatgctgatgctgtgcacctcatctcgcgggtgacatttcactataagagaagcagtctgagttactttggaggtgtctgttctcgcacaagaggagttggtgtgaatgagtatggtcttccaatggcagtggcacaagtattatcgcagagcctggctcaaaaccttggaatccaatgggaaccttctagcagaaagccaaaatgtgactgcacagaatcctggggtggctgcatcatggaggaaacaggggtgtcccattctcgaaaattttcaaagtgcagcattttggagtatagagactttttacagagaggaggtggagcctgccttttcaacaggccaacaaagctatttgagcccacggaatgtggaaatggatacgtggaagctggggaggagtgtgattgtggttttcatgtggaatgctatggattatgctgtaagaaatgttccctctccaacggggctcactgcagcgacgggccctgctgtaacaatacctcatgtctttttcagccacgagggtatgaatgccgggatgctgtgaacgagtgtgatattactgaatattgtactggagactctggtcagtgcccaccaaatcttcataagcaagacggatatgcatgcaatcaaaatcagggccgctgctacaatggcgagtgcaagaccagagacaaccagtgtcagtacatctggggaacaaaggctgcagggtctgacaagttctgctatgaaaagctgaatacagaaggcactgagaagggaaactgcgggaaggatggagaccggtggattcagtgcagcaaacatgatgtgttctgtggattcttactctgtaccaatcttactcgagctccacgtattggtcaacttcagggtgagatcattccaacttccttctaccatcaaggccgggtgattgactgcagtggtgcccatgtagttttagatgatgatacggatgtgggctatgtagaagatggaacgccatgtggcccgtctatgatgtgtttagatcggaagtgcctacaaattcaagccctaaatatgagcagctgtccactcgattccaagggtaaagtctgttcgggccatggggtgtgtagtaatgaagccacctgcatttgtgatttcacctgggcagggacagattgcagtatccgggatccagttaggaaccttcacccccccaaggatgaaggacccaagggtcctagtgccaccaatctcataataggctccatcgctggtgccatcctggtagcagctattgtccttgggggcacaggctggggatttaaaaatgtcaagaagagaaggttcgatcctactcagcaaggccccatctgaatcagctgcgctggatggacaccgccttgcactgttggattctgggtatgacatactcgcagcagtgttactggaactattaagtttgtaaacaaaacctttgggtggtaatgactacggagctaaagttggggtgacaaggatggggtaaaagaaaactgtctcttttggaaataatgtcaaagaacacctttcaccacctgtcagtaaacgggggagggggcaaaagaccatgctataaaaagaactgttccagaatcttttttttccctaatggacgaaggaacaacacacacacaaaaattaaatgcaataaaggaatcattaaaaaaaaaaa

SEQ ID NO: 164 - DNA, 3220 bp, Homo sapiens
tcactggagaggaggcagggacagacccagcagcacccacctgagcgagaagagcagacaccgtgctcctggaatcacccagcatgttgcaaggtctcctgccagtcagtctcctcctctctgttgcagtaagtgctataaaagaactccctggggtgaagaagtatgaagtggtttatcctataagacttcatccactgcataaaagagaggccaaagagccagagcaacaggaacaatttgaaactgaattaaagtataaaatgacaattaatggaaaaattgcagtgctttatttgaaaaaaaacaagaacctccttgcaccaggctacacggaaacatattataattccactggaaaggagatcaccacaagcccacaaattatggatgattgttattatcaaggacatattcttaatgaaaaggtttctgacgctagcatcagcacatgtaggggtctaaggggctacttcagtcagggggatcaaagatactttattgaacctttaagccccatacatcgggatggacaggagcatgcactcttcaagtataaccctgatgaaaagaattatgacagcacctgtgggatggatggtgtgttgtgggcccacgatttgcagcagaacattgccctacctgccaccaaactagtaaaattgaaagacaggaaggttcaggaacatgagaaatacatagaatattatttggtcctggataatggtgagtttaaaaggtacaatgagaatcaagatgagatcagaaagagggtatttgagatggctaattatgtcaacatgctttataaaaagctcaatactcatgtggccttagttggtatggaaatctggactgacaaggataagataaagataaccccaaatgcaagcttcaccttggagaatttttctaaatggagggggagtgttctctcaagaagaaagcgtcatgatattgctcagttaatcacagcaacagaacttgctggaacgactgtgggtcttgcatttatgtctacaatgtgttctccttattctgttggcgttgttcaggaccacagcgataatcttcttagagttgcagggacaatggcacatgaaatgggccacaactttggaatgtttcatgacgactattcttgcaagtgtccttctacaatatgtgtgatggacaaagcactgagcttctatatacccacagacttcagttcctgcagccgtctcagctatgacaagttttttgaagataaattatcaaattgcctctttaatgctccattgcctacagatatcatatccactccaatttgtgggaaccagttggtggaaatgggagaggactgtgattgtgggacatctgaggaatgtaccaatatttgctgtgatgctaagacatgtaaaatcaaagcaacttttcaatgtgcattaggagaatgttgtgaaaaatgccaatttaaaaaggctgggatggtgtgcagaccagcaaaagatgagtgcgacctgcctgaaatgtgtaatggtaaatctggtaattgtcctgatgatagattccaagtcaatggcttcccttgccatcacgggaagggccactgcttgatggggacatgccccacactgcaggagcagtgcacagagctgtggggaccaggaactgaggttgcagataagtcatgttacaacaggaatgaaggtgggtcaaagtacgggtactgtcgcagagtggatgacacactcattccctgcaaagcaaatgataccatgtgtgggaagttgttctgtcaaggtgggtcggataatttgccctggaaaggacggatagtgactttcctgacatgtaaaacatttgatcctgaagacacaagtcaagaaataggcatggtggccaatggaactaagtgtggcgataacaaggtttgcattaatgcagaatgtgtggatattgagaaagcctacaaatcaaccaattgctcatctaagtgcaaaggacatgctgtgtgtgaccatgagctccagtgtcaatgtgaggaaggatggatccctcccgactgcgatgactcctcagtggtcttccacttctccattgtggttggggtgctgttcccaatggcggtcatttttgtggtggttgctatggtaatccggcaccagagctccagagaaaagcagaagaaagatcagaggccactatctaccactggcaccaggccacacaaacagaagaggaaaccccagatggtaaaggctgttcaaccccaagagatgagtcagatgaagccccatgtgtatgatctgccagtagaaggcaatgagcccccagcctcttttcataaagacacaaacgcacttccccctactgttttcaaggataatccagtgtctacacctaaggactcaaatccaaaagcatgaagcaacagctaagcaagaactaatggctaaattatcaacttggaaaactggaaaatctggatggcagagaaatatactatctatctcaccagtatttgctctcgactcaagaaggttaacattttctgattcatgttagactttgaagagactaaagaaaattttcaagaggaacatatgcctgagaacctttgcatgaatttaaaatttcaattatccattcttataagaaggaagatgattgtaaagaaatatctccgaagttaaaatctgtaataggaattgattcattctctaatgaaaacaaaacataaaaacatcacactaatcttggaggaataagaaaaattgtacatccattaaatgtacaattgattgcaacatcttgattgttttaaccattaacttgtcaaattacaatcacagttaagaaaatgatgtaaaattctgttttgtggatctctttcctagattagcttctgaaatcattattagctatatcatttgaggttttctacaatttggtataactaagaatttaaaaatgttttatcatatatatttgtataattaattactggcatggttaaagtggttttcactttttaaatggagaaaatttcagttaaattaataggataaaccaggttgcgaactggtgacctgtaggccatgtttgcactgcaaatatatttggtctgaatgatattgatattggacacatagtacttttacatgttttgaatgtattgctaatatttaaaaattgagagatcttgcataaacaatagattcccagctttgtcaga

SEQ ID NO: 165 - DNA, 3325 bp, Homo sapiens
gtgacatcacatccactaaccaaatggggtggtgtgagtatctcctataaaataaaagctctcctgatggcctgttcctgcacatttcctgaggacgccctcgatcacaagcagcttgctggatgaataaaggaaagaaggtttatatacactaataatagaaatgttttgggatggttcatgatttcgaaaagcttcagagaaaataaagatgcttaatgttagagtacaaaacattctccctgcagtctcacgaactgtgaacaaaaactgaagtgaaaactcatagtgcataactcgtcaatactcctgtgatcgtataaccatcagcaagaaaacaaatttgattgagcccccatccagtcctctttgcgtggaatcagacctcttttgcagtggaaaggagcagagaatgtgactttcctgaacaacaggagcaagaatcaatgccagattctcttctctaaaaataatcaatttgttactacagtgctgcagctctgatggttcaactctgccaaaagatggatctttaatgattagcactacacactgaccaactcagaagaaggagccacaccacctgtgactccagccctgacttctgctctggaccagtgtttccataacagggacttcaaaatcactgtgatttgaagcctttttgaacatgaagatgttactcctgctgcattgccttggggtgtttctgtcctgttctggacacatccaggatgagcacccccaatatcacagccctccggatgtggtgattcctgtgaggataactggcaccaccagaggcatgacacctccaggctggctctcctatatcctgccctttggaggccagaaacacattatccacataaaggtcaagaagcttttgttttccaaacacctccctgtgttcacctacacagaccagggtgctatccttgaggaccagccatttgtccagaataactgctactatcatggttatgtggaaggggacccagaatccctggtttccctcagtacctgttttgggggttttcaaggaatattacagataaatgactttgcttatgaaatcaagcccctagcattttctaccacgtttgaacatctggtatacaagatggacagtgaggagaaacaattttcaaccatgagatccggatttatgcaaaatgaaataacatgccgaatggaatttgaagaaattgataattccactcagaagcaaagttcttatgtgggctggtggatccattttaggattgttgaaattgtagtcgtcattgataattatctgtacattcgttatgaaaggaacgactcaaagttgctggaggatctatatgttattgttaatatagtggattccattttggatgtcattggtgttaaggtgttattatttggtttggagatctggaccaataaaaacctcattgtagtagatgatgtaaggaaatctgtgcacctgtattgcaagtggaagtcggagaacattacgccccggatgcaacatgacacctcacatcttttcacaactctaggattaagagggttaagtggcataggagcttttagaggaatgtgtacaccacaccgtagttgtgcaattgttactttcatgaacaaaactttgggcactttttcaattgcagtggctcatcatctaggtcataatttgggcatgaaccatgatgaggatacatgtcgttgttcacaacctagatgcataatgcatgaaggcaacccaccaataactaaatttagcaattgtagttatggtgatttttgggaatatactgtagagaggacaaagtgtttgcttgaaacagtacacacaaaggacatctttaatgtgaagcgctgtgggaatggtgttgttgaagaaggagaagagtgtgactgtggacctttaaagcattgtgcaaaagatccctgctgtctgtcaaattgcactctgactgatggttctacttgtgcttttgggctttgttgcaaagactgcaagttcctaccatcagggaaagtgtgtagaaaggaggtcaatgaatgtgatcttccagagtggtgcaatggtacttcccataagtgcccagatgacttttatgtggaagatggaattccctgtaaggagaggggctactgctatgaaaagagctgtcatgaccgcaatgaacagtgtaggaggatttttggtgcaggcgcaaatactgcaagtgagacttgctacaaagaattgaacaccttaggtgaccgtgttggtcactgtggtatcaaaaatgctacatatataaagtgtaatatctcagatgtccagtgtggaagaattcagtgtgagaatgtgacagaaattcccaatatgagtgatcatactactgtgcattgggctcgcttcaatgacataatgtgctggagtactgattaccatttggggatgaagggacctgatattggtgaagtgaaagatggaacagagtgtgggatagatcatatatgcatccacaggcactgtgtccatataaccatcttgaatagtaattgctcacctgcattttgtaacaagaggggcatctgcaacaataaacatcactgccattgcaattatctgtgggaccctcccaactgcctgataaaaggctatggaggtagtgttgacagtggcccaccccctaagagaaagaagaaaaagaagttctgttatctgtgtatattgttgcttattgttttgtttattttattatgttgtctttatcgactttgtaaaaaaagtaaaccaataaaaaagcagcaagatgttcaaactccatctgcaaaagaagaggaaaaaattcagcgtcgacctcatgagttacctccccagagtcaaccttgggtgatgccttcccagagtcaacctcctgtgacgccttcccagagtcatcctcaggtgatgccttcccagagtcaacctcctgtgacaccctcccagagtcaacctcgggtgatgccttctcagagtcaacctcctgtgatgccttcccagagtcatcctcagttgacgccttcccagagtcaacctcctgtgacaccctcccagaggcaacctcagttgatgccttcccagagtcaacctcctgtgacgccctcctagagccaacctcagttgatgccttcccagagtcaacctcctgtgacgccctcccagagccaacctcgggtgacaccctcccagagtcaacctcatgtgacaccttaccggagtaaaagtggtaaacaaaagcaatcagtaccaattccaaaaactgtatccagaaaaggtacattaaaaaaataattcctagta

SEQ ID NO: 166 - DNA, 2972 bp, Homo sapiens
gagccctgggaggctacgggctcccccggaaaccctgccaggggagccgggttttgagctcaggcgcctctagcggcggcccccagaaatctgactcgcgaggccagagttgcagggactgaatagcaaactgaggctgagtagggaacagaccatgaggtcagtgcagatcttcctctcccaatgccgtttgctccttctactagttccgacaatgctccttaagtctcttggcgaagatgtaatttttcaccctgaaggggagtttgactcgtatgaagtcaccattcctgagaagctgagcttccggggagaggtgcagggtgtggtcagtcccgtgtcctacctactgcagttaaaaggcaagaagcacgtcctccatttgtggcccaagagacttctgttgccccgacatctgcgcgttttctccttcacagaacatggggaactgctggaggatcatccttacataccaaaggactgcaactacatgggctccgtgaaagagtctctggactctaaagctactataagcacatgcatggggggtctccgaggtgtatttaacattgatgccaaacattaccaaattgagcccctcaaggcctctcccagttttgaacatgtcgtctatctcctgaagaaagagcagtttgggaatcaggtttgtggcttaagtgatgatgaaatagaatggcagatggccccttatgagaataaggcgaggctaagggactttcctggatcctataaacacccaaagtacttggaattgatcctactctttgatcaaagtaggtataggtttgtgaacaacaatctttctcaagtcatacatgatgccattcttttgactgggattatggacacctactttcaagatgttcgtatgaggatacacttaaaggctcttgaagtatggacagattttaacaaaatacgcgttggatatccagagttagctgaagttttaggcagatttgtaatatataaaaaaagtgtattaaatgctcgcctgtcatcagattgggcacatttatatcttcaaagaaaatataatgatgctcttgcatggtcgtttggaaaagtgtgttctctagaatatgctggatcagtgagtactttactagatacaaatatccttgcccctgctacctggtctgctcatgagctgggtcatgctgtaggaatgtcacatgatgaacaatactgccaatgtaggggtaggcttaattgcatcatgggctcaggacgcactgggtttagcaattgcagttatatctctttttttaaacatatctcttcgggagcaacatgtctaaataatatcccaggactaggttatgtgcttaagagatgtggaaacaaaattgtggaggacaatgaggaatgtgactgtggttccacagaggagtgtcagaaagatcggtgttgccaatcaaattgtaagttgcaaccaggtgccaactgtagcattggactttgctgtcatgattgtcggtttcgtccatctggatacgtgtgtaggcaggaaggaaatgaatgtgaccttgcagagtactgcgacgggaattcaagttcctgcccaaatgacgtttataagcaggatggaaccccttgcaagtatgaaggccgttgtttcaggaaggggtgcagatccagatatatgcagtgccaaagcatttttggacctgatgccatggaggctcctagtgagtgctatgatgcagttaacttaataggtgatcaatttggtaactgtgagattacaggaattcgaaattttaaaaagtgtgaaagtgcaaattcaatatgtggcaggctacagtgtataaatgttgaaaccatccctgatttgccagagcatacgactataatttctactcatttacaggcagaaaatctcatgtgctggggcacaggctatcatctatccatgaaacccatgggaatacctgacctaggtatgataaatgatggcacctcctgtggagaaggccgggtatgttttaaaaaaaattgcgtcaatagctcagtcctgcagtttgactgtttgcctgagaaatgcaatacccggggtgtttgcaacaacagaaaaaactgccactgcatgtatgggtgggcacctccattctgtgaggaagtggggtatggaggaagcattgacagtgggcctccaggactgctcagaggggcgattccctcgtcaatttgggttgtgtccatcataatgtttcgccttattttattaatcctttcagtggtttttgtgtttttccggcaagtgataggaaaccacttaaaacccaaacaggaaaaaatgccactatccaaagcaaaaactgaacaggaagaatctaaaacaaaaactgtacaggaagaatctaaaacaaaaactggacaggaagaatctgaagcaaaaactggacaggaagaatctaaagcaaaaactggacaggaagaatctaaagcaaacattgaaagtaaacgacccaaagcaaagagtgtcaagaaacaaaaaaagtaaccgggcaatccatactcattcagtaacacaggctcatttatttaaccagctaatcatttatccaaaggctttccattcttctcccaatatttttttactttaatttttcccacaagttttgatcagcaaataaacagcattcttgttttggaaacaaaccagtgcattctacttctcgagtgttcacttgcccctcagtttgtgaccaagttgtgggtattctgagaatcatgctctaatggcttttccatacactaaagctctgaaagtagtgagtttcctagaattaccatgctattcaaggtatagtcttgtttctcagaatcccagggaacaaagcaattgtctgcctctccctctgcctccctcaacttgatccatgtgaacatccgcacctgcacccatgctgatcaataaatgccgacaacacaaaaaaaaaaaaaaa

SEQ ID NO: 167 - DNA, 2742 bp, Homo sapiens
tggctccagcaaccacgcggctggggtgcgccgggaagggagctggatgttttagcctcggggcgcacgctgcgggcccttcgtgttccggacgctaaacaccgagagcaccccgtctccggggcctccggagaacgctgtcccatgaacgtgcggggagcggcccccggcgtccgcgcgtccccgcgtccctggcaattcccgacttcccaacggcttcccgctggcagccccgaagccgcaccatgttccgcctctggttgctgctggccgggctctgcggcctcctggcgtcaagacccggttttcaaaattcacttctacagatcgtaattccagagaaaatccaaacaaatacaaatgacagttcagaaatagaatatgaacaaatatcctatattattccaatagatgagaaactgtacactgtgcaccttaaacaaagatattttttagcagataattttatgatctatttgtacaatcaaggatctatgaatacttattcttcagatattcagactcaatgctactatcaaggaaatattgaaggatatccagattccatggtcacactcagcacgtgctctggactaagaggaatactgcaatttgaaaatgtttcttatggaattgagcctctggaatctgcagttgaatttcagcatgttctttacaaattaaagaatgaagacaatgatattgcaatttttattgacagaagcctgaaagaacaaccaatggatgacaacatttttataagtgaaaaatcagaaccagctgttccagatttatttcctctttatctagaaatgcatattgtggtggacaaaactttgtatgattactggggctctgatagcatgatagtaacaaataaagtcatcgaaattgttggccttgcaaattcaatgttcacccaatttaaagttactattgtgctgtcatcattggagttatggtcagatgaaaataagatttctacagttggtgaggcagatgaattattgcaaaaatttttagaatggaaacaatcttatcttaacctaaggcctcatgatattgcatatctactaatttatatggattatcctcgttatttgggagcagtgtttcctggaacaatgtgtattactcgttattctgcaggagttgcattgtaccccaaggagataactctggaggcatttgcagttattgtcacccagatgctggcactcagtctgggaatatcatatgacgacccaaagaaatgtcaatgttcagaatccacctgtataatgaatccagaagttgtgcaatccaatggtgtgaagacttttagcagttgcagtttgaggagctttcaaaatttcatttcaaatgtgggtgtcaaatgtcttcagaataagccacaaatgcaaaaaaaatctccgaaaccagtctgtggcaatggcagattggagggaaatgaaatctgtgattgtggtactgaggctcaatgtggacctgcaagctgttgtgattttcgaacttgtgtactgaaagacggagcaaaatgttataaaggactgtgctgcaaagactgtcaaattttacaatcaggcgttgaatgtaggccgaaagcacatcctgaatgtgacatcgctgaaaattgtaatggaagctcaccagaatgtggtcctgacataactttaatcaatggactttcatgcaaaaataataagtttatttgttatgacggagactgccatgatctcgatgcacgttgtgagagtgtatttggaaaaggttcaagaaatgctccatttgcctgctatgaagaaatacaatctcaatcagacagatttgggaactgtggtagggatagaaataacaaatatgtgttctgtggatggaggaatcttatatgtggaagattagtttgtacctaccctactcgaaagcctttccatcaagaaaatggtgatgtgatttatgctttcgtacgagattctgtatgcataactgtagactacaaattgcctcgaacagttccagatccactggctgtcaaaaatggctctcagtgtgatattgggagggtttgtgtaaatcgtgaatgtgtagaatcaaggataattaaggcttcagcacatgtttgttcacaacagtgttctggacatggagtgtgtgattccagaaacaagtgccattgttcgccaggctataagcctccaaactgccaaatacgttccaaaggattttccatatttcctgaggaagatatgggttcaatcatggaaagagcatctgggaagactgaaaacacctggcttctaggtttcctcattgctcttcctattctcattgtaacaaccgcaatagttttggcaaggaaacagttgaaaaagtggttcgccaaggaagaggaattcccaagtagcgaatctaaatcggaaggtagcacacagacatatgccagccaatccagctcagaaggcagcactcagacatatgccagccaaaccagatcagaaagcagcagtcaagctgatactagcaaatccaaatcagaagatagtgctgaagcatatactagcagatccaaatcacaggacagtacccaaacacaaagcagtagtaactagtgattccttcagaaggcaacggataacatcgagagtctcgctaagaaatgaaaattctgtctttccttccgtggtcacagctgaaagaaacaataaattgagtgtggatcaatttgcaaaaaaaaaaaaaaaa

SEQ ID NO: 168 - DNA, 3594 bp, Homo sapiens
gctgcaccgggcacgggtcggccgcaatccagcctgggcggagccggagttgcgagccgctgcctagaggccgaggagctcacagctatgggctggaggccccggagagctcgggggaccccgttgctgctgctgctactactgctgctgctctggccagtgccaggcgccggggtgcttcaaggacatatccctgggcagccagtcaccccgcactgggtcctggatggacaaccctggcgcaccgtcagcctggaggagccggtctcgaagccagacatggggctggtggccctggaggctgaaggccaggagctcctgcttgagctggagaagaaccacaggctgctggccccaggatacatagaaacccactacggcccagatgggcagccagtggtgctggcccccaaccacacggatcattgccactaccaagggcgagtaaggggcttccccgactcctgggtagtcctctgcacctgctctgggatgagtggcctgatcaccctcagcaggaatgccagctattatctgcgtccctggccaccccggggctccaaggacttctcaacccacgagatctttcggatggagcagctgctcacctggaaaggaacctgtggccacagggatcctgggaacaaagcgggcatgaccagccttcctggtggtccccagagcaggggcaggcgagaagcgcgcaggacccggaagtacctggaactgtacattgtggcagaccacaccctgttcttgactcggcaccgaaacttgaaccacaccaaacagcgtctcctggaagtcgccaactacgtggaccagcttctcaggactctggacattcaggtggcgctgaccggcctggaggtgtggaccgagcgggaccgcagccgcgtcacgcaggacgccaacgccacgctctgggccttcctgcagtggcgccgggggctgtgggcgcagcggccccacgactccgcgcagctgctcacgggccgcgccttccagggcgccacagtgggcctggcgcccgtcgagggcatgtgccgcgccgagagctcgggaggcgtgagcacggaccactcggagctccccatcggcgccgcagccaccatggcccatgagatcggccacagcctcggcctcagccacgaccccgacggctgctgcgtggaggctgcggccgagtccggaggctgcgtcatggctgcggccaccgggcacccgtttccgcgcgtgttcagcgcctgcagccgccgccagctgcgcgccttcttccgcaaggggggcggcgcttgcctctccaatgccccggaccccggactcccggtgccgccggcgctctgcgggaacggcttcgtggaagcgggcgaggagtgtgactgcggccctggccaggagtgccgcgacctctgctgctttgctcacaactgctcgctgcgcccgggggcccagtgcgcccacggggactgctgcgtgcgctgcctgctgaagccggctggagcgctgtgccgccaggccatgggtgactgtgacctccctgagttttgcacgggcacctcctcccactgtcccccagacgtttacctactggacggctcaccctgtgccaggggcagtggctactgctgggatggcgcatgtcccacgctggagcagcagtgccagcagctctgggggcctggctcccacccagctcccgaggcctgtttccaggtggtgaactctgcgggagatgctcatggaaactgcggccaggacagcgagggccacttcctgccctgtgcagggagggatgccctgtgtgggaagctgcagtgccagggtggaaagcccagcctgctcgcaccgcacatggtgccagtggactctaccgttcacctagatggccaggaagtgacttgtcggggagccttggcactccccagtgcccagctggacctgcttggcctgggcctggtagagccaggcacccagtgtggacctagaatggtgtgccagagcaggcgctgcaggaagaatgccttccaggagcttcagcgctgcctgactgcctgccacagccacggggtttgcaatagcaaccataactgccactgtgctccaggctgggctccacccttctgtgacaagccaggctttggtggcagcatggacagtggccctgtgcaggctgaaaaccatgacaccttcctgctggccatgctcctcagcgtcctgctgcctctgctcccaggggccggcctggcctggtgttgctaccgactcccaggagcccatctgcagcgatgcagctggggctgcagaagggaccctgcgtgcagtggccccaaagatggcccacacagggaccaccccctgggcggcgttcaccccatggagttgggccccacagccactggacagccctggcccctggaccctgagaactctcatgagcccagcagccaccctgagaagcctctgccagcagtctcgcctgacccccaagcagatcaagtccagatgccaagatcctgcctctggtgagaggtagctcctaaaatgaacagatttaaagacaggtggccactgacagccactccaggaacttgaactgcaggggcagagccagtgaatcaccggacctccagcacctgcaggcagcttggaagtttcttccccgagtggagcttcgacccacccactccaggaacccagagccacattagaagttcctgagggctggagaacactgctgggcacactctccagctcaataaaccatcagtcccagaagcaaaggtcacacagcccctgacctccctcaccagtggaggctgggtagtgctggccatcccaaaagggctctgtcctgggagtctggtgtgtctcctacatgcaatttccacggacccagctctgtggagggcatgactgctggccagaagctagtggtcctggggccctatggttcgactgagtccacactcccctggagcctggctggcctctgcaaacaaacataattttggggaccttccttcctgtttcttcccaccctgtcttctcccctaggtggttcctgagcccccacccccaatcccagtgctacacctgaggttctggagctcagaatctgacagcctctcccccattctgtgtgtgtcggggggacagagggaaccatttaagaaaagataccaaagtagaagtcaaaagaaagacatgttggctataggcgtggtggctcatgcctataatcccagcactttgggaagccggggtaggaggatcaccagaggccagcaggtccacaccagcctgggcaacacagcaagacaccgcatctacagaaaaattttaaaattagctgggcgtggtggtgtgtacctgtaggcctagctgctcaggaggctgaagcaggaggatcacttgagcctgagttcaacactgcagtgagctatggtggcaccactgcactccagcctgggtgacagagcaagaccctgtctctaaaataaattttaaaaagacataaaaaaaaaaaaaaaaaaaaaaaaaaaaaaaaaaaaaaaaaaaaaaaaaaaaaaaaaaaaaaaaaaaaaaaaaaaa

SEQ ID NO: 169 - protein, 735 aa, Homo sapiens
MWRVLFLLSGLGGLRMDSNFDSLPVQITVPEKIRSIIKEGIESQASYKIVIEGKPYTVNLMQKNFLPHNFRVYSYSGTGIMKPLDQDFQNFCHYQGYIEGYPKSVVMVSTCTGLRGVLQFENVSYGIEPLESSVGFEHVIYQVKHKKADVSLYNEKDIESRDLSFKLQSVEPQQDFAKYIEMHVIVEKQLYNHMGSDTTVVAQKVFQLIGLTNAIFVSFNITIILSSLELWIDENKIATTGEANELLHTFLRWKTSYLVLRPHDVAFLLVYREKSNYVGATFQGKMCDANYAGGVVLHPRTISLESLAVILAQLLSLSMGITYDDINKCQCSGAVCIMNPEAIHFSGVKIFSNCSFEDFAHFISKQKSQCLHNQPRLDPFFKQQAVCGNAKLEAGEECDCGTEQDCALIGETCCDIATCRFKAGSNCAEGPCCENCLFMSKERMCRPSFEECDLPEYCNGSSASCPENHYVQTGHPCGLNQWICIDGVCMSGDKQCTDTFGKEVEFGPSECYSHLNSKTDVSGNCGISDSGYTQCEADNLQCGKLICKYVGKFLLQIPRATIIYANISGHLCIAVEFASDHADSQKMWIKDGTSCGSNKVCRNQRCVSSSYLGYDCTTDKCNDRGVCNNKKHCHCSASYLPPDCSVQSDLWPGGSIDSGNFPPVAIPARLPERRYIENIYHSKPMRWPFFLFIPFFIIFCVLIAIMVKVNFQRKKWRTEDYSSDEQPESESEPKG

SEQ ID NO: 170 - protein, 754 aa, Homo sapiens
MLPGCIFLMILLIPQVKEKFILGVEGQQLVRPKKLPLIQKRDTGHTHDDDILKTYEEELLYEIKLNRKTLVLHLLRSREFLGSNYSETFYSMKGEAFTRHPQIMDHCFYQGSIVHEYDSAASISTCNGLRGFFRINDQRYLIEPVKYSDEGEHLVFKYNLRVPYGANYSCTELNFTRKTVPGDNESEEDSKIKGIHDEKYVELFIVADDTVYRRNGHPHNKLRNRIWGMVNFVNMIYKTLNIHVTLVGIEIWTHEDKIELYSNIETTLLRFSFWQEKILKTRKDFDHVVLLSGKWLYSHVQGISYPGGMCLPYYSTSIIKDLLPDTNIIANRMAHQLGHNLGMQHDEFPCTCPSGKCVMDSDGSIPALKFSKCSQNQYHQYLKDYKPTCMLNIPFPYNFHDFQFCGNKKLDEGEECDCGPAQECTNPCCDAHTCVLKPGFTCAEGECCESCQIKKAGSICRPAKDECDFPEMCTGHSPACPKDQFRVNGFPCKNSEGYCFMGKCPTREDQCSELFDDEAIESHDICYKMNTKGNKFGYCKNKENRFLPCEEKDVRCGKIYCTGGELSSLLGEDKTYHLKDPQKNATVKCKTIFLYHDSTDIGLVASGTKCGEGMVCNNGECLNMEKVYISTNCPSQCNENPVDGHGLQCHCEEGQAPVACEETLHVTNITILVVVLVLVIVGIGVLILLVRYRKCIKLKQVQSPPTETLGVENKGYFGDEQQIRTEPILPEIHFLNKPASKDSRGIADPNQSAK

SEQ ID NO: 171 - protein, 824 aa, Homo sapiens
MRGLGLWLLGAMMLPAIAPSRPWALMEQYEVVLPWRLPGPRVRRALPSHLGLHPERVSYVLGATGHNFTLHLRKNRDLLGSGYTETYTAANGSEVTEQPRGQDHCFYQGHVEGYPDSAASLSTCAGLRGFFQVGSDLHLIEPLDEGGEGGRHAVYQAEHLLQTAGTCGVSDDSLGSLLGPRTAAVFRPRPGDSLPSRETRYVELYVVVDNAEFQMLGSEAAVRHRVLEVVNHVDKLYQKLNFRVVLVGLEIWNSQDRFHVSPDPSVTLENLLTWQARQRTRRHLHDNVQLITGVDFTGTTVGFARVSAMCSHSSGAVNQDHSKNPVGVACTMAHEMGHNLGMDHDENVQGCRCQERFEAGRCIMAGSIGSSFPRMFSDCSQAYLESFLERPQSVCLANAPDLSHLVGGPVCGNLFVERGEQCDCGPPEDCRNRCCNSTTCQLAEGAQCAHGTCCQECKVKPAGELCRPKKDMCDLEEFCDGRHPECPEDAFQENGTPCSGGYCYNGACPTLAQQCQAFWGPGGQAAEESCFSYDILPGCKASRYRADMCGVLQCKGGQQPLGRAICIVDVCHALTTEDGTAYEPVPEGTRCGPEKVCWKGRCQDLHVYRSSNCSAQCHNHGVCNHKQECHCHAGWAPPHCAKLLTEVHAASGSLPVFVVVVLVLLAVVLVTLAGIIVYRKARSRILSRNVAPKTTMGRSNPLFHQAASRVPAKGGAPAPSRGPQELVPTTHPGQPARHPASSVALKRPPPAPPVTVSSPPFPVPVYTRQAPKQVIKPTFAPPVPPVKPGAGAANPGPAEGAVGPKVALKPPIQRKQGAGAPTAP

SEQ ID NO: 172 - protein, 819 aa, Homo sapiens
MGSGARFPSGTLRVRWLLLLGLVGPVLGAARPGFQQTSHLSSYEIITPWRLTRERREAPRPYSKQVSYVIQAEGKEHIIHLERNKDLLPEDFVVYTYNKEGTLITDHPNIQNHCHYRGYVEGVHNSSIALSDCFGLRGLLHLENASYGIEPLQNSSHFEHIIYRMDDVYKEPLKCGVSNKDIEKETAKDEEEEPPSMTQLLRRRRAVLPQTRYVELFIVVDKERYDMMGRNQTAVREEMILLANYLDSMYIMLNIRIVLVGLEIWTNGNLINIVGGAGDVLGNFVQWREKFLITRRRHDSAQLVLKKGFGGTAGMAFVGTVCSRSHAGGINVFGQITVETFASIVAHELGHNLGMNHDDGRDCSCGAKSCIMNSGASGSRNFSSCSAEDFEKLTLNKGGNCLLNIPKPDEAYSAPSCGNKLVDAGEECDCGTPKECELDPCCEGSTCKLKSFAECAYGDCCKDCRFLPGGTLCRGKTSECDVPEYCNGSSQFCQPDVFIQNGYPCQNNKAYCYNGMCQYYDAQCQVIFGSKAKAAPKDCFIEVNSKGDRFGNCGFSGNEYKKCATGNALCGKLQCENVQEIPVFGIVPAIIQTPSRGTKCWGVDFQLGSDVPDPGMVNEGTKCGAGKICRNFQCVDASVLNYDCDVQKKCHGHGVCNSNKNCHCENGWAPPNCETKGYGGSVDSGPTYNEMNTALRDGLLVFFFLIVPLIVCAIFIFIKRDQLWRSYFRKKRSQTYESDGKNQANPSRQPGSVPRHVSPVTPPREVPIYANRFAVPTYAAKQPQQFPSRPPPPQPKVSSQGNLIPARPAPAPPLYSSLT

SEQ ID NO: 173 - protein, 748 aa, Homo sapiens
MVLLRVLILLLSWAAGMGGQYGNPLNKYIRHYEGLSYNVDSLHQKHQRAKRAVSHEDQFLRLDFHAHGRHFNLRMKRDTSLFSDEFKVETSNKVLDYDTSHIYTGHIYGEEGSFSHGSVIDGRFEGFIQTRGGTFYVEPAERYIKDRTLPFHSVIYHEDDINYPHKYGPQGGCADHSVFERMRKYQMTGVEEVTQIPQEEHAANGPELLRKKRTTSAEKNTCQLYIQTDHLFFKYYGTREAVIAQISSHVKAIDTIYQTTDFSGIRNISFMVKRIRINTTADEKDPTNPFRFPNIGVEKFLELNSEQNHDDYCLAYVFTDRDFDDGVLGLAWVGAPSGSSGGICEKSKLYSDGKKKSLNTGIITVQNYGSHVPPKVSHITFAHEVGHNFGSPHDSGTECTPGESKNLGQKENGNYIMYARATSGDKLNNNKFSLCSIRNISQVLEKKRNNCFVESGQPICGNGMVEQGEECDCGYSDQCKDECCFDANQPEGRKCKLKPGKQCSPSQGPCCTAQCAFKSKSEKCRDDSDCAREGICNGFTALCPASDPKPNFTDCNRHTQVCINGQCAGSICEKYGLEECTCASSDGKDDKELCHVCCMKKMDPSTCASTGSVQWSRHFSGRTITLQPGSPCNDFRGYCDVFMRCRLVDADGPLARLKKAIFSPELYENIAEWIVAHWWAVLLMGIALIMLMAGFIKICSVHTPSSNPKLPPPKPLPGTLKRRRPPQPIQQPQRQRPRESYQMGHMRR

SEQ ID NO: 174 - protein, 769 aa, Homo sapiens
MRLLRRWAFAALLLSLLPTPGLGTQGPAGALRWGGLPQLGGPGAPEVTEPSRLVRESSGGEVRKQQLDTRVRQEPPGGPPVHLAQVSFVIPAFNSNFTLDLELNHHLLSSQYVERHFSREGTTQHSTGAGDHCYYQGKLRGNPHSFAALSTCQGLHGVFSDGNLTYIVEPQEVAGPWGAPQGPLPHLIYRTPLLPDPLGCREPGCLFAVPAQSAPPNRPRLRRKRQVRRGHPTVHSETKYVELIVINDHQLFEQMRQSVVLTSNFAKSVVNLADVIYKEQLNTRIVLVAMETWADGDKIQVQDDLLETLARLMVYRREGLPEPSDATHLFSGRTFQSTSSGAAYVGGICSLSHGGGVNEYGNMGAMAVTLAQTLGQNLGMMWNKHRSSAGDCKCPDIWLGCIMEDTGFYLPRKFSRCSIDEYNQFLQEGGGSCLFNKPLKLLDPPECGNGFVEAGEECDCGSVQECSRAGGNCCKKCTLTHDAMCSDGLCCRRCKYEPRGVSCREAVNECDIAETCTGDSSQCPPNLHKLDGYYCDHEQGRCYGGRCKTRDRQCQVLWGHAAADRFCYEKLNVEGTERGSCGRKGSGWVQCSKQDVLCGFLLCVNISGAPRLGDLVGDISSVTFYHQGKELDCRGGHVQLADGSDLSYVEDGTACGPNMLCLDHRCLPASAFNFSTCPGSGERRICSHHGVCSNEGKCICQPDWTGKDCSIHNPLPTSPPTGETERYKGPSGTNIIIGSIAGAVLVAAIVLGGTGWGFKNIRRGRSGGA

SEQ ID NO: 175 - protein, 909 aa, Homo sapiens
MAARPLPVSPARALLLALAGALLAPCEARGVSLWNQGRADEVVSASVGSGDLWIPVKSFDSKNHPEVLNIRLQRESKELIINLERNEGLIASSFTETHYLQDGTDVSLARNYTVILGHCYYHGHVRGYSDSAVSLSTCSGLRGLIVFENESYVLEPMKSATNRYKLFPAKKLKSVRGSCGSHHNTPNLAAKNVFPPPSQTWARRHKRETLKATKYVELVIVADNREFQRQGKDLEKVKQRLIEIANHVDKFYRPLNIRIVLVGVEVWNDMDKCSVSQDPFTSLHEFLDWRKMKLLPRKSHDNAQLVSGVYFQGTTIGMAPIMSMCTADQSGGIVMDHSDNPLGAAVTLAHELGHNFGMNHDTLDRGCSCQMAVEKGGCIMNASTGYPFPMVFSSCSRKDLETSLEKGMGVCLFNLPEVRESFGGQKCGNRFVEEGEECDCGEPEECMNRCCNATTCTLKPDAVCAHGLCCEDCQLKPAGTACRDSSNSCDLPEFCTGASPHCPANVYLHDGHSCQDVDGYCYNGICQTHEQQCVTLWGPGAKPAPGICFERVNSAGDPYGNCGKVSKSSFAKCEMRDAKCGKIQCQGGASRPVIGTNAVSIETNIPLQQGGRILCRGTHVYLGDDMPDPGLVLAGTKCADGKICLNRQCQNISVFGVHECAMQCHGRGVCNNRKNCHCEAHWAPPFCDKFGFGGSTDSGPIRQADNQGLTIGILVTILCLLAAGFVVYLKRKTLIRLLFTNKKTTIEKLRCVRPSRPPRGFQPCQAHLGHLGKGLMRKPPDSYPPKDNPRRLLQCQNVDISRPLNGLNVPQPQSTQRVLPPLHRAPRAPSVPARPLPAKPALRQAQGTCKPNPPQKPLPADPLARTTRLTHALARTPGQWETGLRLAPLRPAPQYPHQVPRSTHTAYIK

SEQ ID NO: 176 - protein, 863 aa, Homo sapiens
MRLALLWALGLLGAGSPLPSWPLPNIGGTEEQQAESEKAPREPLEPQVLQDDLPISLKKVLQTSLPEPLRIKLELDGDSHILELLQNRELVPGRPTLVWYQPDGTRVVSEGHTLENCCYQGRVRGYAGSWVSICTCSGLRGLVVLTPERSYTLEQGPGDLQGPPIISRIQDLHLPGHTCALSWRESVHTQKPPEHPLGQRHIRRRRDVVTETKTVELVIVADHSEAQKYRDFQHLLNRTLEVALLLDTFFRPLNVRVALVGLEAWTQRDLVEISPNPAVTLENFLHWRRAHLLPRLPHDSAQLVTGTSFSGPTVGMAIQNSICSPDFSGGVNMDHSTSILGVASSIAHELGHSLGLDHDLPGNSCPCPGPAPAKTCIMEASTDFLPGLNFSNCSRRALEKALLDGMGSCLFERLPSLPPMAAFCGNMFVEPGEQCDCGFLDDCVDPCCDSLTCQLRPGAQCASDGPCCQNCQLRPSGWQCRPTRGDCDLPEFCPGDSSQCPPDVSLGDGEPCAGGQAVCMHGRCASYAQQCQSLWGPGAQPAAPLCLQTANTRGNAFGSCGRNPSGSYVSCTPRDAICGQLQCQTGRTQPLLGSIRDLLWETIDVNGTELNCSWVHLDLGSDVAQPLLTLPGTACGPGLVCIDHRCQRVDLLGAQECRSKCHGHGVCDSNRHCYCEEGWAPPDCTTQLKATSSLTTGLLLSLLVLLVLVMLGASYWYRARLHQRLCQLKGPTCQYRAAQSGPSERPGPPQRALLARGTKQASALSFPAPPSRPLPPDPVSKRLQAELADRPNPPTRPLPADPVVRSPKSQGPAKPPPPRKPLPADPQGRCPSGDLPGPGAGIPPLVVPSRPAPPPPTVSSLYL

SEQ ID NO: 177 - protein, 824 aa, Homo sapiens
MRQSLLFLTSVVPFVLAPRPPDDPGFGPHQRLEKLDSLLSDYDILSLSNIQQHSVRKRDLQTSTHVETLLTFSALKRHFKLYLTSSTERFSQNFKVVVVDGKNESEYTVKWQDFFTGHVVGEPDSRVLAHIRDDDVIIRINTDGAEYNIEPLWRFVNDTKDKRMLVYKSEDIKNVSRLQSPKVCGYLKVDNEELLPKGLVDREPPEELVHRVKRRADPDPMKNTCKLLVVADHRFYRYMGRGEESTTTNYLIELIDRVDDIYRNTSWDNAGFKGYGIQIEQIRILKSPQEVKPGEKHYNMAKSYPNEEKDAWDVKMLLEQFSFDIAEEASKVCLAHLFTYQDFDMGTLGLAYVGSPRANSHGGVCPKAYYSPVGKKNIYLNSGLTSTKNYGKTILTKEADLVTTHELGHNFGAEHDPDGLAECAPNEDQGGKYVMYPIAVSGDHENNKMFSNCSKQSIYKTIESKAQECFQERSNKVCGNSRVDEGEECDPGIMYLNNDTCCNSDCTLKEGVQCSDRNSPCCKNCQFETAQKKCQEAINATCKGVSYCTGNSSECPPPGNAEDDTVCLDLGKCKDGKCIPFCEREQQLESCACNETDNSCKVCCRDLSGRCVPYVDAEQKNLFLRKGKPCTVGFCDMNGKCEKRVQDVIERFWDFIDQLSINTFGKFLADNIVGSVLVFSLIFWIPFSILVHCVDKKLDKQYESLSLFHPSNVEMLSSMDSASVRIIKPFPAPQTPGRLQPAPVIPSAPAAPKLDHQRMDTIQEDPSTDSHMDEDGFEKDPFPNSSTAAKSFEDLTDHPVTRSEKAASFKLQRQNRVDSKETEC

SEQ ID NO: 178 - protein, 739 aa, Homo sapiens
MFLLLALLTELGRLQAHEGSEGIFLHVTVPRKIKSNDSEVSERKMIYIITIDGQPYTLHLGKQSFLPQNFLVYTYNETGSLHSVSPYFMMHCHYQGYAAEFPNSFVTLSICSGLRGFLQFENISYGIEPVESSARFEHIIYQMKNNDPNVSILAVNYSHIWQKDQPYKVPLNSQIKNLSKLLPQYLEIYIIVEKALYDYMGSEMMAVTQKIVQVIGLVNTMFTQFKLTVILSSLELWSNENQISTSGDADDILQRFLAWKRDYLILRPHDIAYLLVYRKHPKYVGATFPGTVCNKSYDAGIAMYPDAIGLEGFSVIIAQLLGLNVGLTYDDITQCFCLRATCIMNHEAVSASGRKIFSNCSMHDYRYFVSKFETKCLQKLSNLQPLHQNQPVCGNGILESNEECDCGNKNECQFKKCCDYNTCKLKGSVKCGSGPCCTSKCELSIAGTPCRKSIDPECDFTEYCNGTSSNCVPDTYALNGRLCKLGTAYCYNGQCQTTDNQCAKIFGKGAQGAPFACFKEVNSLHERSENCGFKNSQPLPCERKDVLCGKLACVQPHKNANKSDAQSTVYSYIQDHVCVSIATGSSMRSDGTDNAYVADGTMCGPEMYCVNKTCRKVHLMGYNCNATTKCKGKGICNNFGNCQCFPGHRPPDCKFQFGSPGGSIDDGNFQKSGDFYTEKGYNTHWNNWFILSFCIFLPFFIVFTTVIFKRNEISKSCNRENAEYNRNSSVVSESDDVGH

SEQ ID NO: 179 - protein, 918 aa, Homo sapiens
MPGGAGAARLCLLAFALQPLRPRAAREPGWTRGSEEGSPKLQHELIIPQWKTSESPVREKHPLKAELRVMAEGRELILDLEKNEQLFAPSYTETHYTSSGNPQTTTRKLEDHCFYHGTVRETELSSVTLSTCRGIRGLITVSSNLSYVIEPLPDSKGQHLIYRSEHLKPPPGNCGFEHSKPTTRDWALQFTQQTKKRPRRMKREDLNSMKYVELYLVADYLEFQKNRRDQDATKHKLIEIANYVDKFYRSLNIRIALVGLEVWTHGNMCEVSENPYSTLWSFLSWRRKLLAQKYHDNAQLITGMSFHGTTIGLAPLMAMCSVYQSGGVNMDHSENAIGVAATMAHEMGHNFGMTHDSADCCSASAADGGCIMAAATGHPFPKVFNGCNRRELDRYLQSGGGMCLSNMPDTRMLYGGRRCGNGYLEDGEECDCGEEEECNNPCCNASNCTLRPGAECAHGSCCHQCKLLAPGTLCREQARQCDLPEFCTGKSPHCPTNFYQMDGTPCEGGQAYCYNGMCLTYQEQCQQLWGPGARPAPDLCFEKVNVAGDTFGNCGKDMNGEHRKCNMRDAKCGKIQCQSSEARPLESNAVPIDTTIIMNGRQIQCRGTHVYRGPEEEGDMLDPGLVMTGTKCGYNHICFEGQCRNTSFFETEGCGKKCNGHGVCNNNQNCHCLPGWAPPFCNTPGHGGSIDSGPMPPESVGPVVAGVLVAILVLAVLMLMYYCCRQNNKLGQLKPSALPSKLRQQFSCPFRVSQNSGTGHANPTFKLQTPQGKRKVINTPEILRKPSQPPPRPPPDYLRGGSPPAPLPAHLSRAARNSPGPGSQIERTESSRRPPPSRPIPPAPNCIVSQDFSRPRPPQKALPANPVPGRRSLPRPGGASPLRPPGAGPQQSRPLAALAPKFPEYRSQRAGGMISSKI

SEQ ID NO: 180 - protein, 776 aa, Homo sapiens
MVQLHQDTDPQIPKGQPCTLNSSEGGARPAVPHTLFSSALDRWLHNDSFIMAVGEPLVHIRVTLLLLWFGMFLSISGHSQARPSQYFTSPEVVIPLKVISRGRGAKAPGWLSYSLRFGGQRYIVHMRVNKLLFAAHLPVFTYTEQHALLQDQPFIQDDCYYHGYVEGVPESLVALSTCSGGFLGMLQINDLVYEIKPISVSATFEHLVYKIDSDDTQFPPMRCGLTEEKIAHQMELQLSYNFTLKQSSFVGWWTHQRFVELVVVVDNIRYLFSQSNATTVQHEVFNVVNIVDSFYHPLEVDVILTGIDIWTASNPLPTSGDLDNVLEDFSIWKNYNLNNRLQHDVAHLFIKDTQGMKLGVAYVKGICQNPFNTGVDVFEDNRLVVFAITLGHELGHNLGMQHDTQWCVCELQWCIMHAYRKVTTKFSNCSYAQYWDSTISSGLCIQPPPYPGNIFRLKYCGNLVVEEGEECDCGTIRQCAKDPCCLLNCTLHPGAACAFGICCKDCKFLPSGTLCRQQVGECDLPEWCNGTSHQCPDDVYVQDGISCNVNAFCYEKTCNNHDIQCKEIFGQDARSASQSCYQEINTQGNRFGHCGIVGTTYVKCWTPDIMCGRVQCENVGVIPNLIEHSTVQQFHLNDTTCWGTDYHLGMAIPDIGEVKDGTVCGPEKICIRKKCASMVHLSQACQPKTCNMRGICNNKQHCHCNHEWAPPYCKDKGYGGSADSGPPPKNNMEGLNVMGKLRYLSLLCLLPLVAFLLFCLHVLFKKRTKSKEDEEG

SEQ ID NO: 181 - protein, 722 aa, Homo sapiens
MAVDGTLVYIRVTLLLLWLGVFLSISGYCQAGPSQHFTSPEVVIPLKVISRGRSAKAPGWLSYSLRFGGQKHVVHMRVKKLLVSRHLPVFTYTDERALLEDQLFIPDDCYYHGYVEGAPESLVVFSACFGGFRGVLKISGLTYEIEPIRHSATFEHLVYKVNSNETQFPAMRCGLTEKEVARQQLEFEEAENSALEPKSAGDWWTHAWFLELVVVVNHDFFIYSQSNISKVQEDVFLVVNIVDSMYQQLGTYIILIGIEIWNQGNVFPMTSIEQVLNDFSQWKQISLSQLQHDAAHMFIKNSLISILGLAYVAGICRPPIDCGVDNFQGDTWSLFANTVAHELGHTLGMQHDEEFCFCGERGCIMNTFRVPAEKFTNCSYADFMKTTLNQGSCLHNPPRLGEIFMLKRCGNGVVEREEQCDCGSVQQCEQDACCLLNCTLRPGAACAFGLCCKDCKFMPSGELCRQEVNECDLPEWCNGTSHQCPEDRYVQDGIPCSDSAYCYQKRCNNHDQHCREIFGKDAKSASQNCYKEINSQGNRFGHCGINGTTYLKCHISDVFCGRVQCENVRDIPLLQDHFTLQHTHINGVTCWGIDYHLRMNISDIGEVKDGTVCGPGKICIHKKCVSLSVLSHVCLPETCNMKGICNNKHHCHCGYGWSPPYCQHRGYGGSIDSGPASAKRGVFLPLIVIPSLSVLTFLFTVGLLMYLRQCSGPKETKAHSSG

SEQ ID NO: 182 - protein, 906 aa, Homo sapiens
MQAAVAVSVPFLLLCVLGTCPPARCGQAGDASLMELEKRKENRFVERQSIVPLRLIYRSGGEDESRHDALDTRVRGDLGGPQLTHVDQASFQVDAFGTSFILDVVLNHDLLSSEYIERHIEHGGKTVEVKGGEHCYYQGHIRGNPDSFVALSTCHGLHGMFYDGNHTYLIEPEENDTTQEDFHFHSVYKSRLFEFSLDDLPSEFQQVNITPSKFILKPRPKRSKRQLRRYPRNVEEETKYIELMIVNDHLMFKKHRLSVVHTNTYAKSVVNMADLIYKDQLKTRIVLVAMETWATDNKFAISENPLITLREFMKYRRDFIKEKSDAVHLFSGSQFESSRSGAAYIGGICSLLKGGGVNEFGKTDLMAVTLAQSLAHNIGIISDKRKLASGECKCEDTWSGCIMGDTGYYLPKKFTQCNIEEYHDFLNSGGGACLFNKPSKLLDPPECGNGFIETGEECDCGTPAECVLEGAECCKKCTLTQDSQCSDGLCCKKCKFQPMGTVCREAVNDCDIRETCSGNSSQCAPNIHKMDGYSCDGVQGICFGGRCKTRDRQCKYIWGQKVTASDKYCYEKLNIEGTEKGNCGKDKDTWIQCNKRDVLCGYLLCTNIGNIPRLGELDGEITSTLVVQQGRTLNCSGGHVKLEEDVDLGYVEDGTPCGPQMMCLEHRCLPVASFNFSTCLSSKEGTICSGNGVCSNELKCVCNRHWIGSDCNTYFPHNDDAKTGITLSGNGVAGTNIIIGIIAGTILVLALILGITAWGYKNYREQRQLPQGDYVKKPGDGDSFYSDIPPGVSTNSASSSKKRSNGLSHSWSERIPDTKHISDICENGRPRSNSWQGNLGGNKKKIRGKRFRPRSNSTETLSPAKSPSSSTGSIASSRKYPYPMPPLPDEDKKVNRQSARLWETSI

SEQ ID NO: 183 - protein, 832 aa, Homo sapiens
MKPPGSSSRQPPLAGCSLAGASCGPQRGPAGSVPASAPARTPPCRLLLVLLLLPPLAASSRPRAWGAAAPSAPHWNETAEKNLGVLADEDNTLQQNSSSNISYSNAMQKEITLPSRLIYYINQDSESPYHVLDTKARHQQKHNKAVHLAQASFQIEAFGSKFILDLILNNGLLSSDYVEIHYENGKPQYSKGGEHCYYHGSIRGVKDSKVALSTCNGLHGMFEDDTFVYMIEPLELVHDEKSTGRPHIIQKTLAGQYSKQMKNLTMERGDQWPFLSELQWLKRRKRAVNPSRGIFEEMKYLELMIVNDHKTYKKHRSSHAHTNNFAKSVVNLVDSIYKEQLNTRVVLVAVETWTEKDQIDITTNPVQMLHEFSKYRQRIKQHADAVHLISRVTFHYKRSSLSYFGGVCSRTRGVGVNEYGLPMAVAQVLSQSLAQNLGIQWEPSSRKPKCDCTESWGGCIMEETGVSHSRKFSKCSILEYRDFLQRGGGACLFNRPTKLFEPTECGNGYVEAGEECDCGFHVECYGLCCKKCSLSNGAHCSDGPCCNNTSCLFQPRGYECRDAVNECDITEYCTGDSGQCPPNLHKQDGYACNQNQGRCYNGECKTRDNQCQYIWGTKAAGSDKFCYEKLNTEGTEKGNCGKDGDRWIQCSKHDVFCGFLLCTNLTRAPRIGQLQGEIIPTSFYHQGRVIDCSGAHVVLDDDTDVGYVEDGTPCGPSMMCLDRKCLQIQALNMSSCPLDSKGKVCSGHGVCSNEATCICDFTWAGTDCSIRDPVRNLHPPKDEGPKGPSATNLIIGSIAGAILVAAIVLGGTGWGFKNVKKRRFDPTQQGPI

SEQ ID NO: 184 - protein, 775 aa, Homo sapiens
MLQGLLPVSLLLSVAVSAIKELPGVKKYEVVYPIRLHPLHKREAKEPEQQEQFETELKYKMTINGKIAVLYLKKNKNLLAPGYTETYYNSTGKEITTSPQIMDDCYYQGHILNEKVSDASISTCRGLRGYFSQGDQRYFIEPLSPIHRDGQEHALFKYNPDEKNYDSTCGMDGVLWAHDLQQNIALPATKLVKLKDRKVQEHEKYIEYYLVLDNGEFKRYNENQDEIRKRVFEMANYVNMLYKKLNTHVALVGMEIWTDKDKIKITPNASFTLENFSKWRGSVLSRRKRHDIAQLITATELAGTTVGLAFMSTMCSPYSVGVVQDHSDNLLRVAGTMAHEMGHNFGMFHDDYSCKCPSTICVMDKALSFYIPTDFSSCSRLSYDKFFEDKLSNCLFNAPLPTDIISTPICGNQLVEMGEDCDCGTSEECTNICCDAKTCKIKATFQCALGECCEKCQFKKAGMVCRPAKDECDLPEMCNGKSGNCPDDRFQVNGFPCHHGKGHCLMGTCPTLQEQCTELWGPGTEVADKSCYNRNEGGSKYGYCRRVDDTLIPCKANDTMCGKLFCQGGSDNLPWKGRIVTFLTCKTFDPEDTSQEIGMVANGTKCGDNKVCINAECVDIEKAYKSTNCSSKCKGHAVCDHELQCQCEEGWIPPDCDDSSVVFHFSIVVGVLFPMAVIFVVVAMVIRHQSSREKQKKDQRPLSTTGTRPHKQKRKPQMVKAVQPQEMSQMKPHVYDLPVEGNEPPASFHKDTNALPPTVFKDNPVSTPKDSNPKA

SEQ ID NO: 185 - protein, 820 aa, Homo sapiens
MKMLLLLHCLGVFLSCSGHIQDEHPQYHSPPDVVIPVRITGTTRGMTPPGWLSYILPFGGQKHIIHIKVKKLLFSKHLPVFTYTDQGAILEDQPFVQNNCYYHGYVEGDPESLVSLSTCFGGFQGILQINDFAYEIKPLAFSTTFEHLVYKMDSEEKQFSTMRSGFMQNEITCRMEFEEIDNSTQKQSSYVGWWIHFRIVEIVVVIDNYLYIRYERNDSKLLEDLYVIVNIVDSILDVIGVKVLLFGLEIWTNKNLIVVDDVRKSVHLYCKWKSENITPRMQHDTSHLFTTLGLRGLSGIGAFRGMCTPHRSCAIVTFMNKTLGTFSIAVAHHLGHNLGMNHDEDTCRCSQPRCIMHEGNPPITKFSNCSYGDFWEYTVERTKCLLETVHTKDIFNVKRCGNGVVEEGEECDCGPLKHCAKDPCCLSNCTLTDGSTCAFGLCCKDCKFLPSGKVCRKEVNECDLPEWCNGTSHKCPDDFYVEDGIPCKERGYCYEKSCHDRNEQCRRIFGAGANTASETCYKELNTLGDRVGHCGIKNATYIKCNISDVQCGRIQCENVTEIPNMSDHTTVHWARFNDIMCWSTDYHLGMKGPDIGEVKDGTECGIDHICIHRHCVHITILNSNCSPAFCNKRGICNNKHHCHCNYLWDPPNCLIKGYGGSVDSGPPPKRKKKKKFCYLCILLLIVLFILLCCLYRLCKKSKPIKKQQDVQTPSAKEEEKIQRRPHELPPQSQPWVMPSQSQPPVTPSQSHPQVMPSQSQPPVTPSQSQPRVMPSQSQPPVMPSQSHPQLTPSQSQPPVTPSQRQPQLMPSQSQPPVTPS

SEQ ID NO: 186 - protein, 790 aa, Homo sapiens
MRSVQIFLSQCRLLLLLVPTMLLKSLGEDVIFHPEGEFDSYEVTIPEKLSFRGEVQGVVSPVSYLLQLKGKKHVLHLWPKRLLLPRHLRVFSFTEHGELLEDHPYIPKDCNYMGSVKESLDSKATISTCMGGLRGVFNIDAKHYQIEPLKASPSFEHVVYLLKKEQFGNQVCGLSDDEIEWQMAPYENKARLRDFPGSYKHPKYLELILLFDQSRYRFVNNNLSQVIHDAILLTGIMDTYFQDVRMRIHLKALEVWTDFNKIRVGYPELAEVLGRFVIYKKSVLNARLSSDWAHLYLQRKYNDALAWSFGKVCSLEYAGSVSTLLDTNILAPATWSAHELGHAVGMSHDEQYCQCRGRLNCIMGSGRTGFSNCSYISFFKHISSGATCLNNIPGLGYVLKRCGNKIVEDNEECDCGSTEECQKDRCCQSNCKLQPGANCSIGLCCHDCRFRPSGYVCRQEGNECDLAEYCDGNSSSCPNDVYKQDGTPCKYEGRCFRKGCRSRYMQCQSIFGPDAMEAPSECYDAVNLIGDQFGNCEITGIRNFKKCESANSICGRLQCINVETIPDLPEHTTIISTHLQAENLMCWGTGYHLSMKPMGIPDLGMINDGTSCGEGRVCFKKNCVNSSVLQFDCLPEKCNTRGVCNNRKNCHCMYGWAPPFCEEVGYGGSIDSGPPGLLRGAIPSSIWVVSIIMFRLILLILSVVFVFFRQVIGNHLKPKQEKMPLSKAKTEQEESKTKTVQEESKTKTGQEESEAKTGQEESKAKTGQEESKANIESKRPKAKSVKKQKK

SEQ ID NO: 187 - protein, 787 aa, Homo sapiens
MFRLWLLLAGLCGLLASRPGFQNSLLQIVIPEKIQTNTNDSSEIEYEQISYIIPIDEKLYTVHLKQRYFLADNFMIYLYNQGSMNTYSSDIQTQCYYQGNIEGYPDSMVTLSTCSGLRGILQFENVSYGIEPLESAVEFQHVLYKLKNEDNDIAIFIDRSLKEQPMDDNIFISEKSEPAVPDLFPLYLEMHIVVDKTLYDYWGSDSMIVTNKVIEIVGLANSMFTQFKVTIVLSSLELWSDENKISTVGEADELLQKFLEWKQSYLNLRPHDIAYLLIYMDYPRYLGAVFPGTMCITRYSAGVALYPKEITLEAFAVIVTQMLALSLGISYDDPKKCQCSESTCIMNPEVVQSNGVKTFSSCSLRSFQNFISNVGVKCLQNKPQMQKKSPKPVCGNGRLEGNEICDCGTEAQCGPASCCDFRTCVLKDGAKCYKGLCCKDCQILQSGVECRPKAHPECDIAENCNGSSPECGPDITLINGLSCKNNKFICYDGDCHDLDARCESVFGKGSRNAPFACYEEIQSQSDRFGNCGRDRNNKYVFCGWRNLICGRLVCTYPTRKPFHQENGDVIYAFVRDSVCITVDYKLPRTVPDPLAVKNGSQCDIGRVCVNRECVESRIIKASAHVCSQQCSGHGVCDSRNKCHCSPGYKPPNCQIRSKGFSIFPEEDMGSIMERASGKTENTWLLGFLIALPILIVTTAIVLARKQLKKWFAKEEEFPSSESKSEGSTQTYASQSSSEGSTQTYASQTRSESSSQADTSKSKSEDSAEAYTSRSKSQDSTQTQSSSN

SEQ ID NO: 188 - protein, 813 aa, Homo sapiens
MGWRPRRARGTPLLLLLLLLLLWPVPGAGVLQGHIPGQPVTPHWVLDGQPWRTVSLEEPVSKPDMGLVALEAEGQELLLELEKNHRLLAPGYIETHYGPDGQPVVLAPNHTDHCHYQGRVRGFPDSWVVLCTCSGMSGLITLSRNASYYLRPWPPRGSKDFSTHEIFRMEQLLTWKGTCGHRDPGNKAGMTSLPGGPQSRGRREARRTRKYLELYIVADHTLFLTRHRNLNHTKQRLLEVANYVDQLLRTLDIQVALTGLEVWTERDRSRVTQDANATLWAFLQWRRGLWAQRPHDSAQLLTGRAFQGATVGLAPVEGMCRAESSGGVSTDHSELPIGAAATMAHEIGHSLGLSHDPDGCCVEAAAESGGCVMAAATGHPFPRVFSACSRRQLRAFFRKGGGACLSNAPDPGLPVPPALCGNGFVEAGEECDCGPGQECRDLCCFAHNCSLRPGAQCAHGDCCVRCLLKPAGALCRQAMGDCDLPEFCTGTSSHCPPDVYLLDGSPCARGSGYCWDGACPTLEQQCQQLWGPGSHPAPEACFQVVNSAGDAHGNCGQDSEGHFLPCAGRDALCGKLQCQGGKPSLLAPHMVPVDSTVHLDGQEVTCRGALALPSAQLDLLGLGLVEPGTQCGPRMVCQSRRCRKNAFQELQRCLTACHSHGVCNSNHNCHCAPGWAPPFCDKPGFGGSMDSGPVQAENHDTFLLAMLLSVLLPLLPGAGLAWCCYRLPGAHLQRCSWGCRRDPACSGPKDGPHRDHPLGGVHPMELGPTATGQPWPLDPENSHEPSSHPEKPLPAVSPDPQADQVQMPRSCLW

(19) United States
(12) Patent Application Publication
(10) Pub. No.: US 2013/0045244 A1
(43) Pub. Date: Feb. 21, 2013

(54) MODIFIED ADAM DISINTEGRIN DOMAIN POLYPEPTIDES AND USES THEREOF

(71) Applicants: Radu O. Minea, Arcadia, CA (US); Stephen D. Swensen, Arcadia, CA (US); Francis S. Markland, JR., Manhattan Beach, CA (US)

(72) Inventors: Radu O. Minea, Arcadia, CA (US); Stephen D. Swensen, Arcadia, CA (US); Francis S. Markland, JR., Manhattan Beach, CA (US)

(73) Assignee: University of Southern California

(21) Appl. No.: 13/578,551

(22) PCT Filed: Feb. 9, 2011
(86) PCT No.: PCT/US11/24243

§ 371 Date: Oct. 29, 2012

Related U.S. Application Data
(60) Provisional application No. 61/303,631, filed on Feb. 11, 2010.

(51) Int. Cl. C12N 9/50; C12N 15/57; C12N 15/62; C12N 15/70; C12N 1/21; C12N 5/071; G01N 33/574; G01N 23/00; G01N 21/64; C12N 11/02; A61K 38/48; A61P 35/00; A61P 35/02; A61K 9/00; C12N 9/96

(52) U.S. Cl. 424/400; 435/219; 435/188; 536/23.2; 536/23.4; 435/320.1; 435/252.33; 435/375; 435/7.23; 435/177; 424/94.67

(57) ABSTRACT

Modified ADAM (A Disintegrin and Metalloproteinase) Polypeptides (MAPs) are provided. Methods are provided for administering MAPs for anti-angiogenesis and anti-tumor growth activity. Compositions of the invention are also useful for treating endothelial cell dysfunction and for diagnosis of integrin-related conditions.

### CROSS-REFERENCE TO RELATED APPLICATIONS

This application claims the benefit of priority under 35 U.S.C. §119(e) to U.S. Provisional Application Ser. No. 61/303,631, filed Feb. 11, 2010, which is incorporated by reference herein in its entirety including all figures and tables.

### FIELD OF INVENTION

The invention relates to a class of engineered polypeptides that are derived from the ADAM (A Disintegrin and Metalloproteinase) mammalian family of multirole proteases and methods of making same. The invention also relates to the use of these engineered polypeptides for anti-angiogenesis and anti-tumor growth activity. The invention also relates to administering the engineered polypeptides for endothelial cell dysfunction and for diagnosis of integrin-related conditions.

### BACKGROUND OF THE INVENTION

The invention is related to U.S. Publication no. 20060246541 by Minea et al., and titled “Method of expressing proteins with disulfide bridges” and to PCT Patent Application No. PCT/US09/64256, filed Nov. 12, 2009, and titled “Method of expressing proteins with disulfide bridges with enhanced yields and activity.” The contents of both are incorporated herein by reference thereto including all figures.

The ADAMs are class of multidomain mammalian transmembrane or secreted proteins that are involved in every step of embryonic development (where they control cell proliferation, cell migration, cell specification, axon elongation and organ morphogenesis) as well as in multiple physiological and pathological processes in the adult life (from wound healing to various inflammatory processes and from angiogenesis and metastasis to organ repair and regeneration) [1-5]. Numerous reports show that many proteolytic members of ADAM family appear to be overexpressed in human malignancies which indicates that these proteases may play important roles in tumor progression [6-15]. Interestingly, while the overexpression of catalytically active ADAMs appears to be generally linked to poor outcomes in cancers, the non-proteolytic ones seem to play a role as tumor inhibitors. For instance, the overexpression of ADAM22 in human gliomas has been shown to correlate with tumor growth inhibition [16].

The structure of ADAM proteins closely resembles that of their orthologues, the PIII-class snake venom metalloproteinases. Like the PIII-class of snake venom metalloproteinases (SVMPs), the ADAMs are multidomain proteins that possess metalloprotease, disintegrin and cysteine-rich domains [17, 28]. The ADAM scaffold contains several inter- and intra-domain cysteine residues, among which is a very important one located in the disintegrin domain at the tip of a structural element called the disintegrin loop. The inter-domain cysteine residues are located in the spacer regions between the metalloprotease and disintegrin domains and between the disintegrin and cysteine-rich domains [20]. These cysteine residues, which are found in ADAMs and PIII-SVMPs but generally not in snake venom disintegrins, form the disulfide bridges that link the inter-domain (spacer) regions with the metalloprotease, disintegrin and cysteine-rich domains in ADAMs and PIII-SVMPs [20]. These spacer-domain disulfide bridges in the ADAM scaffold represent the structural elements that lock the three domains together in a tightly folded structure allowing these multidomain proteins to better survive in the extracellular environment (i.e., more resistant to a proteolytic attack etc). This stabilized ADAM scaffold naturally evolved through disulfide bond engineering into a newer fold, the PII-class SVMPs, that gives rise to free disintegrins through a mechanism that involves a proteolytic attack. As a result of molecular evolution, several of the cysteine residues characteristic of the ADAM scaffold (i.e., the ones that participate in spacer-domain disulfide bridges) were either mutated to a different residue or deleted. The consequence of these mutations was that disulfide bridges could no longer form between the spacer region and both the metalloprotease and disintegrin domains in these newer proteins (the PIIclass SVMPs) which renders their inter-domain regions susceptible to proteolysis and makes possible the release of the individual domains as free domains of snake venom disintegrins [20]. Therefore, the PII snake venom disintegrins emerged as a class of free disintegrin domain polypeptides that have become the most potent natural platelet aggregation inhibitors through their high-affinity interaction with the platelet specific integrin alphaIIbbeta3. In contrast to the ADAM scaffold disintegrin domains, these later evolving PII class SVMPs are released as free polypeptides in the venom of hematotoxic snakes and possess a novel 11-amino acid disintegrin loop, the unique structural element characteristic to this class of molecules and naturally engineered to act as a potent soluble integrin ligand by mimicking the action of extracellular matrix protein motifs (ECM-mimetic) [19]. This loop freely protrudes from the disulfide stabilized polypeptide core and interacts with integrin receptors via a tripeptide motif (usually an Arg-Gly-Asp motif) that is displayed at the tip of the loop [21].

There are 24 members in the human integrin family of which 23 have a complex expression pattern, tissue distribution and physiological functions whereas the alphaIIbbeta3 integrin is a platelet specific receptor instrumental to blood clot formation. When deregulated in different tissues or organs, by being either abnormally expressed, or mislocalized at the plasma membrane or inappropriately activated or a combination of these mechanisms, the abnormal functioning of many of these receptors is linked to a diverse pathology ranging from neoplasia to inflammatory diseases and to complex physiological responses such as wound healing and tissue regeneration [22-25].

Snake venom disintegrins have therapeutic potential as anticancer agents when delivered in a liposomal formulation [21, 26, 27]. Aside from their natural biological activity (i.e., the inhibition of platelet aggregation through a high affinity interaction with integrin alphaIIbbeta3), it has been shown that snake venom disintegrins disrupt tumor-associated processes such as metastasis and angiogenesis by their ability to engage a defined set of integrins (e.g., alphavbeta3, alphavbeta5, and alpha5beta1) that are mechanistically involved in the pathogenesis of these processes [21]. Despite these favorable attributes, these polypeptides still possess potentially negative immunological characteristics due to their derivation from snake venom. Free disintegrins are not known to be present in humans or any other mammals—the only human disintegrins identified so far exist as subdomains buried within the larger sequence of the ADAM family protein members [17]. There are over 30 ADAM proteins indentified in the mammalian kingdom (of which humans possess 20 genes and 3 pseudogenes) and all of them possess disintegrin domains [1]. Of 23 ADAM transcripts identified in humans, 3 are from pseudogenes (ADAM 1, 3 and 6) which do not translate into functional proteins.

### DESCRIPTION OF THE FIGURES

FIG. 1 shows the amino acid sequences of the disintegrin-like domains of the 23 human ADAM proteins (APs). FIG. 1A shows the sequence alignment of disintegrin-like domains of human ADAM proteins. The amino acid residues that are crossed out indicate amino acids that are removed in the corresponding MAP polypeptides. The amino acids in bold in APs 1 and 17 were replaced with another amino acid in the corresponding MAPs to conserve the medium-sized snake venom disintegrin cysteine pattern. FIG. 1B shows the tripeptide motifs (boxed amino acid residues) that are displayed at the tips of each of the APs' disintegrin loops of the disintegrin-like domain. FIG. 1C shows the disintegrin loops (boxed amino acid residues) of the disintegrin-like domains.

FIG. 2 shows the amino acid sequences of the MAP polypeptides. FIG. 2A shows the amino acid sequences of the 23 MAP polypeptides aligned against the sequence of trimestatin, a prototypical medium-size snake venom disintegrin (purified from the venom of Trimeresurus flavoviridis snake). The cysteine residues are aligned and underlined. The replacement amino acid residues corresponding to those in AP1 and AP17 for MAP1 and MAP17, respectively are shown in bold. In addition, the N-terminus residue in APs 1, 3, 6, 18, 21, 30, and 32 was replaced by a glycine residue in their corresponding MAPs. FIG. 2B shows the tripeptide motifs (boxed amino acids) that are displayed at the tips of each of the MAPs' disintegrin loops of the disintegrin-like domain and by medium-size snake venom trimestatin. FIG. 2C identifies the disintegrin loops (boxed amino acids) of MAPs and trimestatin.

FIG. 3 shows sequence alignment of snake venom disintegrins with the disintegrin-like domains of human ADAM proteins. The disintegrin-like sequences of PIII-class snake venom metalloproteases (VAP1 and Catrocollastatin) were aligned with long-size snake venom disintegrin sequences (Salmosin3 and Bitistatin), a prototypical medium-size snake venom disintegrin (Trimestatin) and several human ADAM disintegrin-like domains (AP7, AP8, AP12, AP19, AP28, and AP33). The amino acid residues in disintegrin-like sequences (from both snake and human origin) that can be modified (e.g. deleted) in order to generate the corresponding MAP polypeptide are shown crossed out. The tripeptide amino acid motifs displayed at the tip of disintegrin loops in these disintegrin and disintegrin-like sequences are shown in a box.

FIG. 4 compares expression of Trx-D9 (native human ADAM9 disintegrin-like domain sequence-thioredoxin fusion polypeptide) to Trx-MAP9 (MAP9-thioredoxin fusion polypeptide) in different E. coli hosts (BL21 versus Origami B) analyzed by SDS-PAGE. From left to right, lanes represent: PageRuler™ Plus Prestained Protein Ladder (Fermentas, Burlington, ON), lysates from Trx-D9-transformed BL21 (DE3) cells grown and induced in Carbenicillin, lysates from Trx-D9-transformed Origami B (DE3) cells initially plated on 3 AB (Carbenicillin, Kanamycin and Tetracycline), but further expanded and induced in Carbenicillin only, lysates from Trx-MAP9-transformed BL21 (DE3) cells grown and induced in Carbenicillin, and lysates from Trx-MAP9-transformed Origami B (DE3) cells initially plated on 3 AB (Carbenicillin, Kanamycin and Tetracycline), but further expanded and induced in Carbenicillin only.

FIG. 5 compares expression of Trx-D15 (native human ADAM15 disintegrin-like domain sequence-thioredoxin fusion polypeptide) to Trx-MAP15 (MAP15-thioredoxin fusion polypeptide) in different E. coli hosts (BL21 versus Origami B) analyzed by SDS-PAGE. From left to right, lanes represent: PageRuler™ Plus Prestained Protein Ladder (Fermentas, Burlington, ON), lysates from Trx-D15-transformed BL21 (DE3) cells grown and induced in Carbenicillin, lysates from Trx-D15-transformed Origami B (DE3) cells initially plated on 3 AB (Carbenicillin, Kanamycin and Tetracycline), but further expanded and induced in Carbenicillin only, lysates from Trx-MAP15-transformed BL21 (DE3) cells grown and induced in Carbenicillin, and lysates from Trx-MAP15-transformed Origami B (DE3) cells initially plated on 3 AB (Carbenicillin, Kanamycin and Tetracycline), but further expanded and induced in Carbenicillin only.

FIG. 6 shows the nucleic acid sequences of MAPs inserted into a pET32a expression vector 3′ to bacterial TrxA. FIG. 6A shows the DNA sequences of MAPs 1, 2, 3, and 6. FIG. 6B shows the nucleic acid sequences of MAPs 7, 8, 9, and 10. FIG. 6C shows the nucleic acid sequences of MAPs 11, 12, 15, and 17. FIG. 6D shows the nucleic acid sequences of MAPs 18, 19, 20, and 21. FIG. 6E shows the nucleic acid sequences of MAPs 22, 23, 28, and 29. FIG. 6F shows the nucleic acid sequences of MAPs 30, 32, and 33.

FIG. 7 shows the oligonucleotide primer sequences used for cloning of MAP constructs into a pET32a expression vector. FIG. 7A shows the oligonucleotide primers used for cloning of MAPs 1, 2, 3, 6, 7, 8, 9, 10, 11, 12, 15, 17, and 18. FIG. 7B shows the oligonucleotide primers used for cloning of MAPs 19, 20, 21, 22, 23, 28, 29, 30, 32, and 33.

FIG. 8 shows the amino acid sequences of all TrxA-MAP constructs that were expressed in E. coli Origami B (DE3). The active site of TrxA (CGPC) is italicized, the tripeptide motif at the tip of the disintegrin loop is underlined, the TEV cleavage recognition site is highlighted in a box and the linker region between TrxA and various MAP constructs is in bold and italicized. The amino acid residues introduced to replace the residues in AP1 and AP17 in the corresponding MAP1 and MAP17 are highlighted in bold double-underlined.

FIG. 9 shows expression of Trx-MAP2 (MAP2-thioredoxin fusion polypeptide), Trx-MAP7 (MAP7-thioredoxin fusion polypeptide), Trx-MAP8 (MAP8-thioredoxin fusion polypeptide), and Trx-MAP9 (MAP9-thioredoxin fusion polypeptide) expression levels in Origami B host analyzed by SDS-PAGE. From left to right, lanes represent: PageRuler™ Plus Prestained Protein Ladder (Fermentas, Burlington, ON), lysates from Trx-MAP2-transformed Origami B (DE3) cells, lysates from Trx-MAP7-transformed Origami B (DE3) cells, lysates from Trx-MAP8-transformed Origami B (DE3) cells, and lysates from Trx-MAP9-transformed Origami B (DE3). All transformants were initially plated on 3 antibiotics (Carbenicillin, Kanamycin and Tetracycline), but further expanded and induced in Carbenicillin only.

FIG. 10 shows expression of Trx-MAP10 (MAP10-thioredoxin fusion polypeptide), Trx-MAP12 (MAP12-thioredoxin fusion polypeptide), Trx-MAP15 (MAP15-thioredoxin fusion polypeptide), and Trx-MAP17 (MAP17-thioredoxin fusion polypeptide) expression levels in Origami B host analyzed by SDS-PAGE. From left to right, lanes represent: PageRuler™ Plus Prestained Protein Ladder (Fermentas, Burlington, ON), lysates from Trx-MAP10-transformed Origami B (DE3) cells, lysates from Trx-MAP12-transformed Origami B (DE3) cells, lysates from Trx-MAP15-transformed Origami B (DE3) cells, and lysates from Trx-MAP17-transformed Origami B (DE3). All transformants were initially plated on 3 antibiotics (Carbenicillin, Kanamycin and Tetracycline), but further expanded and induced in Carbenicillin only.

FIG. 11 shows expression of Trx-MAP19 (MAP19-thioredoxin fusion polypeptide), Trx-MAP23 (MAP23-thioredoxin fusion polypeptide), Trx-MAP28 (MAP28-thioredoxin fusion polypeptide), and Trx-MAP33 (MAP33-thioredoxin fusion polypeptide) expression levels in Origami B host analyzed by SDS-PAGE. From left to right, lanes represent: PageRuler™ Plus Prestained Protein Ladder (Fermentas, Burlington, ON), lysates from Trx-MAP19-transformed Origami B (DE3) cells, lysates from Trx-MAP23-transformed Origami B (DE3) cells, lysates from Trx-MAP28-transformed Origami B (DE3) cells, and lysates from Trx-MAP33-transformed Origami B (DE3). All transformants were initially plated on 3 antibiotics (Carbenicillin, Kanamycin and Tetracycline), but further expanded and induced in Carbenicillin only.

FIG. 12 shows flow cytometry detection of binding of various Trx-MAPs, as indicated in the figure, to MDA-MB-231 cells (human breast carcinoma).

FIG. 13 shows flow cytometry detection of binding of various Trx-MAPs, as indicated in the figure, to a bone-homing subclone of MDA-MB-231 cells (human breast carcinoma).

FIG. 14 shows flow cytometry detection of binding of various Trx-MAPs, as indicated in the figure, to a brain-homing subclone of MDA-MB-231 cells (human breast carcinoma).

FIG. 15 shows flow cytometry detection of binding of various Trx-MAPs, as indicated in the figure, to a Jurkat cells (human T-cell leukemia cell line).

FIG. 16 shows flow cytometry detection of binding of MAP9 and MAP15 to HUVEC (Human Umbilical Vein Endothelial Cells), MDA-MB-435 (human breast carcinoma), MDA-MB-231 (human breast carcinoma), and a glioblastoma multiforme cancer stem cell line (GBM-CSC).

FIG. 17 shows HUVEC tube formation assays in the presence of 10 nM MAP9 or MAP15. Panel A—untreated control; panel B—100 μM Suramin; panel C—10 nM MAP9; panel D—10 nM MAP15. Cells were stained with Calcein AM and imaged using confocal microscopy. All images were taken at the same magnification (scale bar=50 μm).

FIG. 18 shows tumor growth inhibition (panel A) and survival (panel B) in MDA-MB-231 xenografts by MAP15 and a liposomal formulation of MAP15 treatment as compared to other anticancer treatments (Avastin and Docetaxel).

FIG. 19 shows microvessel inhibition in a tumor angiogenesis assay in photomicrographs (panel A) and by quantitation of microvessel density from random photomicrographs (panel B) in MDA-MB-231 xenografts by a liposomal formulation of MAP15 treatments as compared to other anticancer treatments (Avastin and Docetaxel) and in combination with other anticancer treatments.

FIG. 20 shows the nucleic acid sequence corresponding to the ADAM1 transcript.

FIG. 21 shows the nucleic acid sequence corresponding to the ADAM2 transcript.

FIG. 22 shows the nucleic acid sequence corresponding to the ADAM3 transcript, variant 1.

FIG. 23 shows the nucleic acid sequence corresponding to the ADAM6 transcript.

FIG. 24 shows the nucleic acid sequence corresponding to the ADAM7 transcript.

FIG. 25 shows the nucleic acid sequence corresponding to the ADAM8 transcript, variant 1.

FIG. 26 shows the nucleic acid sequence corresponding to the ADAM9 transcript, variant 1.

FIG. 27 shows the nucleic acid sequence corresponding to the ADAM10 transcript.

FIG. 28 shows the nucleic acid sequence corresponding to the ADAM11 transcript.

FIG. 29 shows the nucleic acid sequence corresponding to the ADAM12 transcript, variant 1.

FIG. 30 shows the nucleic acid sequence corresponding to the ADAM15 transcript, variant 6.

FIG. 31 shows the nucleic acid sequence corresponding to the ADAM17 transcript.

FIG. 32 shows the nucleic acid sequence corresponding to the ADAM18 transcript, variant 1.

FIG. 33 shows the nucleic acid sequence corresponding to the ADAM19 transcript.

FIG. 34 shows the nucleic acid sequence corresponding to the ADAM20 transcript.

FIG. 35 shows the nucleic acid sequence corresponding to the ADAM21 transcript.

FIG. 36 shows the nucleic acid sequence corresponding to the ADAM22 transcript, variant 1.

FIG. 37 shows the nucleic acid sequence corresponding to the ADAM23 transcript.

FIG. 38 shows the nucleic acid sequence corresponding to the ADAM28 transcript, variant 1.

FIG. 39 shows the nucleic acid sequence corresponding to the ADAM29 transcript, variant 1.

FIG. 40 shows the nucleic acid sequence corresponding to the ADAM30 transcript.

FIG. 41 shows the nucleic acid sequence corresponding to the ADAM32 transcript.

FIG. 42 shows the nucleic acid sequence corresponding to the ADAM33 transcript, variant 1.

FIG. 43 shows the amino acid sequences for ADAM2, ADAM7, ADAM8, ADAM9, ADAM10, ADAM 11, ADAM12, ADAM15, ADAM17, ADAM18, ADAM19, ADAM20, ADAM21, ADAM22, ADAM23, ADAM28, ADAM29, ADAM30, ADAM32, and ADAM33 polypeptides.

### SUMMARY OF THE INVENTION

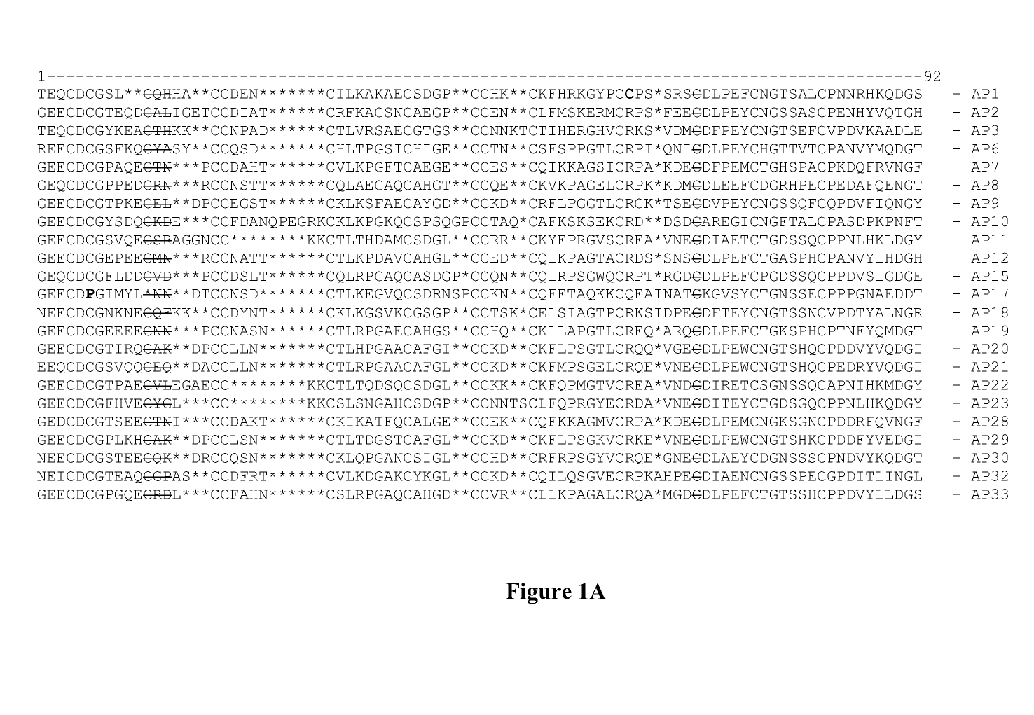

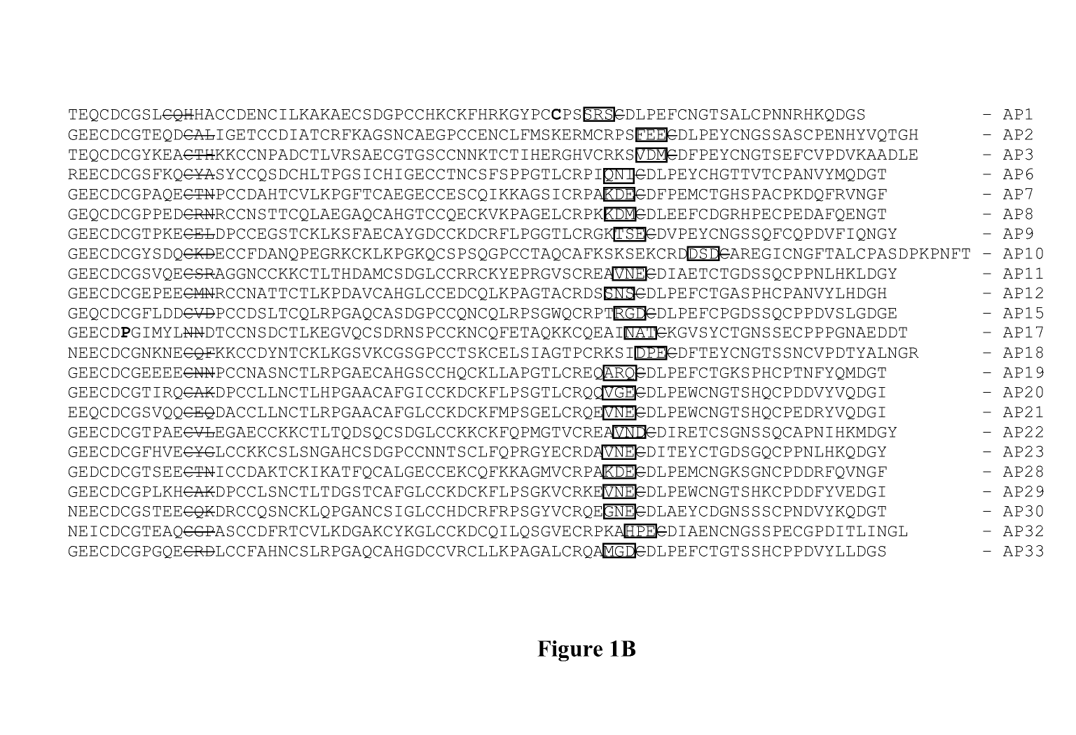

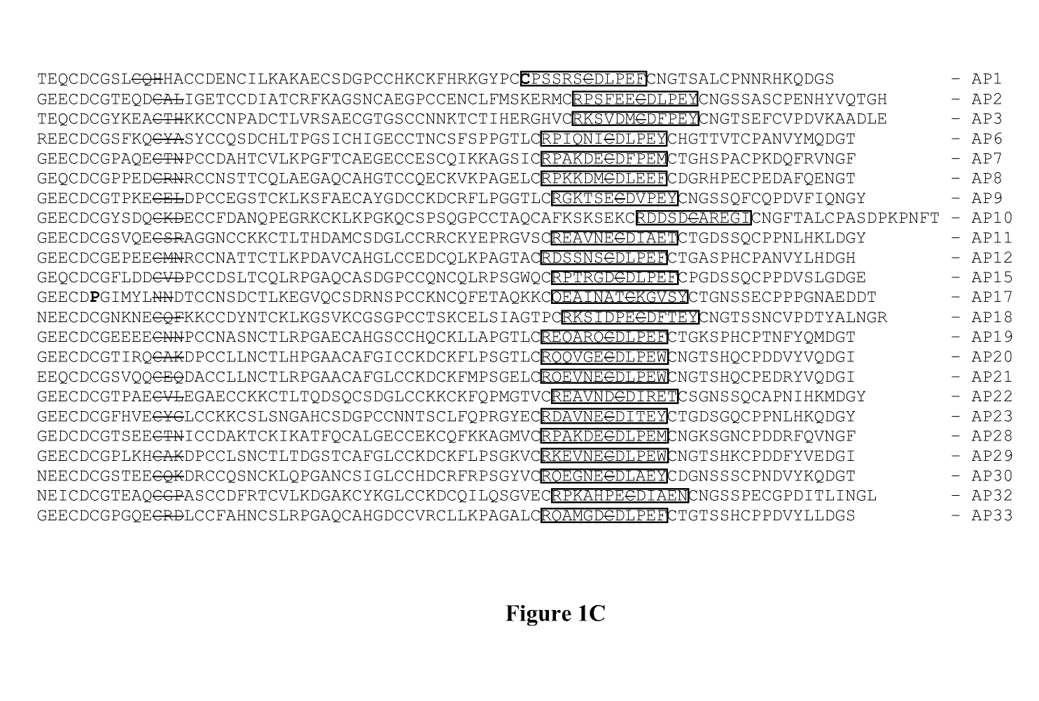

Provided herein compositions, and methods related thereto, of Modified ADAM-derived Polypeptides (MAPs), having an ADAM-derived Polypeptide (AP) that is modified at the following amino acid residues:
a) a first cysteine C-terminal to a Cys-Asp-Cys (CDC) motif;
b) two contiguous amino acids C-terminal to the first cysteine; and,
c) a cysteine C-terminal to a tripeptide motif,
   and wherein the modification of the amino acid residues is independently selected from the following:
a) deletion;
b) substitution; and,
c) chemical modification,
   and, wherein, the AP has a distintegrin-like domain from an ADAM having (i) the Cys-Asp-Cys (CDC) motif, (ii) the tripeptide motif as indicated in FIG. 1 and (iii) lacking all or substantially all of the ADAM metalloprotease domain, cysteine-rich domains, and interdomain segments, and wherein the ADAM is not ADAM17, as ADAM17 has a CDP rather than a CDC motif. Provided herein are nucleic acids that encode these MAPs.

The MAP can be derived from an ADAM (A Disintegrin and Metalloproteinase) which includes ADAM15, ADAM28, ADAM1, ADAM2, ADAM3, ADAM6, ADAM7, ADAM8, ADAM9, ADAM10, ADAM 11, ADAM12, ADAM18, ADAM19, ADAM20, ADAM21, ADAM22, ADAM23, ADAM29, ADAM30, ADAM32, ADAM33. These corresponding MAPs can be MAP15, MAP28, MAP1, MAP2, MAP3, MAP6, MAP7, MAP8, MAP9, MAP10, MAP11, MAP12, MAP18, MAP19, MAP20, MAP21, MAP22, MAP23, MAP29, MAP30, MAP32, MAP33.

Also provided herein are fusion proteins of MAPs with an N-terminal segment of thioredoxin and nucleic acids that code these fusions.

The MAPs can have the properties of inhibiting the movement of HUVEC or MDA-MB-435 cells through a reconstituted basement membrane, increasing the level of phosphorylation of FAK in MDA-MB-435 cells or inhibiting tube formation of HUVECs in culture.

Also provided herein are expression vectors that express MAPs and prokaryotic host cells transformed with these expression vectors. The expression vector can be under inducible control, such as where the host also carries stable mutations in thioredoxin reductase B (trxB) gene and/or the glutathione reductase (gor) gene.

Also provided herein are methods of treating an individual suffering from cancer by administering an effective amount of at least one MAP. The cancer can be an integrin expressing cancer. The cancer can be breast cancer, colorectal cancer, basal cell carcinoma, adenocarcinoma, gastrointestinal cancer, colon cancer, liver cancer, bladder cancer, pancreatic cancer, ovarian cancer, cervical cancer, lung cancer, skin cancer, prostate cancer, renal cell carcinoma, central nervous system (CNS) cancer, and leukemia. The gastrointestinal cancer can be lip cancer, mouth cancer, esophageal cancer, small bowel cancer or stomach cancer. The skin cancer can be squamous cell or basal cell cancer.

Also provided herein are methods of inhibiting the binding of an integrin to a ligand by contacting a cell that expresses the integrin with an effective amount of a MAP or fusion thereof.

Also provided herein are methods of determining the presence of cancer cells in an individual by contacting the cancer cells with at least one MAP and detecting the at least one MAP. The MAP can be labeled and the label can be a Positron Emmission Tomography (PET) probe or a fluorescent probe.

Also provided herein are methods of preparing an artificial ECM scaffold by coating an artificial ECM scaffold with a MAP. The artificial ECM scaffold can further include stem cell precursors. The artificial ECM scaffold can be a urinary bladder scaffold, an esophageal scaffold or an anal scaffold.

Also provided are stents coated with a composition that includes at least one MAP.

Also provided are MAPs that are an AP having a distintegrin-like domain from an ADAM having (i) the Cys-Asp-Cys (CD) motif, (ii) the tripeptide motif as indicated in FIG. 1 and (iii) lacking all or substantially all of the ADAM metalloprotease domain, cysteine-rich domains, and interdomain segments, and further comprising amino acid modification that results in disruption of the interdomain disulfide linkages with the first cysteine C-terminal to the CDC motif and interdomain disulfide linkages with the cysteine C-terminal to the tripeptide motif.

### DETAILED DESCRIPTION OF THE INVENTION

Provided herein are a class of uniquely designed polypeptides, designated MAPs (Modified ADAM-derived Polypeptides), and encoding nucleic acids. As used herein, MAPs refer to a modified form of the native disintegrin domain of an ADAM protein. See Appendix for listing of ADAM encoding nucleic acid and amino acid sequences. As used herein, a “disintegrin domain of an ADAM protein” which may be referred to herein as “AP” (“ADAM derived Polypeptide”) is a disintegrin domain of the ADAM lacking all or substantially all of its metalloprotease and cysteine-rich domains and interdomain segments. Lacking substantially all means that the remaining amino acid sequence no longer retains the function of that domain. Examples of APs are shown in FIG. 1. The N-terminal end of the AP starts at the position 3 amino acid residues N-terminal from the CDC motif up to but not including the first cysteine N-terminal to the CDC. The C-terminal end of the AP ends at the position 10 amino acid residues C-terminal from the 12th cysteine residue from the CDC motif up to but not including the next cysteine C-terminal to said 12^{th} cysteine residue. There are two exceptions: (1) the C-terminal end of AP1 ends at the position 10 amino acid residues C-terminal from the 13th cysteine residue from the CDC motif up to but not including the next cysteine C-terminal to said 13^{th} cysteine residue, and (2) ADAM17 has a CDP motif rather than a CDC motif from which the ends of the corresponding AP (AP17) are delineated.

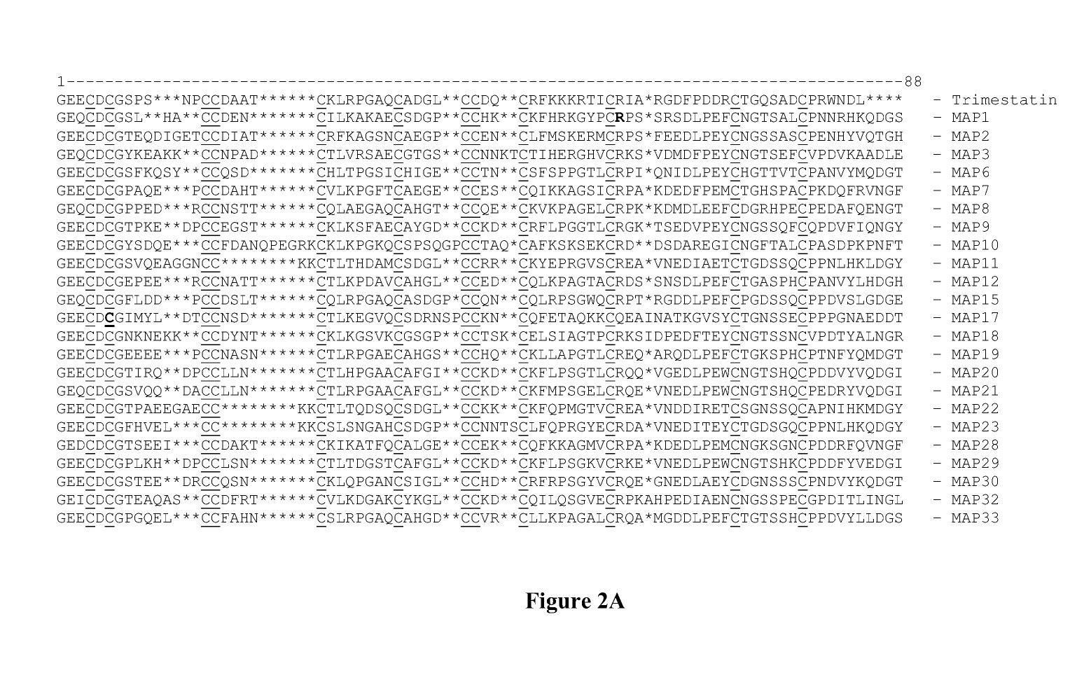

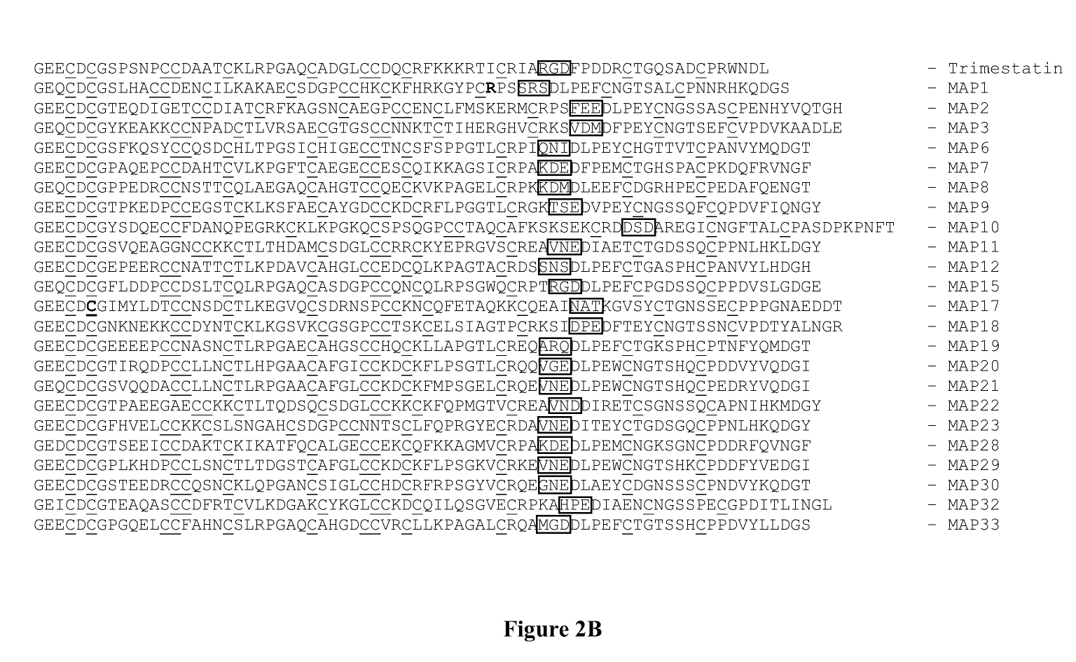

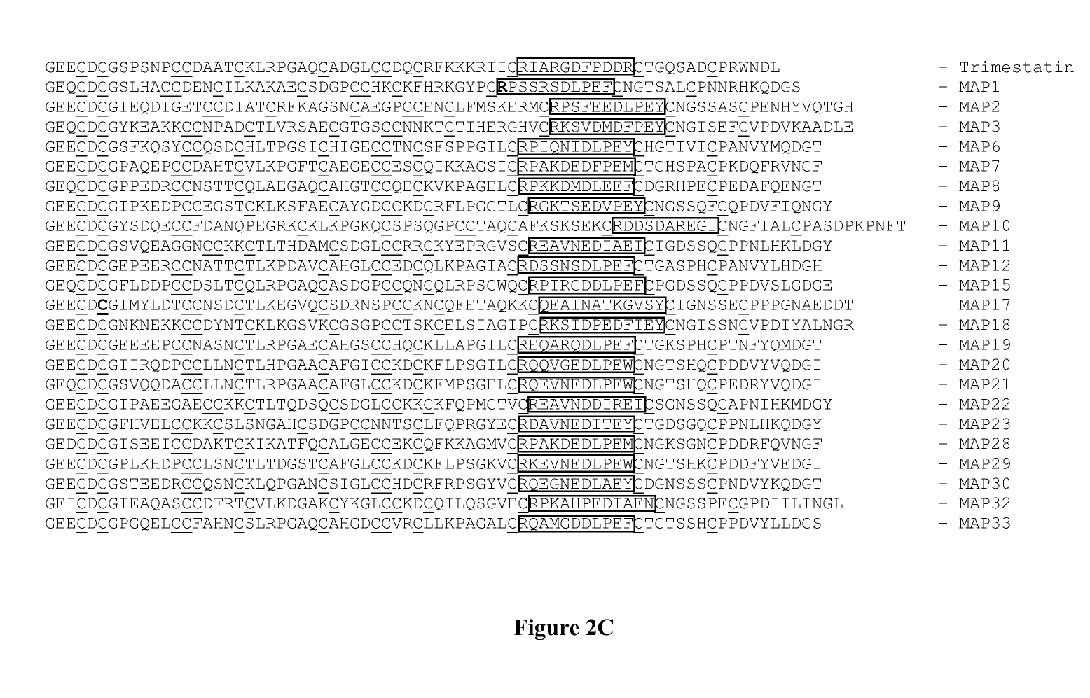

A “MAP” is a “modified” form of an AP, the modifications involving an alteration(s) in the amino acid sequence of the AP to achieve the beneficial properties described herein. MAPs, therefore, have sequences which are modified relative to the sequence normally present in the AP and corresponding sequence of the ADAM polypeptide. As used herein, “modified” means that the amino acid is deleted, substituted or chemically modified and, in an embodiment, the modification results in disruption of interdomain disulfide linkage. Exemplary MAPs are shown in FIG. 2. The MAP sequences are shown aligned with trimestatin, a prototypical medium-size snake venom disintegrin. All MAP constructs were modeled after medium-size snake venom disintegrins and had their sequences modified to fold similarly to these native snake venom molecules. The MAPs (except for MAP17) were constructed such that the first cysteine C-terminal to the CDC motif and two amino acids C-terminal to said cysteine as well as the cysteine C-terminal to the tripeptide motif of the corresponding AP are deleted. Alternatively, the cysteine residues can be substituted with alternate amino acids or the cysteine amino acid residues can be chemically modified so as to prevent disulfide bond formation. The amino acid substitutions can be conservative, e.g. the first cysteine C-terminal to the CDC motif of the AP can be substituted with a serine residue, the amino acid residues C-terminal to the cysteine can be substituted with a charged amino acid, or the cysteine C-terminal to the tripeptide motif can be substituted with a charged amino acid. Such mutational approaches and chemical modifications of amino acid residues are well known in the art. With regard to chemical modifications, an example is the use alkylating agents to react with cysteine residues to prevent formation of disulfide bonds. Except for MAP10, 17, 18 and 32, MAPs display an 11 amino acid disintegrin loop, similar to the native loop of snake venom disintegrins. MAP 10 displays a 10 amino acid integrin loop and MAP17, MAP18, and MAP32 display a 12 amino acid disintegrin loop.

MAPs can be expressed and further purified as stand alone biologically active molecules in a bacterial system that supports both the generation of active soluble disulfide-rich polypeptides and high expression yields for these products. While not wishing to be bound by theory, the MAPs were designed from the native APs so that they could adopt a snake venom disintegrin fold rather than their native ADAM conformations. The MAPs can be expressed with high yields in the Origami B (DE3) E. coli strain and further purified as stable and active free polypeptides that can interact with a class of mammalian cell surface receptors, the integrins, in a manner that is similar to that of native snake venom disintegrins. The MAPs can also retain some of the signaling properties that are characteristic of the APs or disintegrin domain activities from the ADAM polypeptide from which the MAP was derived. For instance, retained characteristics may include signaling attributes related to the putative ability of the ADAM disintegrin domains to engage integrin receptors by utilizing amino acid residues located outside the classical disintegrin loop. Cellular functions of ADAMs are well known [1-5, 34].

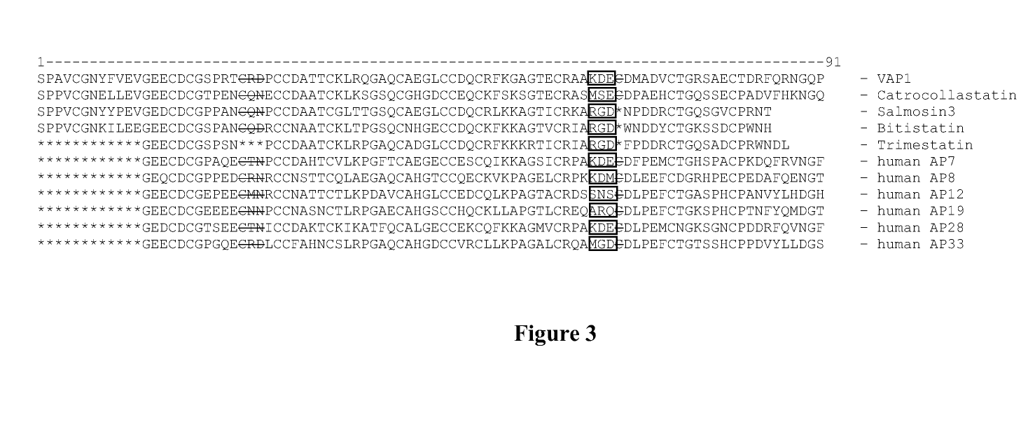

Although not wishing to be bound by theory, it is believed that the PII-class SVMPs that give rise to the prototypical medium-sized snake venom disintegrins (e.g., Trimestatin, Kistrin, Flavoridin etc) fail to form a critical disulfide bridge between the upstream spacer region and the disintegrin domain and thus the proteolytic attack happens in the residues located immediately N-terminal to where the disintegrin domain starts, the consequence of this being that the released medium-sized disintegrins are complete disintegrin domains containing no portion of the upstream spacer region. In contrast, it is believed that the PII-class SVMPs that give rise to the long-sized snake venom disintegrins (e.g., Bitistatin, Salmosin3 etc) fail to form a critical disulfide bridge between the metalloprotease domain and the downstream spacer region and consequently a proteolytic attack happens further N-terminal in the spacer region with the release of a longer disintegrin having a portion of the spacer region attached N-terminally to the freed disintegrin domain (see the sequence alignment of various disintegrin and disintegrin domains in FIG. 3). Moreover, it is also believed that when the PII-SVMPs contain even more mutations and/or deletions, the disulfide bridges fail to form in the same spacer region but also in the N-terminal part of the disintegrin domain and even shorter variants of snake venom disintegrins are released (e.g., either partially truncated disintegrins domains that dimerize like Contortrostatin or, more rarely, extremely truncated polypeptides like Echistatin or Eristostatin). It is further believed that, in almost all cases, the free disintegrin domains display a conserved 11-amino acid disintegrin loop in the C-terminal half of their molecule, which is the hallmark of snake venom disintegrins.

The 23 different ADAM transcripts that have been identified in the human genome (3 of them being pseudogenes that are not normally translated into a protein product) have been used as the basis for creating the encoded MAPs as described herein that adopt the snake venom disintegrin fold.

Several ADAM transcripts encode a number of isoforms. Nonetheless, inside the isoforms of different ADAMs the disintegrin domain's sequence is conserved and therefore there are only 23 different disintegrin domains in the human family of ADAM proteins. When produced recombinantly, the MAPs of the invention can interact in a high affinity manner with a defined integrin set. This property makes these mutant polypeptides broad spectrum integrin ligands for clinical and therapeutic use.

Similar to the other human ADAM member transcripts, the non-functional transcripts do contain complete disintegrin sequences that, if artificially translated in a recombinant system, can generate active polypeptides with novel biological functions. The disintegrin domains of human ADAMs have between 76 to 86 amino acids (the disintegrin domain of ADAM1 is the shortest, whereas that of ADAM10 is the longest), and, with 2 exceptions (ADAMs 1 and 17), they all contain 14 canonical cysteine residues of the ADAM scaffold (see the aligned sequences of human ADAMs below). See FIG. 1. Unlike the snake venom disintegrins, that evolved to function as platelet aggregation inhibitors, most of which contain an RGD tripeptide motif at the tip of their disintegrin loop, the disintegrin loops of ADAMs display much different tripeptide motifs at their tips and therefore are expected to engage a broader range of integrins and in a different manner than their snake venom counterparts. In fact, each of the APs is believed to bind to a defined and unique set of integrin receptors thus signaling in a unique manner (see FIG. 3 for the sequence alignment of ADAM and snake venom disintegrins illustrating the differences in the disintegrin loops). Not wishing to be bound by theory, it is believed that combinations of 2 or more MAPs can be used to determine an “integrin signature” that is characteristic of a particular cell type or disease state for a cell type. An integrin signature means a combination of integrins present on the surface of a cell that is unique to that cell type or the disease state for that cell type.

The disintegrin domain of human ADAM15 contains a RGD tripeptide motif in its disintegrin loop which supports the hypothesis that human ADAM15 plays important regulatory roles in the cardiovascular system. This RGD tripeptide motif in ADAM 15 is shown in AP15 in FIG. 1.

MAPs for each AP portion of all 23 known human ADAM members were generated. The human ADAM disintegrin domain sequences were modified according to the rationale presented above, which includes removing the residues (among which include 2 cysteine residues) in the ADAM disintegrin domain that normally participate in interdomain-disintegrin domain disulfide bridge formation in the native ADAM proteins. Not wishing to be bound by theory, the apparent function of these disulfide bridges is to keep the disintegrin loops in ADAMs tightly packed and unavailable to integrin receptors. By modifying the residues that participate in the formation of these disulfide bridges, such as by deletion, these MAPs acquire the mobility of the canonical 11-amino acid loop and the disintegrin-fold characteristic of snake venom disintegrins. Among the 23 members of the human ADAMs, 6 members perfectly fit the above-mentioned scheme (ADAMs 7, 8, 12, 19, 28 and 33) when aligned with long- and medium-sized snake venom disintegrins as well as with PIII-class SVMPs (see FIG. 3 for an alignment of snake venom disintegrins and human ADAM disintegrin domains). Nonetheless, by introducing these modifications, with the exception of 4 ADAMs (10, 17, 18 and 32), all human ADAM members were converted to MAPs that display a 11-amino acid disintegrin loop. Regarding the 4 exceptions, 3 (ADAMs 17, 18 and 32) were converted to MAPs displaying a slightly longer, 12-amino acid loop, while 1 member (ADAM 10) was converted to a MAP carrying a slightly shorter 10-amino acid disintegrin loop (see AP10 in FIG. 2 for the MAP sequence alignment). Moreover, in the case of 2 APs (ADAMs 1 and 17), one additional native residue in each sequence was replaced with either an arginine residue (to generate MAP1) or a cysteine residue (to generate MAP17) to restore the cysteine pattern characteristic of snake venom disintegrin domains (see FIG. 2 for the MAP sequence alignment).

As used herein, “interdomain regions” or “spacer regions” means the polypeptide portion of an ADAM between the metalloprotease and disintegrin domain (the “MD interdomain region”) and between the disintegrin domain and the cysteine-rich domain (the “DC interdomain region”), respectively, wherein the MD interdomain region starts at least 10 amino acid residues N-terminal to the AP and the DC interdomain region starts at least 10 amino acid residues C-terminal to the AP. Each interdomain is 5 to 15 amino acids in length.

The DNA sequences of all 23 MAPs were de novo synthesized and cloned into the pET32a expression vector [30] downstream of bacterial thioredoxin A (TrxA). The MAPs were produced in the Origami B (DE3) bacterial strain as described in PCT Patent Application No. PCT/US09/64256, filed Nov. 12, 2009, and titled “Method of expressing proteins with disulfide bridges with enhanced yields and activity.” This application describes an improvement upon the expression system disclosed in U.S. Publication no. 20060246541, which includes, as an embodiment, expression of a chimeric snake venom disintegrin Vicrostatin (VCN) in the Origami B (DE3)/pET32a system. The improved method was used to generate increased amounts of correctly-folded active MAPs. This is achieved by growing the Origami B cells in a less selective environment and thus allowing for the generation and expansion of VCN-transformants that display a more optimal redox environment during the induction of the heterologous recombinant protein production. Unlike other E. coli strains, the Origami B is unique in that, by carrying mutations in two key genes, thioredoxin reductase (trxB) and glutathione reductase (gor), that are critically involved in the control of the two major oxido-reductive pathways in E. coli, this bacterium's cytoplasmic microenvironment is artificially shifted to a more oxidative redox state, which is the catalyst state for disulfide bridge formation in proteins [18, 29].

The Origami B strain has growth rates and biomass yields similar to those obtained with wild-type E. coli strains, which makes it an attractive and scalable production alternative for difficult-to-express recombinant proteins like VCN. This strain is also derived from a lacZY mutant of BL21. The lacY1 deletion mutants of BL21 (the original Tuner strains) enable adjustable levels of protein expression by all cells in culture. The lac permease (lacY1) mutation allows uniform entry of IPTG (a lactose derivative) into all cells in the population, which produces a controlled, more homogenous induction. By adjusting the concentration of IPTG, the expression of target proteins can be optimized and theoretically maximal levels could be achieved at significantly lower levels of IPTG. Thus the Origami B combines the desirable characteristics of BL21 (deficient in ompT and lon proteases), Tuner (lacZY mutant) and Origami (trxB/gor mutant) hosts in one strain. As mentioned above, the mutations in both the thioredoxin reductase (trxB) and glutathione reductase (gor) greatly promote disulfide bond formation in the cytoplasm [29].

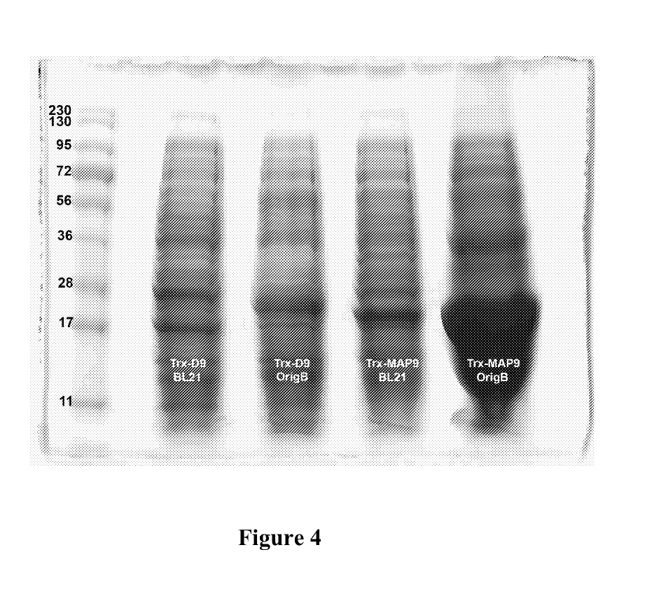

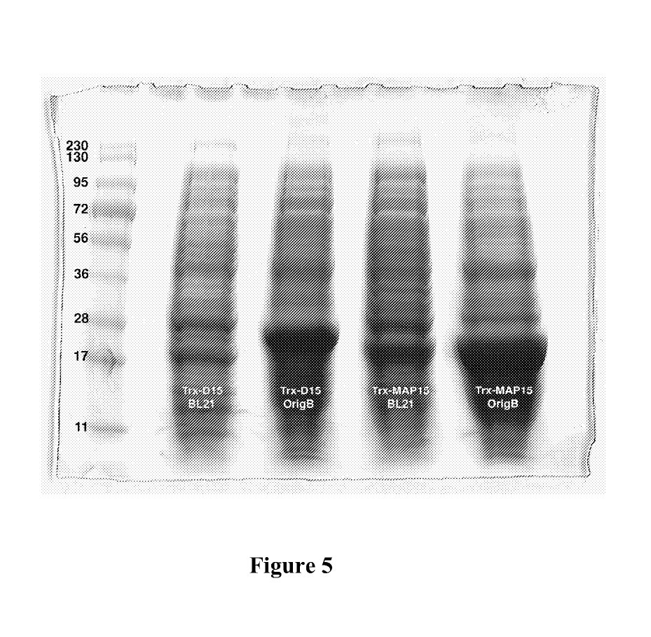

Although the Origami B strain offers a clear advantage over E. coli strains with reducing cytoplasmic environments like BL21 (FIGS. 4 and 5 show a comparison in expression levels between strains), the mere usage of the Origami B strain and the pET32a expression vector does not automatically guarantee the generation of a soluble and/or active product. The generation of disulfide-rich polypeptides in Origami B appears to be sequence dependent. For example, MAPs (e.g. MAP9 and MAP15) can be expressed in Origami B with significantly higher expression yields compared to their corresponding AP versions of human ADAMs 9 and 15 despite the fact that the same system and production technique were employed (FIGS. 4 and 5). Consequently, the modification of APs into MAPs can result in polypeptides having a disintegrin domain with greater expression yield in Origami B cells.

Furthermore, after purifying expressed disintegrin domains (APs) of ADAM 9 and 15, in a process that involves TEV protease treatment and RP-HPLC purification, the collected free polypeptides appeared to be unstable and to precipitate out of solution after reconstitution from lyophilized powder. In contrast, the corresponding MAP polypeptides, generated by employing the same purification steps, appear to be more soluble and stable when reconstituted in water after lyophilization.

MAPs of the invention are prepared so as to be substantially isolated or substantially purified. As used herein, the term “substantially purified” (or isolated) in reference to a MAP does not require absolute purity. Instead, it represents an indication that the MAP is preferably greater than 50% pure, more preferably at least 75% pure, and most preferably at least 95% pure, at least 99% pure and most preferably 100% pure. MAPs can be prepared synthetically or prepared by recombinant expression as described herein.

The term “substantially” as used herein means plus or minus 10% unless otherwise indicated.

Pharmaceutical compositions containing MAPs should comprise at a minimum an amount of the MAP effective to achieve the desired effect (e g inhibit cancer growth or prevent or inhibit cancer metastasis) and include a buffer, salt, and/or suitable carrier or excipient. Generally, in these compositions, MAPs are present in an amount sufficient to provide about 0.01 mg/kg to about 50 mg/kg per day, preferably about 0.1 mg/kg to about 5.0 mg/kg per day, and most preferably about 0.1 mg/kg to about 0.5 mg/kg per day.

MAPs may be administered by a variety of heretofore known means suitable for delivery thereof into the body of a subject (e.g. the blood stream) in substantial amounts. Intravenous administration of MAPs in a suitable liquid vehicle or excipient is presently contemplated as the preferred route of administration. MAPs are soluble in water, and may therefore be effectively administered in a suitable aqueous solution (e.g., phosphate buffered saline). Alternatively, MAPs may be administered orally (in the form of tablets or capsules formulated with a suitable binder or excipient material, or in the form of aqueous or oily suspensions, solutions, emulsions, syrups or elixirs) or as a parenteral suspension. MAPs can also be delivered intraarterially or intraductally, or may be introduced locally at the intended site of action. As is well known in the art, adjuvants or excipients such as local anesthetics, preservatives, buffering agents, lubricants, wetting agents, colorants, flavorings, fillers and diluents may suitably be included in any of these formulations.

MAPs may be delivered by way of liposomes. Liposomal delivery is well known in the art and has been described for delivery of disintegrins. For example, Swenson et al. describes use of intravenous delivery of contortrostatin in liposomes for therapy of breast cancer [26].

MAPs, such as MAP9, display pro-angiogenic effects (see below). Not wishing to be bound by theory, this could represent a more general characteristic of the members of the meltrin class of ADAM family (ADAMs 9, 12, and 19). The meltrins are mesenchymal ADAMs that were originally described to be expressed in the course of differentiation induction of muscle cells where they are involved in cell fusion and other processes and may play an important role in the stabilization of neovessels [33]. Consequently, recombinant polypeptides derived from the meltrin class of ADAM proteins can be used therapeutically to stimulate the formation of new capillary networks and maintain the collateral capillary growth in diseases such as coronary artery disease (CAD) or other ischemic conditions. Therefore, for example, MAPs may be coated on or chemically coupled to stents for use in therapy of CAD and related diseases that utilize stents.

MAPs can also be directly or indirectly conjugated to drugs, toxins, radionuclides and the like, and these conjugates used for diagnostic or therapeutic applications. For instance, a MAP can be used to identify or treat tissues or organs that express a cognate integrin. MAPs or bioactive fragments or portions thereof, can be coupled to detectable or cytotoxic molecules and delivered to a mammal having cells, tissues or organs that express the cognate integrin or integrins.

Suitable detectable molecules may be directly or indirectly attached to a MAP, and include radionuclides, enzymes, substrates, cofactors, inhibitors, fluorescent markers, chemiluminescent markers, magnetic particles and the like. Suitable cytotoxic molecules may be directly or indirectly attached to the polypeptide or antibody, and include bacterial or plant toxins (for instance, diphtheria toxin, Pseudomonas exotoxin, ricin, abrin and the like), as well as therapeutic radionuclides, such as iodine-131, rhenium-188 or yttrium-90 (either directly attached to the polypeptide or antibody, or indirectly attached through means of a chelating moiety, for instance). MAPs may also be conjugated to cytotoxic drugs, such as adriamycin. For indirect attachment of a detectable or cytotoxic molecule, the detectable or cytotoxic molecule can be conjugated with a member of a complementary/anticomplementary pair, where the other member is bound to the polypeptide or antibody portion, such as biotin/streptavidin.

The disclosed compositions can be used either singly or in combination for the treatment of diseases related to endothelial cell dysfunction. In an embodiment, the disclosed compositions are useful for cancer treatment. The cancer can be of epithelial origin. The cancer can be breast cancer, colorectal cancer, basal cell carcinoma, adenocarcinoma, gastrointestinal cancer, such as, for example, lip cancer, mouth cancer, esophageal cancer, small bowel cancer and stomach cancer, colon cancer, liver cancer, bladder cancer, pancreatic cancer, ovarian cancer, cervical cancer, lung cancer, and skin cancer, such as squamous cell and basal cell cancers, prostate cancer, renal cell carcinoma, central nervous system (CNS) cancer, leukemia and other known cancers that effect epithelial cells throughout the body. Not wishing to be bound by theory, in many forms of cancer a pathogenic cross-talk exists between ADAMs (membrane-tethered and/or secreted forms) and their integrin counter-receptors, a process that is mechanistically important for cancer progression. These pathological interactions serve in cancer for the recruitment of ADAM metalloproteinases via integrins into multiprotein complexes (i.e., invadosomes) that are assembled by highly migratory cells (cancer, inflammatory or endothelial cells) in the process of executing some critical integrin-driven steps in tumor progression: the infiltration of tumor stroma by inflammatory cells where they are shown to play important supportive roles, the migratory steps associated with tumor angiogenesis where endothelial cells from preexisting vessels are recruited to assemble neovessels, and, finally, the migratory and invasive events associated with metastasis. The disruption of these pathological processes by MAPs reduce malignancies. In addition to their potential antiangiogenic, antiinflammatory and antimetastatic effects, the broad spectrum anti-integrin MAPs can also impact tumor differentiation. The ability to send the right differentiation signals to cancer stem cells, by forcing them to acquire a more stable and differentiated phenotype during the course of therapy, remains one of the main goals of cancer therapy. For instance, Yuan et al. showed that a cell population with stem characteristics that was isolated from human glioblastoma multiforme (a highly-aggressive form of brain cancer) could be manipulated to differentiate in vitro and adopt more benign features [32]. Differentiation signals coming from soluble integrin ligands (e.g., either single or combinations of MAPs), cancer stem cells from various human malignancies may be induced to fully commit along their differentiation lineages and stay differentiated.

Composition of the invention can also be used for screening a candidate compounds for MAP-specific binding by comparing the relative binding of said candidate compound to an integrin in the presence and in the absence of a MAP, wherein a decrease of integrin binding in the presence of said MAP indicates that said MAP-specific integrin binding molecule is MAP-specific.

Compositions of the invention can be used for early detection of cancer by taking advantage of specific “integrin signatures” displayed by cancer cells. An “integrin signature” or “integrin profile” is the one or more integrins expressed on the surface of a cell. For example, carcinomas and sarcomas can display specific ‘integrin signatures’ based on a tumors' state of differentiation and organ localization. MAP-specific binding can be employed to identify and diagnose various cancer types based on integrin signatures either as in vivo cancer imaging agents (diagnostic imaging) and/or ex vivo molecular tools for tumor specimen staining (diagnostic pathology). In the latter case, staining cancer specimens with different MAPs can lead to characteristic staining patterns depending on various tumors' origin and grading. Compositions of the invention can be used for cancer staging, monitoring cancer cancer progression or therapy. The cancer can be of epithelial origin. The cancer can be breast cancer, colorectal cancer, basal cell carcinoma, adenocarcinoma, gastrointestinal cancer, such as, for example, lip cancer, mouth cancer, esophageal cancer, small bowel cancer and stomach cancer, colon cancer, liver cancer, bladder cancer, pancreatic cancer, ovarian cancer, cervical cancer, lung cancer, and skin cancer, such as squamous cell and basal cell cancers, prostate cancer, renal cell carcinoma, and other known cancers that effect epithelial cells throughout the body. MAPs can be used with PET (Positron Emission Tomography) probes to take advantage of the unique ability of MAPs with known integrin affinities to differentially bind to primary tumors, metastatic foci, as well as the tumor neovasculature based on the specific integrin expression of each particular solid tumor. For example, MAPs can be labeled with ¹⁸F through the amino group using N-succinimidyl-4-¹⁸F-fluorobenzoate (¹⁸F-SFB) under optimized fluorination reaction conditions or by conjugation with the metal chelator DOTA/NOTA for ⁶⁴Cu/⁶⁸Ga-labeling. Furthermore, fluorescently-labeled MAPs may be used in vivo and ex vivo (on biopsy specimens) for tumor integrin expression profile analysis.

Compositions of the invention may be also utilized as molecular tools in regenerative medicine. For example, by coating artificial ECM scaffolds (organ molds) with a MAP or combinations of MAPs, stem cell precursors can be induced into populating these scaffolds and differentiate into desirable epithelial and mesenchymal layers. For example, a urinary bladder scaffold coated with such a composition containing one or more MAPs can be used to direct stem cell progenitors to commit into both urothelium and muscular layers. Other examples include esophageal and rectal scaffolds coated with such a composition containing one or more MAPs can be used to direct stem cell progenitors to commit into the relevant tissue layers. Compositions of the invention can also be used to create defined tissue culture plate coatings that could either support the growth and maintain the stemness of embryonic stem cells in vitro or guide the commitment of these totipotent cells along their differentiation lineages in the same system.

Compositions of the invention may be used as novel drugs for supporting the collateral growth of capillaries in various ischemic conditions, such as drug-eluting stents. For example, MAPs can be conjugated with a radionuclide, such as with a beta-emitting radionuclide, to reduce restenosis. Such therapeutic approach poses less danger to clinicians who administer the radioactive therapy.

### EXAMPLES

### Example 1

### Preparation and Purification of MAPs

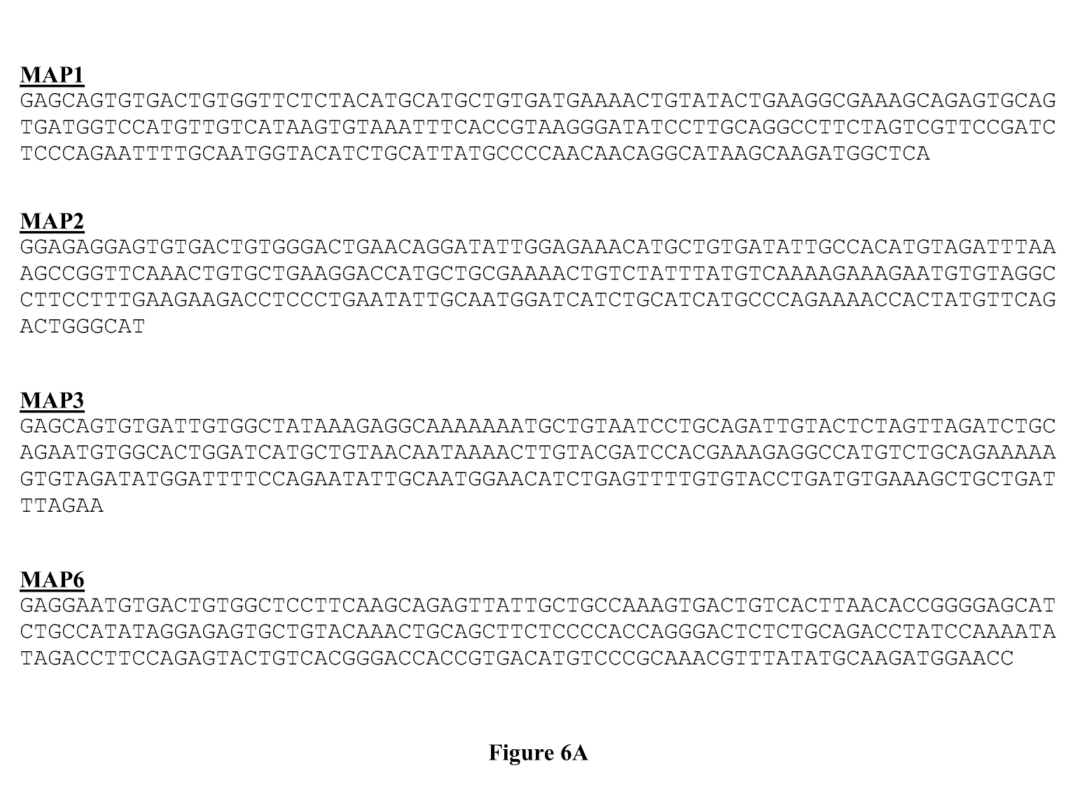

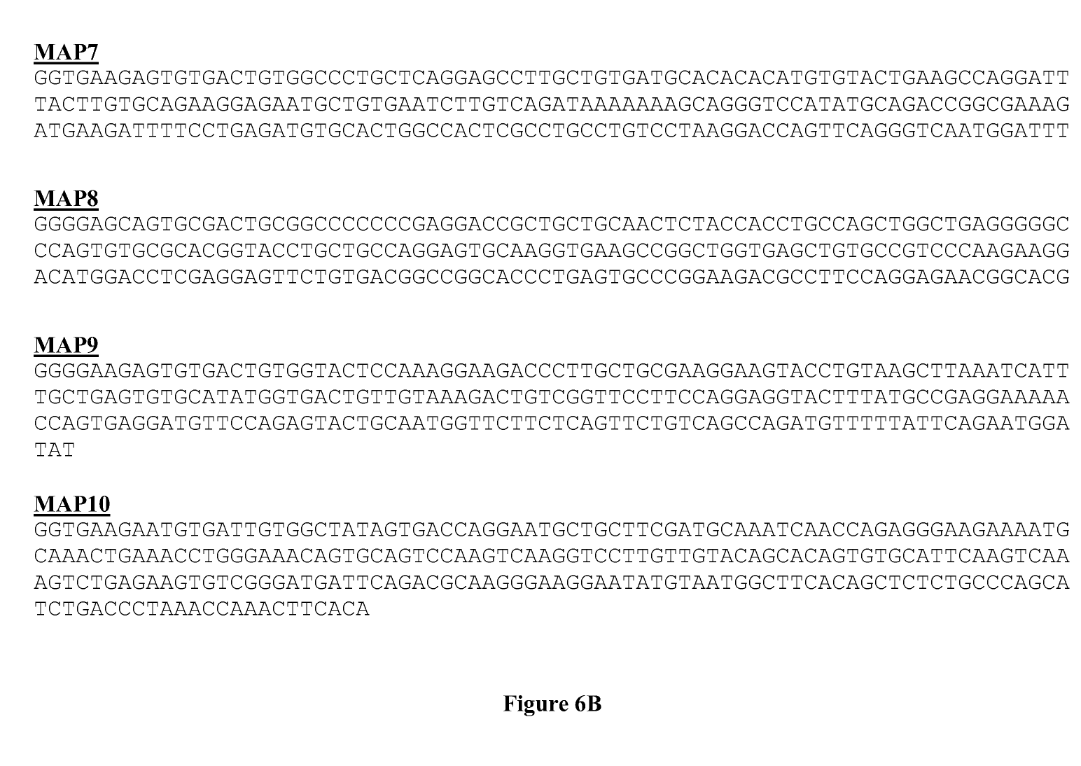

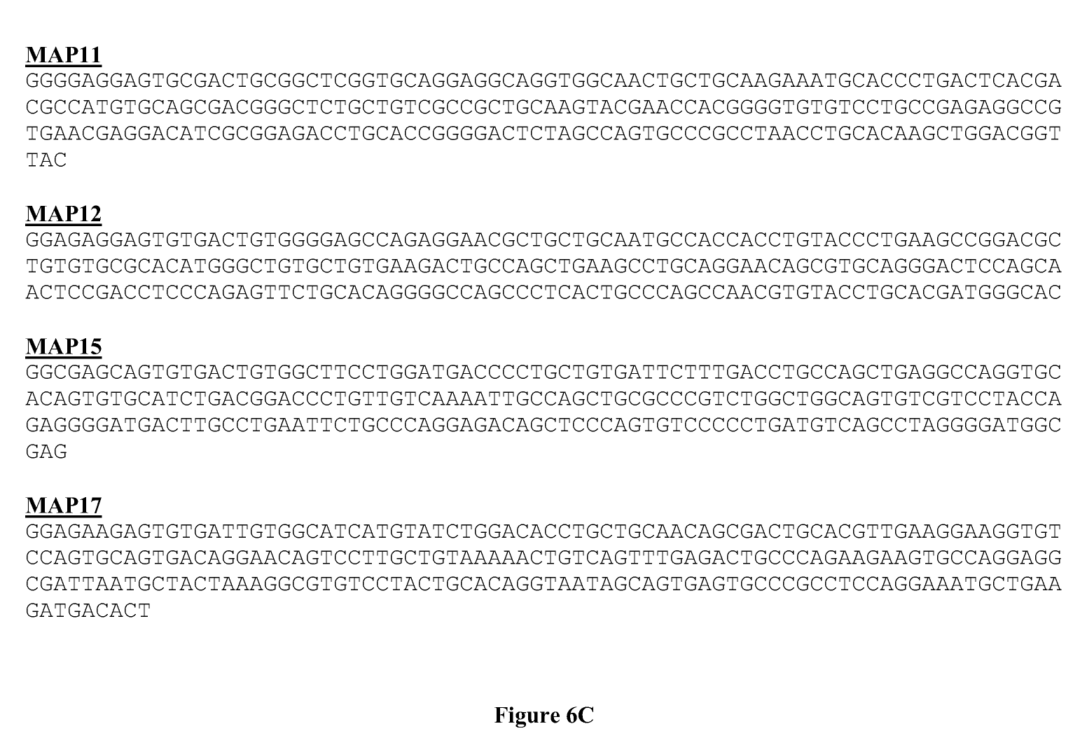

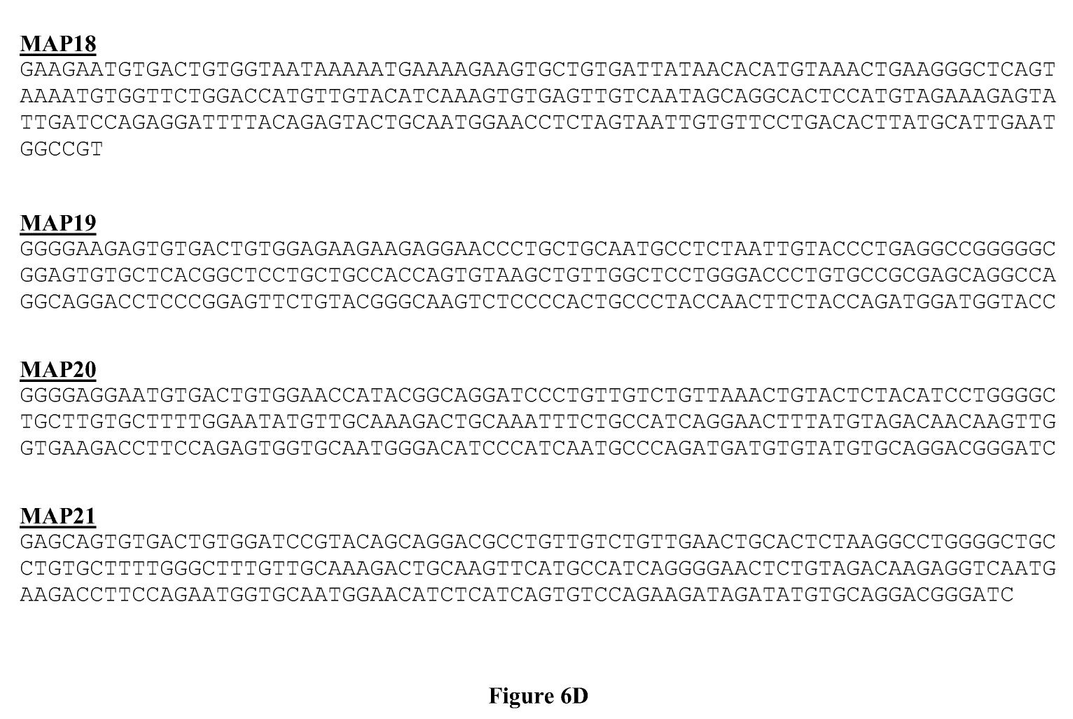

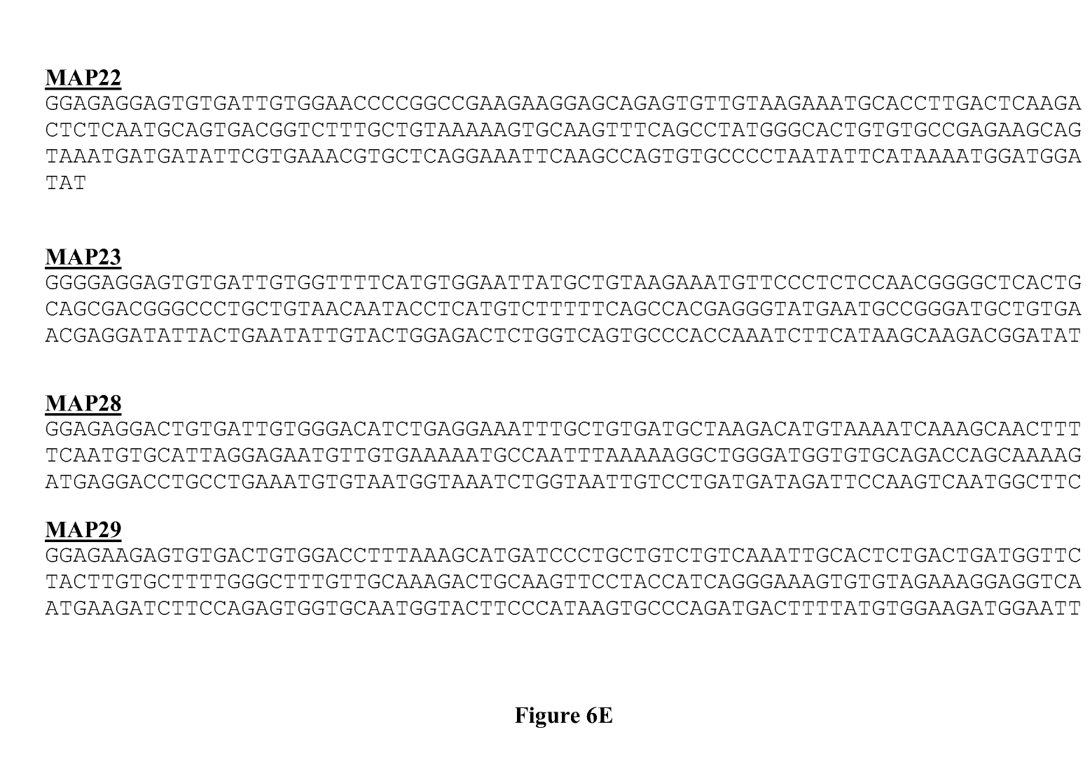

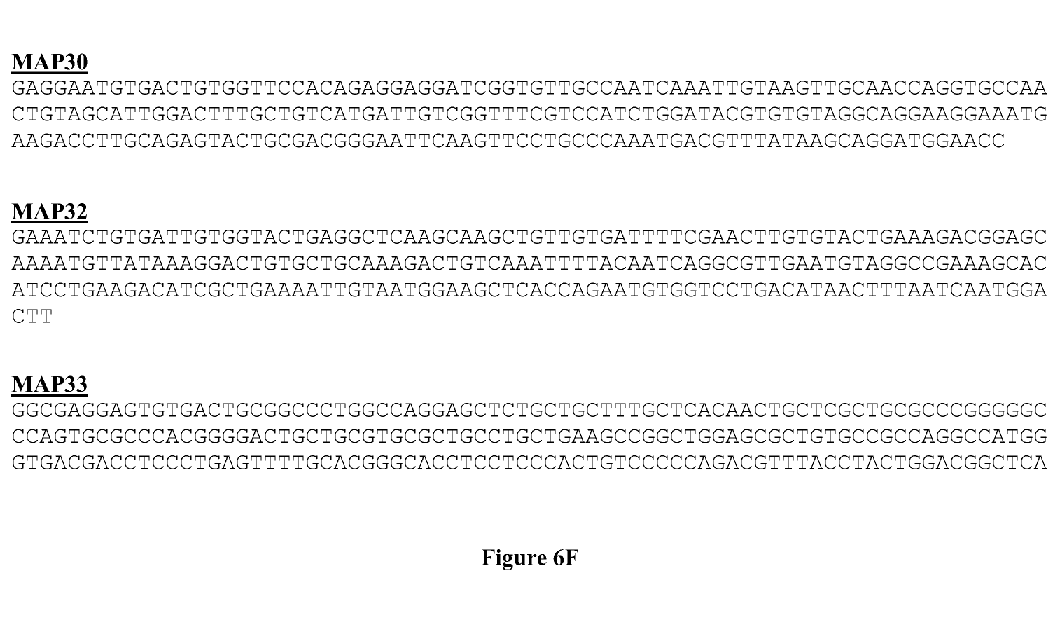

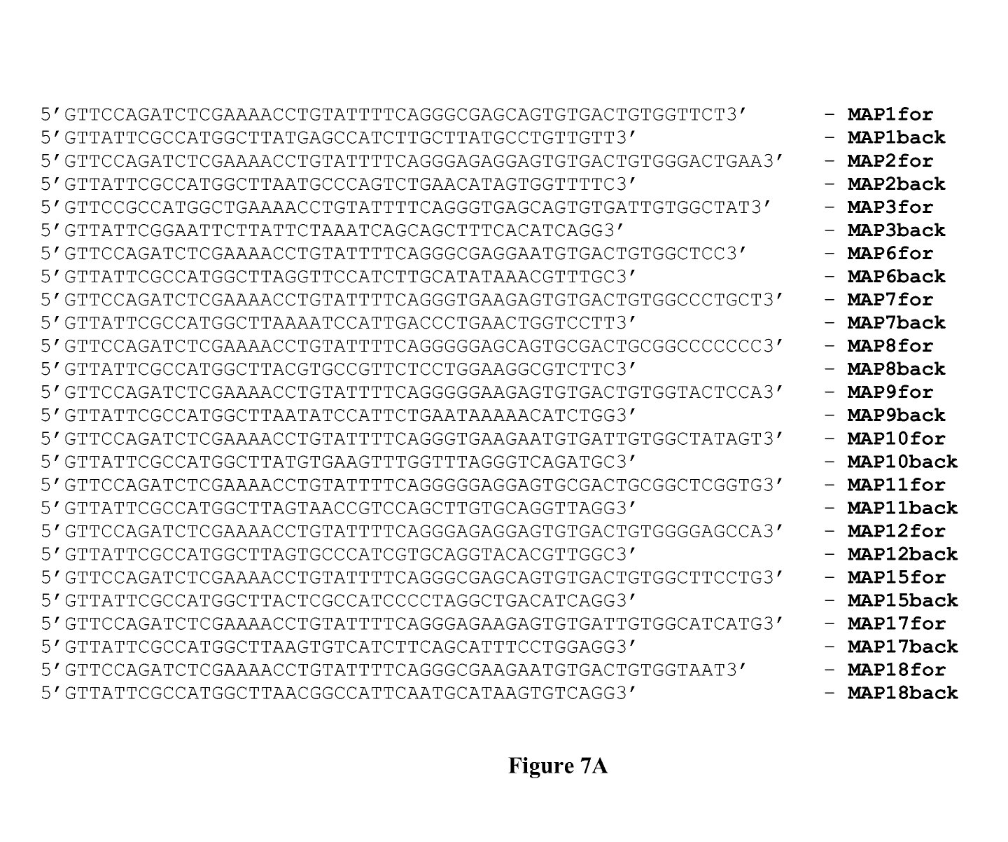

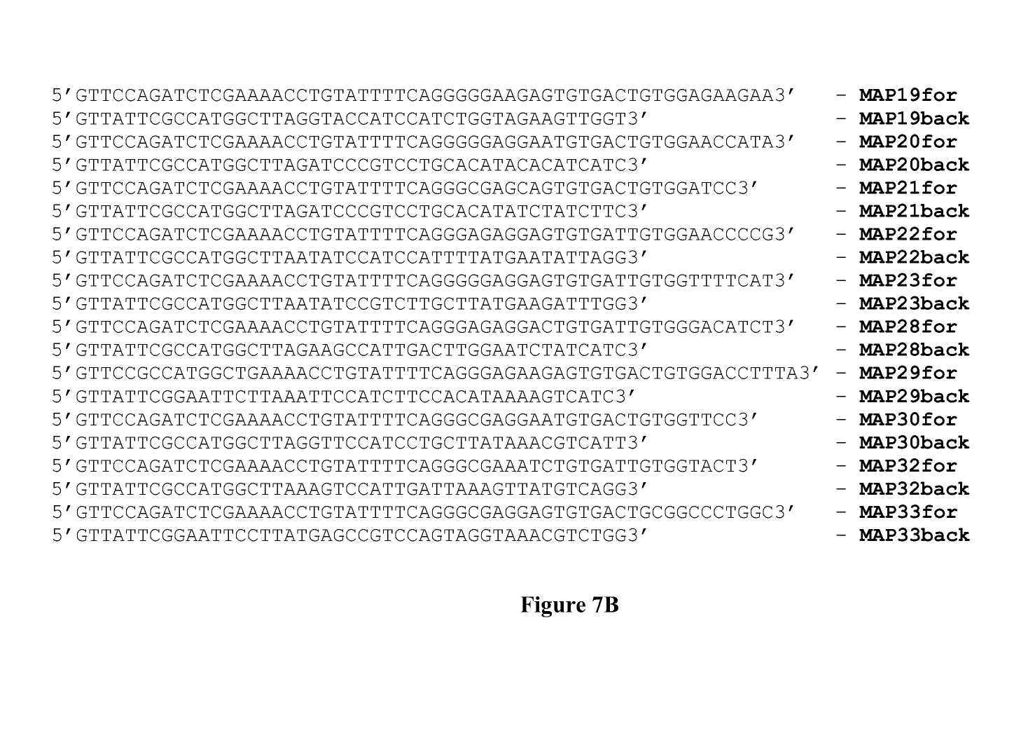

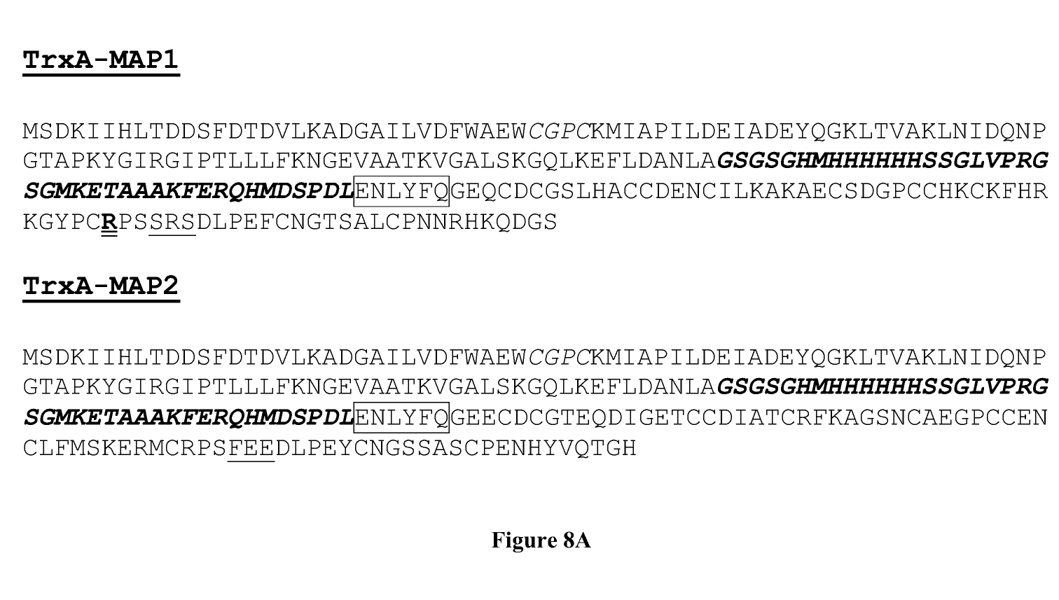

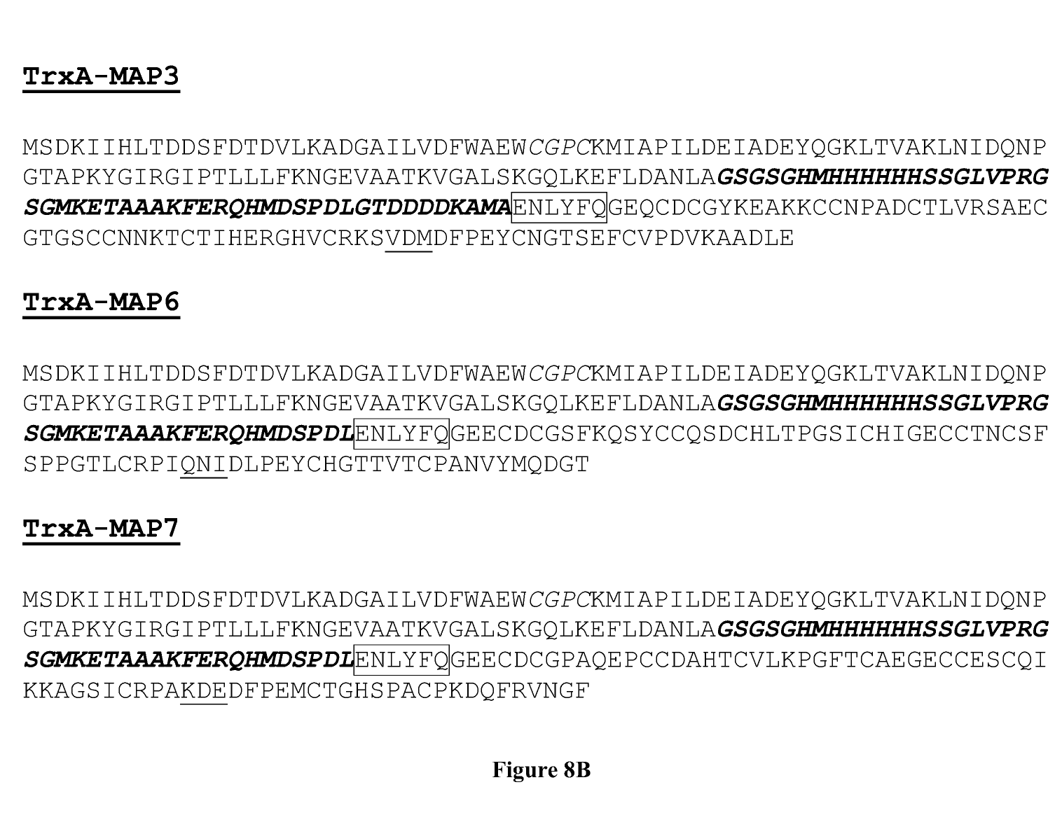

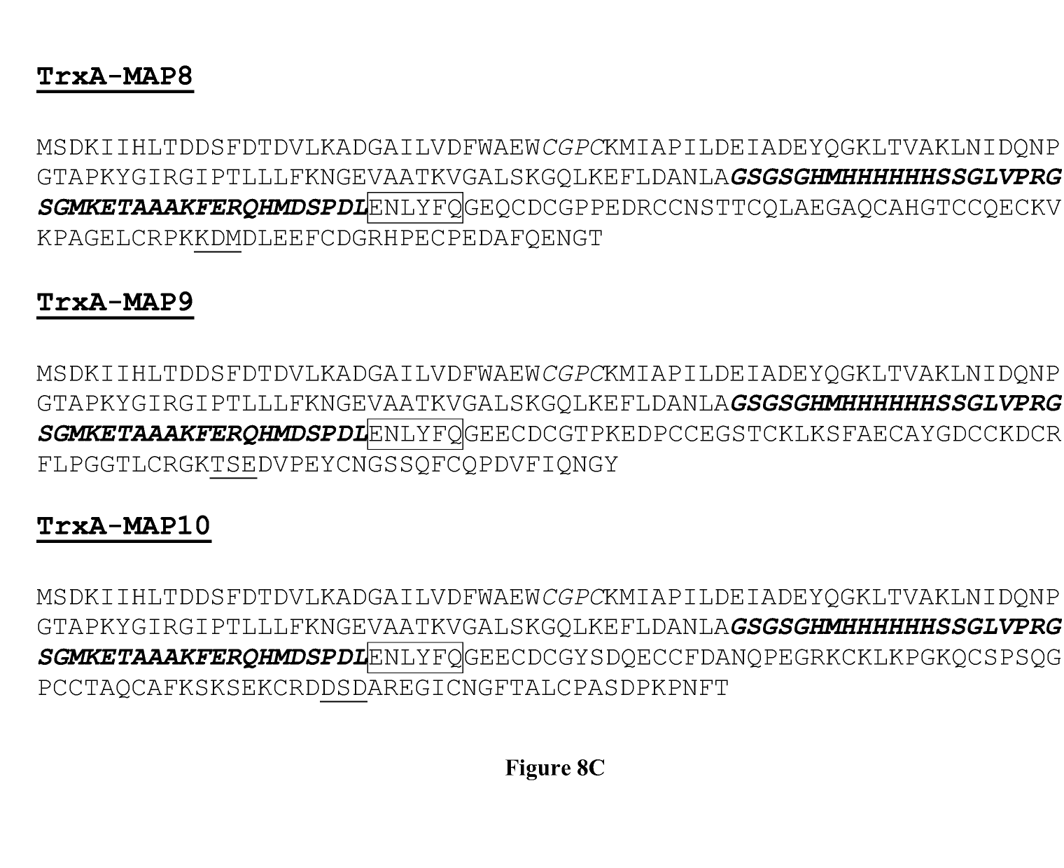

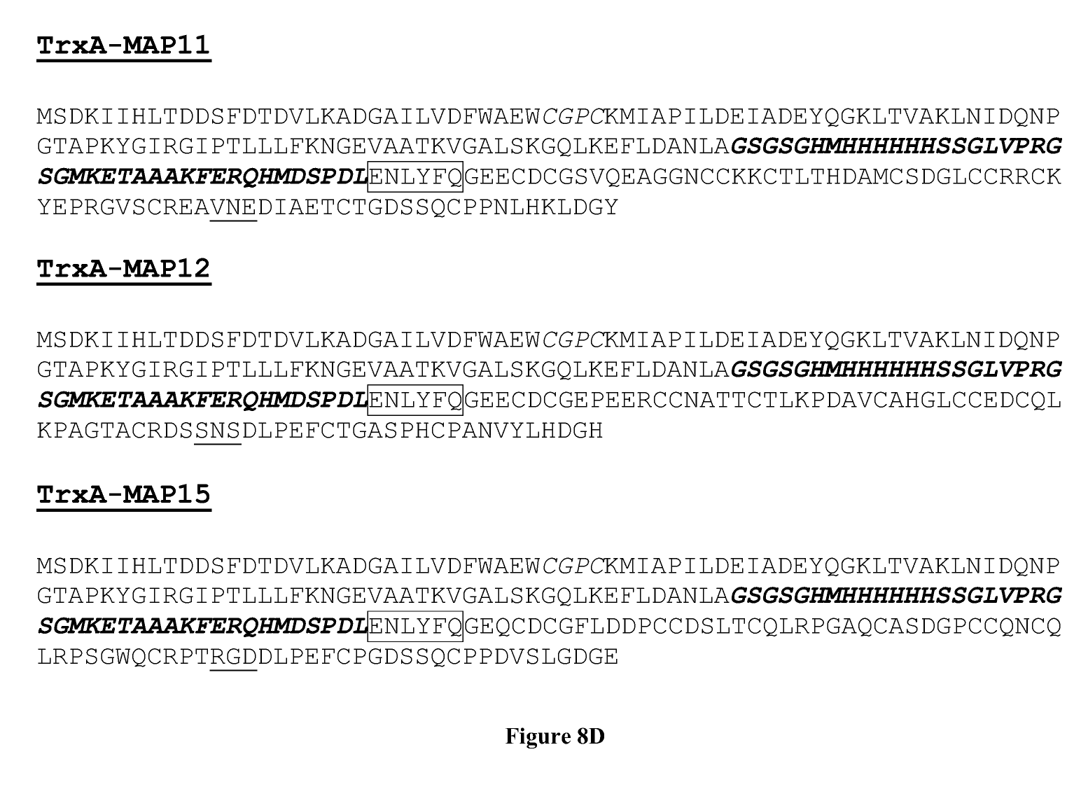

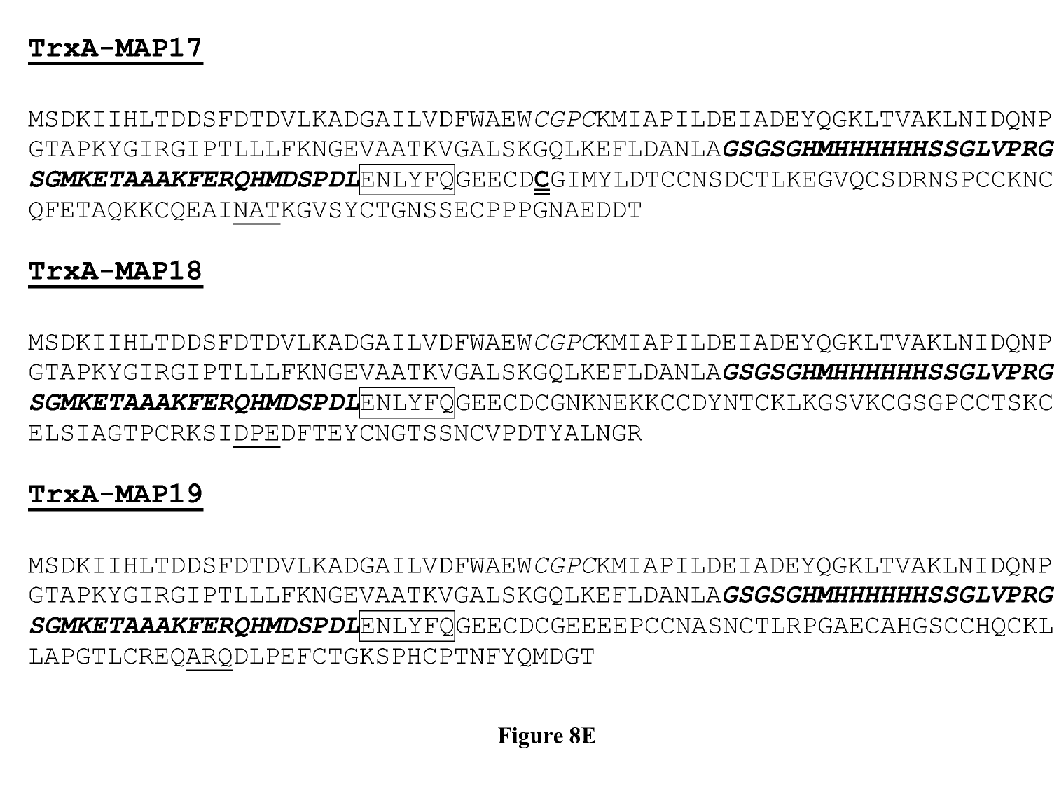

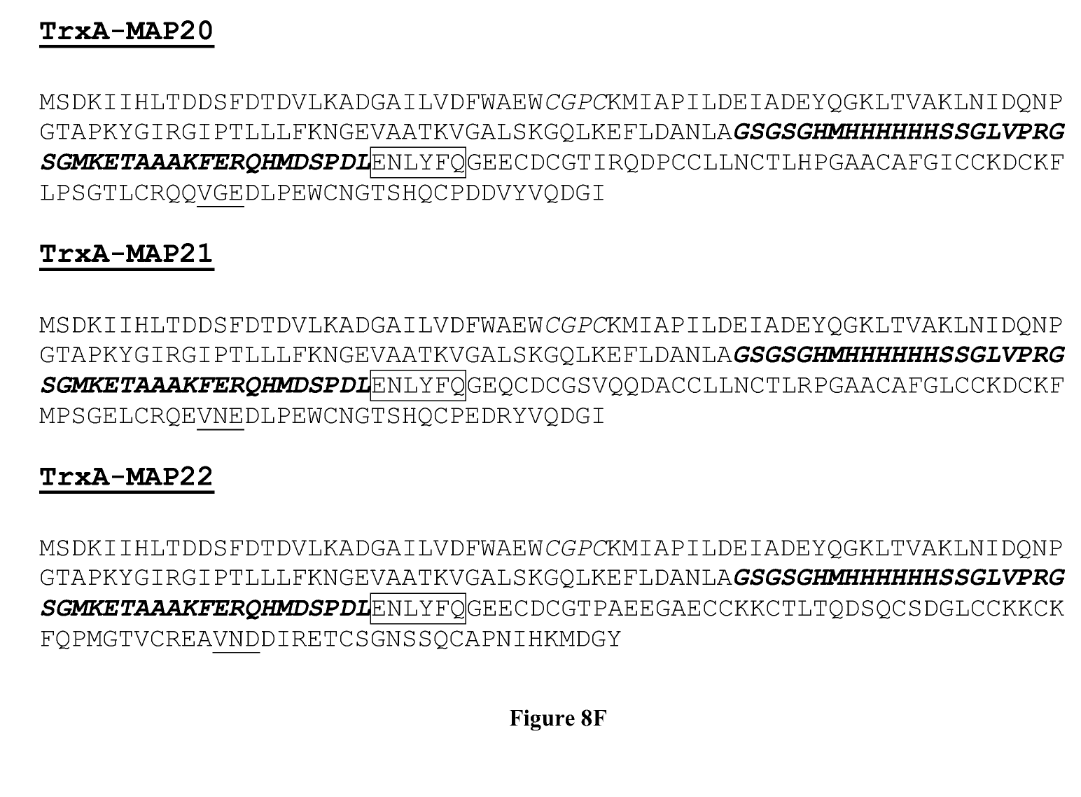

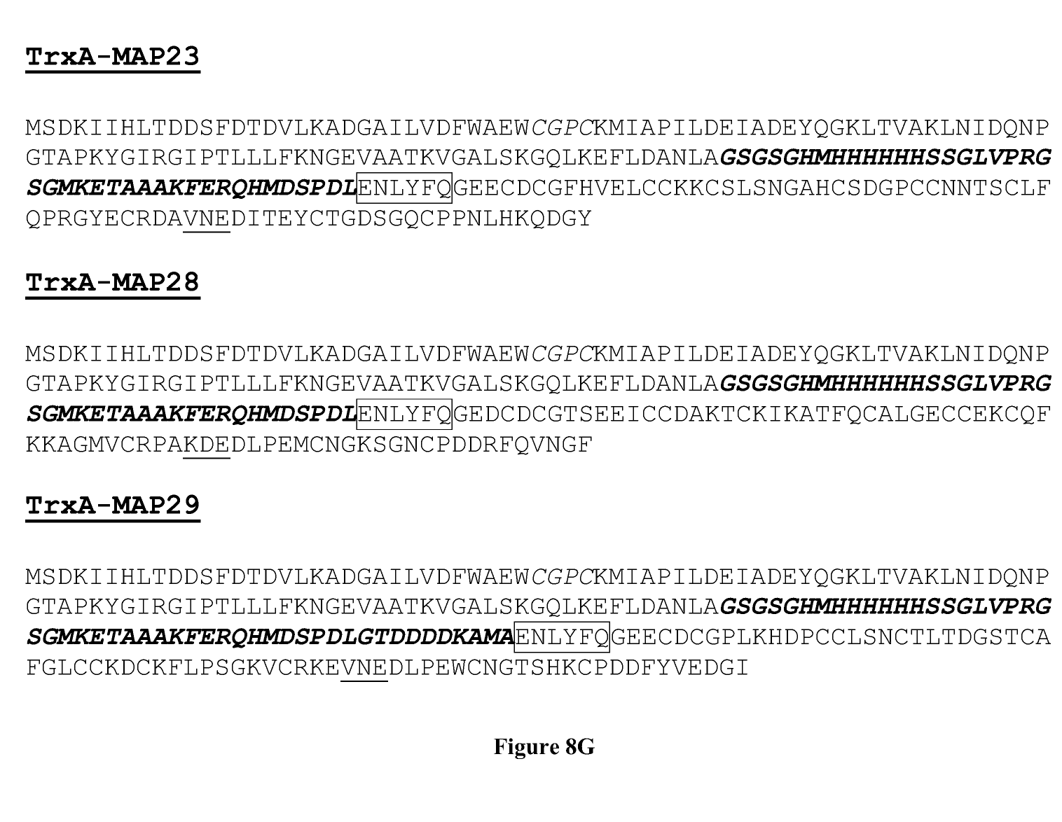

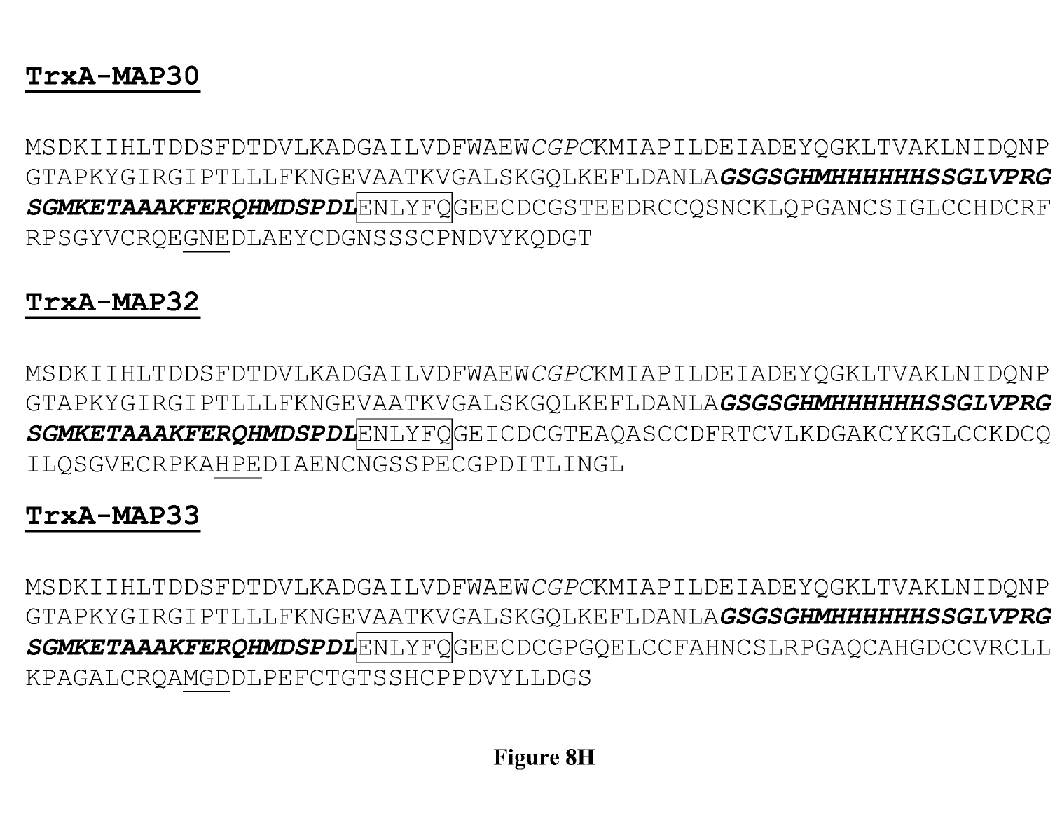

FIG. 6 shows a listing of synthetic MAPs DNA sequences that were cloned into pET32a expression vector. FIG. 7 shows the corresponding list of oligonucleotide primers utilized for MAPs cloning into pET32a vector. FIG. 8 shows the amino acid sequences of all TrxA-MAP constructs that were expressed in Origami B (DE3). The active site of TrxA and the tripeptide motif at the tip of the disintegrin loop are underlined, the TEV cleavage site is highlighted in a box and the linker region between TrxA and various MAP constructs is in bold black and italicized. The new residues introduced to replace the native residues in MAPs 1 and 17 are highlighted in hold double-underlined.

Bacterial cells and reagents. The Origami B (DE3) E. coli strain and pET32a expression vector carrying the bacterial thioredoxin A gene (trxA) were purchased from Novagen (San Diego, Calif.). All 23 MAP DNA sequences were de novo synthesized and inserted into a plasmid by Epoch Biolabs, Inc. (Sugar Land, Tex.). The AP DNA sequences were PCR amplified from cDNA libraries built from several mammalian cell lines including HUVEC (PromoCell GmbH, Heidelberg, Germany), MDA-MB-435 (ATCC, Manassas, Va.), MDA-MB-231 (ATCC, Manassas, Va.), and Jurkat (ATCC, Manassas, Va.). The oligonucleotide primers used for further cloning the APs and MAPs DNA sequences into pET32a expression vector were synthesized by Operon Biotechnologies, Inc. (Huntsville, Ala.). All restriction enzymes and ligases used for cloning the APs and MAPs DNA sequences into pET32a expression vector were purchased from New England Biolabs, Inc. (Ipswich, Mass.). The recombinant TEV protease was purchased from Invitrogen (Carlsbad, Calif.).

Construction of MAP expression vectors and recombinant production. The synthetic MAPs DNA sequences that were cloned into pET32a expression vector downstream of TrxA are listed in FIG. 6A-F. The oligonucleotide primers used for MAPs cloning are listed in FIG. 7A-B. The generated pET32a plasmids carrying the DNA sequences of MAPs cloned downstream of TrxA gene were initially amplified in DH5α E. coli, purified and sequenced before being transferred into Origami B (DE3) E. coli. The transformed cells for each MAP construct were then plated on LB-Agar supplemented with carbenicillin (50 μg/mL), tetracycline (12.5 μg/mL), and kanamycin (15 μg/mL) and grown overnight at 37° C. From these plates, multiple cultures were established for each MAP construct from individual colonies of transformed Origami B by transferring these colonies into LB media containing carbenicillin (50 μg/mL). These initial cultures were grown overnight and further used for the inoculation of bigger volumes of LB media containing carbenicillin (50 μg/mL) that were grown at 37° C. and 250 rpm in a shaker-incubator until they reached an OD600 of 0.6-1. At this point, the cells from individual MAP cultures were induced using 1 mM IPTG and incubated for another 4-5 hours at 37° C. and 250 rpm. At the end of the induction period, the cells from individual MAP cultures were pelleted at 4000×g and lysed in a microfluidizer (Microfluidics M-110L, Microfluidics, Newton, Mass.). The operating conditions of the microfluidizer included applied pressures of 14,000-18,000 psi, bacterial slurry flow rates of 300-400 ml per minute and multiple passes of the slurry through the processor. The insoluble cellular debris from lysates processed from individual MAP cultures was removed by centrifugation (40,000×g) and the soluble material containing Trx-MAPs for each MAP culture was collected. The expressed fusion proteins (i.e., Trx-MAPs) in the collected soluble lysates were then proteolysed by incubation with recombinant TEV protease overnight at room temperature which efficiently cleaved off each individual MAP from its TrxA fusion partner as monitored by SDS-PAGE. When proteolysis was complete, the proteolyzed lysates were passed through a 0.22 μm filter, diluted 1:100 in double distilled H₂O, ultrafiltrated through a 50,000 MWCO cartridge (Biomax50, Millipore) and then reconcentrated against a 5,000 MWCO cartridge (Biomax5, Millipore) using a tangential flow ultrafiltration device (Labscale TFF system, Millipore).

The APs were cloned into pET32a, transformed into Origami B, and expressed using the same procedures described above for MAPs.

Purification of recombinant MAPs. The MAPs were purified from filtrated lysates by employing a high-performance liquid chromatography (HPLC) procedure according to a protocol previously established for snake venom disintegrins [2]. Purification was performed by C18-reverse phase HPLC using the standard elution conditions previously employed for the purification of native CN [2]. Individual filtrated lysates processed as described above were loaded onto a Vydac C18 column (218TP54, Temecula, Calif.). A ten-minute rinse (at 5 ml/min) of the column with an aqueous solution containing 0.1% TFA was followed by a linear gradient (0-100%) elution over 150 min in a mobile phase containing 80% acetonitrile and 0.1% TFA. The MAPs start eluting in 35-40% acetonitrile.

Expression analysis of MAPs. E. coli transformants were grown overnight at 37° C. in shaker flasks were induced in 1 mM IPTG for 5 hours at 37° C. and 250 rpm. At the end of the induction period, the cells were pelleted at 4,000×g, lysed by multiple freeze-thaw cycles, and further centrifuged at 40,000×g to remove the insoluble cell debris. 5 μl of soluble cell lysates from various E. coli hosts were loaded under reducing conditions on a precast 4-20% NuSep iGel (NuSep Inc., Lawrenceville, Ga.) and then Coomassie stained.

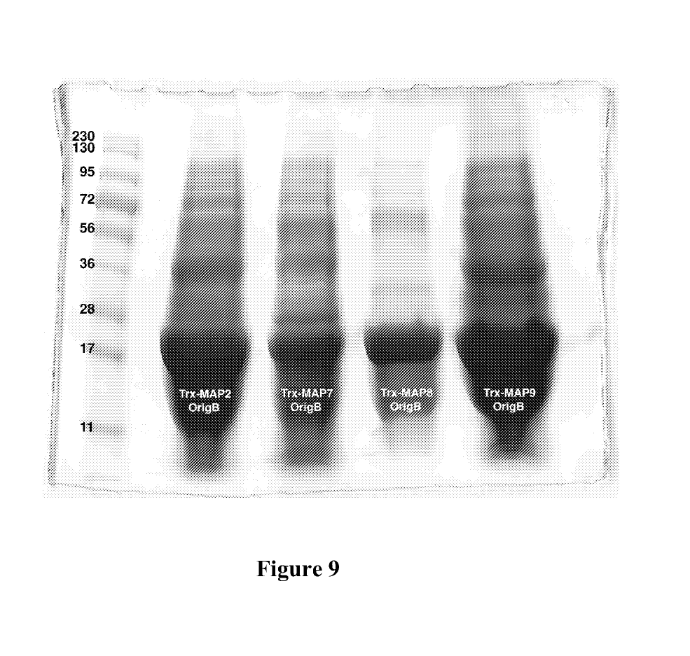

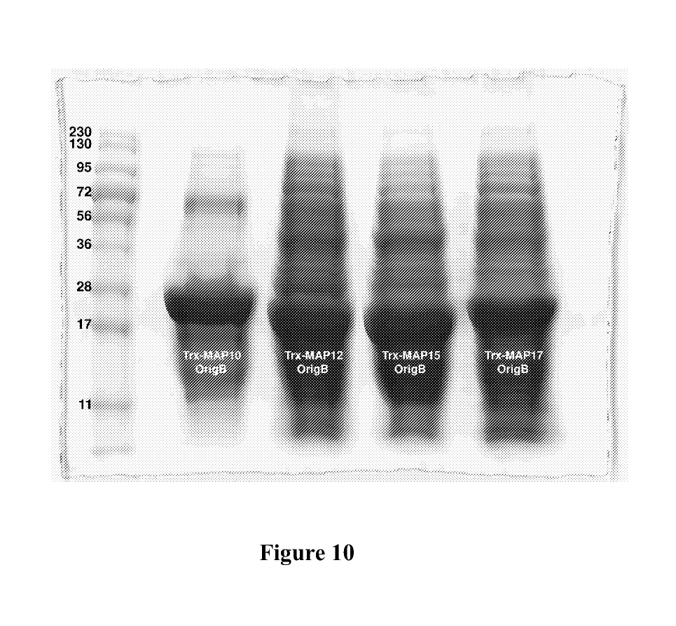

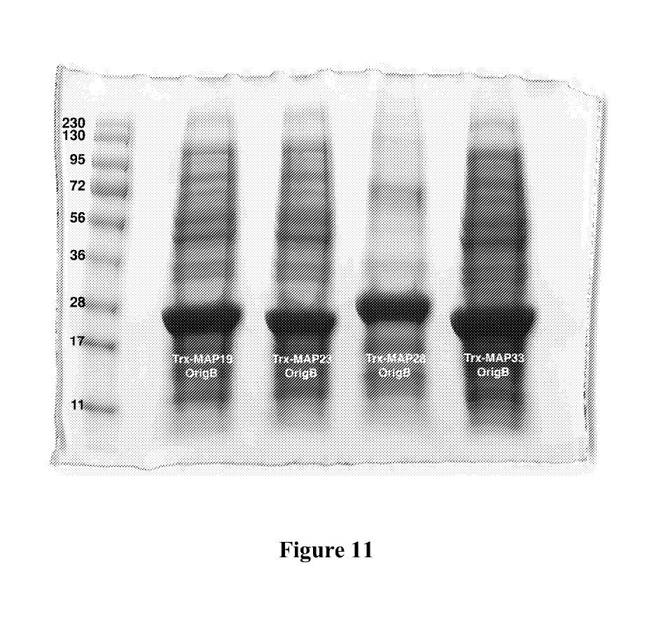

The resultant expression yield of AP9 and AP15 was lower than that compared to their corresponding MAPs (MAP9 and MAP15) (FIGS. 4 and 5). Both MAP9 and MAP15 were generated in Origami B (DE3) with batch-to-batch expression yields ranging from 200 mg to 350 mg of HPLC-purified protein per liter of bacterial culture. These high yields of purified recombinant MAPs were achieved by lysis of the pelleted bacterial transformants at the end of the induction step with a microfluidizer. A similar production technique generated recombinant MAPs with similar expression yields in the Origami B (DE3) system (FIGS. 9-11). In contrast, the expression of MAPs in closely related bacterial hosts that also support the formation of disulfide bridges (e.g., the AD494 (DE3), a K12 derivative that carries the trxB mutation only, or the Rosetta-gami B (DE3), an Origami B derivative optimized for rare codon usage) appeared to offer no additional advantage over the production in Origami B (DE3).

### Example 2

### Differential Cell Binding of MAPs

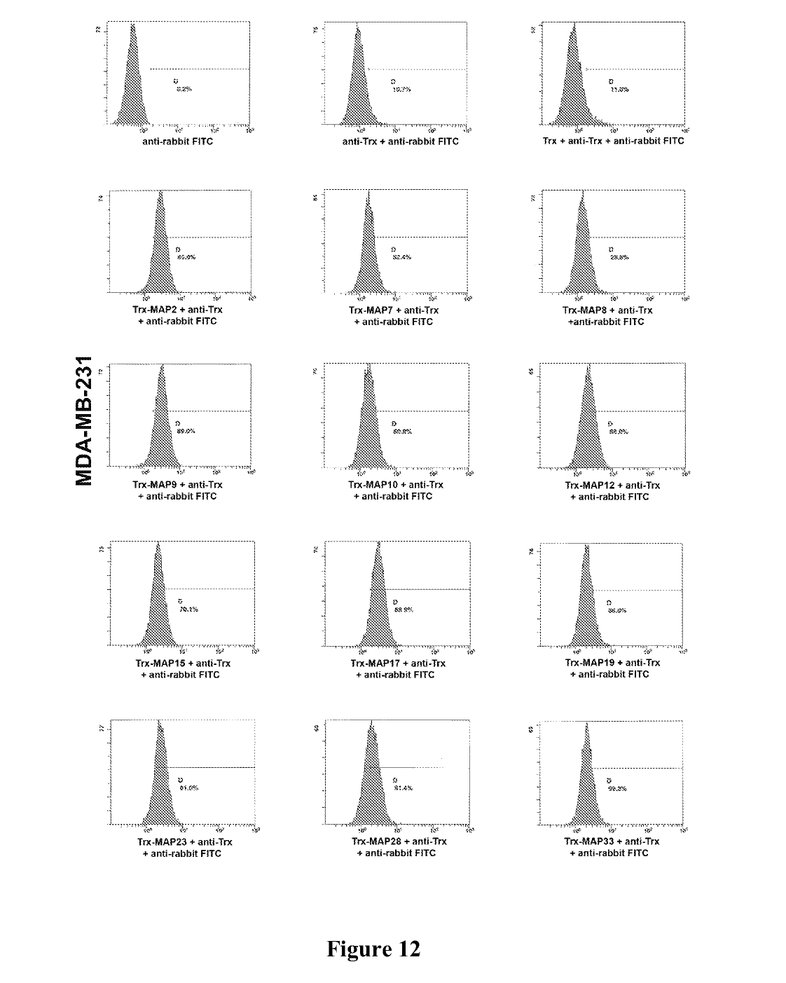

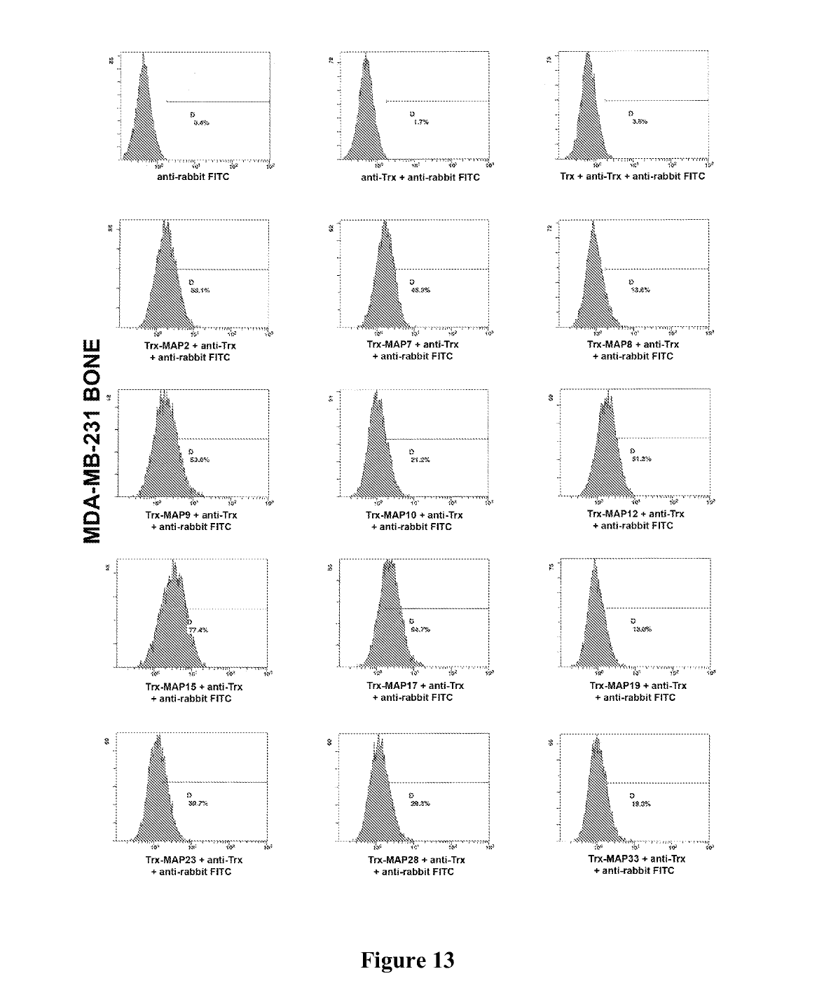

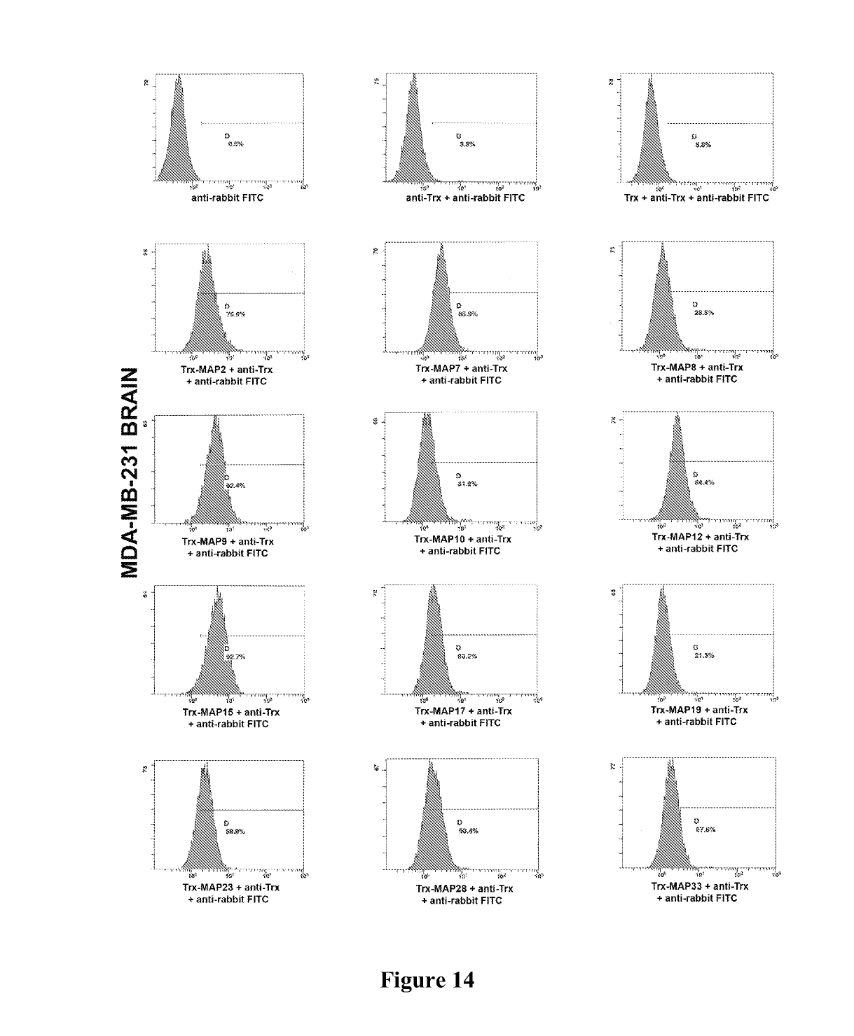

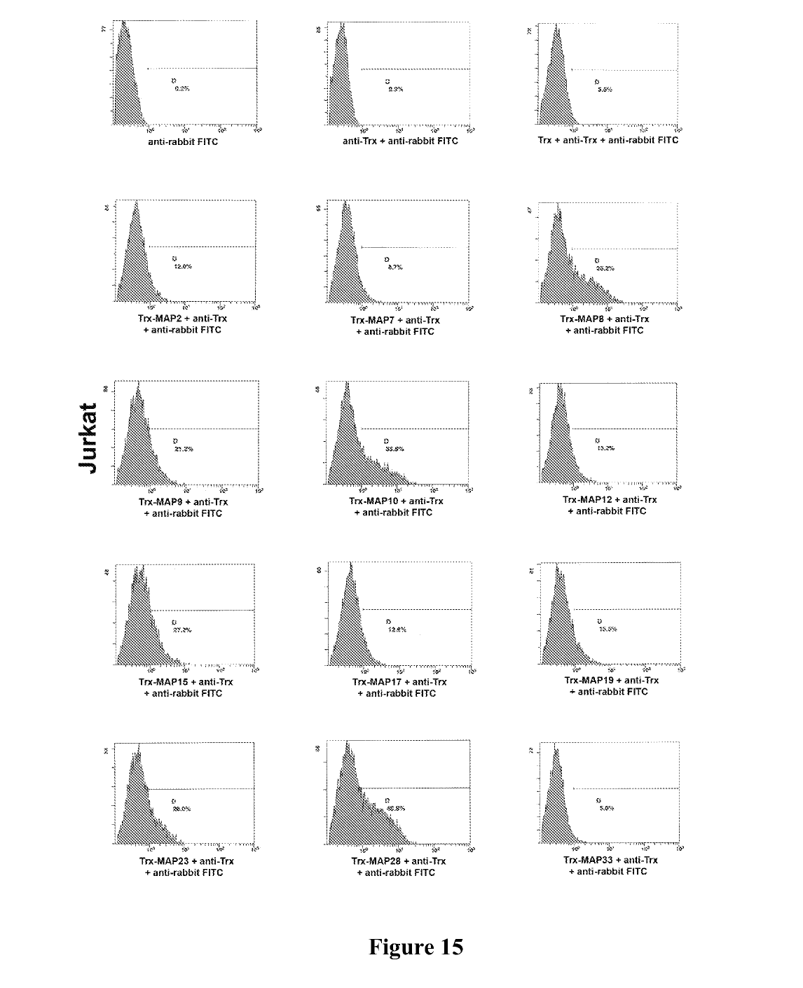

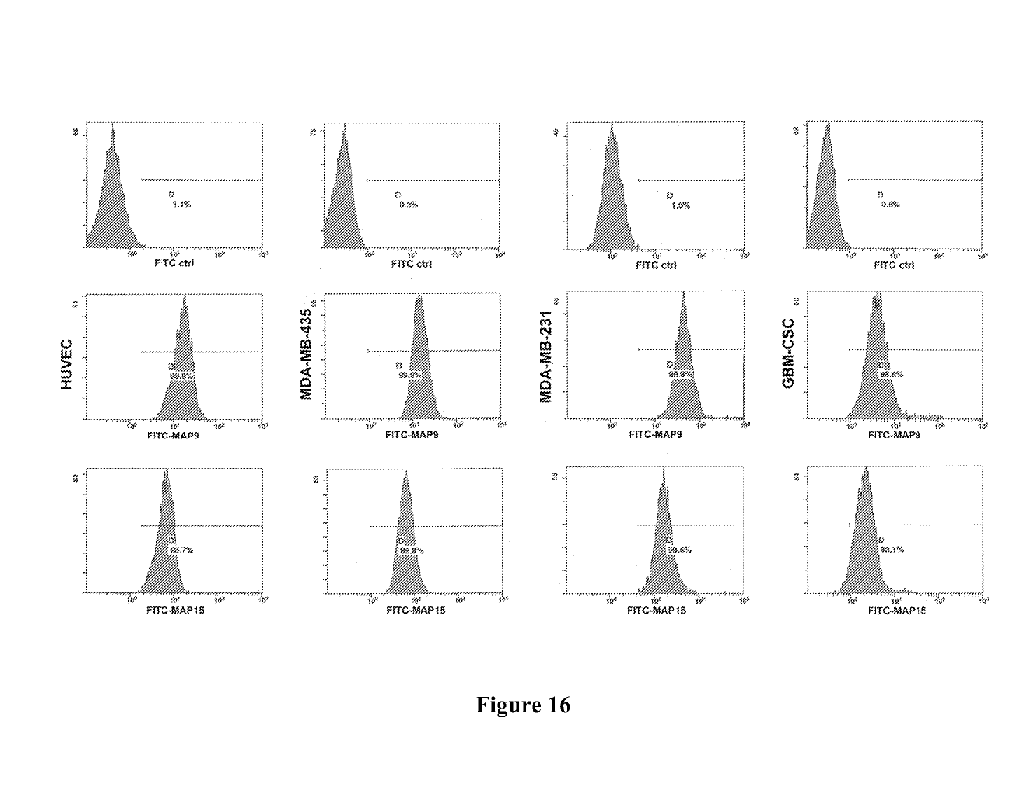

The Trx-MAPs produced in Origami B (DE3) using the method described above were analyzed by flow cytometry for differential binding to multiple cell lines (FIGS. 12-15), which included a human breast carcinoma line (MDA-MB-231) and two metastatic subclones of this line with tropism for different organs based on differential integrin profiles—a bone-homing subclone (MDA-MB-231 BONE) and a brain-homing subclone (MDA-MB-231 BRAIN). The 2 subclones of the MDA-MB-231 line were a gift from the investigators who originally isolated them [31]. Cells were incubated with the indicated Trx-MAPs and probed with the corresponding anti-Trx polyclonal antisera (Sigma-Aldrich, Inc., St. Louis, Mo.). The bound molecules were further detected with an anti-rabbit FITC-labeled antibody. Cells incubated with either the secondary FITC-labeled antibody only or the anti-Trx antisera plus the secondary antibody were used as controls. The Trx-MAPs 8 and 28, which correspond to ADAMs 8 and 28 that have been previously shown to be expressed on different lineages of immune cells and participate in immune responses, preferentially bind to Jurkat cells (a T-cell leukemia cell line) as shown by flow cytometry analysis (FIG. 15). Moreover, two HPLC-purified MAPs (MAP9 and MAP15) were FITC-labeled and analyzed by flow cytometry for direct binding to cancer cells lines (MDA-MB-231, MDA-MB-435), a cancer stem cell population isolated [32] from human glioblastoma (GBM-CSC) as well as human umbilical vein endothelial cells (HUVEC). Flow cytometry data (FIG. 16) shows that the purified MAP9 and MAP15 bind avidly to different cell lines that are expected to display vastly different integrin profiles.

### Example 3

### Antiangiogenic Effect of MAPs

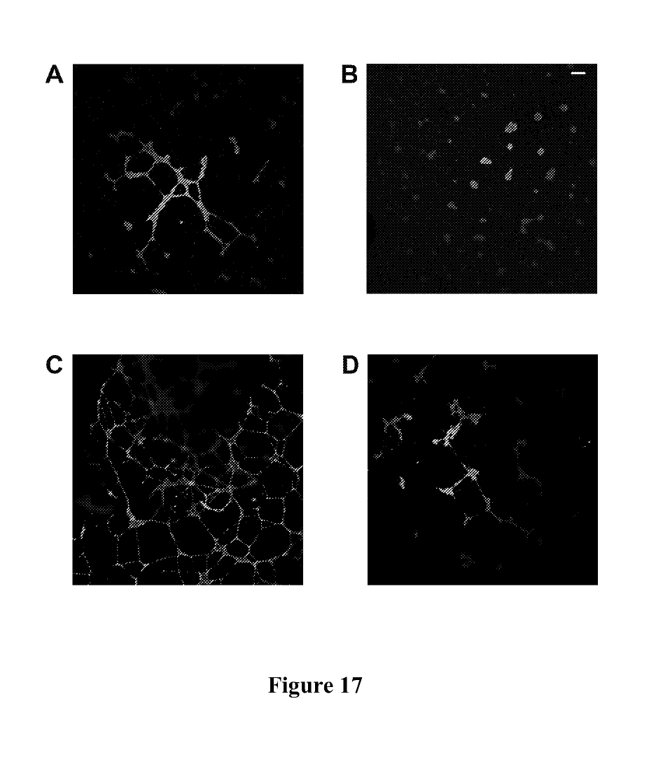

MAPs were then tested for angiogenic activity using the in vitro HUVEC tube formation assay. HUVEC cells were plated on ‘Endothelial Cell Tube Formation’ plates (BD Biosciences) in the presence of 10 nM of either MAP9 or MAP15. A known tube formation inhibitor (Suramin) was used as a negative control. See FIG. 17: Panel A—untreated control; panel B—100 μM Suramin; panel C—10 nM MAP9; panel D—10 nM MAP15. Cells were stained with Calcein AM and imaged using confocal microscopy. All images were taken at the same magnification (scale bar=50 μm). MAP15 showed significant anti-angiogenic activity in this assay. MAP9 appeared to have a pro-angiogenic effect by leading to the formation of an increased number of tubes when compared to the untreated control in this assay. This pro-angiogenic effect of MAP9 is supported by in vivo observations showing that a liposomal formulation of MAP9 promotes tumor growth (i.e., faster tumor growth, bigger tumors and a decreased survival compared to an untreated control) when administered intravenously in the MDA-MB-231 xenograft animal model.

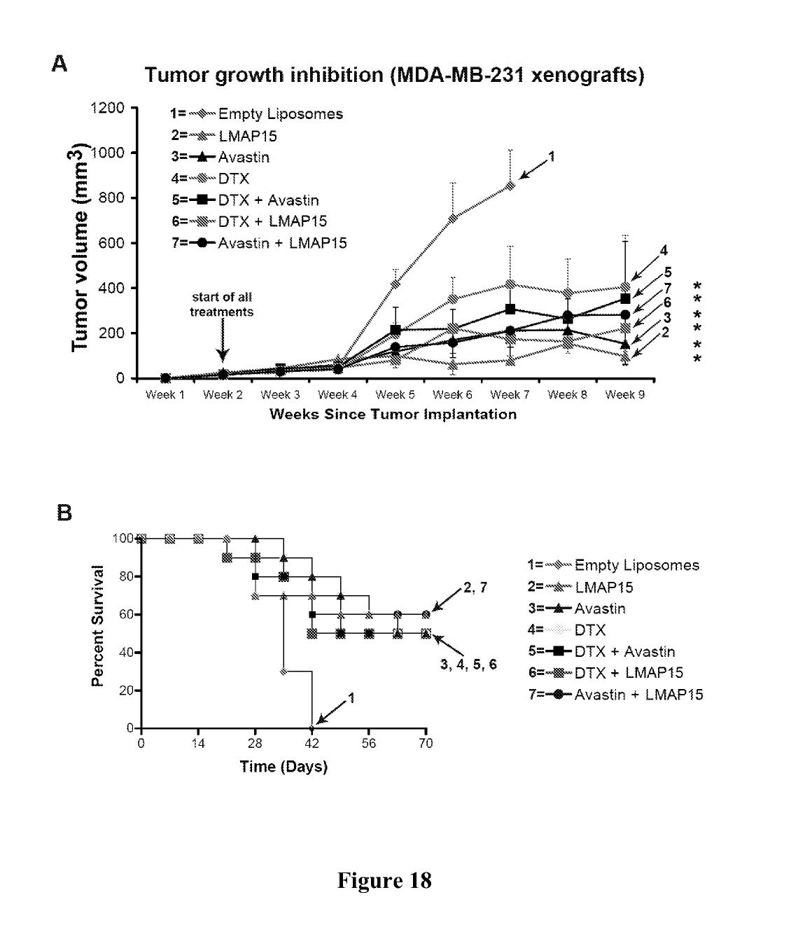

FIG. 18A shows inhibition of tumor growth induced by different treatments in the MDA-MB-231 model. Nude mice inoculated orthotopically (mammary fat pads; 2.5×10⁶ MDA-MB-231 cells per mouse in complete Matrigel) were allowed to grow palpable tumors before treatment was commenced (indicated by the arrow). Groups of animals (n=10) were treated intravenously with LMAP15, the dose-equivalent of 100 μg of MAP15 per injection, administered twice a week, or Avastin (400 μg per injection; approx. 20 μg/gr) administered intravenously once per week, or docetaxel (DTX, 160 μg) administered intraperitoneally once per week, or combinations of these agents. The control group received empty liposomes only. When compared to the control group, a significant delay in tumor growth was observed in all treated groups. The statistical analysis was done using ANOVA with Dunnett's post-hoc multiple comparison tests (* signifies a P<0.001). FIG. 18B shows animal survival data. Treatment groups showed increased survival compared to the control group (all control animals died by week 7). Either LMAP15 or Avastin plus LMAP15 groups had the highest survival. The in vivo efficacy data from this animal model indicates that LMAP15 exhibits similar antitumor potency to either Avastin or Docetaxel. The animals treated with liposomal MAP15 alone showed a better survival rate (i.e., the number of animals still alive at the end of the study) when compared to those treated with either Avastin or Docetaxel or combination therapy in the same xenograft model.

### Example 4

### Antiangiogenic Effect of LMAP15

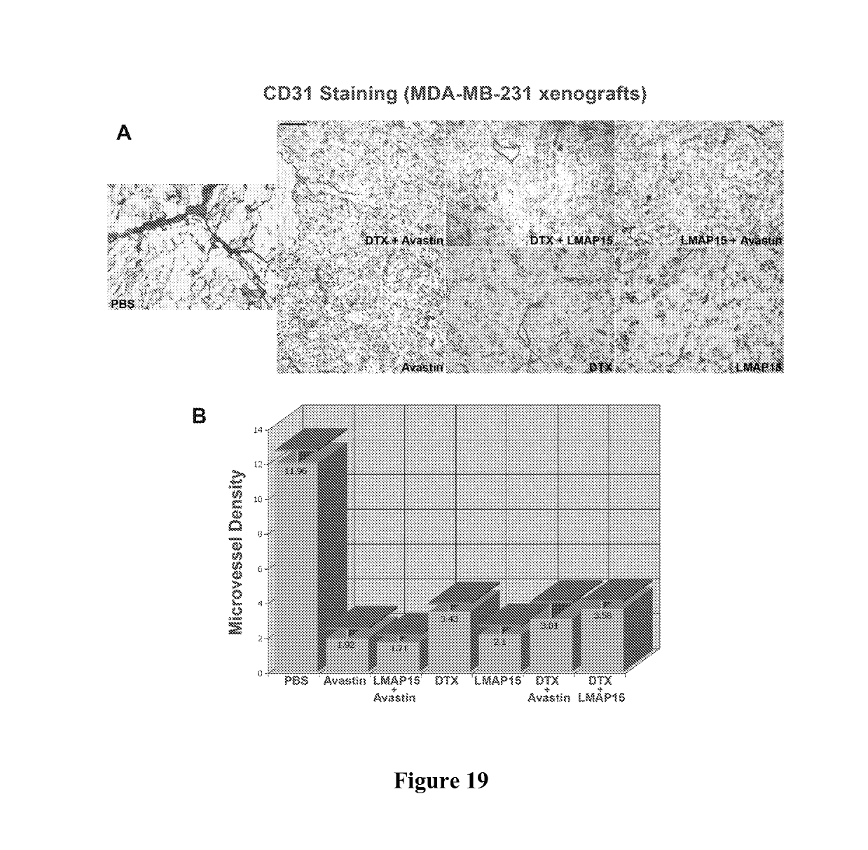

To assess the antiangiogenic effect of LMAP15, administered either alone or in combination, tumors from each group in the MDA-MB-231 study were dissected and extracted from dead or sacrificed animals and subsequently analyzed for microvessel density by immunohistochemistry. The extracted tumors were embedded in Tissue-Tek O.C.T (‘Optimal Cutting Temperature’ compound, Sakura Finetek USA) then frozen in dry ice, cut into 5 micron sections, fixed in acetone and stored at 4° C. until stained. For CD31 staining, the acetone-fixed slides were washed in PBS and blocked in PBS containing 5% goat serum then incubated overnight at room temperature with 200 μl of a rat polyclonal anti-CD31 antibody (BD Biosciences, San Diego, Calif.) diluted 1:50 in PBS and applied to the slides according to the manufacturer's protocol. This was followed by multiple washings in PBS (7 minutes/wash) and addition of 200 μl of a biotinylated secondary goat anti-rat antibody diluted 1:100 and applied for 45 minutes at room temperature. After 3 more washings in PBS, 200 μl of Avidin Binding Complex (Vector Laboratories, Burlingame, Calif.) diluted 1 drop in 2 ml PBS was applied to each slide for 30 minutes at room temperature after which the 3-amino-9-ethylcarbazole chromogen was added to visualize the antibody-stained microvessels. After three more washings in PBS, the slides were counterstained with Myers Hematoxylin and then mounted. To quantitate the CD31-stained microvessels, the slides were subjected to ‘random field’ analysis [35, 36]. Images were captured blindly from random fields on each slide at 200× using an Olympus E20N digital camera (Olympus America, Melville, N.Y.) attached to a microscope. For each group, 4 tumors were stained and 40 random fields analyzed. The CD31-positive areas were computed for each random field as % of total stained area using the ‘SimplePCI’ advanced imaging software (C-Imaging Systems, Cranberry Township, Pa.) and then averaged for each group. To eliminate bias, the random field image capture and the subsequent processing and analysis of the captured images were carried out in a blind fashion. LMAP15 was shown to significantly reduce microvessel density in this xenograft model (FIG. 19) when administered as either monotherapy or in combination with other anti-angiogenics with a different mechanism of action (e.g., Avastin) or chemotherapeutics (e.g., Docetaxel).

### Example 5

### Metastatic Breast Cancer Model

To evaluate the MAPs therapeutic efficacy as anti-invasive/anti-metastatic agents the optical luciferase imaging approach (in vivo bioluminescence) is employed in several animal models of spontaneous metastatic breast cancer. The following cell lines are stably infected with an adenoviral transduction system: a human breast cancer cell line (the MDA-MB-231, a triple-negative cell line), and two murine breast cancer lines (the 4T1, a HER2-negative line, and the D2F2, a HER2-positive line) with both luciferase and green fluorescence protein (GFP) reporter genes. In the human xenograft MDA-MB-231 model, an inoculum of 2×10⁶ cells suspended in complete Matrigel is injected in the mammary fat pads of nude mice and allowed to grow until the formed tumors are palpable (approx. 2 weeks after implantation). The following treatment groups (5 animals per group) are formed: group 1 control (animals receiving no treatments or manipulations), group 2 control (animals receiving empty liposomes intravenously), group 3 control (animals receiving PBS intravenously), group 1 treated (animals receiving Avastin intravenously), group 2 treated (animals receiving Docetaxel intraperitoneally), group 3 treated (animals receiving a combination of Avastin intravenously and Docetaxel intraperitoneally), group 4 treated (animals receiving MAP15 intravenously), group 5 treated (animals receiving a liposomal formulation of MAP15 intravenously), group 6 treated (animals receiving a combination of MAP15 intravenously, Avastin intravenously and Docetaxel intraperitoneally), and group 7 treated (animals receiving a combination of a liposomal formulation of MAP15 intravenously, Avastin intravenously and Docetaxel intraperitoneally). The treatments are administered as follows: Avastin and Docetaxel are administered weekly using the maximum dosages previously reported in the literature as efficacious against primary tumors and metastatic foci in this model, MAP15 is administered at the dose of 100 μg polypeptide/injection every other day, and liposomal MAP15 is administered at the dose equivalent of 100 μg polypeptide/injection twice weekly.

Similar to the MDA-MB-231 model, the MAP15 efficacy as an anti-invasive/anti-metastatic agent is also determined in two murine breast cancer models (the 4T1 and the D2F2 models). In these models an inoculum of 5×10⁵ cells (either 4T1 or D2F2) in PBS is injected in the mammary fat pads of immunocompetent BALB/c mice and allowed to grow tumors that become palpable (approx. 1 to 1.5 weeks after implantation). Once tumors become palpable, the following treatment groups (5 animals per group) are started: group 1 control (animals receiving no treatments or manipulations), group 2 control (animals receiving empty liposomes intravenously), group 3 control (animals receiving PBS intravenously), group 1 treated (animals receiving Avastin intravenously), group 2 treated (animals receiving Lapatinib intravenously), group 3 treated (animals receiving Herceptin intravenously), group 4 treated (animals receiving a combination of Avastin, Lapatinib and Herceptin intravenously), group 5 treated (animals receiving MAP15 intravenously), group 6 treated (animals receiving a liposomal formulation of MAP15 intravenously), group 7 treated (animals receiving a combination of MAP15, Avastin, Lapatinib, and Herceptin intravenously), and group 8 treated (animals receiving a combination of a liposomal formulation of MAP15, Avastin, Lapatinib, and Herceptin intravenously). The treatments are administered as following: Avastin, Lapatinib and Herceptin are administered weekly using the maximum dosages previously reported in the literature as efficacious against primary tumors and metastatic foci in these models, MAP15 is administered at the dose of 100 μg polypeptide/injection every other day, and liposomal MAP15 is administered at the dose equivalent of 100 μg polypeptide/injection twice weekly.

The tumor growth before and after initiating the treatments in mammary fat pads and at distant sites is monitored in all models by weekly Xenogen bioluminescence imaging. Primary tumor size is also measured by caliper and volumes calculated on the basis of the formula: 1×w2×0.5. For the weekly bioluminescence (luciferase) imaging, mice are injected via an intraperitoneal route with a Luciferin solution (15 mg/mL or 30 mg/kg, in PBS, dose of 5-50 mg/kg) which is allowed to distribute in non-anesthetized animals for about 5-15 minutes. The mice are then placed into a clear Plexiglas anesthesia box (2-4% isofluorane) which allows unimpeded visual inspection of the animals and monitoring of their breathing status. In this setting, the anesthesia delivery tube that supplies the anesthesia to the box is split so that the same concentration of anesthesia is delivered to the anesthesia manifold located inside the imaging chamber on the Xenogen IVIS 100 imaging instrument. After the mice are fully anesthetized, they are transferred from the anesthesia box to the anesthesia nose cones attached to the manifold in the imaging chamber, the door is closed, and images are acquired using Xenogen instrument. The imaging time is between one to five minutes per side (dorsal/ventral), depending on the experiment. When the animals are turned from dorsal to ventral (or vice versa) they are monitored for any signs of distress or changes in vitality. The acquired images are evaluated by comparing the level luciferase activity at the site of the control and treated tumors, and by comparing the distribution area under the curve associated with the luminescence from tumors in each treatment group. By employing the above approach the efficacy of MAP15 against primary tumors and bioluminescent metastatic foci is determined. Animal weights are also measured twice weekly and animals are observed visually for any signs of stress response or malaise. Animals that showed severe signs of impairment are excluded from the experiment.

At the conclusion of these studies (after 8 weeks of treatment in the MDA-MB-231 model, and after 3 weeks of treatment in the 4T1 and D2F2 models) a determination of whether MAP15 alone is as efficacious as or better than any of the monotherapies tested (either Avastin or Docetaxel or Lapatinib or Herceptin) at inhibiting primary tumor growth is made, reducing the number and size or both of metastatic foci, and prolonging animal survival in these tumor models. In addition, when administered in combination with either Avastin plus Docetaxel in the MDA-MB-231 model or in combination with Avastin plus Lapatinib in the 4T1 model or in combination with Avastin plus Lapatinib plus Herceptin in the D2F2 model, MAP15 proved to be more efficacious than any monotherapy or other combinations at inhibiting primary tumor growth, reducing the number and size or both of metastatic foci, and prolonging survival in these tumor models.

### Appendix

### ADAM Nucleic Acid and Amino Acid Sequences

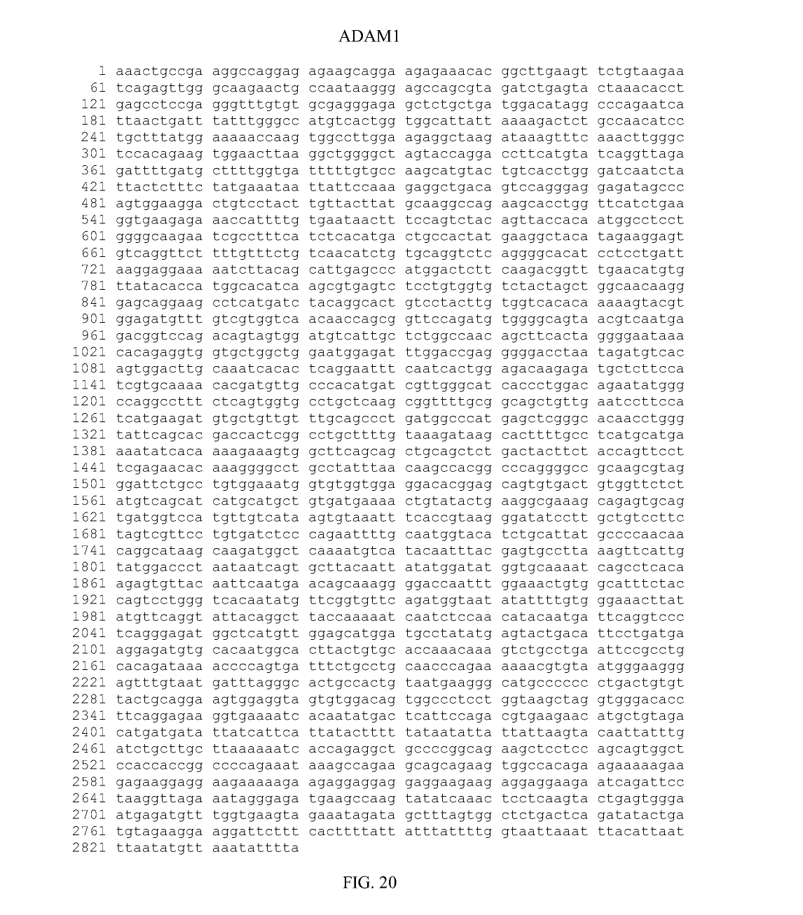

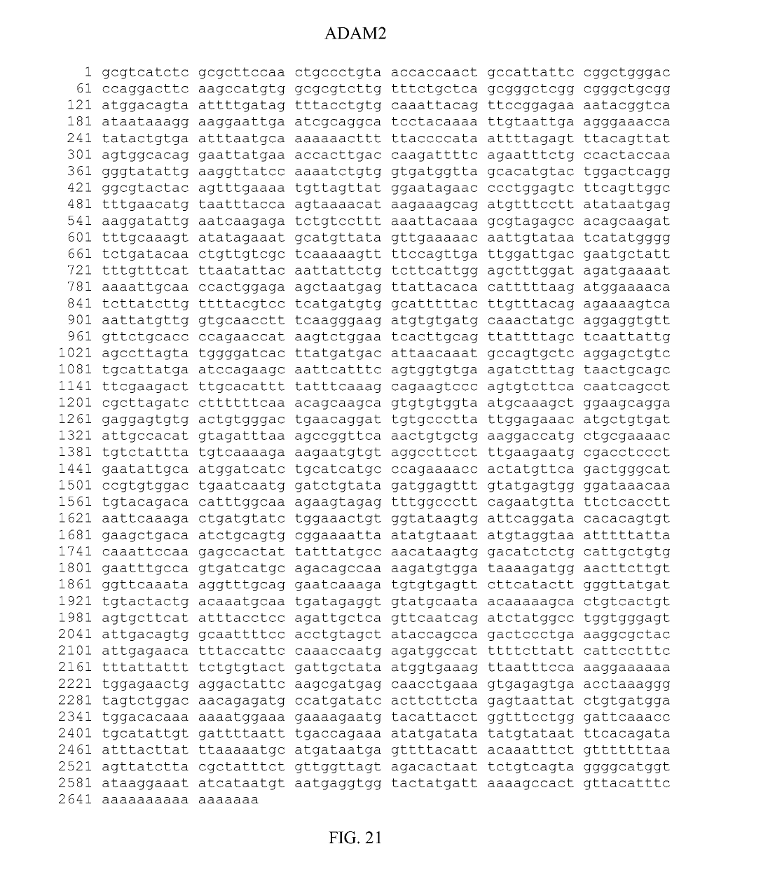

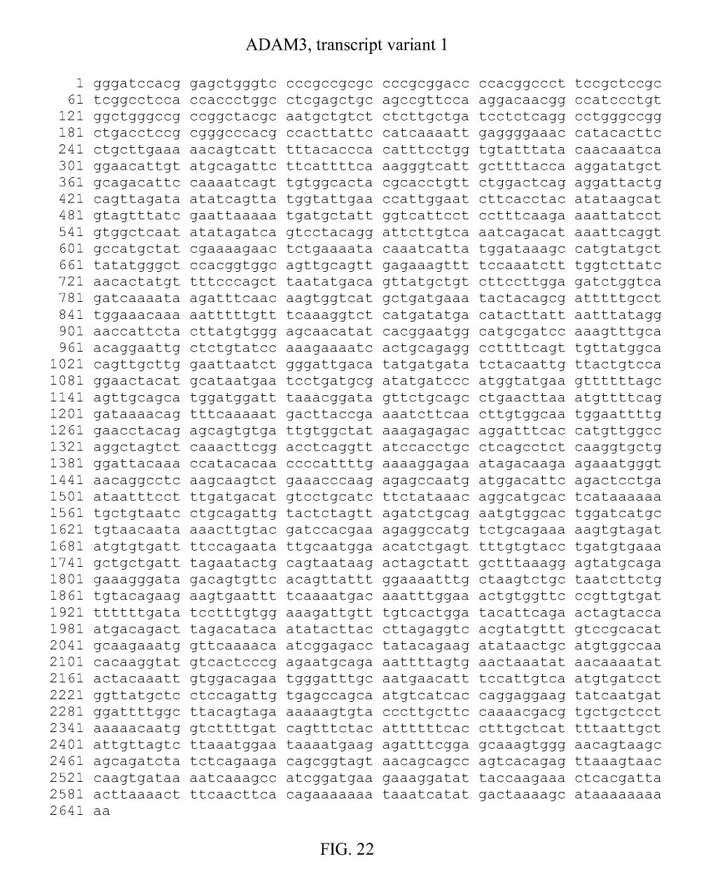

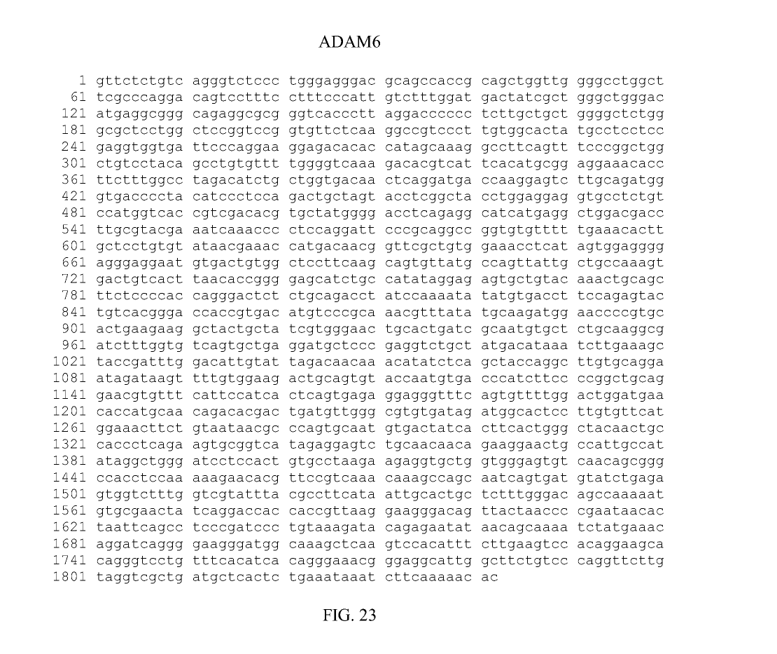

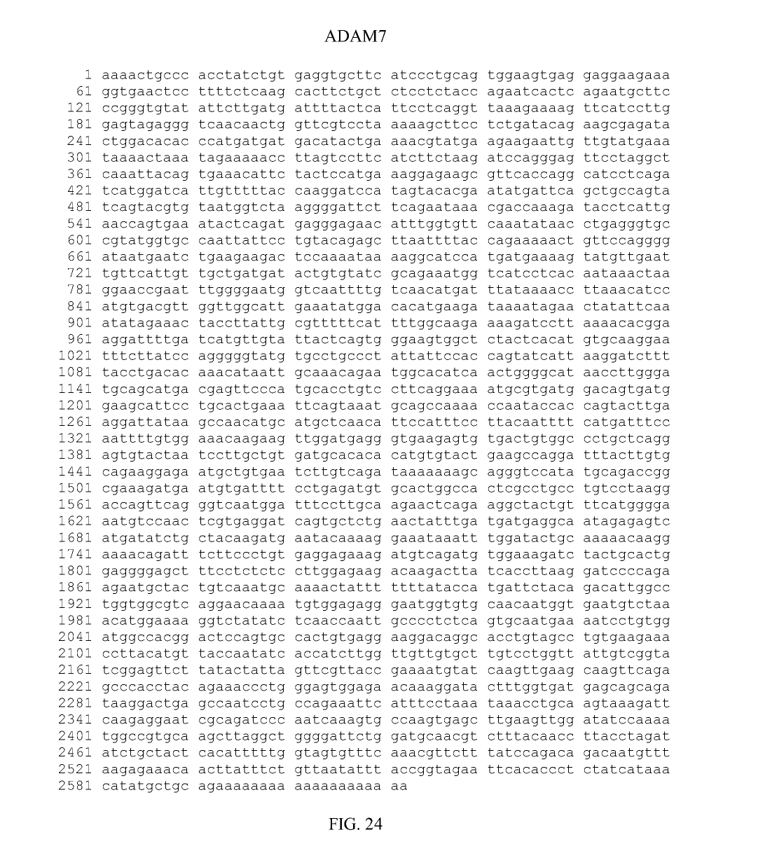

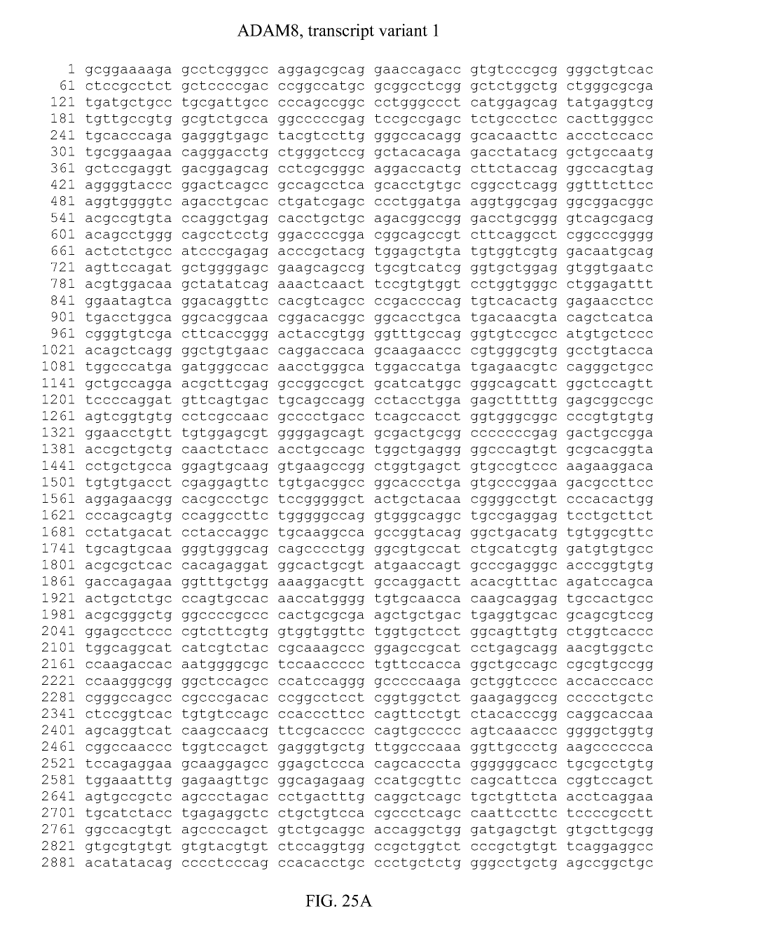

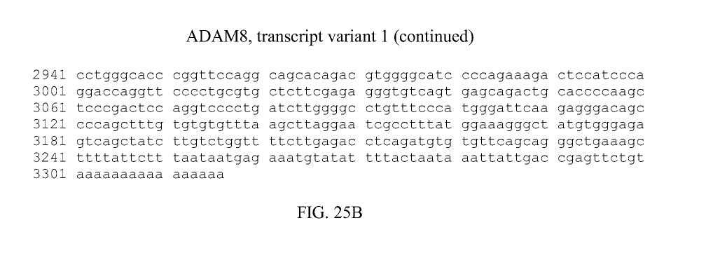

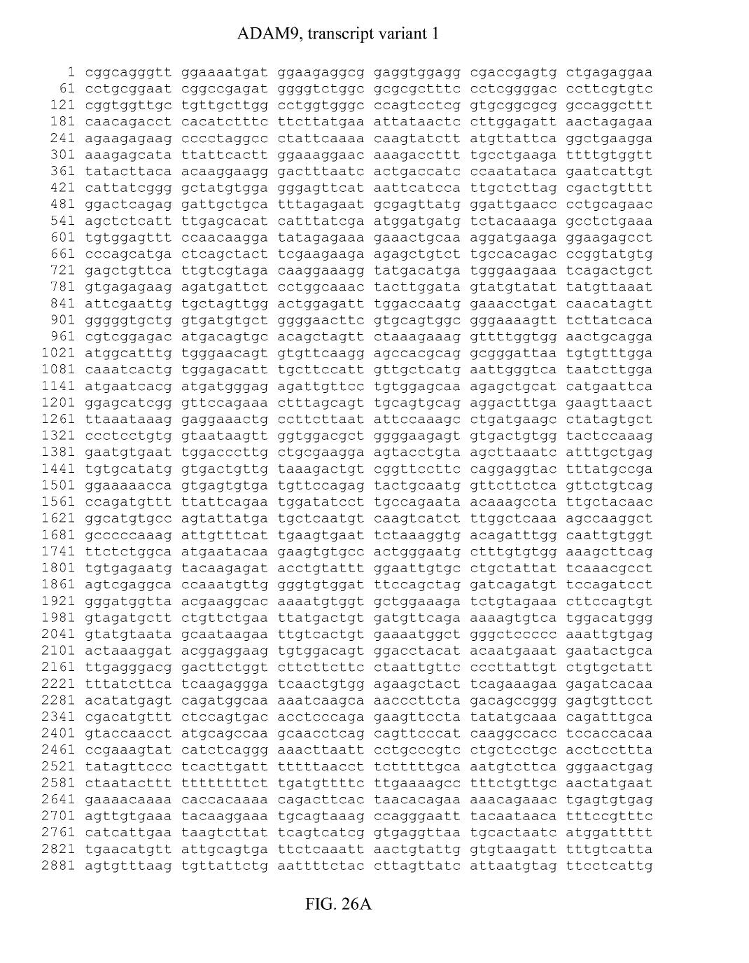

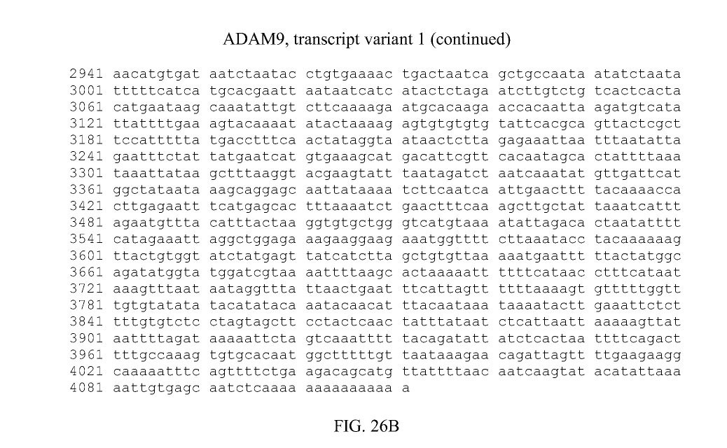

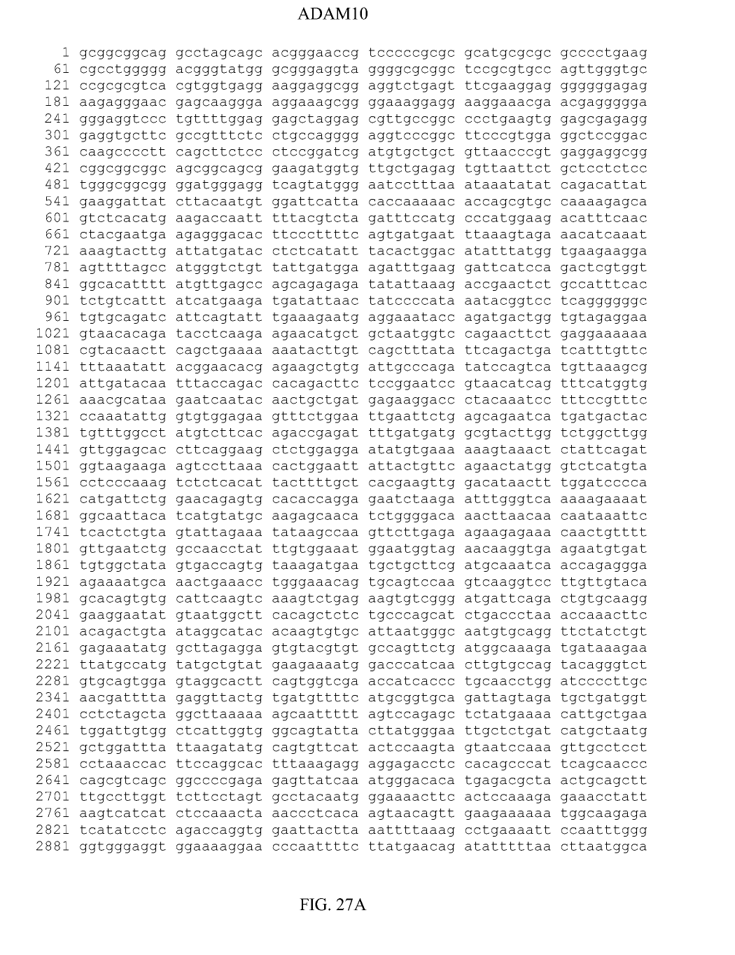

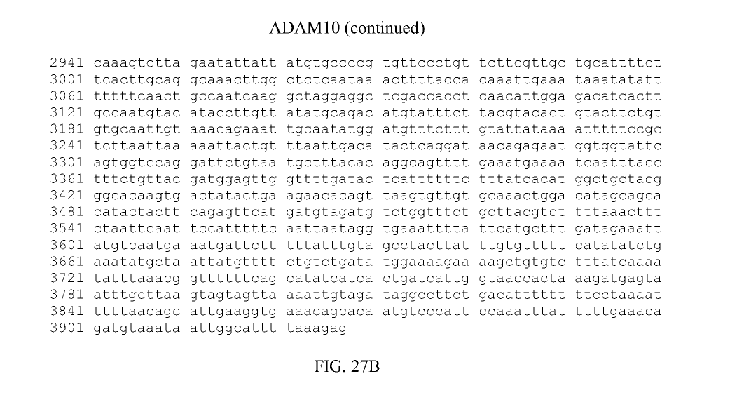

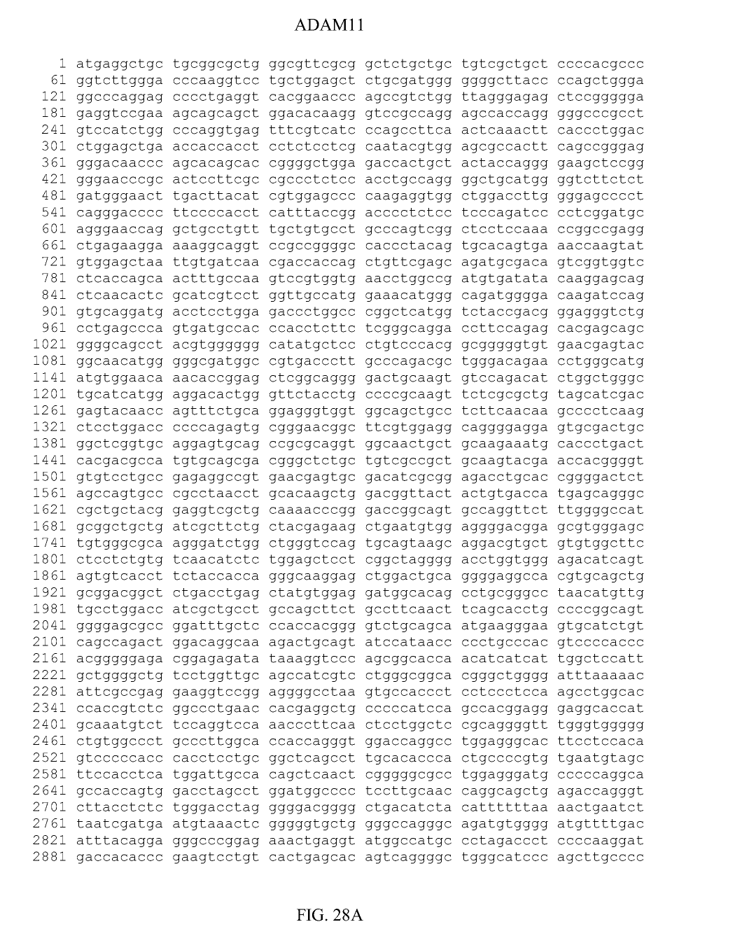

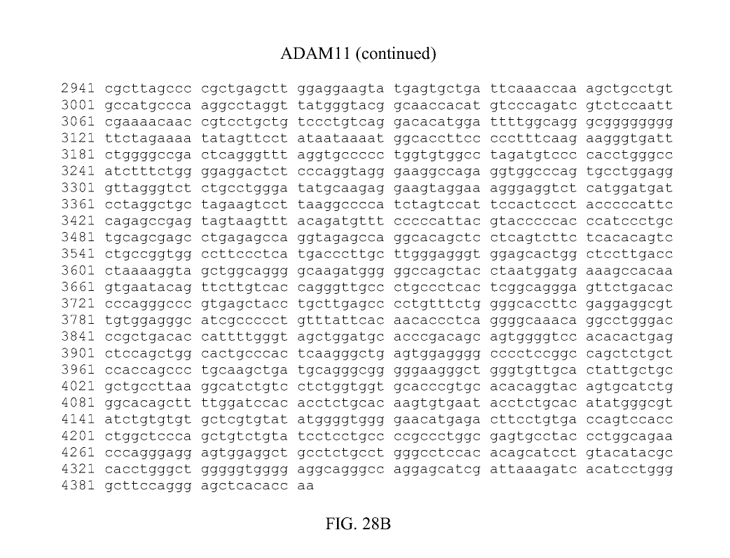

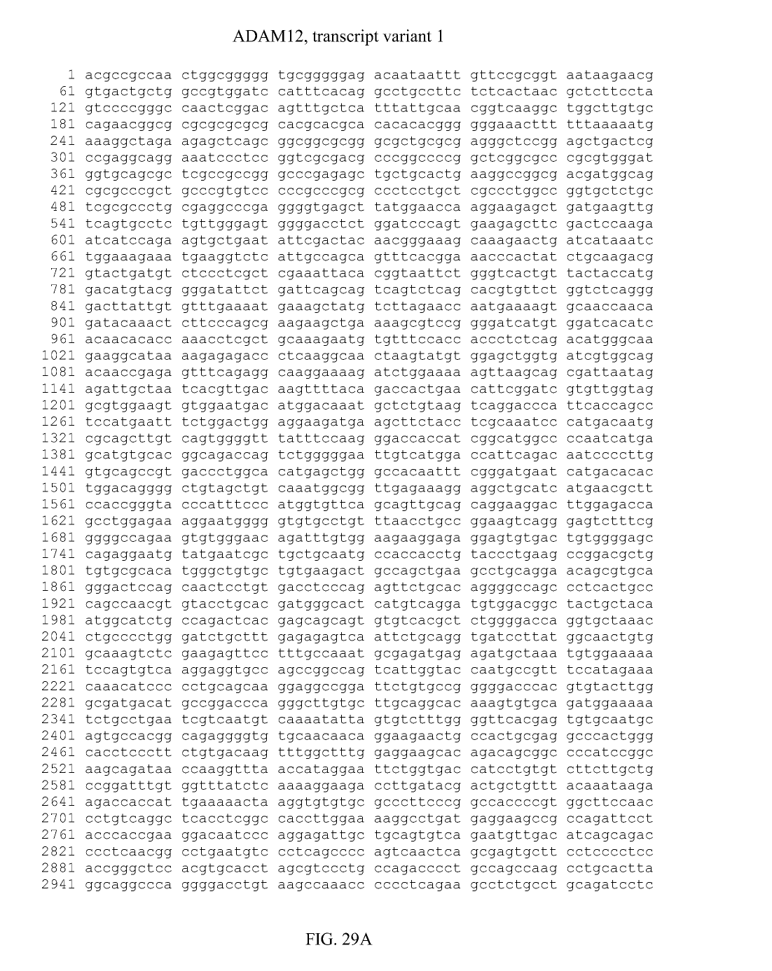

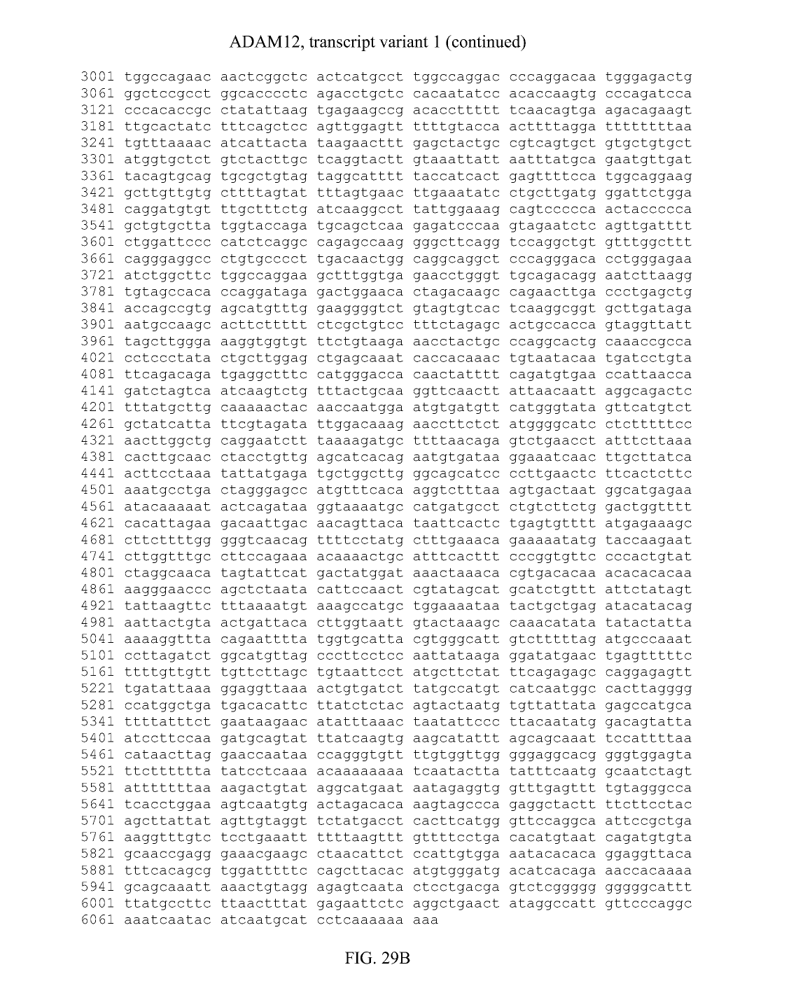

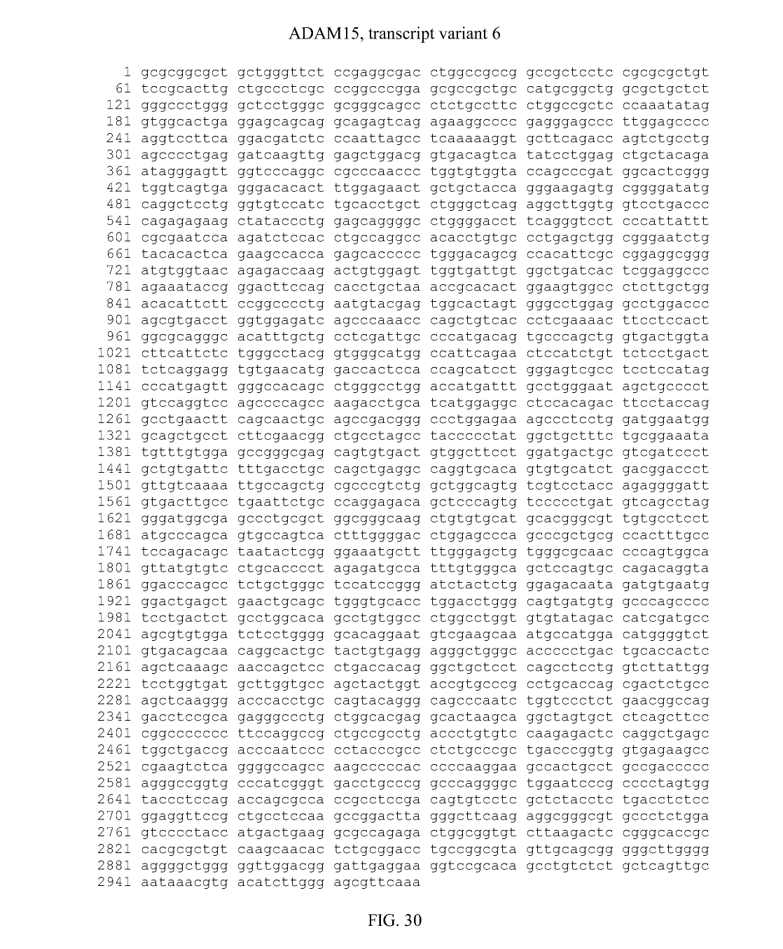

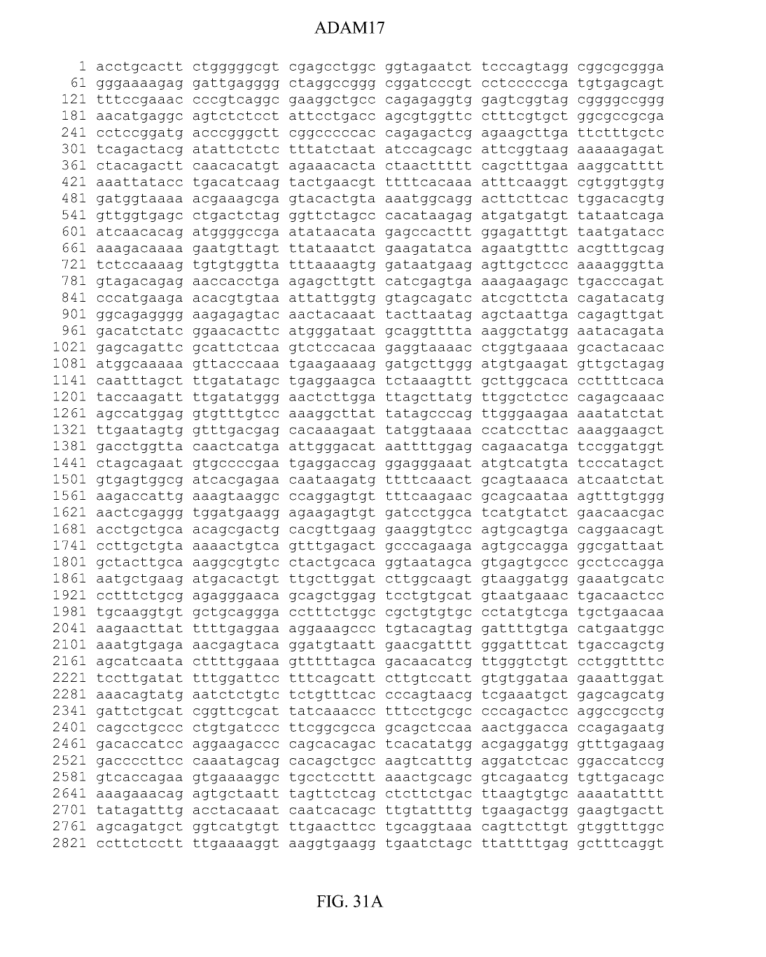

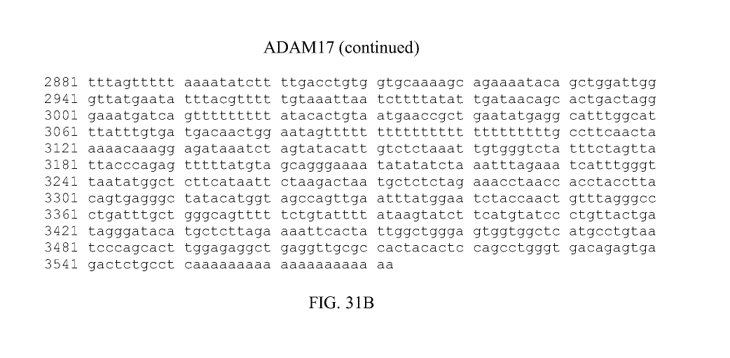

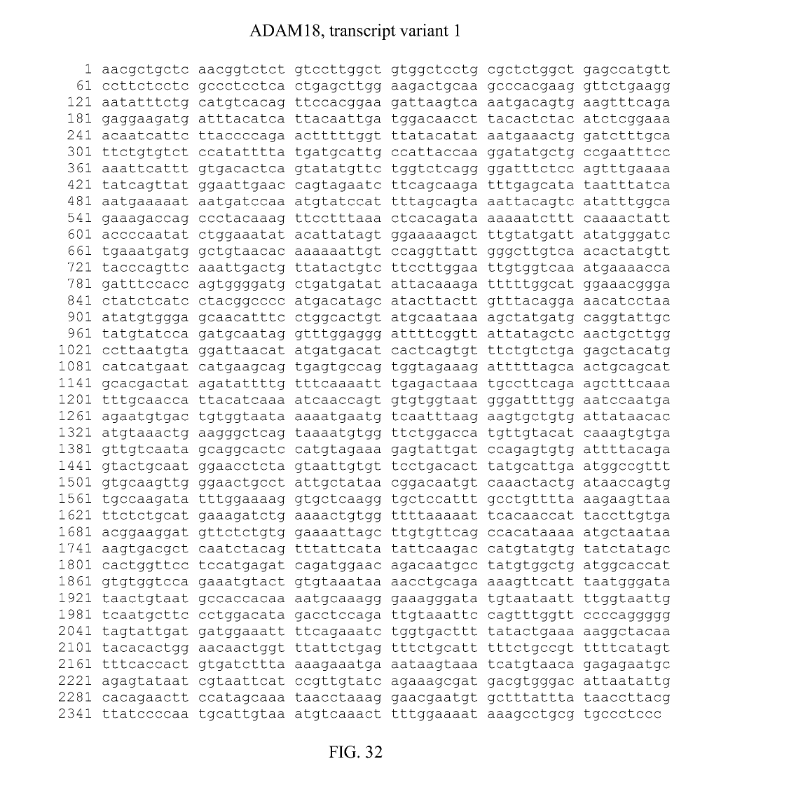

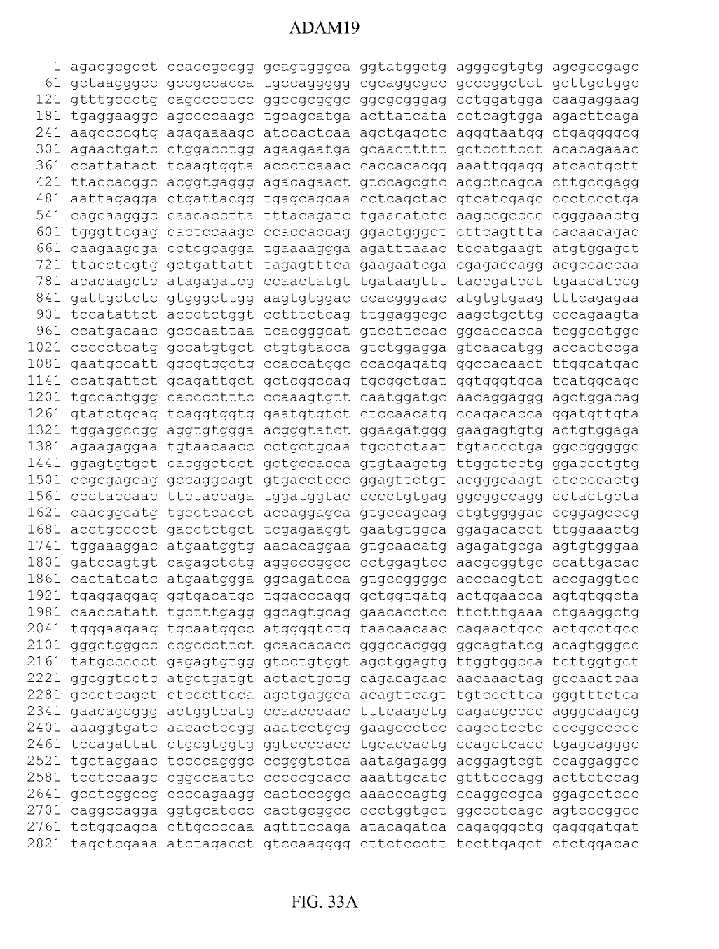

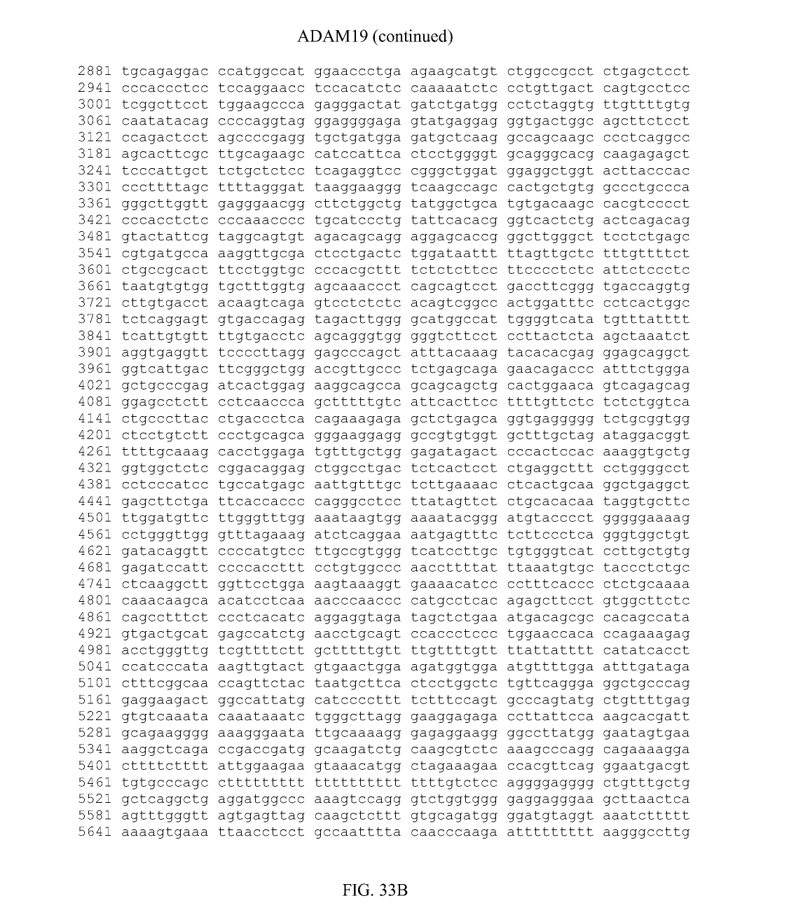

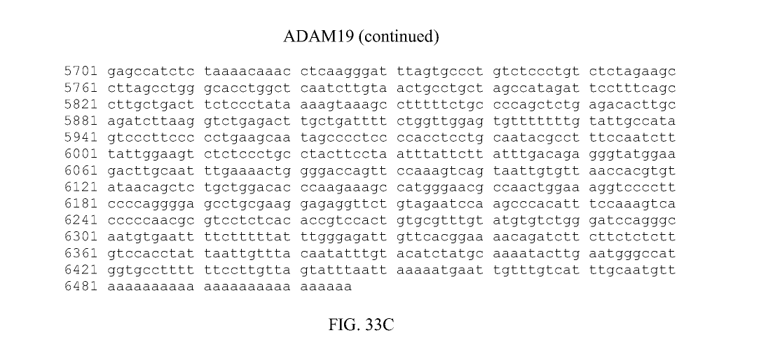

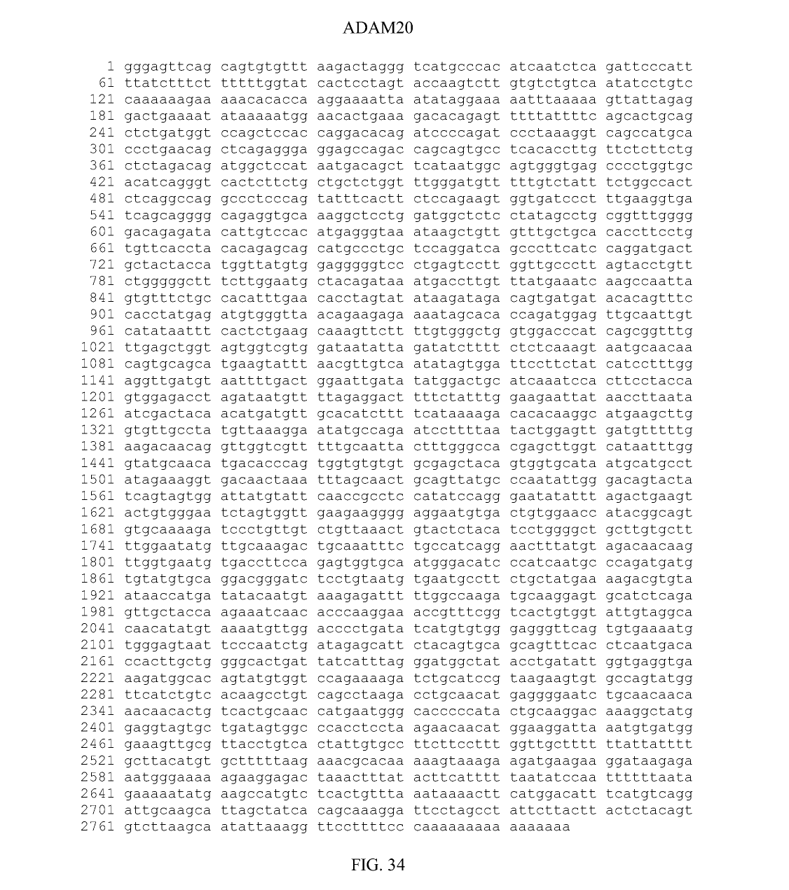

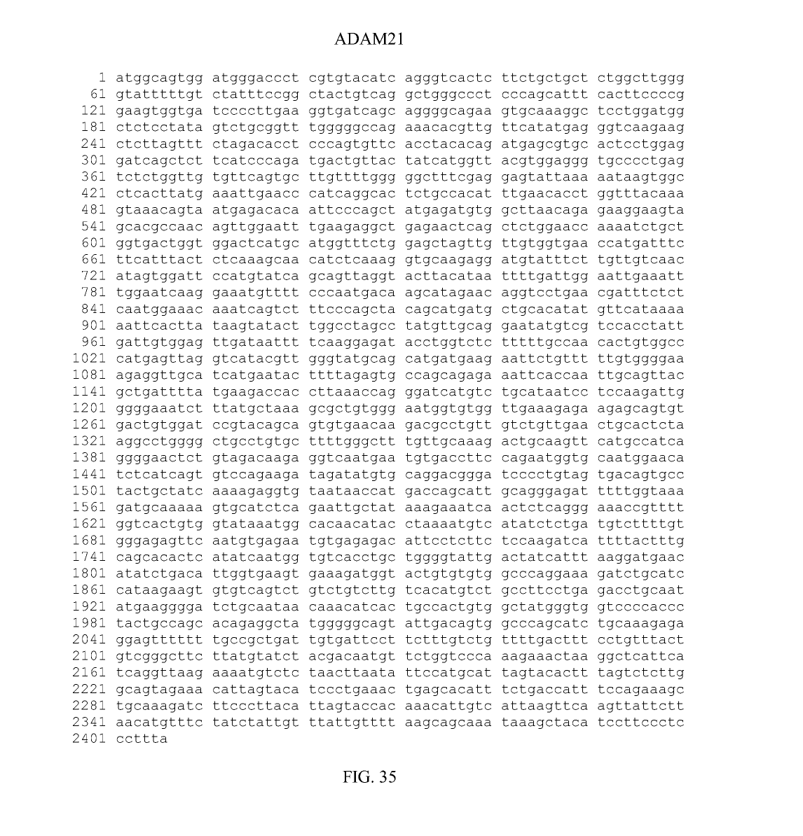

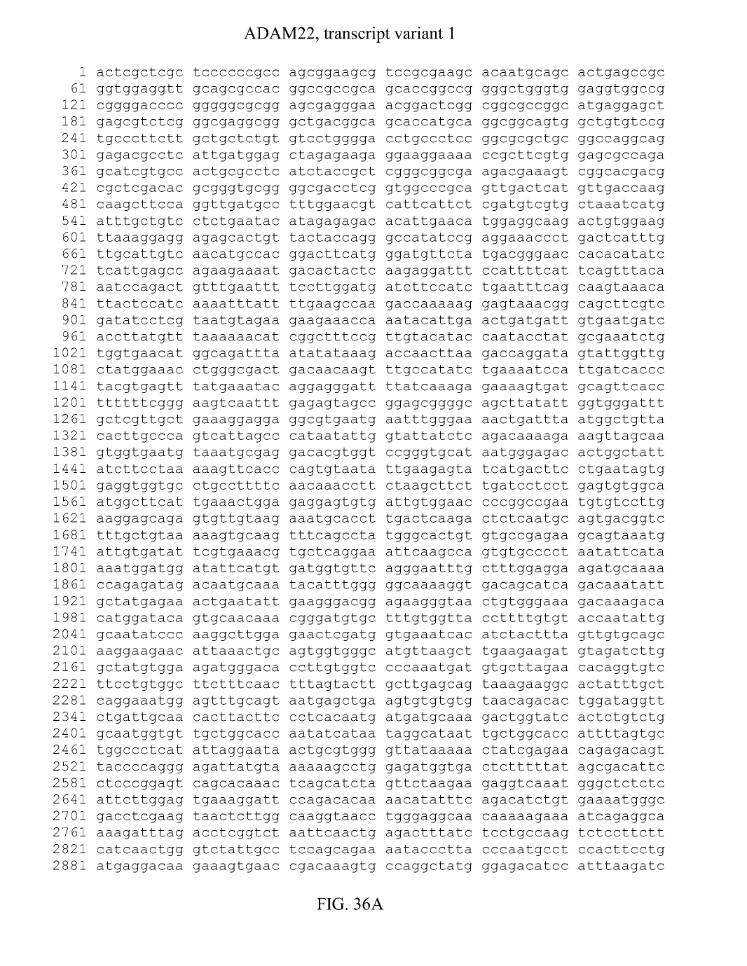

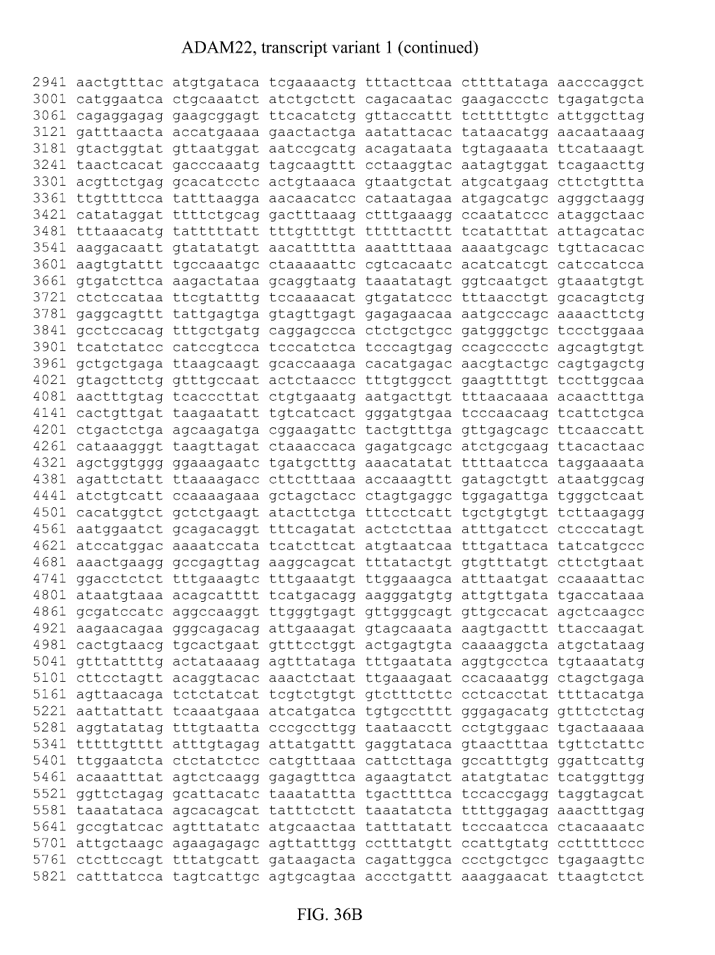

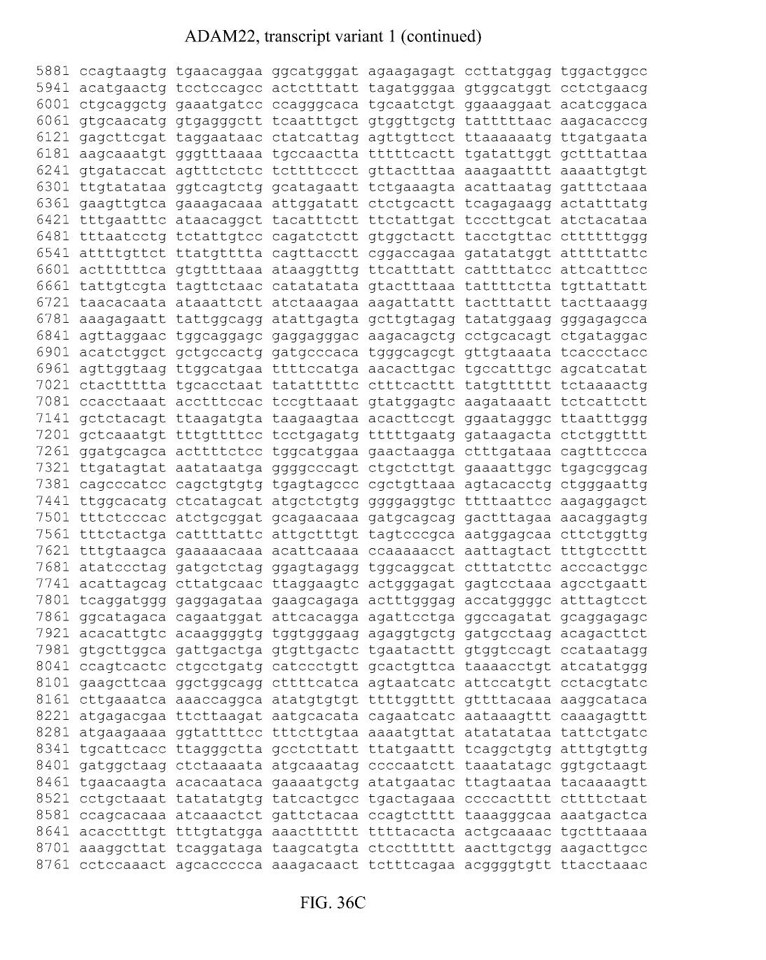

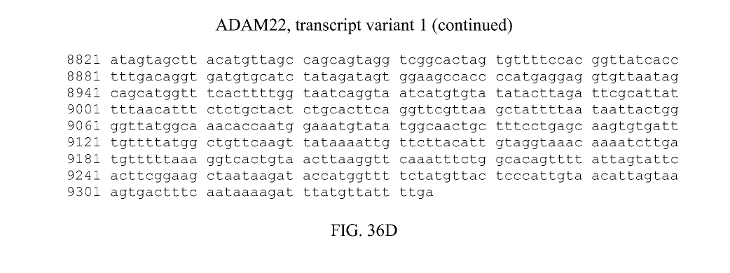

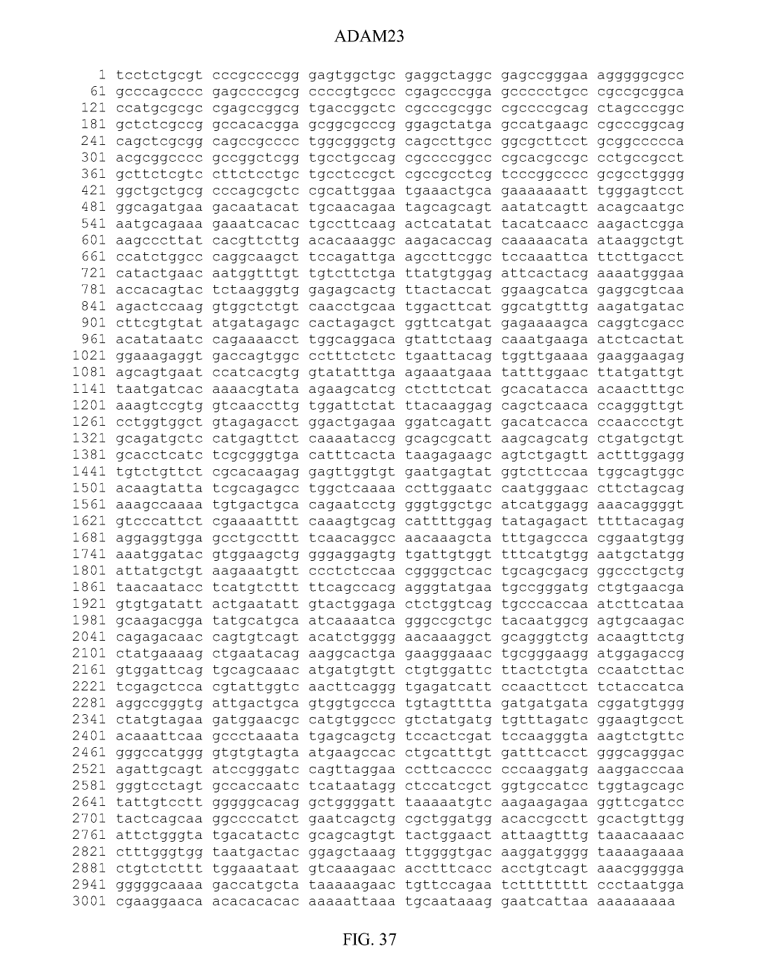

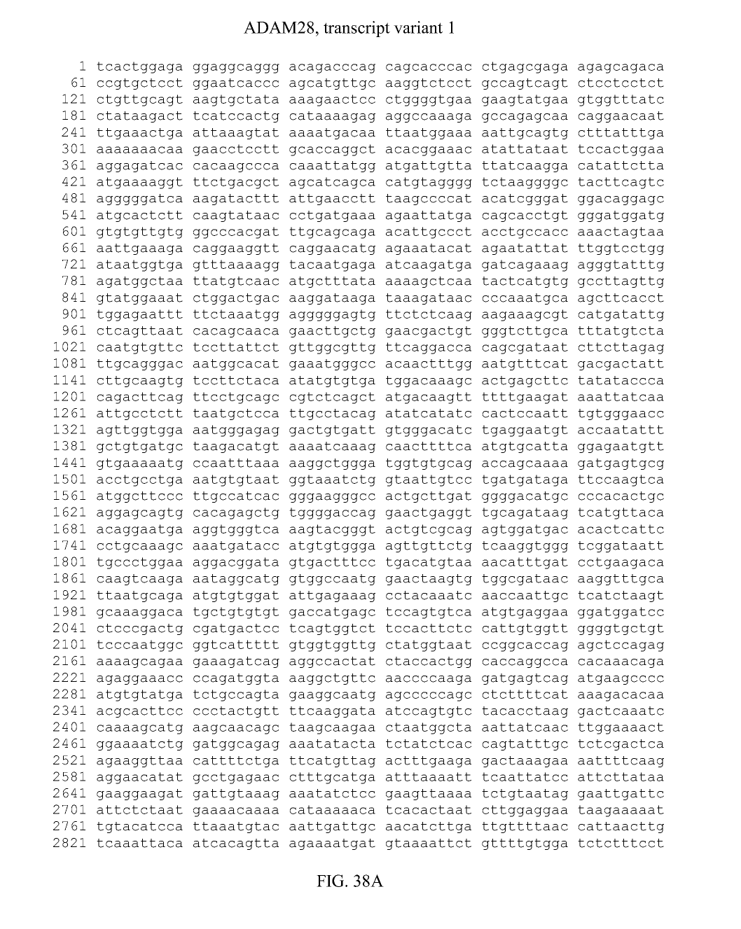

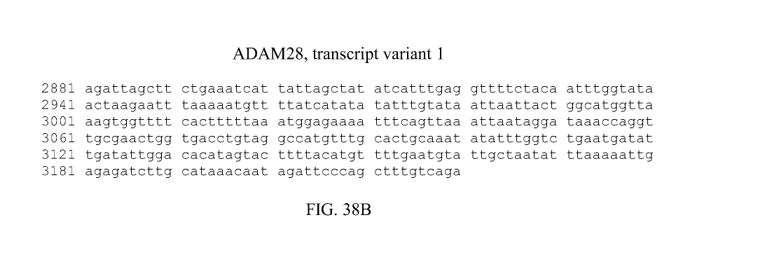

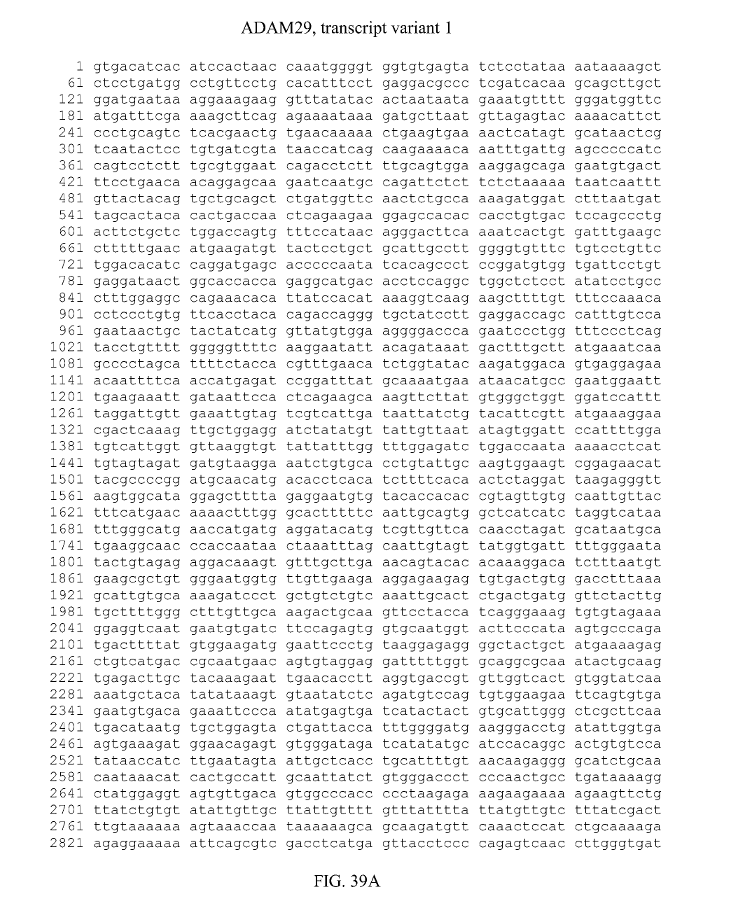

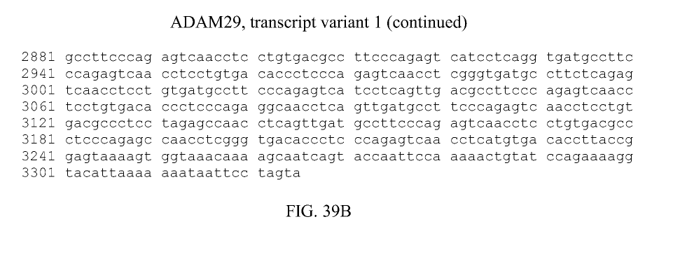

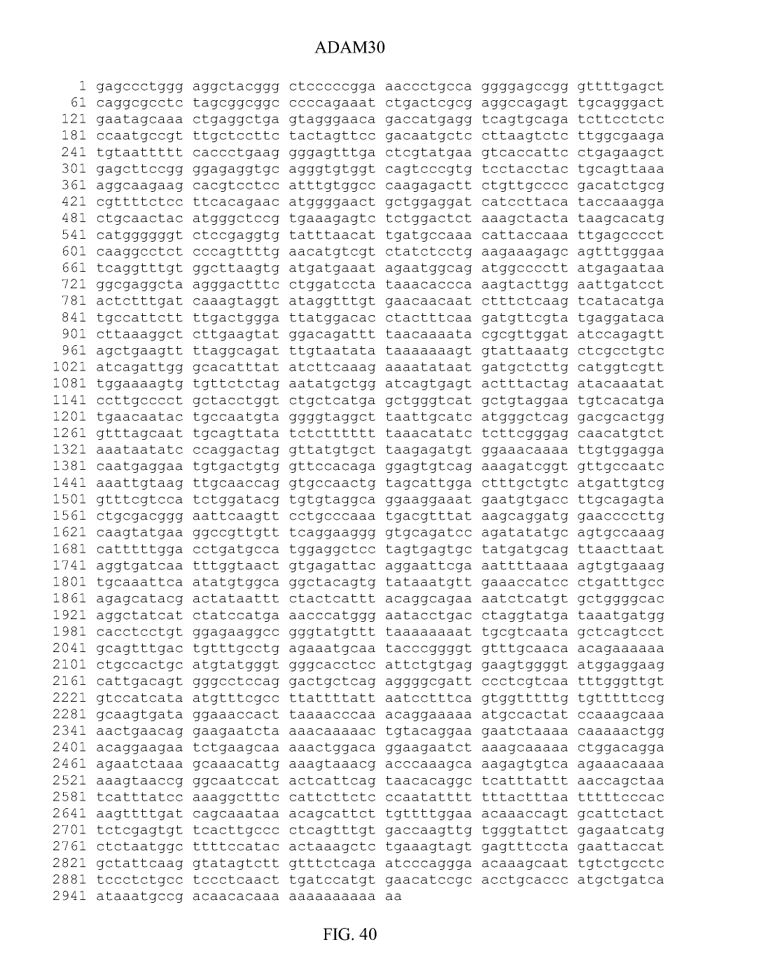

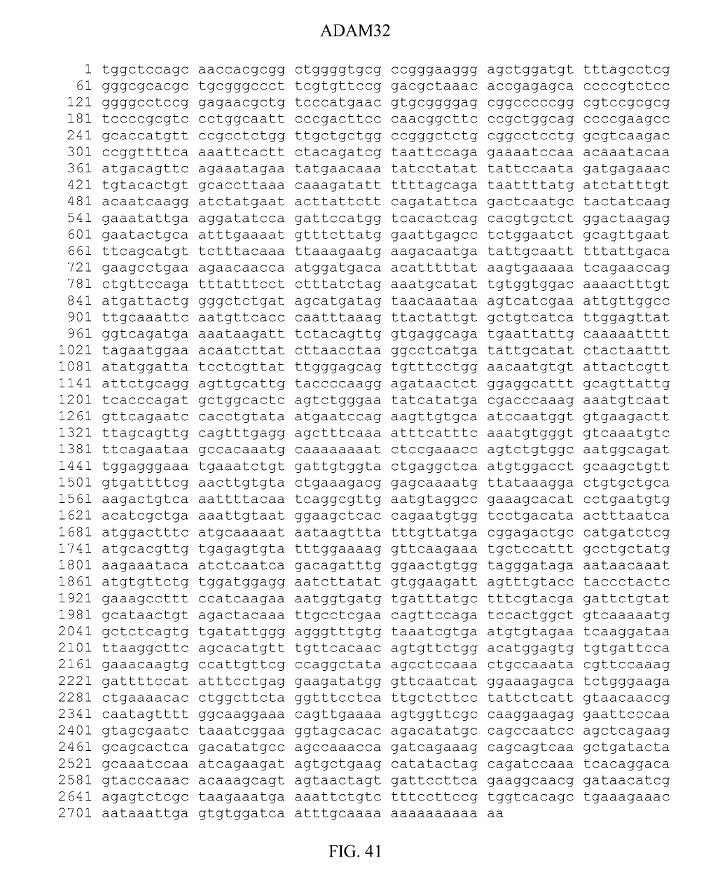

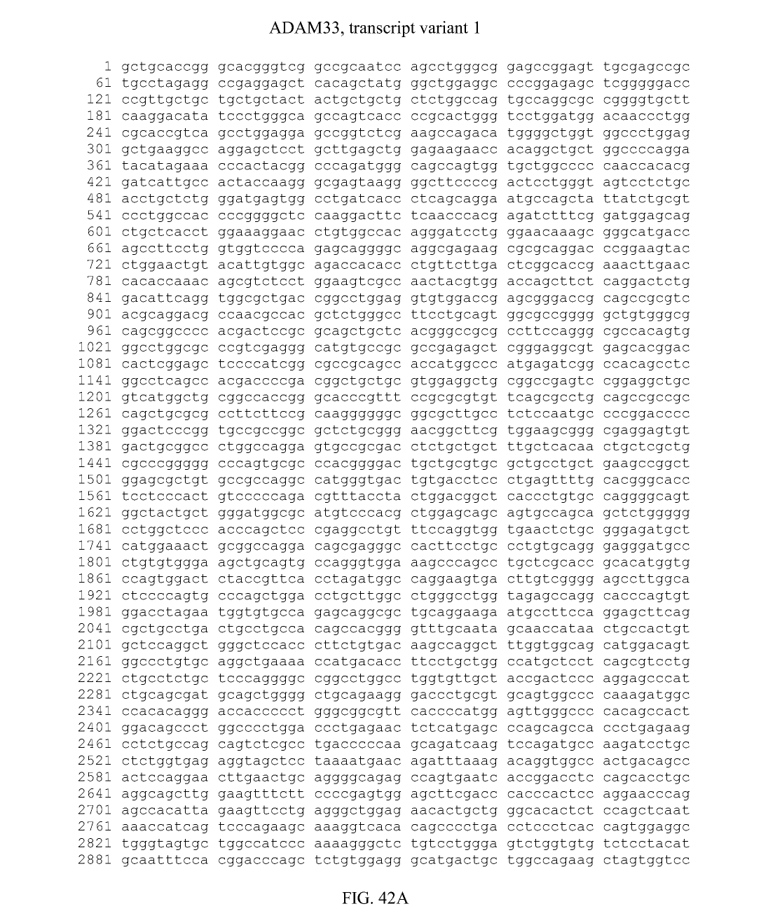

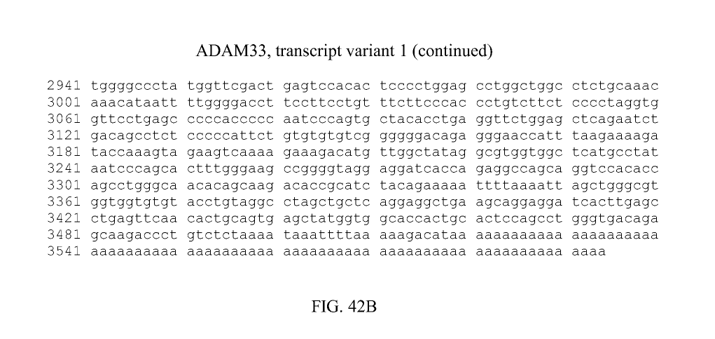

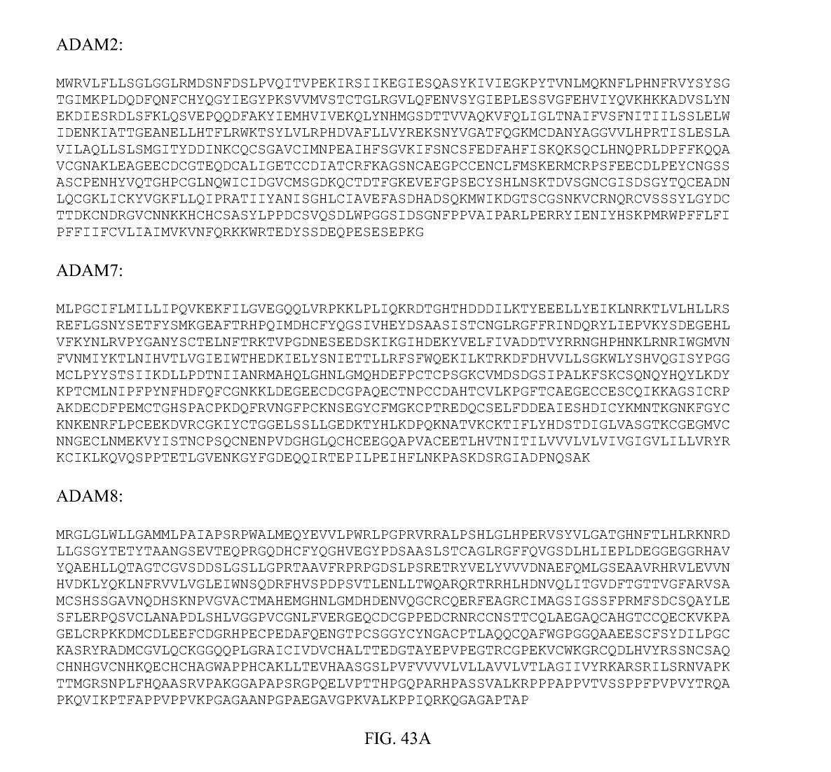

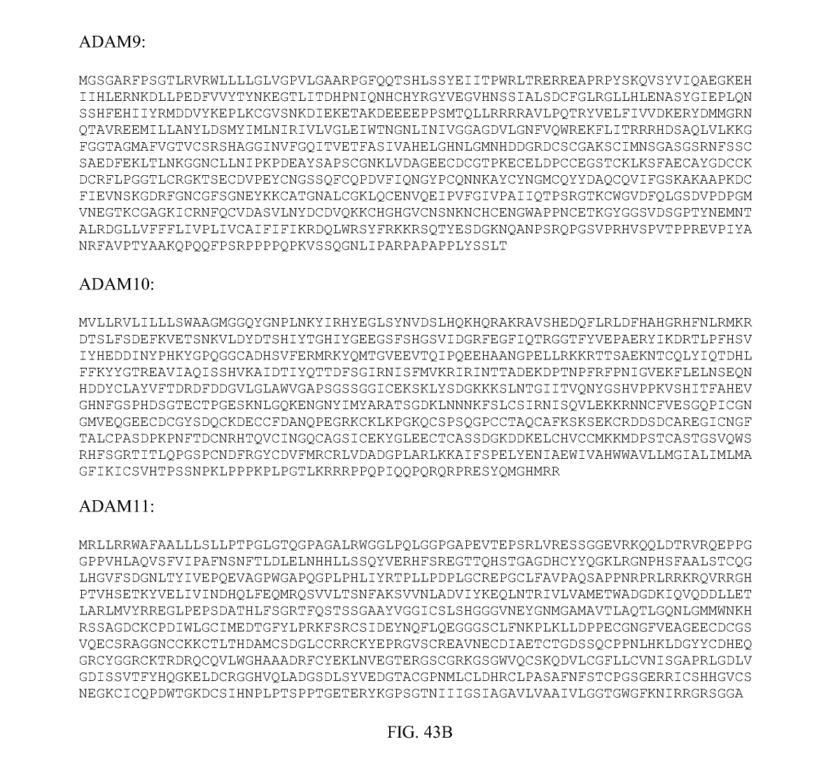

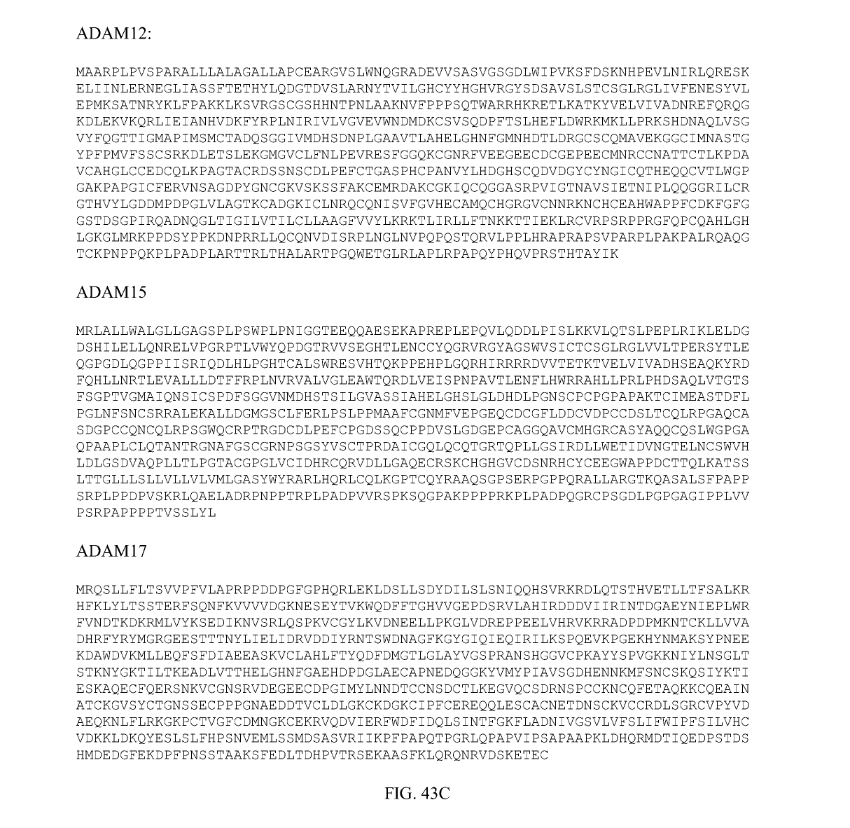

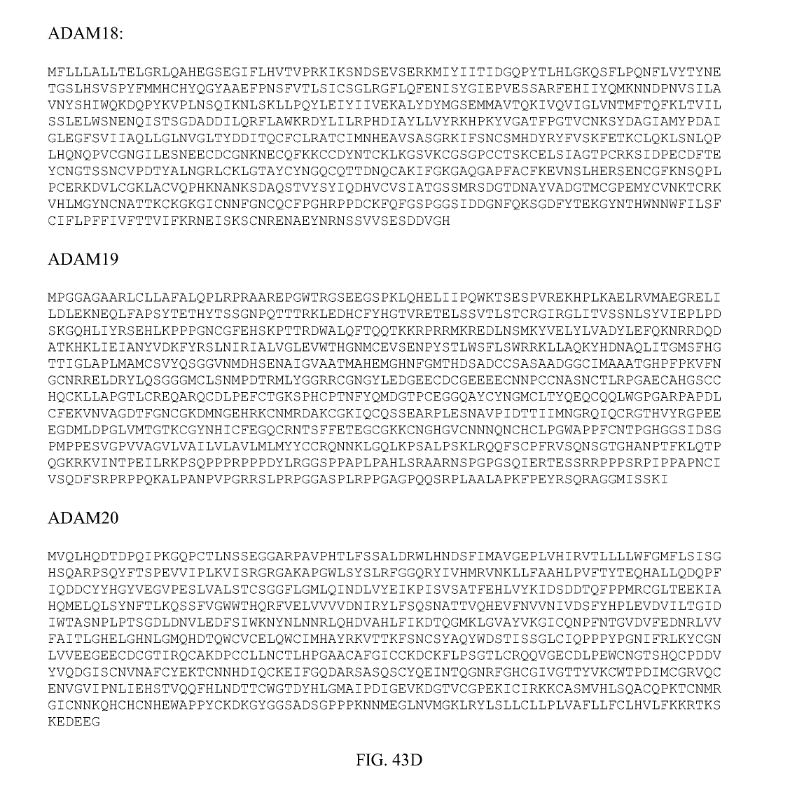

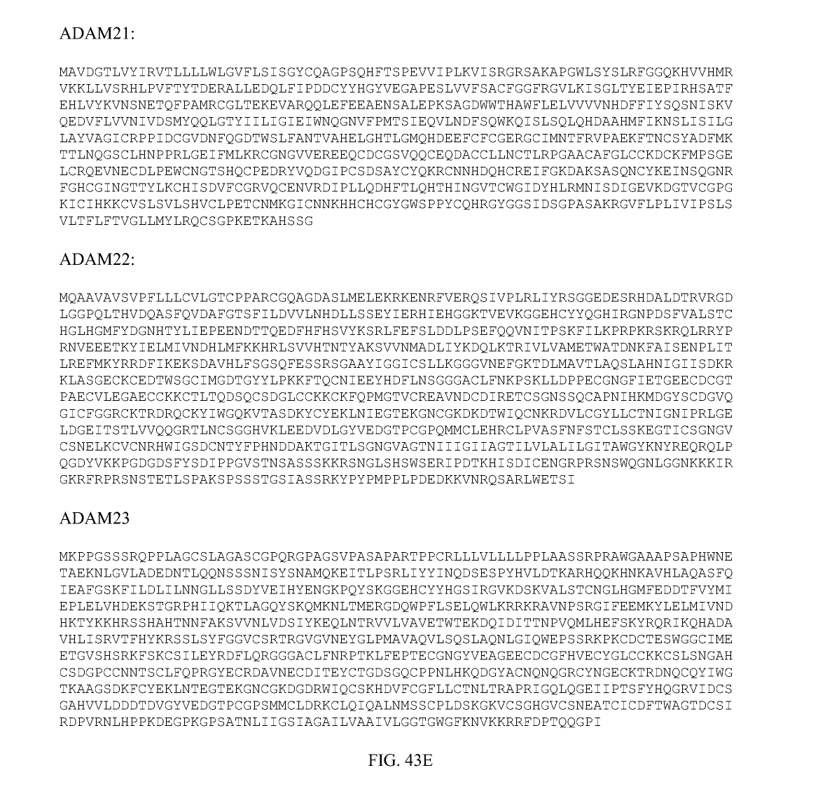

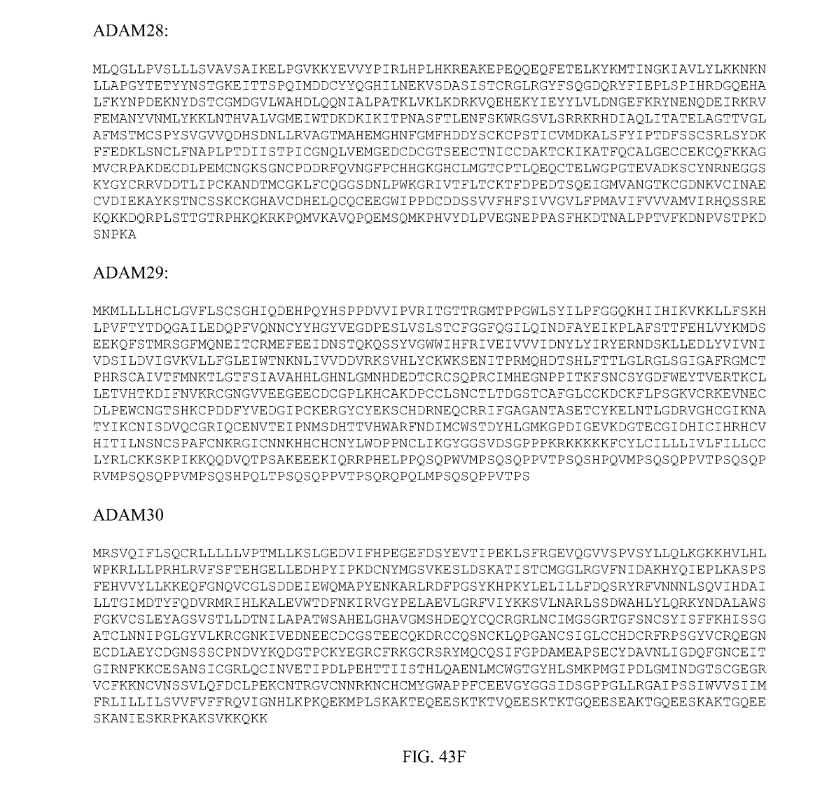

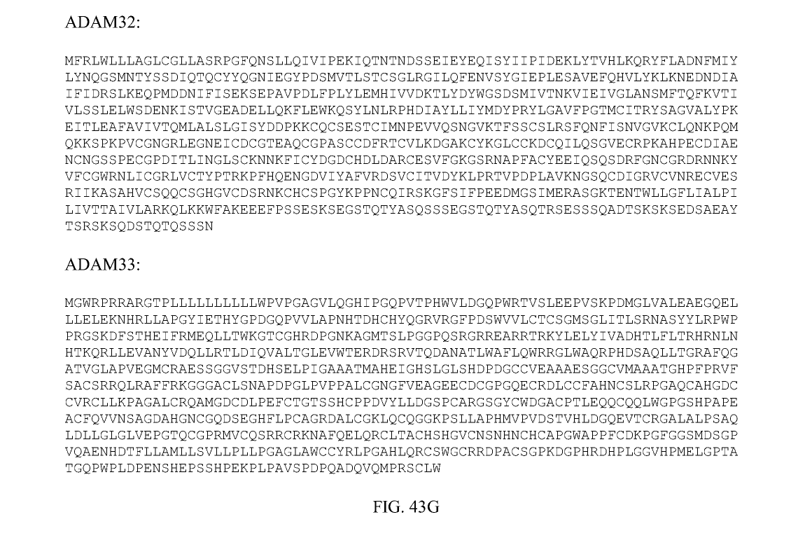

Nucleic acid sequences corresponding to ADAM RNA transcripts are provided in FIGS. 20-42. Amino acid sequences for the corresponding ADAM polypeptides are provided in FIG. 43.

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

All publications, patent applications, patents, and other references mentioned herein are expressly incorporated by reference in their entirety, including all formulas and figures, to the same extent as if each were incorporated by reference individually. In case of conflict, the present specification, including definitions, will control.

Other embodiments are set forth within the following claims.

### CITED REFERENCES

1. Edwards, D. R., M. M. Handsley, and C. J. Pennington, The ADAM metalloproteinases. Mol Aspects Med, 2008. 29(5): p. 258-89.
2. Mochizuki, S. and Y. Okada, ADAMs in cancer cell proliferation and progression. Cancer Sci, 2007. 98(5): p. 621-8.
3. Reiss, K., A. Ludwig, and P. Saftig, Breaking up the tie: disintegrin-like metalloproteinases as regulators of cell migration in inflammation and invasion. Pharmacol Ther, 2006. 111(3): p. 985-1006.
4. Tousseyn, T., et al., (Make) stick and cut loose—disintegrin metalloproteases in development and disease. Birth Defects Res C Embryo Today, 2006. 78(1): p. 24-46.
5. Arribas, J., J. J. Bech-Serra, and B. Santiago-Josefat, ADAMs, cell migration and cancer. Cancer Metastasis Rev, 2006. 25(1): p. 57-68.
6. Blanchot-Jossic, F., et al., Up-regulated expression of ADAM17 in human colon carcinoma: co-expression with EGFR in neoplastic and endothelial cells. J Pathol, 2005. 207(2): p. 156-63.
7. Lendeckel, U., et al., Increased expression of ADAM family members in human breast cancer and breast cancer cell lines. J Cancer Res Clin Oncol, 2005. 131(1): p. 41-8.
8. Mazzocca, A., et al., A secreted form of ADAM9 promotes carcinoma invasion through tumor-stromal interactions. Cancer Res, 2005. 65(11): p. 4728-38.
9. McGowan, P. M., et al., ADAM-17 predicts adverse outcome in patients with breast cancer. Ann Oncol, 2008. 19(6): p. 1075-81.
10. McGowan, P. M., et al., ADAM-17 expression in breast cancer correlates with variables of tumor progression. Clin Cancer Res, 2007. 13(8): p. 2335-43.
11. Mitsui, Y., et al., ADAM28 is overexpressed in human breast carcinomas: implications for carcinoma cell proliferation through cleavage of insulin-like growth factor binding protein-3. Cancer Res, 2006. 66(20): p. 9913-20.
12. Najy, A. J., K. C. Day, and M. L. Day, ADAM15 supports prostate cancer metastasis by modulating tumor cell endothelial cell interaction. Cancer Res, 2008. 68(4): p. 1092-9.
13. O'Shea, C., et al., Expression of ADAM-9 mRNA and protein in human breast cancer. Int J Cancer, 2003. 105(6): p.754-61.
14. Ringel, J., et al., Aberrant expression of a disintegrin and metalloproteinase 17/tumor necrosis factor-alpha converting enzyme increases the malignant potential in human pancreatic ductal adenocarcinoma. Cancer Res, 2006. 66(18): p. 9045-53.
15. Wildeboer, D., et al., Metalloproteinase disintegrins ADAM8 and ADAM19 are highly regulated in human primary brain tumors and their expression levels and activities are associated with invasiveness. J Neuropathol Exp Neurol, 2006. 65(5): p. 516-27.
16. D'Abaco, G. M., et al., ADAM22, expressed in normal brain but not in high-grade gliomas, inhibits cellular proliferation via the disintegrin domain. Neurosurgery, 2006. 58(1): p. 179-86; discussion 179-86.
17. Takeda, S., Three-dimensional domain architecture of the ADAM family proteinases. Semin Cell Dev Biol, 2008.
18. Bessette et al., Efficient folding of proteins with multiple disulfide bonds in the Escherichia coli cytoplasm. Proc. Natl. Acad. Sci (USA) 1999, 96(24):13703-8.
19. Calvete, J. J., et al., Snake venom disintegrins: novel dimeric disintegrins and structural diversification by disulphide bond engineering. Biochem J, 2003. 372(Pt 3): p. 725-34.
20. Juarez, P., et al., Evolution of snake venom disintegrins by positive darwinian selection. Mol Biol Evol, 2008. 25(11): p. 2391-407.
21. McLane, M. A., T. Joerger, and A. Mahmoud, Disintegrins in health and disease. Front Biosci, 2008. 13: p. 6617-37.
22. Shimaoka, M. and T. A. Springer, Therapeutic antagonists and conformational regulation of integrin function. Nat Rev Drug Discov, 2003. 2(9): p. 703-16.
23. Silva, R., et al., Integrins: the keys to unlocking angiogenesis. Arterioscler Thromb Vasc Biol, 2008. 28(10): p. 1703-13.
24. Hood, J. D. and D. A. Cheresh, Role of integrins in cell invasion and migration. Nat Rev Cancer, 2002. 2(2): p. 91-100.
25. Mizejewski, G. J., Role of integrins in cancer: survey of expression patterns. Proc Soc Exp Biol Med, 1999. 222(2): p.124-38.
26. Swenson, S., et al., Intravenous liposomal delivery of the snake venom disintegrin contortrostatin limits breast cancer progression. Mol Cancer Ther, 2004. 3(4): p. 499-511.
27. Minea, R., et al., Development of a novel recombinant disintegrin, contortrostatin, as an effective anti-tumor and antiangiogenic agent. Pathophysiol Haemost Thromb, 2005. 34(4-5): p. 177-83.
28. Igarashi, T., et al., Crystal structures of catrocollastatin/VAP2B reveal a dynamic, modular architecture of ADAM/adamalysin/reprolysin family proteins. FEBS Lett, 2007. 581(13): p. 2416-22. Page 9 of 43 Page 14
29. Prinz, W. A., et al., The role of the thioredoxin and glutaredoxin pathways in reducing protein disulfide bonds in the Escherichia coli cytoplasm. J Biol Chem, 1997. 272(25): p. 15661-7.
30. LaVallie, E. R., et al., A thioredoxin gene fusion expression system that circumvents inclusion body formation in the E. coli cytoplasm. Biotechnology (N Y), 1993. 11(2): p. 187-93.
31. Yoneda, T., et al., A bone-seeking clone exhibits different biological properties from the MDA-MB-231 parental human breast cancer cells and a brain-seeking clone in vivo and in vitro. J Bone Miner Res, 2001. 16(8): p. 1486-95.
32. Yuan, X., et al., Isolation of cancer stem cells from adult glioblastoma multiforme. Oncogene, 2004. 23(58): p. 400.
33. Alfandari, D., C. McCusker, and H. Cousin, ADAM function in embryogenesis. Semin Cell Dev Biol, 2008.
34. Duffy M. J. et al., Role of ADAMs in cancer formation and progression. Clin Cancer Res. 2009 Feb. 15; 15(4):1140-4.
35. Protopapa, E. et al., Vascular density and the response of breast carcinomas to mastectomy and adjuvant chemotherapy. Eur J Cancer, 1993. 29A(10): p. 1391-3.
36. Fox, S. B. and A. L. Harris, Histological quantitation of tumour angiogenesis. Apmis, 2004. 112(7-8): p. 413-30.

That which is claimed is:

1. A Modified ADAM-derived Polypeptide (MAP), comprising an ADAM-derived Polypeptide (AP) that is modified at the following amino acid residues:
a) a first cysteine C-terminal to a Cys-Asp-Cys (CDC) motif;
b) two contiguous amino acids C-terminal to said first cysteine; and,
c) a cysteine C-terminal to a tripeptide motif,
and wherein the modification of said amino acid residues is independently selected from the group consisting of:
a) deletion;
b) substitution; and,
c) chemical modification,
and wherein ADAM means “A Disintegrin and Metalloproteinase” and, wherein, said AP comprises a distintegrin-like domain from an ADAM comprising (i) the Cys-Asp-Cys (CDC) motif, (ii) the tripeptide motif as indicated in FIG. 1 and (iii) lacking all or substantially all of the ADAM metalloprotease domain, cysteine-rich domains, and interdomain segments, and wherein said ADAM is not ADAM17.

2. The MAP of claim 1 wherein said MAP is derived from an ADAM selected from the group consisting of: ADAM15, ADAM28, ADAM1, ADAM2, ADAM3, ADAM6, ADAM7, ADAMS, ADAM9, ADAM10, ADAM11, ADAM12, ADAM18, ADAM19, ADAM20, ADAM21, ADAM22, ADAM23, ADAM29, ADAM30, ADAM32, ADAM33.

3. The MAP of claim 1 wherein said MAP is selected from the group consisting of: MAP15, MAP28, MAP1, MAP2, MAP3, MAP6, MAP7, MAP8, MAP9, MAP10, MAP11, MAP12, MAP18, MAP19, MAP20, MAP21, MAP22, MAP23, MAP29, MAP30, MAP32, MAP33.

4. The MAP of claim 1 wherein said modifications of said amino acid residues are deletions.

5. A fusion protein comprising an N-terminal segment encoding thioredoxin and a C-terminal segment encoding a modified ADAM-derived polypeptide (MAP) as specified in claim 1.

6. The MAP of claim 1 wherein:
a) said MAP is capable of inhibiting the movement of HUVEC or MDA-MB-435 cells through a reconstituted basement membrane;
b) said MAP is capable of increasing the level of phosphorylation of FAK in MDA-MB-435 cells; or,
c) said MAP is capable of inhibiting tube formation of HUVECs in culture.

7. A nucleic acid encoding a MAP of claim 1.

8. The nucleic acid of claim 7, further comprising a nucleic acid sequence encoding thioredoxin 5′ to the nucleic acid encoding said MAP, wherein said nucleic acid encodes a thioredoxin-MAP fusion protein.

9. An expression vector comprising the nucleic acid of claim 7.

10. Prokaryotic host cells transformed with said expression vector of claim 9, wherein said expression vector is under inducible control, wherein said host also carries stable mutations in thioredoxin reductase B (trxB) gene and/or the glutathione reductase (gor) gene and wherein said trxB and gor mutations are selectable to maintain the expression vector and trxB and gor mutations in said host cells during growth.

11. A method of treating an individual suffering from cancer, said method comprising administering to said individual an effective amount of at least one MAP as specified in claim 1.

12. The method of claim 11 wherein said cancer is an integrin expressing cancer.

13. The method of claim 12 wherein said cancer is selected from the group consisting of breast cancer, colorectal cancer, basal cell carcinoma, adenocarcinoma, gastrointestinal cancer, colon cancer, liver cancer, bladder cancer, pancreatic cancer, ovarian cancer, cervical cancer, lung cancer, skin cancer, prostate cancer, renal cell carcinoma, central nervous system (CNS) cancer, and leukemia.

14. The method of claim 13, wherein said gastrointestinal cancer is selected from the group consisting of lip cancer, mouth cancer, esophageal cancer, small bowel cancer and stomach cancer.

15. The method of claim 13, wherein said skin cancer is selected from the group consisting of squamous cell and basal cell cancer.

16. A method of inhibiting the binding of an integrin to a ligand comprising contacting a cell that expresses the integrin with an effective amount of a MAP according to claim 1.

17. The method of claim 16 wherein said MAP is selected from the group consisting of MAP15, MAP28, MAP1, MAP2, MAP3, MAP6, MAP7, MAP8, MAP9, MAP10, MAP11, MAP12, MAP18, MAP19, MAP20, MAP21, MAP22, MAP23, MAP29, MAP30, MAP32, MAP33.

18. The method of claim 16 wherein said MAP further comprises a fusion of an N-terminal segment of thioredoxin.

19. The method of claim 16 wherein:
a) said MAP is capable of inhibiting the movement of HUVEC or MDA-MB-435 cells through a reconstituted basement membrane;
b) said MAP is capable of increasing the level of phosphorylation of FAK in MDA-MB-435 cells; or,
c) said MAP is capable of inhibiting tube formation of HUVECs in culture.

20. A method of determining the presence of cancer cells in an individual, said method comprising contacting said cancer cells with at least one MAP according to claim 1 and detecting said at least one MAP.

21. The method of claim 20 wherein said cancer is an integrin expressing cancer.

22. The method of claim 20 wherein said cancer is selected from the group consisting of breast cancer, colorectal cancer, basal cell carcinoma, adenocarcinoma, gastrointestinal cancer, colon cancer, liver cancer, bladder cancer, pancreatic cancer, ovarian cancer, cervical cancer, lung cancer, skin cancer, prostate cancer, renal cell carcinoma, central nervous system (CNS) cancer, and leukemia.

23. The method of claim 22, wherein said gastrointestinal cancer is selected from the group consisting of lip cancer, mouth cancer, esophageal cancer, small bowel cancer and stomach cancer.

24. The method of claim 22, wherein said skin cancer is selected from the group consisting of squamous cell and basal cell cancer.

25. The method of claim 20, wherein said MAP is labeled.

26. The method of claim 25, wherein said label is a Positron Emmission Tomography probe or a fluorescent probe.

27. A method of preparing an artificial ECM scaffold comprising coating said artificial ECM scaffold with a MAP according to claim 1.

28. The method of claim 27, further comprising the introduction of stem cell precursors to the artificial ECM scaffold.

29. The method of claim 27, where said artificial ECM scaffold comprises a urinary bladder scaffold, an esophageal scaffold or an anal scaffold.

30. A method of expressing a MAPs of claim 1 in prokaryotic host cells, said method comprising:
a) growing the prokaryotic host cells of claim 10, wherein said expression vector has an antibiotic resistance gene which makes it selectable on a first antibiotic, and wherein said trxB and gor mutations are selectable on at least one additional antibiotic to maintain the expression vector and trxB and gor mutations in said host cells during growth, in the presence of the first and said at least one additional antibiotic to obtain a sufficient number of cells suitable to seed a reactor in which host cells will be grown and the fusion protein expression induced; and
b) seeding the reactor with the cells of step a) and growing the cells and inducing expression of the fusion protein, wherein said cells in the reactor are grown in the presence of the first antibiotic and in the absence of said at least one additional antibiotic.

31. The method of claim 30 wherein the host cells express mutant products of both the trxB and gor genes.

32. The method of claim 31 wherein said host cells are mutant in both trxB and gor genes.

33. The method of claim 31 wherein the trxB and gor genes are selectable on different antibiotics.

34. The method of claim 30 wherein the thioredoxin portion of the fusion protein has the sequence:
MSDKIIHLTDDSFDTDVLKADGAILVDFWAEWCGPCKMIAPILDEIADEY
QGKLTVAKLNIDQNPGTAPKYG IRGIPTLLLFKNGEVAATKVGALSKGQ
LKEF LDANLA.

35. The method of claim 30 wherein the host is deficient in any one or more of ompT or lon gene products.

36. The method of claim 30 wherein a sequence encoding a cleavage site is located between the sequence encoding thioredoxin and the sequence encoding the disulfide rich protein.

37. The method of claim 30 wherein the fusion protein further includes a peptide sequence which is a ligand for a receptor.

38. The method of claim 30 wherein said prokaryotic host cell is an Origami strain.

39. A stent coated with the composition of claim 1.

40. A Modified ADAM-derived Polypeptide (MAP), comprising an ADAM-derived Polypeptide (AP) comprising a distintegrin-like domain from an ADAM comprising (i) the Cys-Asp-Cys (CDC) motif, (ii) the tripeptide motif as indicated in FIG. 1 and (iii) lacking all or substantially all of the ADAM metalloprotease domain, cysteine-rich domains, and interdomain segments, and further comprising one or more amino acid modifications that result in disruption of the interdomain disulfide linkages with the first cysteine C-terminal to the CDC motif and interdomain disulfide linkages with the cysteine C-terminal to the tripeptide motif, and wherein ADAM means “A Disintegrin and Metalloproteinase.”

41. A polypeptide according to any of the polypeptide sequences in FIG. 2

42. A nucleic acid encoding a polypeptide according to claim 41.
